SEQUENCE LISTING (11 sequences)

SEQ ID NO: 1 - protein, 464 aa, Scenedesmus obliquus
MHSRTNCLQTSVRAPQPHFRPFTAVKTCRQRCSTTAAAAKRDQAQEQQPWIQVGLGLAAAATAVAVGLGAAALPAQAVTSEQLLFLEAWRAVDRAYVDKSFNGQSWFKLRETYLKKEPMDRRAQTYDAIRKLLAVLDDPFTRFLEPSRLAALRRGTAGSVTGVGLEITYDGGSGKDVVVLTPAPGGPAEKAGARAGDVIVTVDGTAVKGLSLYDVSDLLQGEADSQVEVVLHAPGAPSNTRTLQLTRQKVTINPVTFTTCSNVAAAALPPGAAKQQLGYVRLATFNSNTTAAAQQAFTELSKQGVAGLVLDIRNNGGGLFPAGVNVARMLVDRGDLVLIADSQGIRDIYSADGNSIDSATPLVVLVNRGTASASEVLAGALKDSKRGLIAGERTFGKGLIQTVVDLSDGSGVAVTVARYQTPAGVDINKIGVSPDVQLDPEVLPTDLEGVCRVLGSDAAPRLFG

SEQ ID NO: 2 - DNA, 32 bp, Artificial Sequence
  Description of Artificial Sequence  primer
atgaccatggtgacaagcgagcagctgctgtt

SEQ ID NO: 3 - DNA, 37 bp, Artificial Sequence
  Description of Artificial Sequence  primer
agctgatgcggatccttacccaaacagccgcggcgca

SEQ ID NO: 4 - protein, 389 aa, Scenedesmus obliquus
AMVTSEQLLFLEAWRAVDRAYVDKSFNGQSWFKLRETYLKKEPMDRRAQTYDAIRKMLAVLDDPFTRFLEPSRLAALRRGTAGSVTGVGLEITYDGGSGKDVVVLTPAPGGPAEKAGARAGDVIVTVDGTAVKGMSLYDVSDLLQGEADSQVEVVLHAPGAPSNTRTLQLTRQKVTINPVTFTTCSNVAAAALPPGAAKQQLGYVRLATFNSNTTAAAQQAFTELSKQGVAGLVLDIRNNGGGLFPAGVNVARMLVDRGDLVLIADSQGIRDIYSADGNSIDSATPLVVLVNRGTASASEVLAGALKDSKRGLIAGERTFGKGLIQTVVDLSDGSGVAVTVARYQTPAGVDINKIGVSPDVQLDPEVLPTDLEGVCRVLGSDAAPRLFG

SEQ ID NO: 5 - DNA, 33 bp, Artificial Sequence
  Description of Artificial Sequence  primer
gatgccatccgcaagatgctggcggtgctggac

SEQ ID NO: 6 - DNA, 33 bp, Artificial Sequence
  Description of Artificial Sequence  primer
gtccagcaccgccagcatcttgcggatggcatc

SEQ ID NO: 7 - DNA, 33 bp, Artificial Sequence
  Description of Artificial Sequence  primer
acggctgtgaaggggatgtcgctgtatgacgtg

SEQ ID NO: 8 - DNA, 33 bp, Artificial Sequence
  Description of Artificial Sequence  primer
cacgtcatacagcgacatccccttcacagccgt

SEQ ID NO: 9 - protein, 388 aa, Triticum sp.
LTEENLLFLEAWRAVDRAYYDKSFNGQSWFRYRERALRDDPMNTRQETYAAIKKMLATLDDPFTRLLEPEKFKSLRSGTQGALTGVGLSIGYPLALKGSPAGLSVMSAAPGGPAEKAGIVSGDVILAIDDTSAQDMDIYDAADRLQGPEGSSIDLTILSGADTRHVVLKRERYTLNPVRSRMCEIPGSEDSSKIGYIKLTTFNQNAAGSVKEAIKKLREKNVKAFVLDLRNNSGGLFPEGIEIAKIWMDKGVIVYICDSRGVRDIYEADGASTIAASEPLVVLVNKGTASASEILAGALKDNKRAVVYGEPTYGKGKIQSVFALSDGSGLAVTVARYETPAHTDIDKVGVTPDRPLPASFPTDEDGFCSCLRDPASCNLNAARLFVRS

SEQ ID NO: 10 - protein, 389 aa, Scenedesmus obliquus
AMVTSEQLLFLEAWRAVDRAYVDKSFNGQSWFKLRETYLKKEPMDRRAQTYDAIRKLLAVLDDPFTRFLEPSRLAALRRGTAGSVTGVGLEITYDGGSGKDVVVLTPAPGGPAEKAGARAGDVIVTVDGTAVKGLSLYDVSDLLQGEADSQVEVVLHAPGAPSNTRTLQLTRQKVTINPVTFTTCSNVAAAALPPGAAKQQLGYVRLATFNSNTTAAAQQAFTELSKQGVAGLVLDIRNNGGGLFPAGVNVARMLVDRGDLVLIADSQGIRDIYSADGNSIDSATPLVVLVNRGTASASEVLAGALKDSKRGLIAGERTFGKGLIQTVVDLSDGSGVAVTVARYQTPAGVDINKIGVSPDVQLDPEVLPTDLEGVCRVLGSDAAPRLFG

SEQ ID NO: 11 - protein, 4 aa, Artificial Sequence
  Description of Artificial Sequence tetrapeptide
LDLA

(19) United States
(12) Patent Application Publication
(10) Pub. No.: US 2003/0175800 A1
(43) Pub. Date: Sep. 18, 2003

(54) D1-C-terminal processing protease: methods for three dimensional structural determination and rational inhibitor design

(76) Inventors: Bruce A. Diner, Chadds Ford, PA (US); Doug B. Jordan, Wilmington, DE (US); Der-Ing Liao, Newark, DE (US); Mark J. Nelson, Newark, DE (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 09/999,536

(22) Filed: Nov. 15, 2001

Related U.S. Application Data
(62) Division of application No. 09/564,335, filed on May 2, 2000, now abandoned.
(60) Provisional application No. 60/133,047, filed on May 7, 1999.

(51) Int. Cl. G01N 33/53; G06F 19/00; G01N 33/48; G01N 33/50

(52) U.S. Cl. 435/7.1; 702/19

(57) ABSTRACT

The present invention provides atomic coordinate/x-ray diffraction data defining the three dimensional structure of D1 protease. The present invention further provides methods for identifying ligands that bind to D1 protease.

This application claims the benefit of U.S. Provisional Application No. 60/133,047, filed May 7, 1999.

### FIELD OF THE INVENTION

The present invention is in the field of three-dimensional protein structure determination, the modeling of new structures, and inhibitor identification and design using three-dimensional protein structures.

### BACKGROUND OF THE INVENTION

D1-C-terminal processing (D1) protease is responsible for C-terminal processing of the carboxy-terminal extension of the precursor form of the D1 polypeptide of the Photosystem II reaction center (Marder et al., J. Biol. Chem. 259:3900-3908 (1984); Metz et al., FEBS Lett. 205:269-274 (1986); Diner et al., J. Biol. Chem. 263:8972-8980 (1988); Taylor et al., FEBS Lett. 235:109-116 (1988); Takahashi et al., FEBS Lett. 240:6-8 (1988); a Anbudurai et al., Proc. Natl. Acad. Sci. USA 91:8082-8086 (1994); Trost et al., J. Biol. Chem. 272:20348-20356 (1997)). This processing is essential for the assembly of the manganese cluster, responsible for photosynthetic water oxidation and the source of electrons to the photosynthetic electron transport chain (Metz et al., Biochem. Biophys. Res. Commun. 94:560-566 (1980); Bowyer et al., J. Biol. Chem. 267:5424-5433 (1992); Nixon et al., Biochemistry 31:10859-10871 (1992)). Because of the essential nature of the D1 protease for photosynthesis, it is a potential target for inhibitors with utility as commercial herbicides. Until now, the three-dimensional structure of this enzyme as well as of any homologous proteins has not been determined. There are also no publicly known inhibitors of this enzyme. The instant invention reports the three-dimensional structure of D1 protease from Scenedesmus obliquus at 1.8 Å resolution.

### SUMMARY OF THE INVENTION

The present invention provides a computer readable medium having stored thereon atomic coordinate/X-ray diffraction data defining the three dimensional structure of Scenedesmus obliquus D1 protease or a fragment thereof. Additionally the invention provides a computer readable medium having stored thereon atomic coordinate data defining the three dimensional structure of wheat D1 protease or a fragment thereof.

The invention further provides a computer readable medium having stored thereon the computer model output data defining the three dimensional structure of Scenedesmus obliquus D1 protease or a fragment thereof. Similarly, it is an object of the invention to provide a computer readable medium having stored thereon the computer model output data defining the three dimensional structure of a wheat. D1 protease or a fragment thereof.

Additionally the present invention provides a method for identifying a ligand of D1 protease or a fragment thereof, the method comprising: (a) providing a computer readable medium having stored thereon computer model output data defining the three dimensional structure of a D1 protease; (b) providing a computer readable medium having stored thereon computer model output data defining the three dimensional structure of a potential ligand that binds to D1 protease or a fragment thereof, (c) providing a computer system comprising a computer and a computer algorithm, the computer system capable of processing the computer model output data of step (a) and step (b); (d) processing the computer model output data of step (a) and step (b) using the computer system of step (c) wherein the processing calculates the ability of the potential ligand to bind to D1 protease or a fragment thereof; and (e) identifying a potential ligand of D1 protease or a fragment thereof.

It is a further object of the present invention to provide a crystal of a D1 protease wherein the crystal effectively diffracts X-rays for the determination of the atomic coordinates of a D1 protease or a fragment thereof to a resolution equal or better than 3.5 Angstroms.

The present invention further provides a method of identifying a D1 protease ligand comprising: (a) selecting a potential ligand by performing rational compound design with the three-dimensional structure determined for the crystal of the Scendesmus obliquus D1 protease enzyme, wherein said selecting is performed in conjunction with computer modeling; (b) contacting the potential ligand with the ligand binding domain of D1 protease; and (c) detecting the binding of the potential ligand for the ligand binding domain; wherein a potential ligand is selected on the basis of its having a greater affinity for the ligand binding domain of D1 protease than that of the natural substrate for the ligand binding domain of D1 protease.

The invention additionally provides methods of obtaining coordinate data defining the three dimensional structure of a D1 protease enzyme comprising performing molecular modeling using; (i) the coordinate/X-ray diffraction data defining the three dimensional structure of Scenedesmus obliquus D1 protease or a fragment thereof; and (ii) the amino acid sequence of a D1 protease enzyme; and optionally the X-ray diffraction data from a crystallized D1 protease enzyme, wherein said molecular modeling produces predicted coordinate data defining the three dimensional structure of the D1 protease enzyme. This method may optionally be accomplished using homology modeling or molecular replacement and the D1 protease may be isolated from plants selected from the group consisting of wheat, corn, soybean, barley, and rice.

### BRIEF DESCRIPTION OF THE FIGURES AND SEQUENCE LISTING

The invention can be more fully understood from the following detailed description and the accompanying figures and Sequence Listing which form a part of this application.

FIG. 1 presents the atomic coordinates derived from X-ray diffraction data defining the three-dimensional structure of D1 protease isolated from Scenedesmus obliquus.

FIG. 2 illustrates site-directed mutagenesis of D1 protease.

FIG. 3 presents an amino acid comparison of wheat and Scenedesmus obliquus D1 protease.

FIG. 4 presents the predicted atomic coordinates of the resulting three-dimensional model of D1 protease isolated from wheat.

FIG. 5 presents the atomic coordinates derived from X-ray diffraction data defining the three-dimensional structure of the C2I form of the native D1 protease isolated from Scenedesmus obliquus.

FIG. 6 presents the atomic coordinates derived from X-ray diffraction data defining the three-dimensional structure of the R32 form of the native D1 protease isolated from Scenedesmus obliquus.

FIG. 7 presents the atomic coordinates derived from X-ray diffraction data defining the three-dimensional structure of the D1 protease derivatized by peptide chloromethylketone inhibitor.

FIG. 8 presents the computer model of the active site lysine covalently modified by the peptide chloromethylketone inhibitor.

The following sequence descriptions and sequence listings attached hereto comply with the rules governing nucleotide and/or amino acid sequence disclosures in patent applications as set forth in 37 C.F.R. §1.821-1.825. The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219(2):345-373 (1984) which are herein incorporated by reference. The symbols and format used for nucleotide and amino acid sequence data comply with the rules set forth in 37 C.F.R. §1.822.

SEQ ID NO: 1 is the amino acid sequence of D1 protease from Scenedesmus obliquus.

SEQ ID NO: 2 is the 5′ primer sequence used for cloning Scenedesmus obliquus D1 protease gene.

SEQ ID NO: 3 is the 3′ primer sequence used for cloning Scenedesmus obliquus D1 protease gene.

SEQ ID NO: 4 is the amino acid sequence of D1 protease from Scenedesmus obliquus which has undergone site-directed mutagenesis and which lacks the signal peptide.

SEQ ID NO: 5 is the L132-fwd primer.

SEQ ID NO: 6 is the L132-rev primer.

SEQ ID NO: 7 is the L210-fwd primer.

SEQ ID NO: 8 is the L210-rev primer.

SEQ ID NO: 9 is the amino acid sequence of D1 protease from wheat.

SEQ ID NO: 10 is the amino acid sequence of the wildtype D1 protease from Scenedesmus obliquus lacking the signal peptide.

SEQ ID NO: 11 is the tetrapeptide chloromethylketone D1 protease ligand.

### DETAILED DESCRIPTION OF THE INVENTION

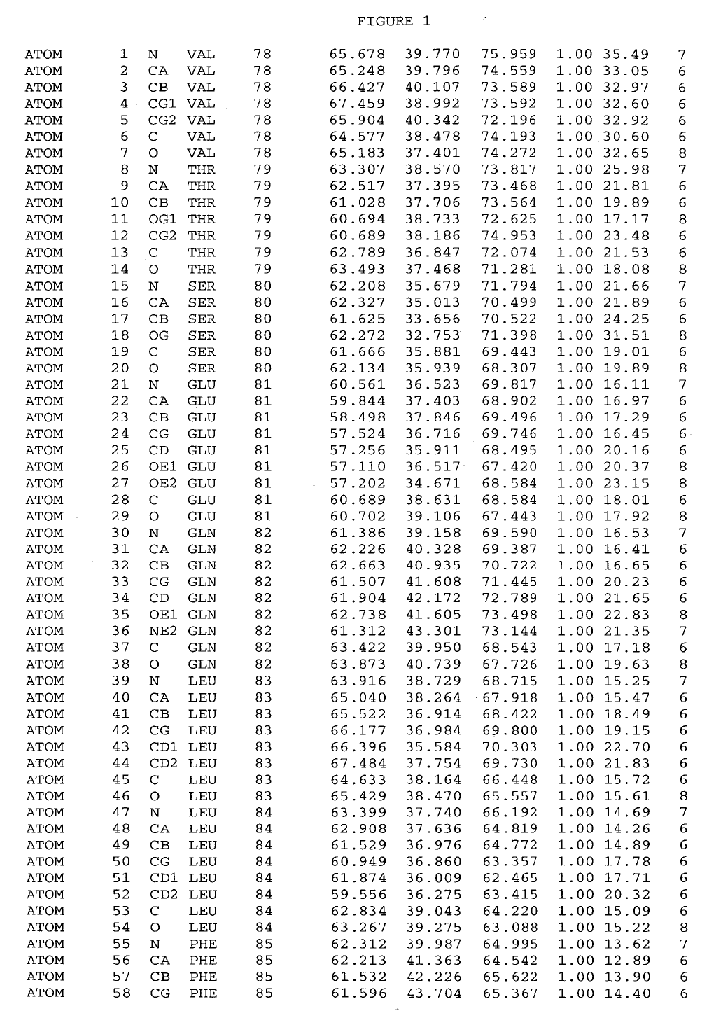

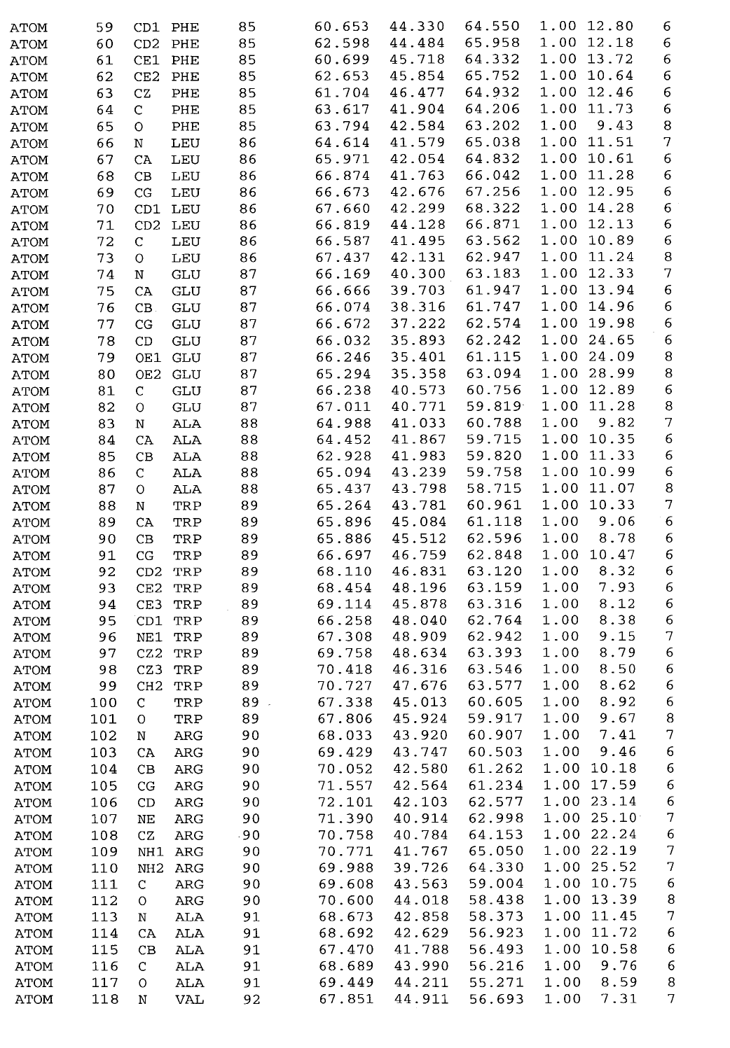

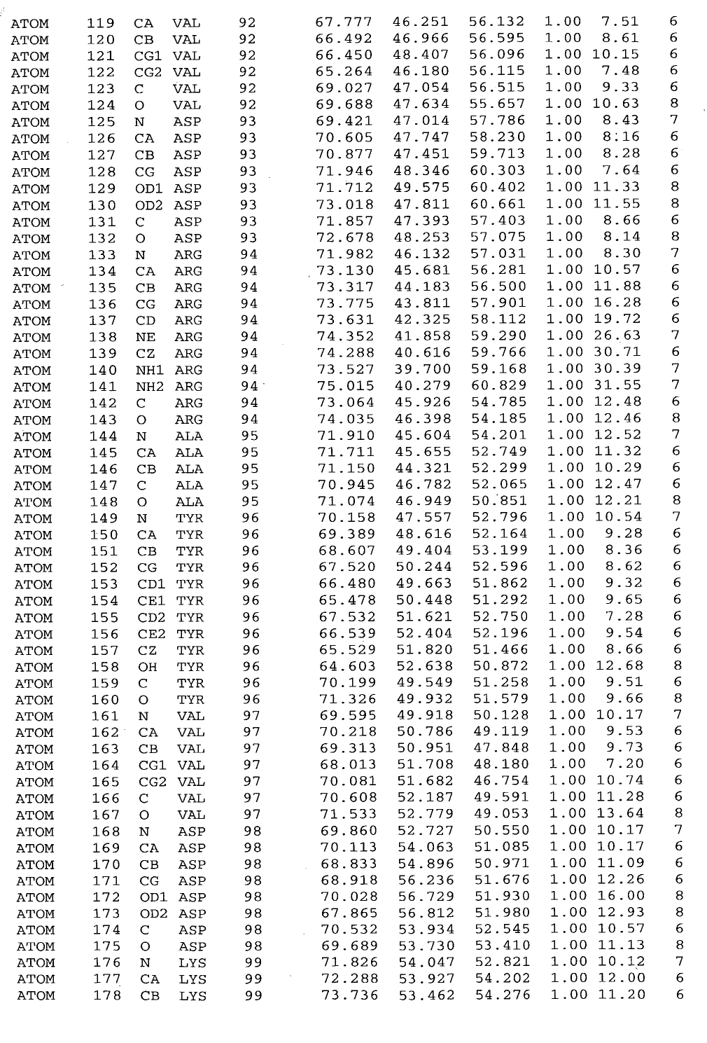

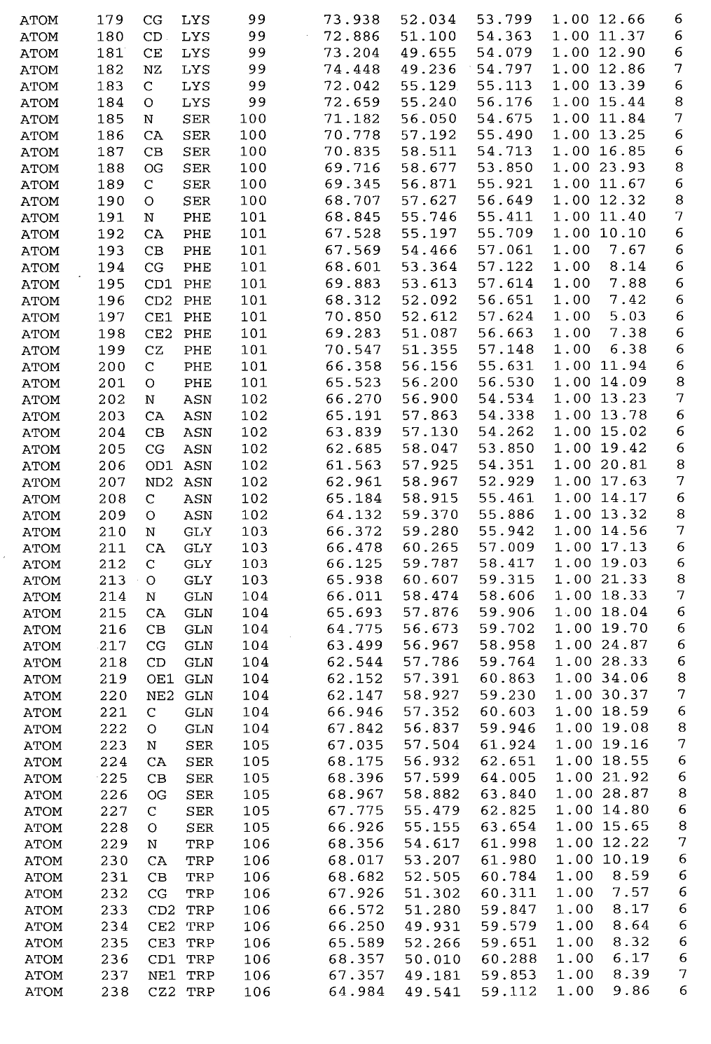

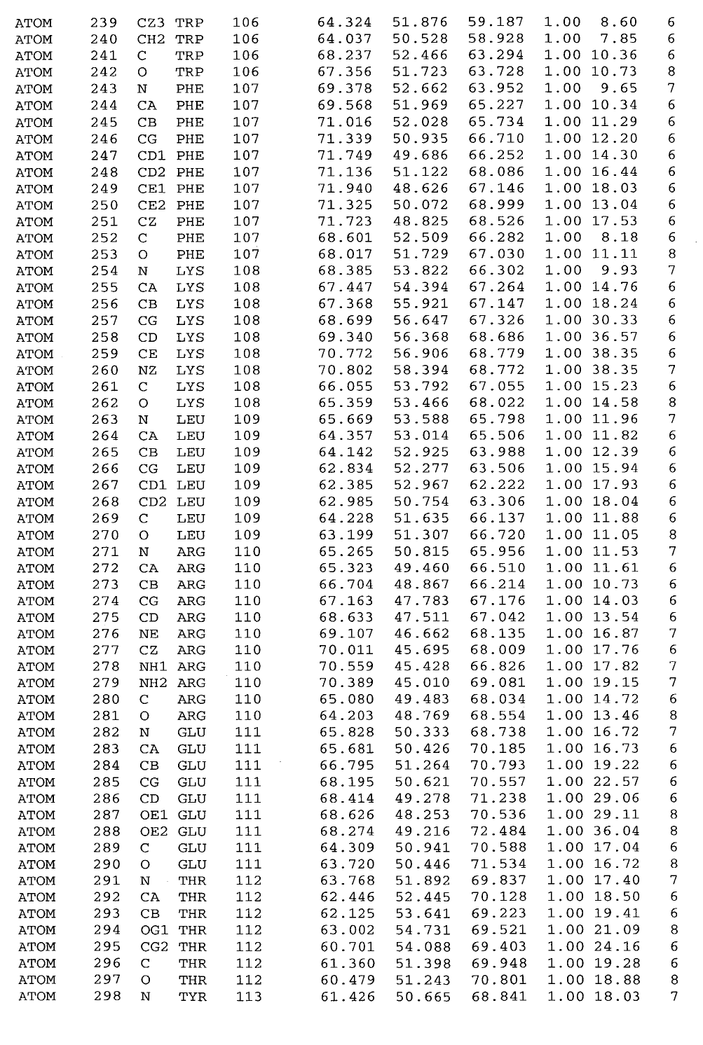

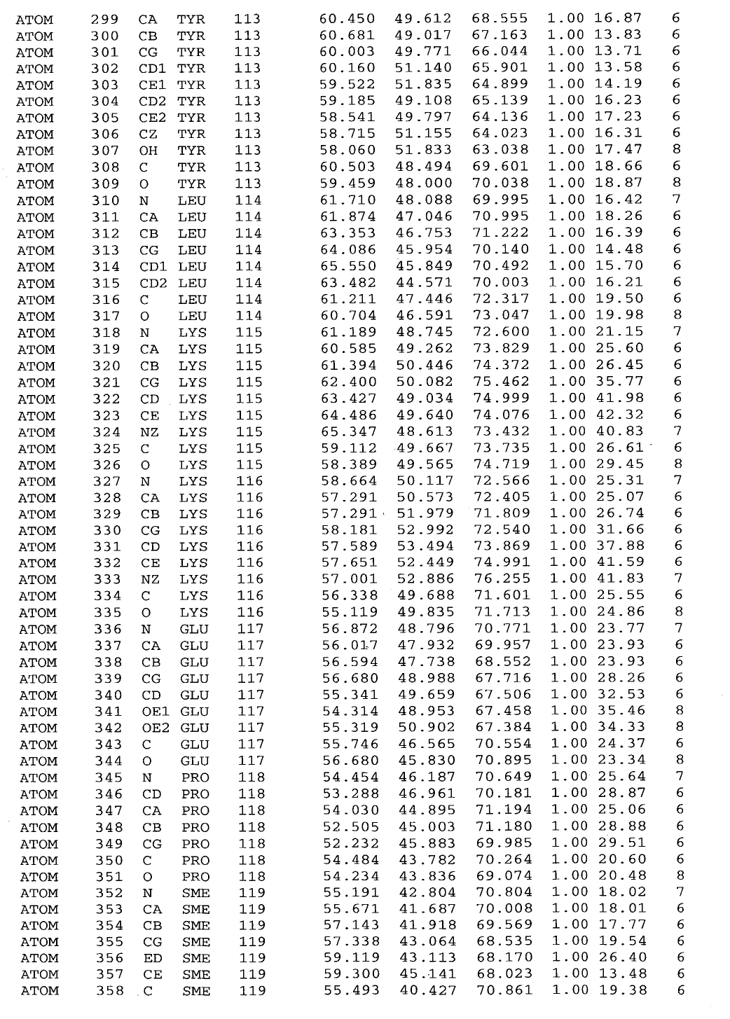

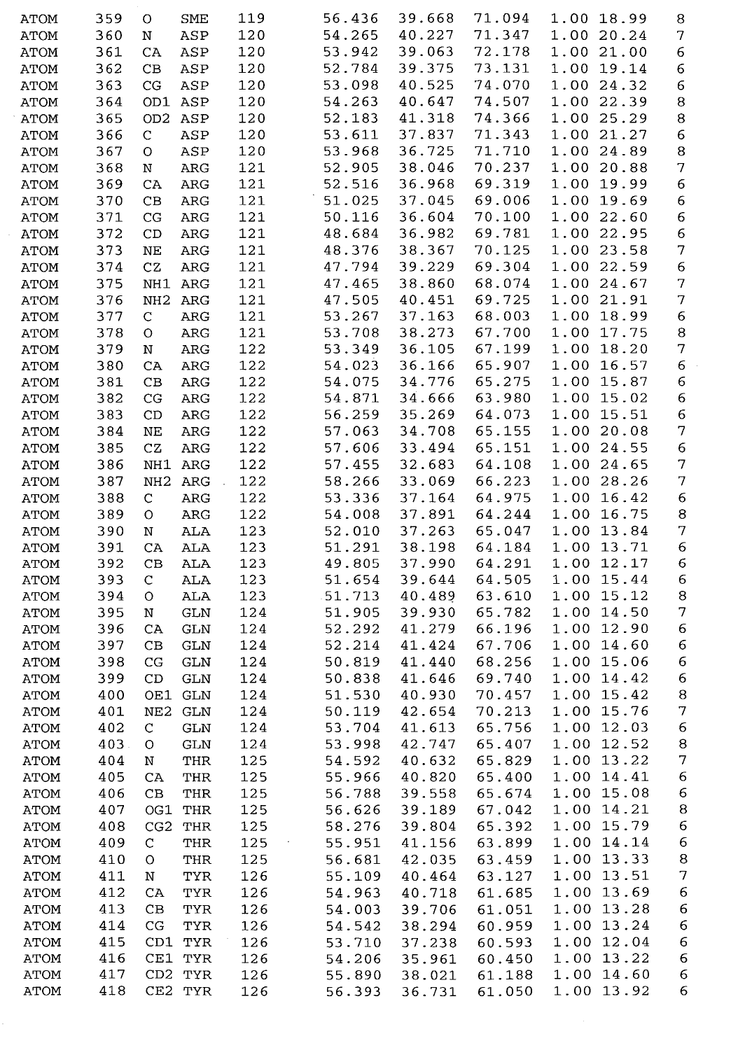

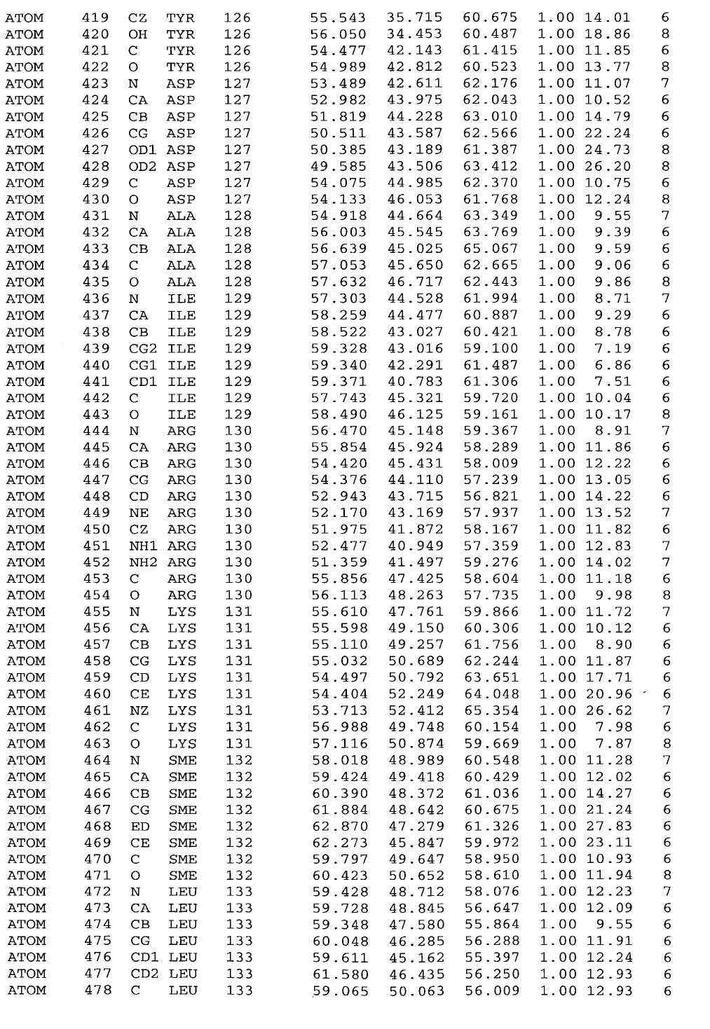

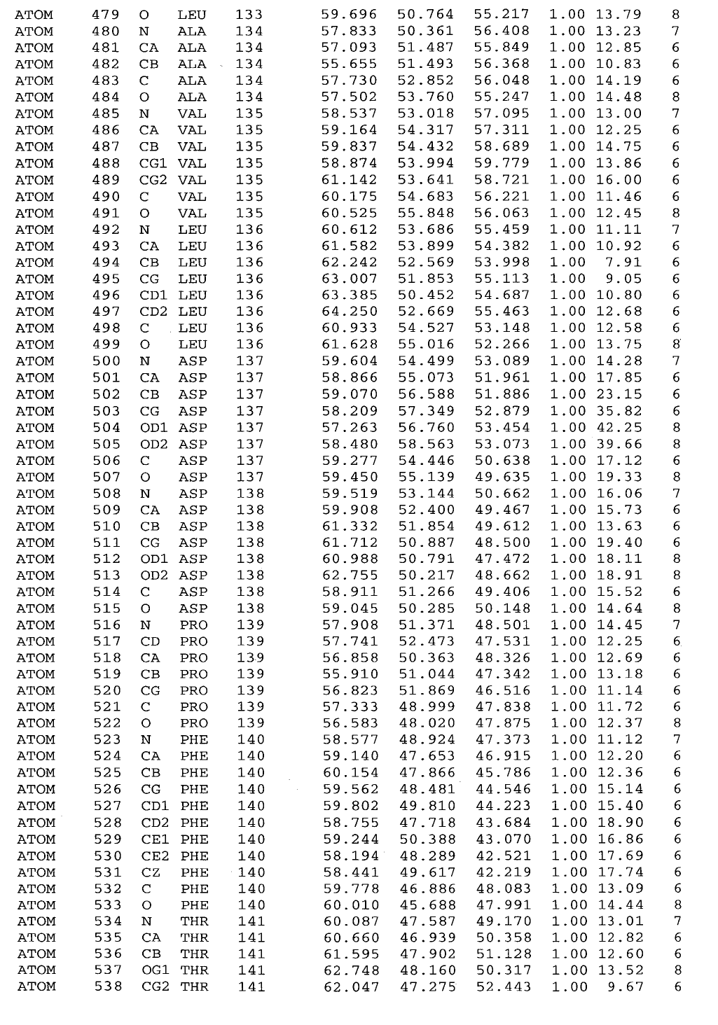

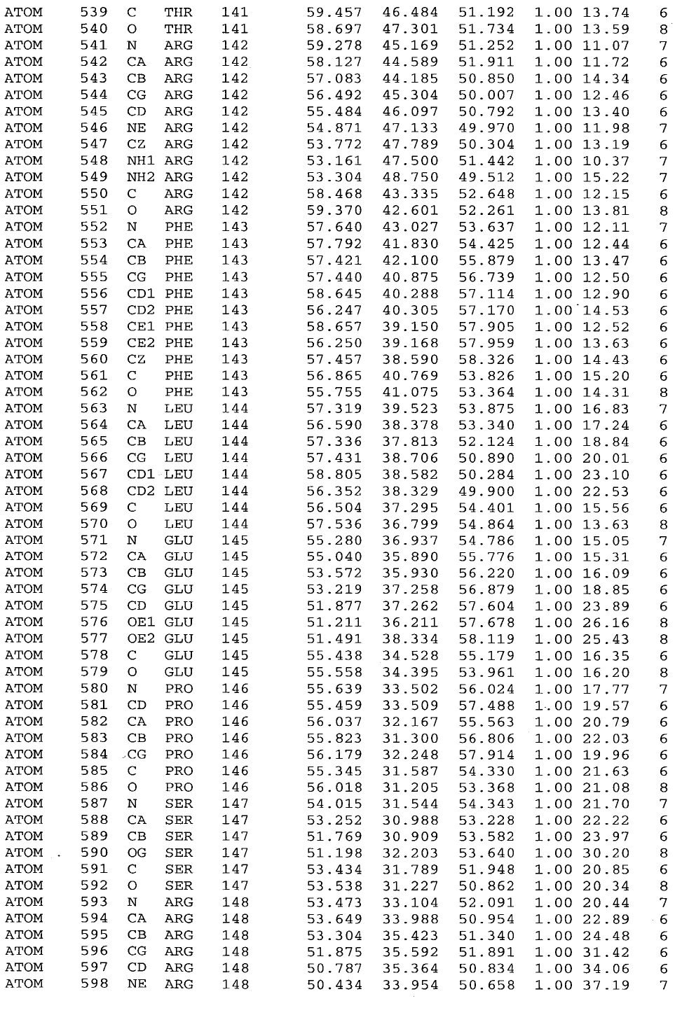

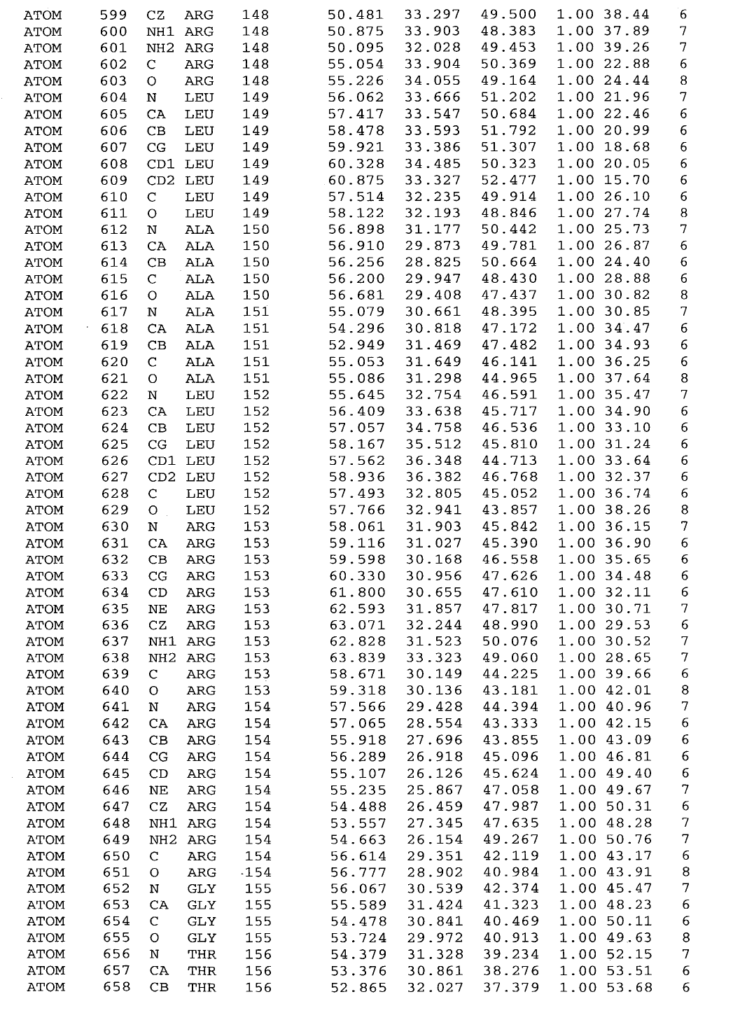

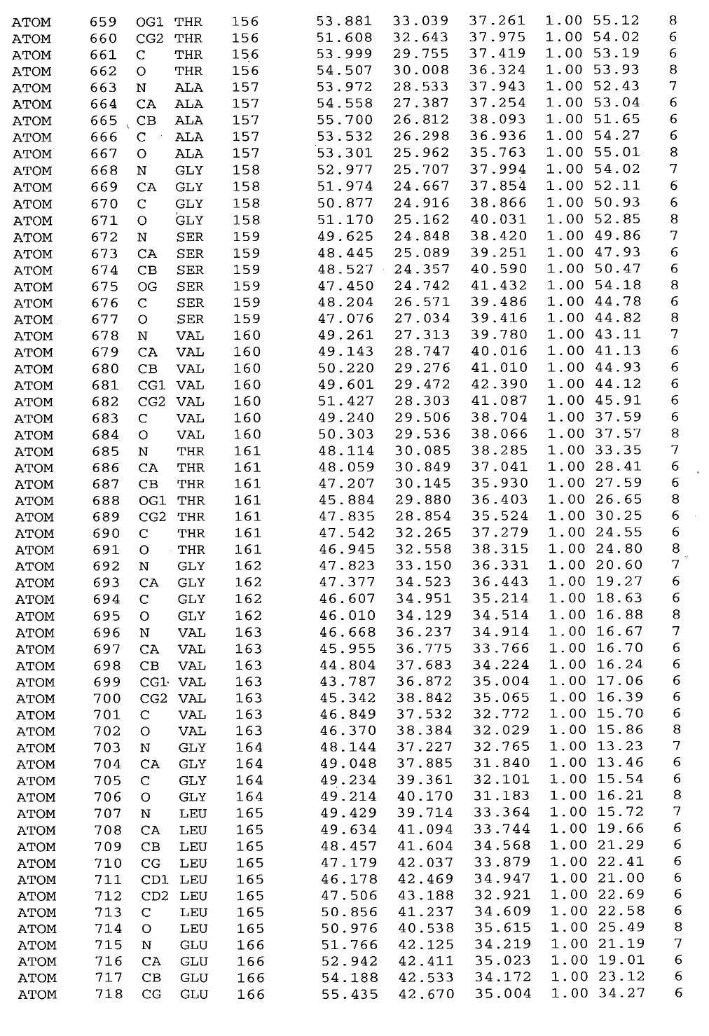

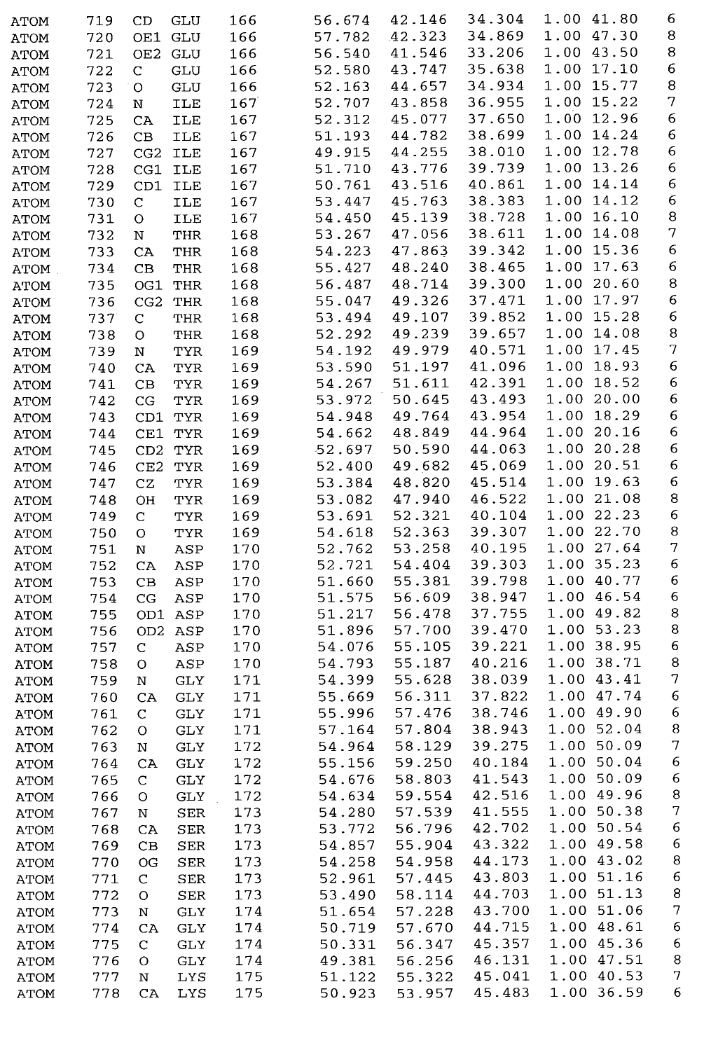

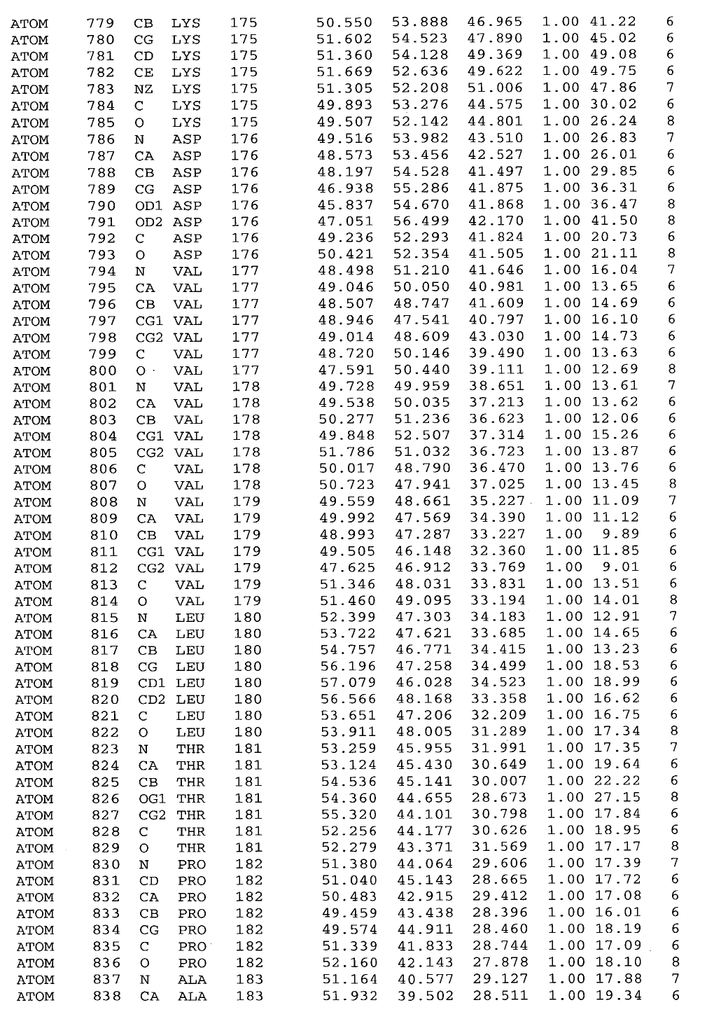

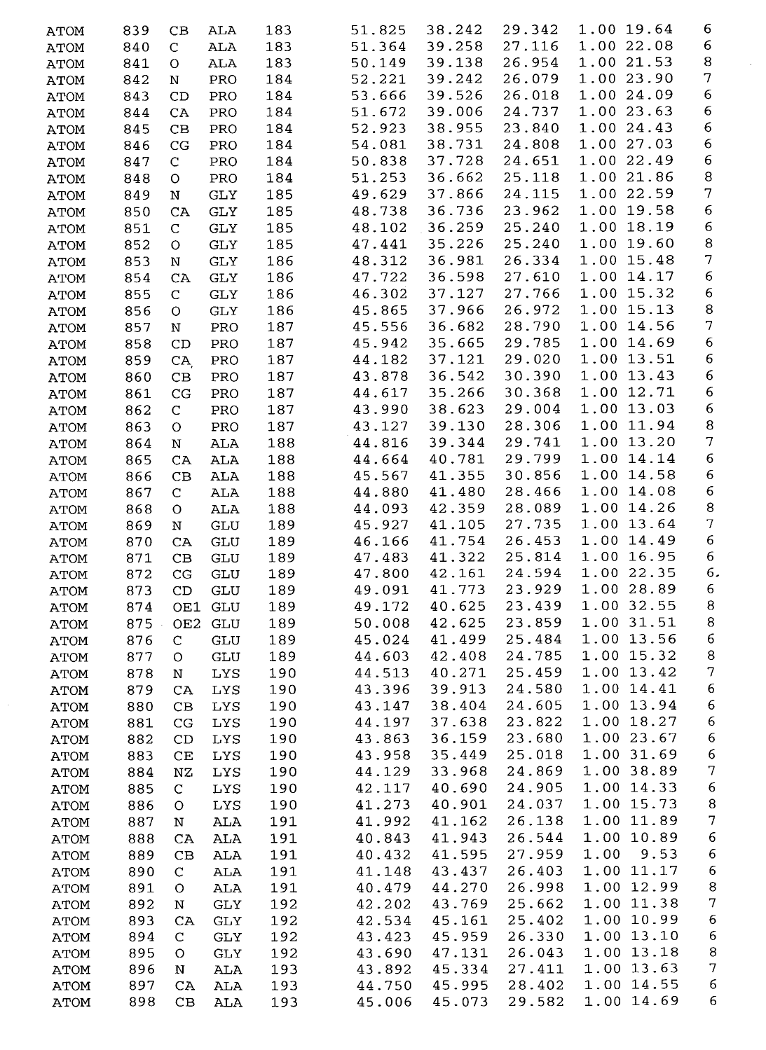

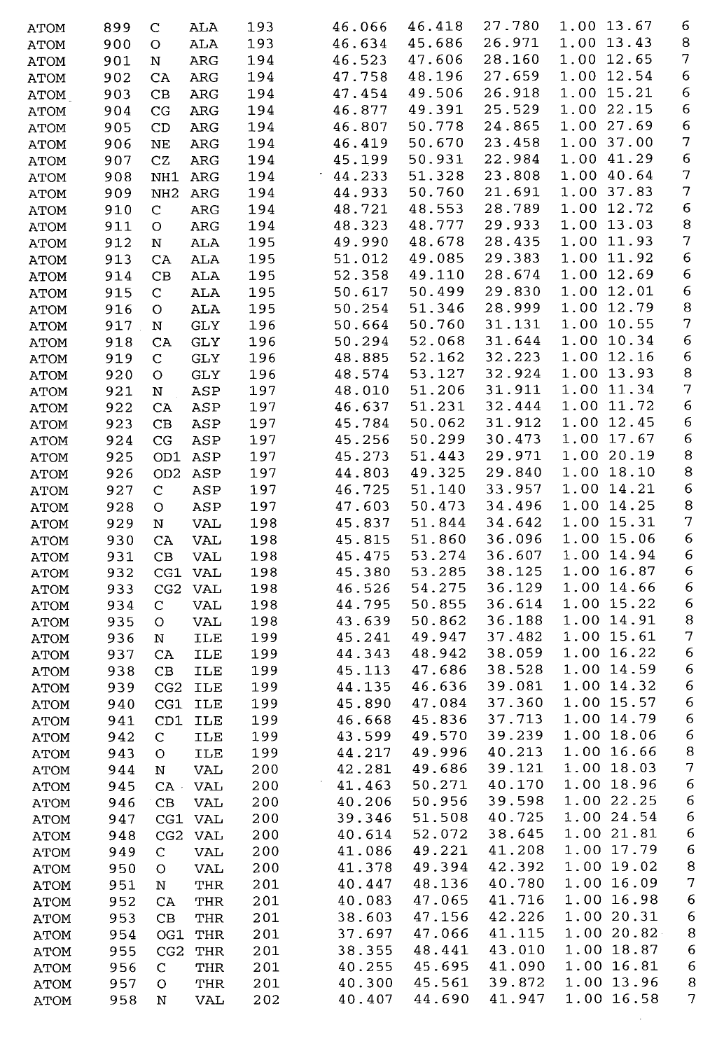

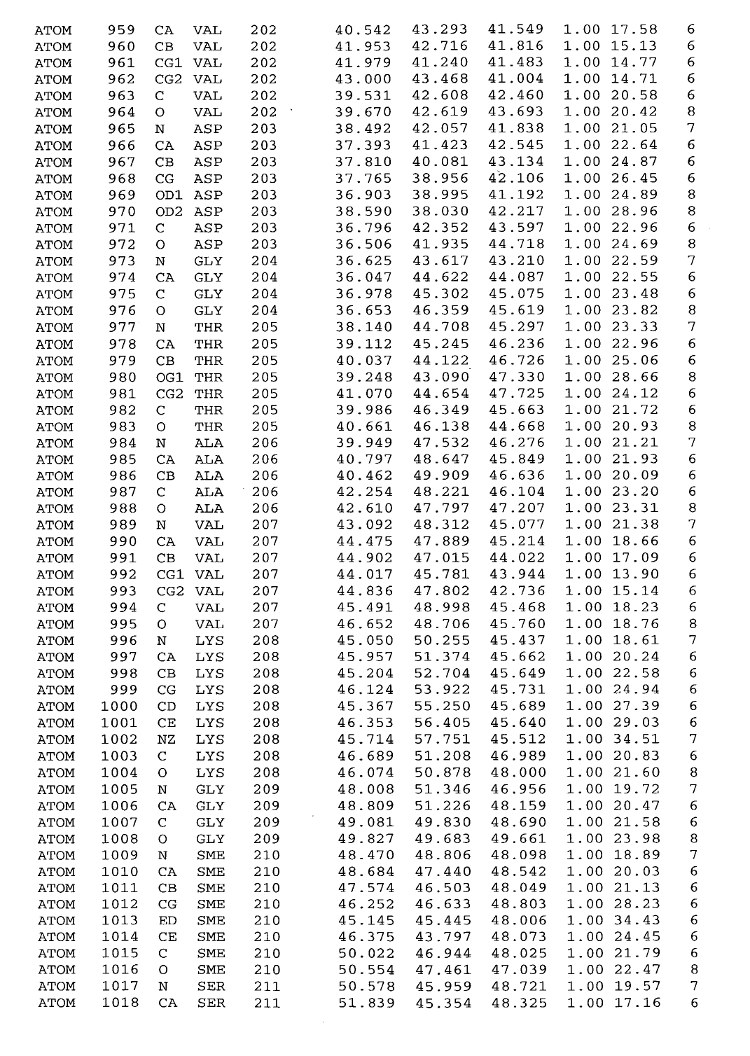

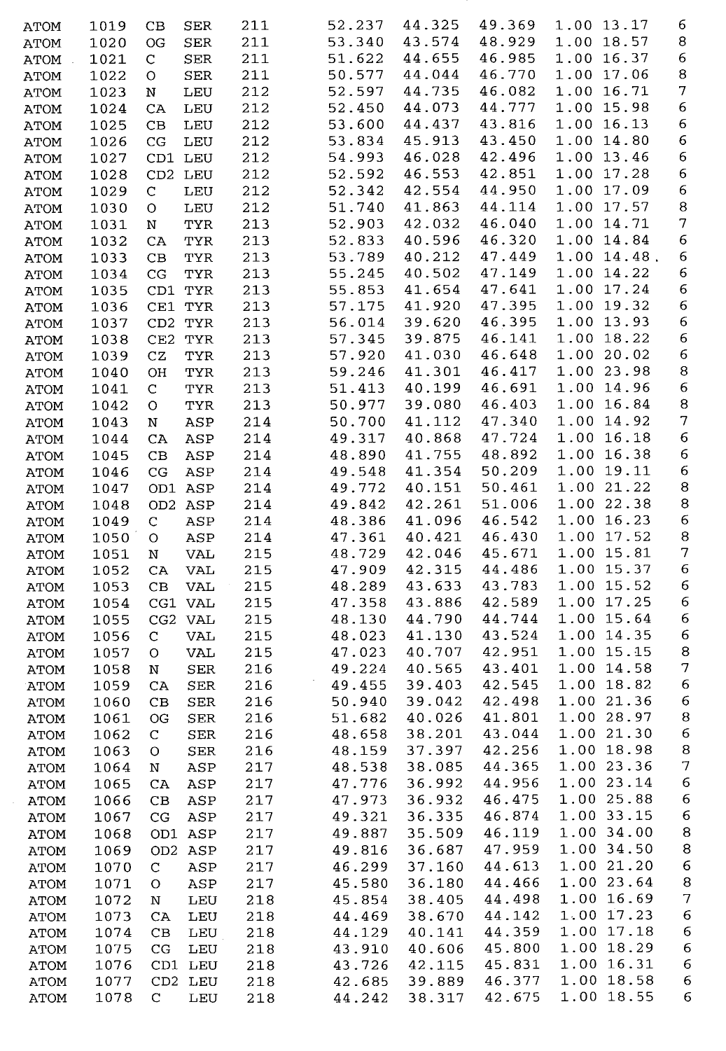

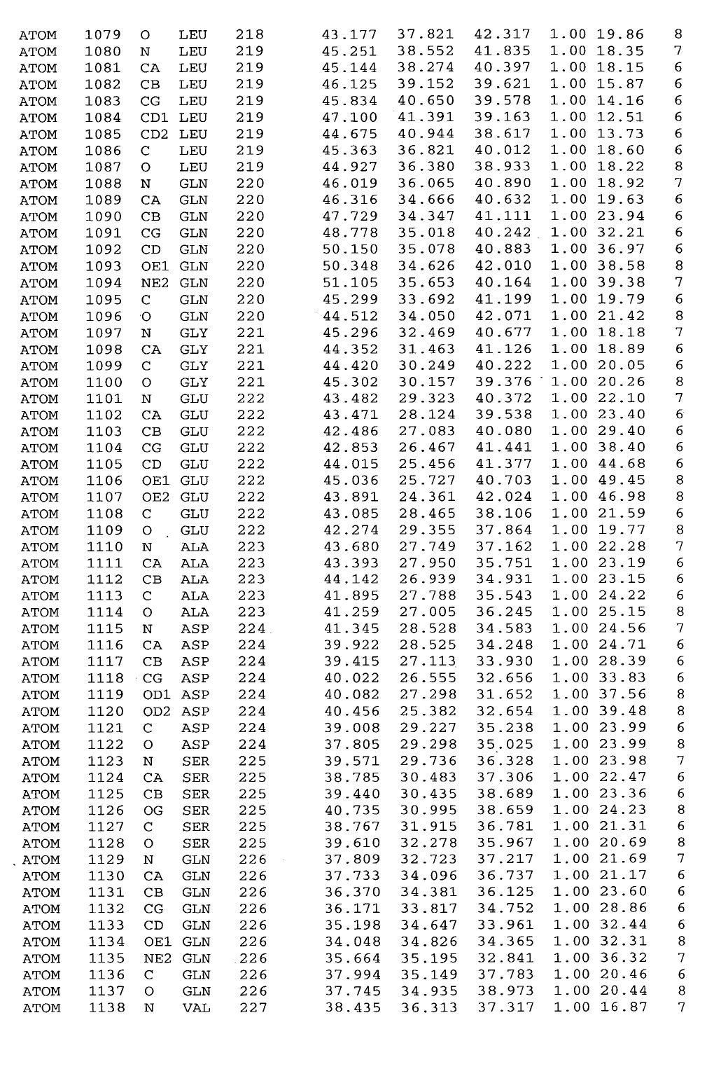

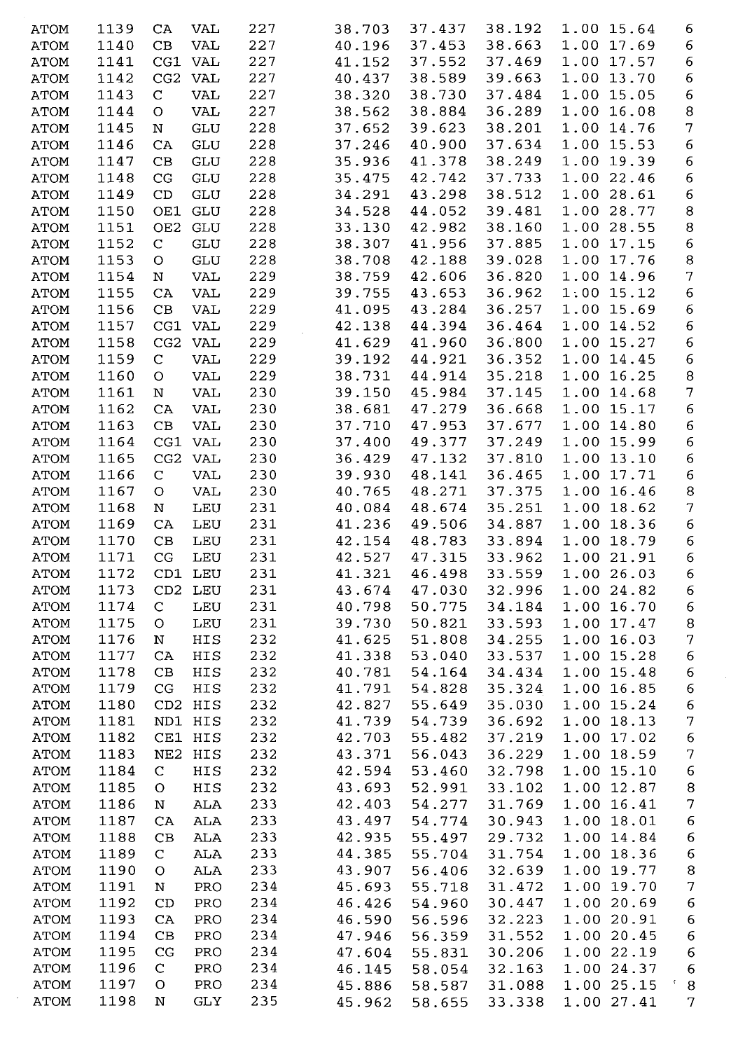

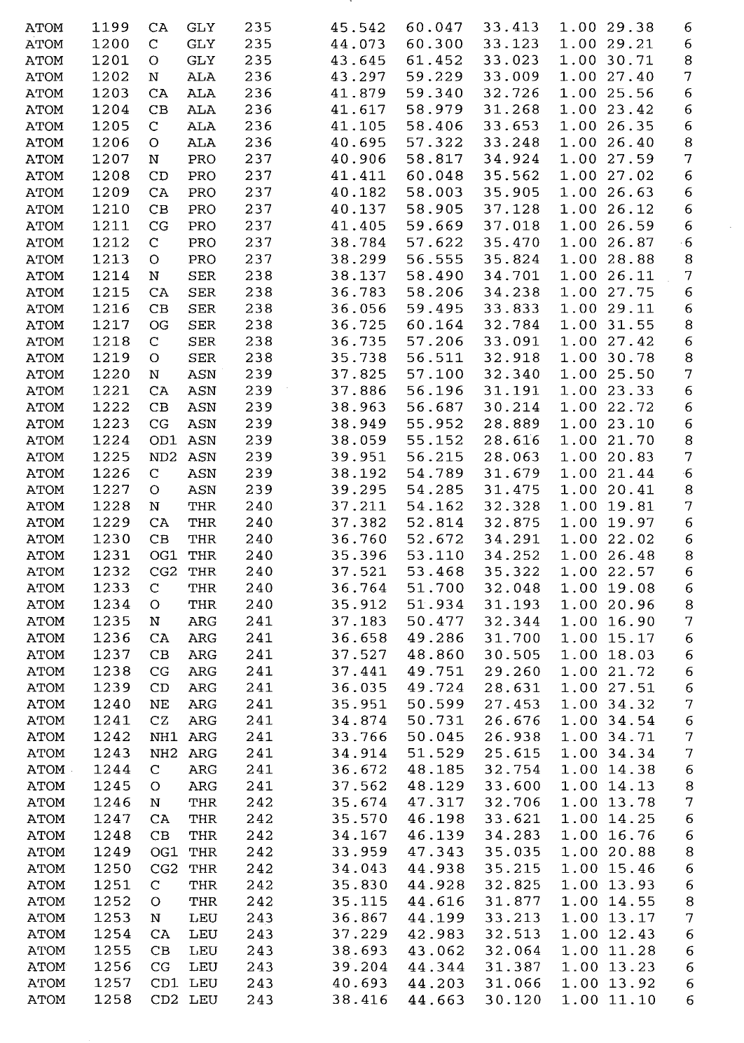

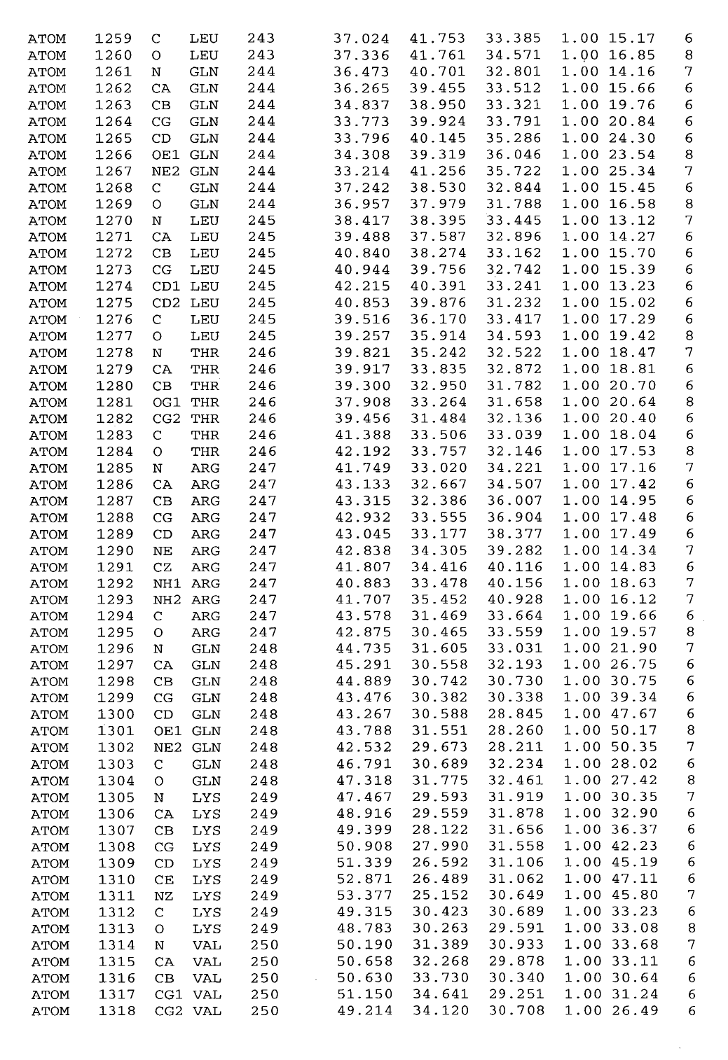

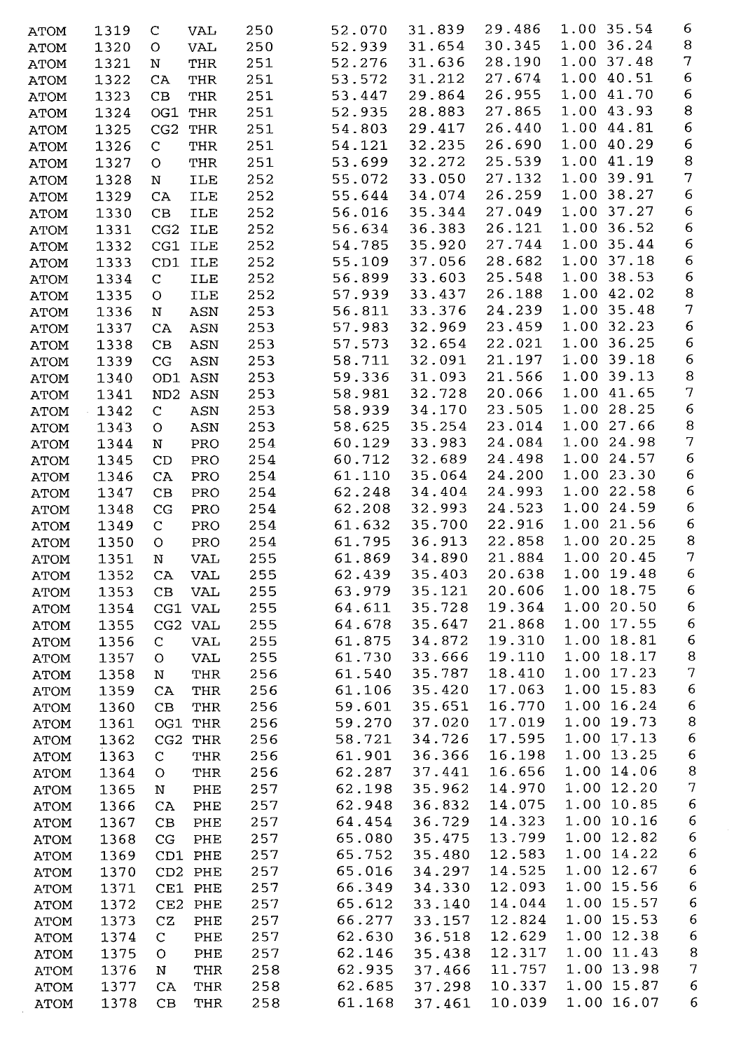

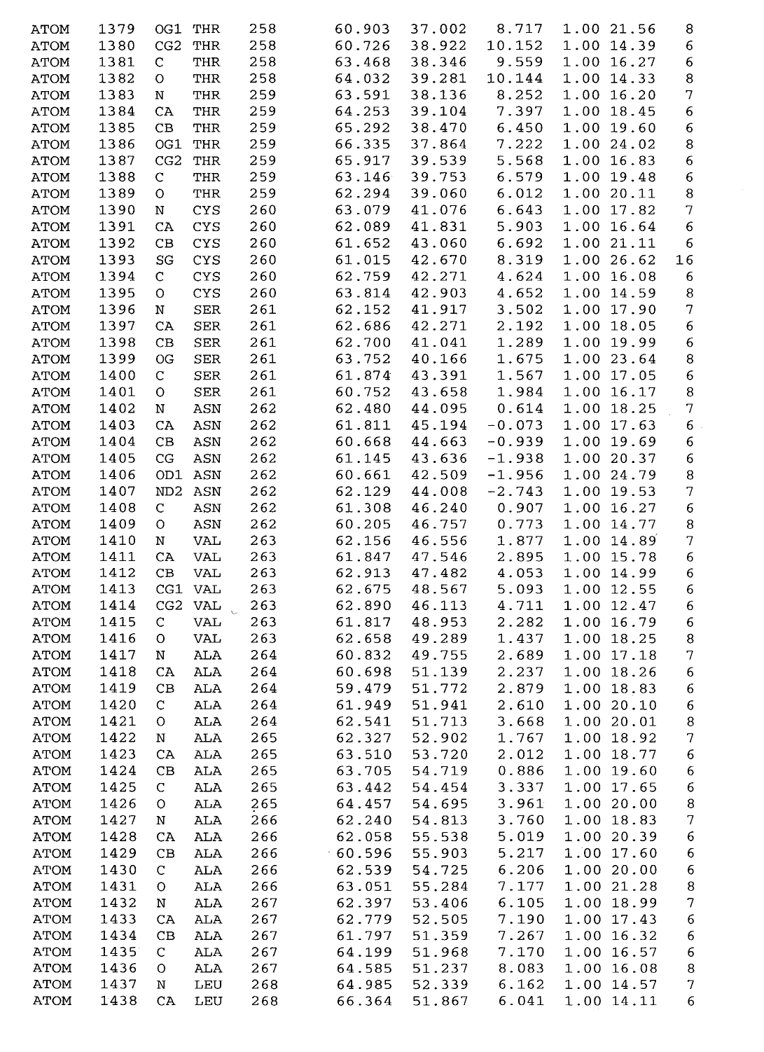

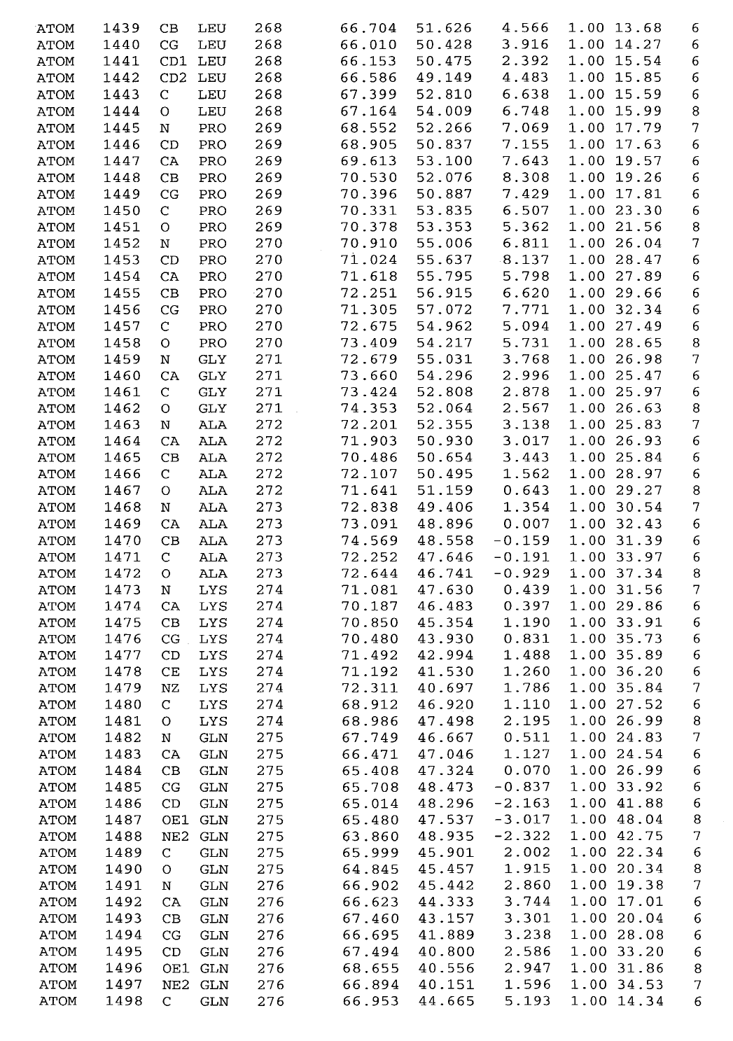

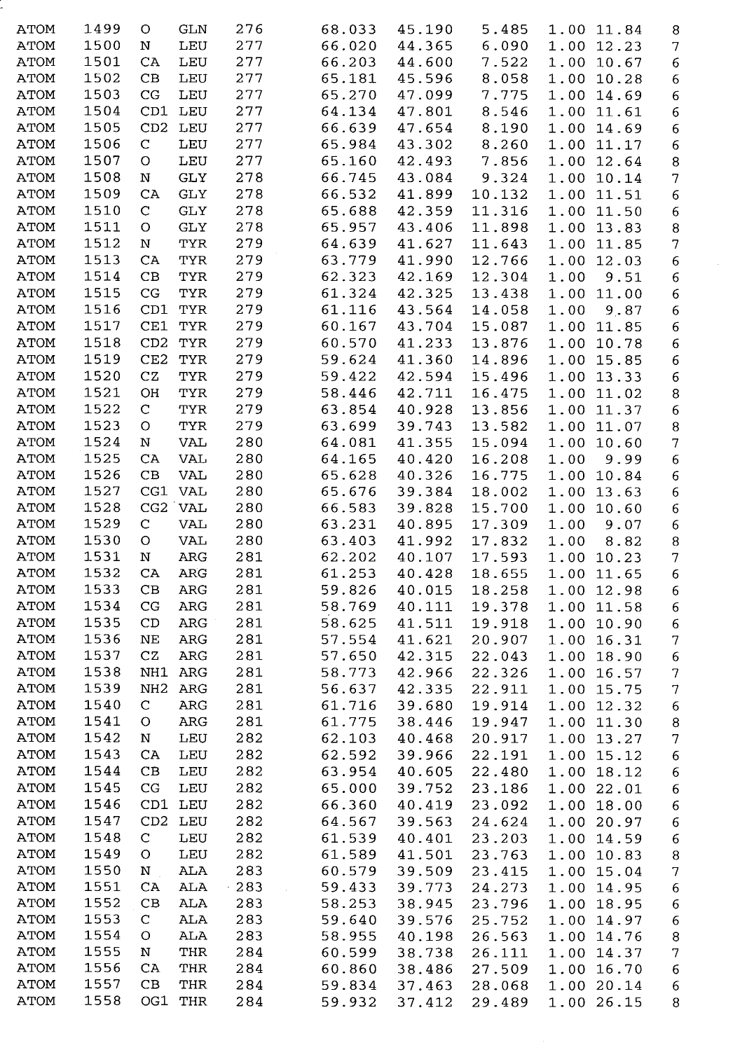

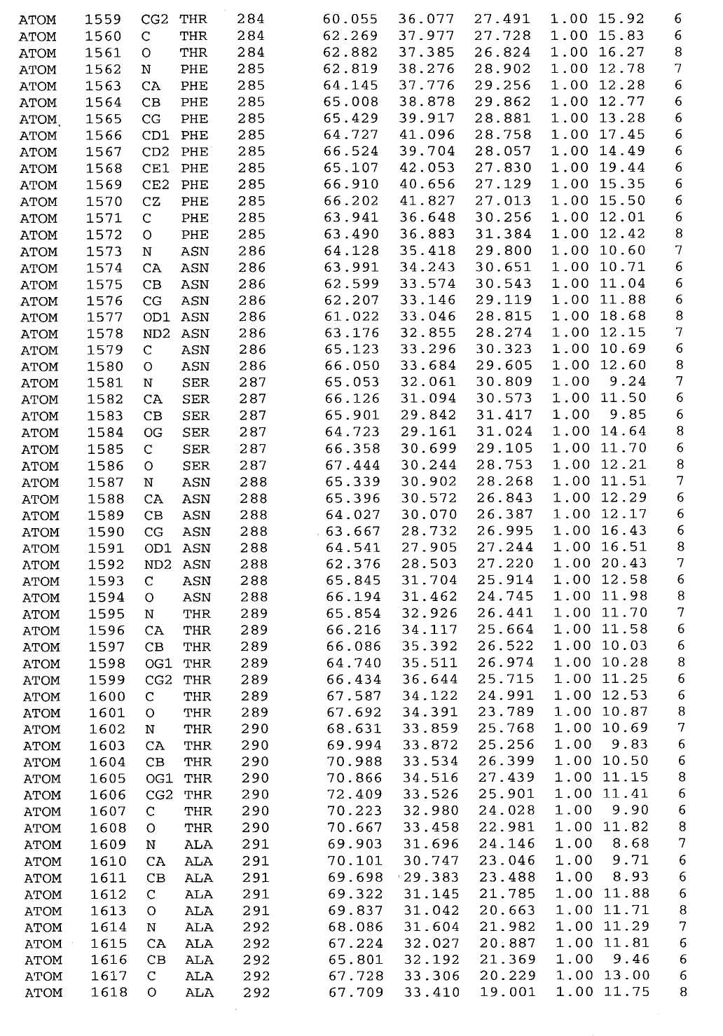

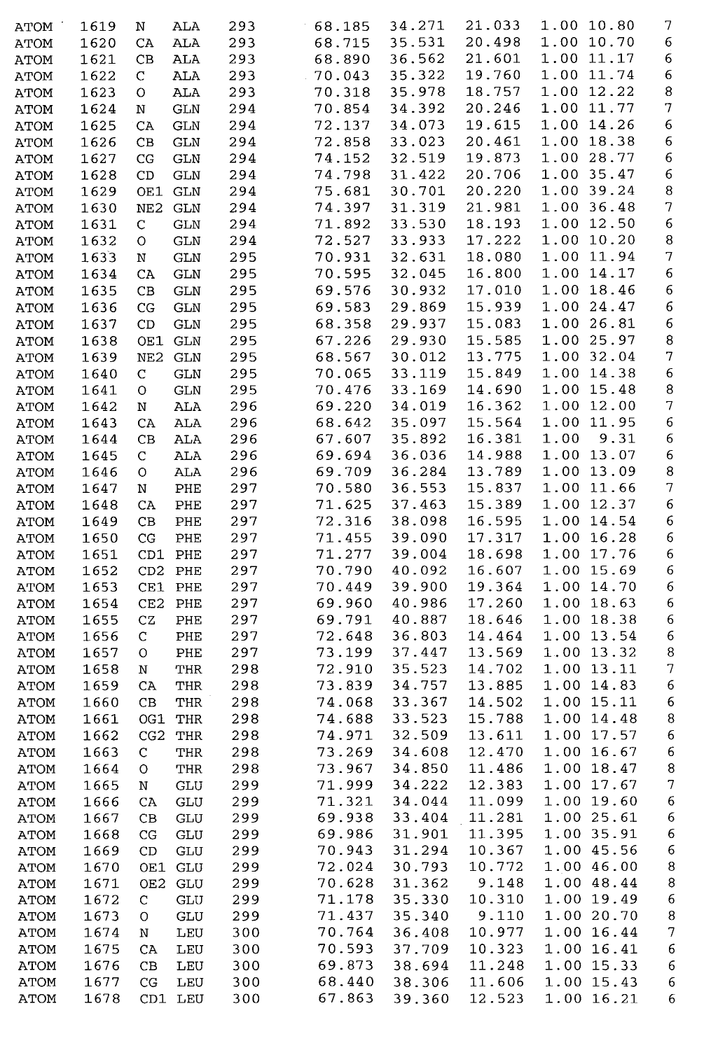

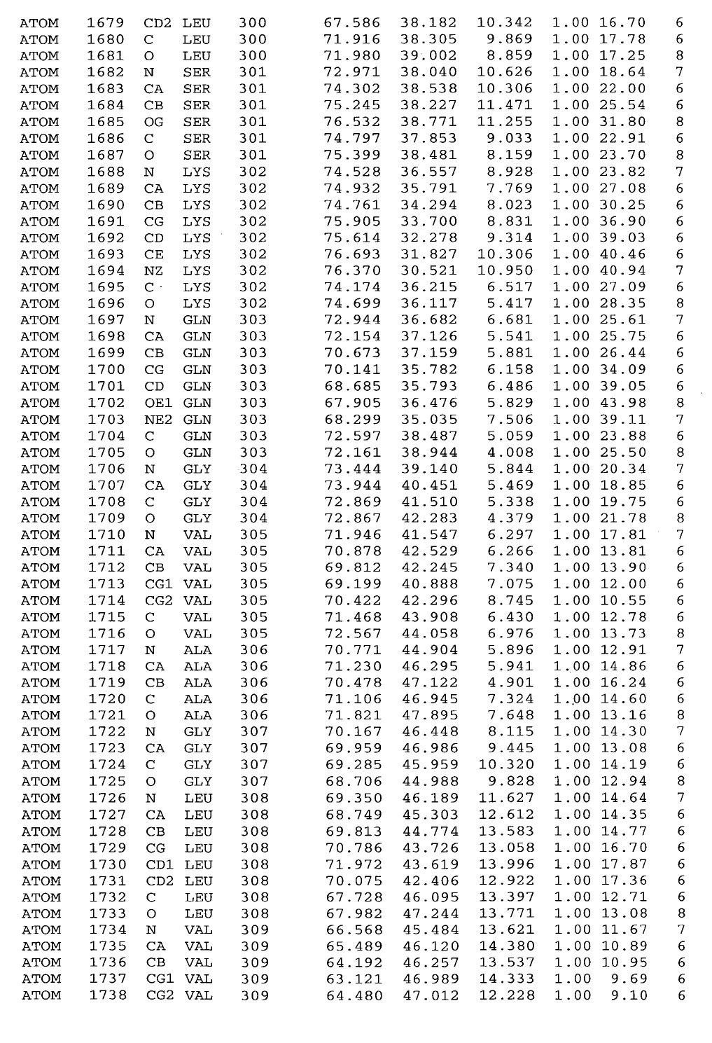

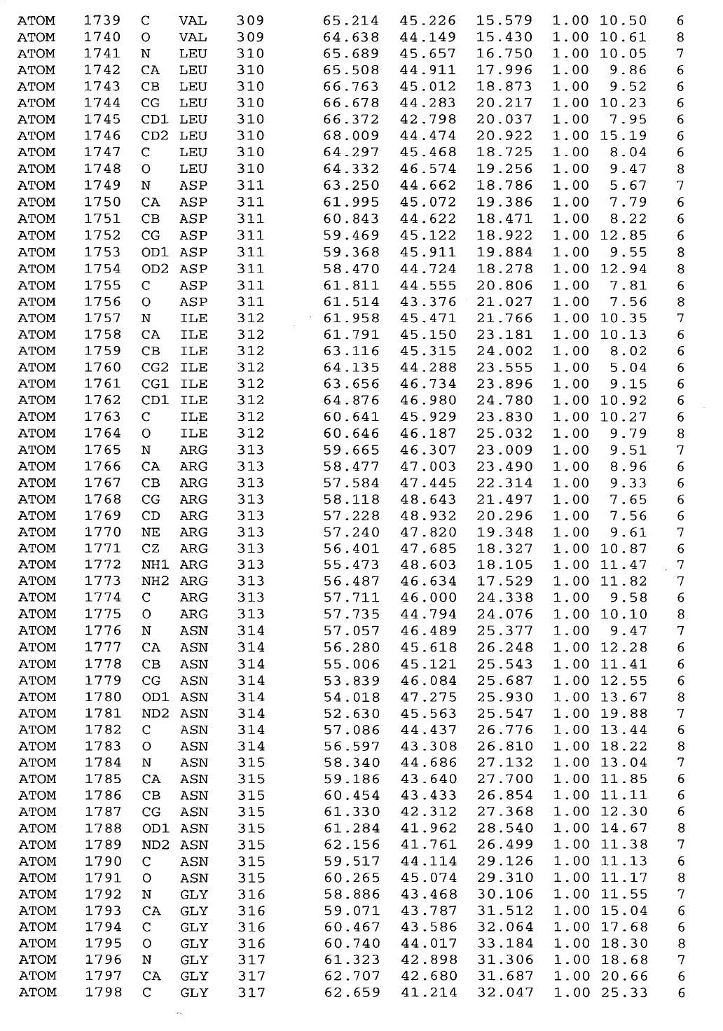

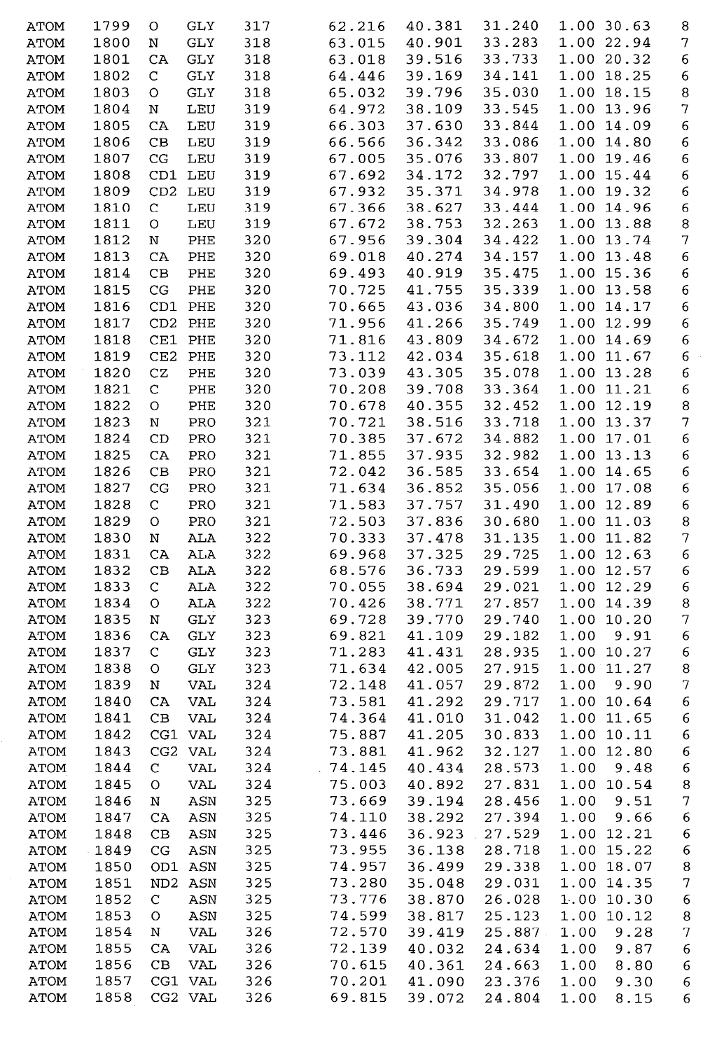

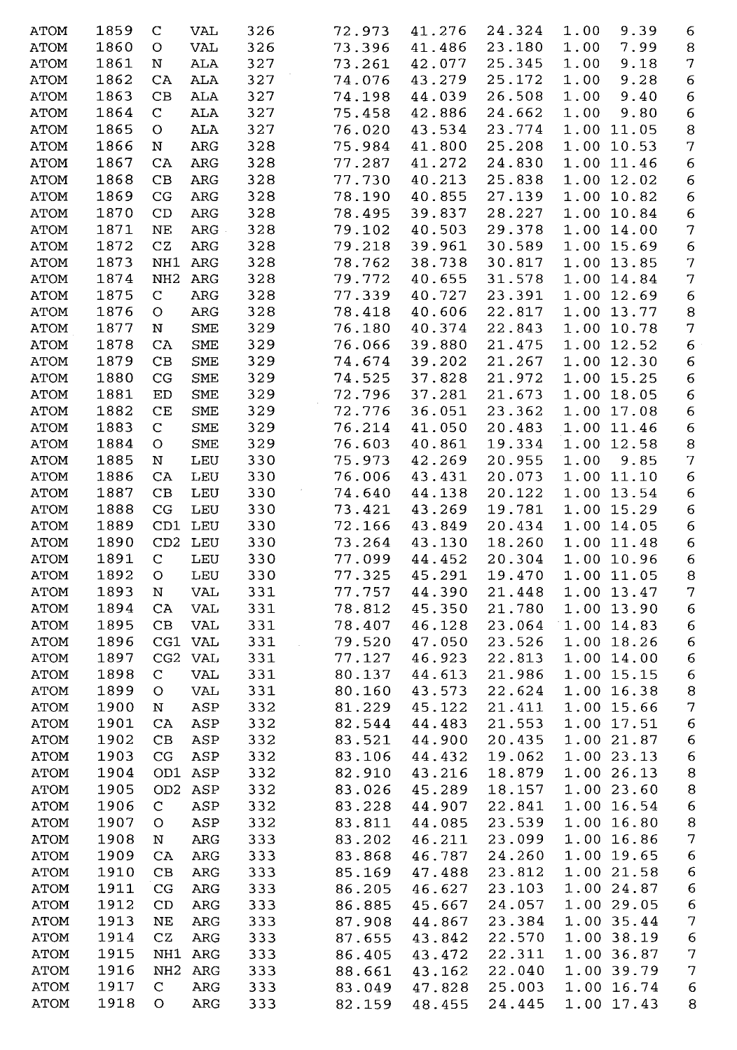

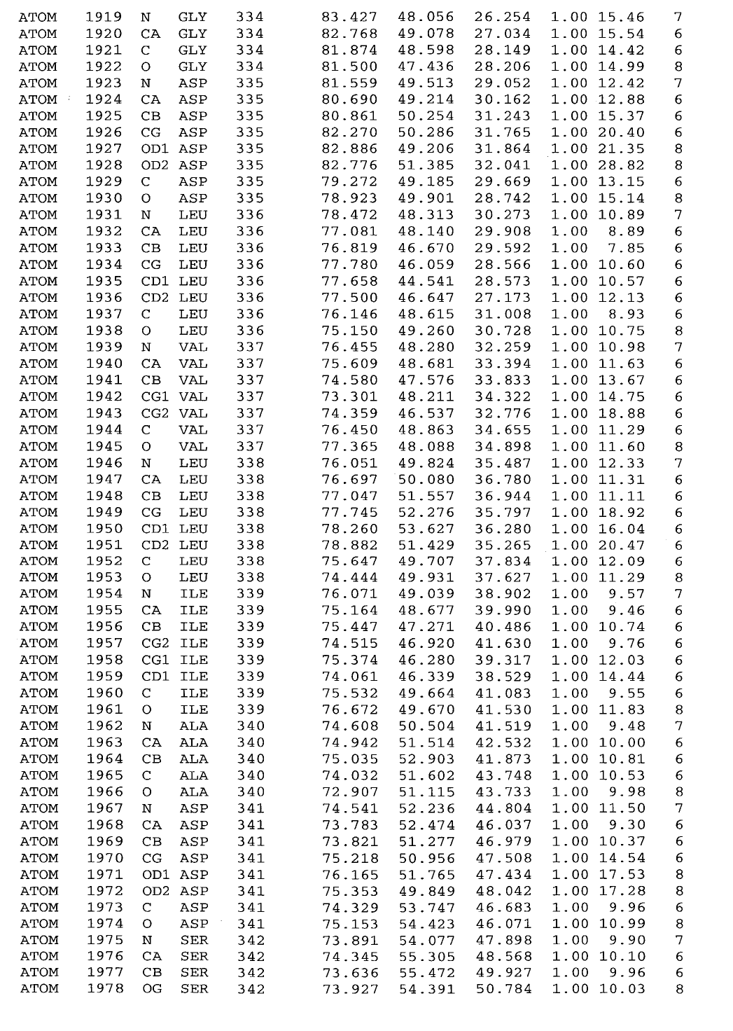

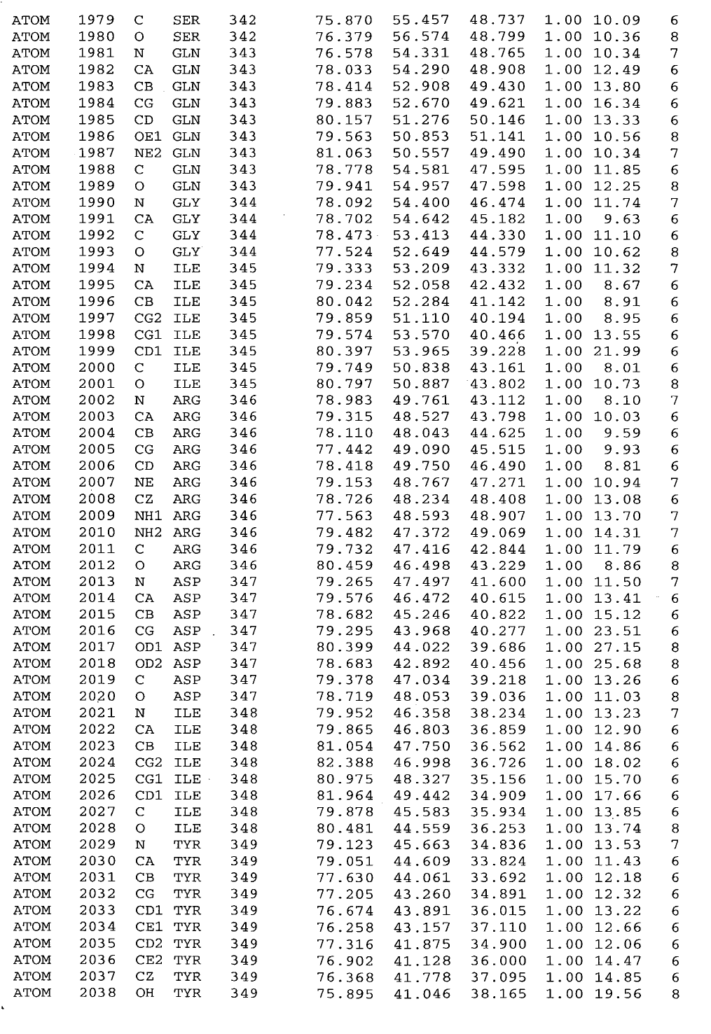

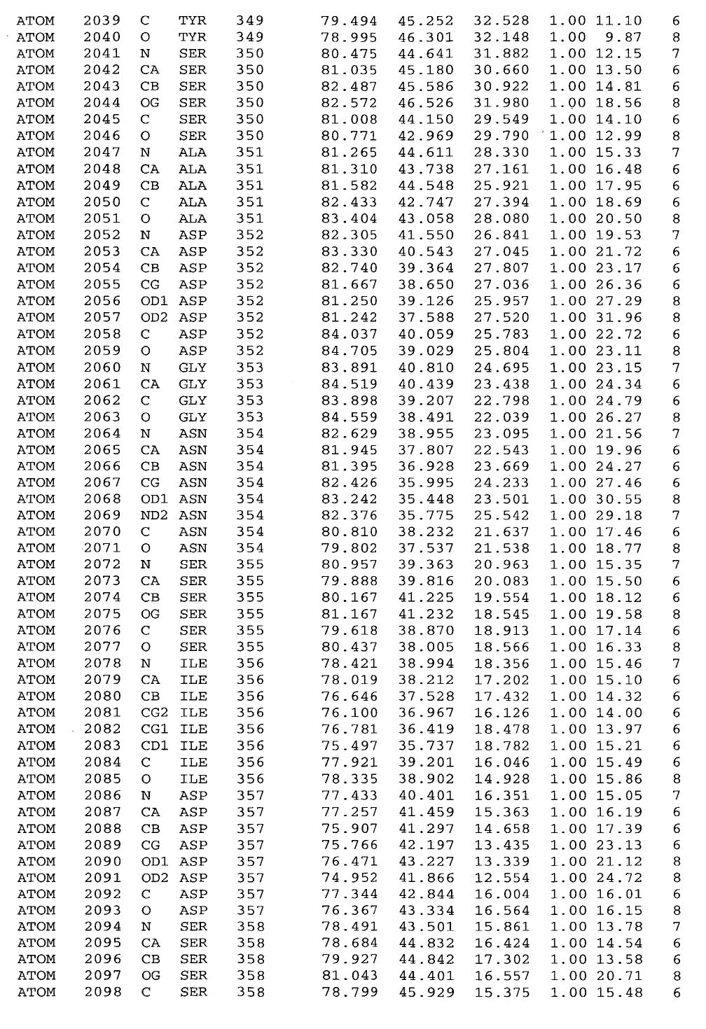

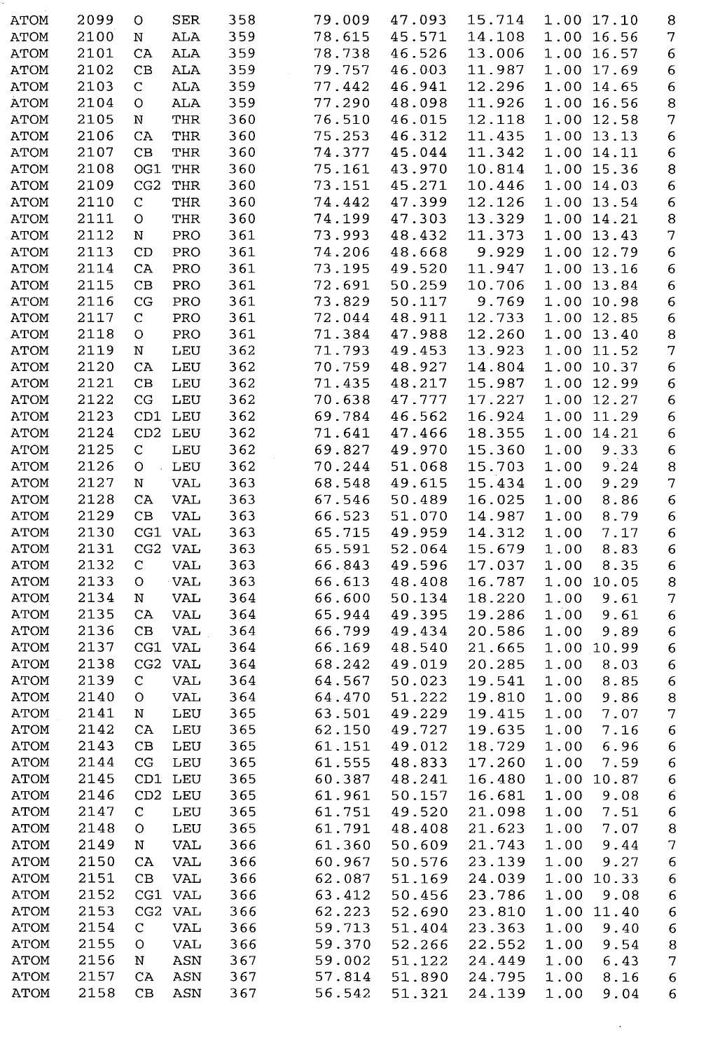

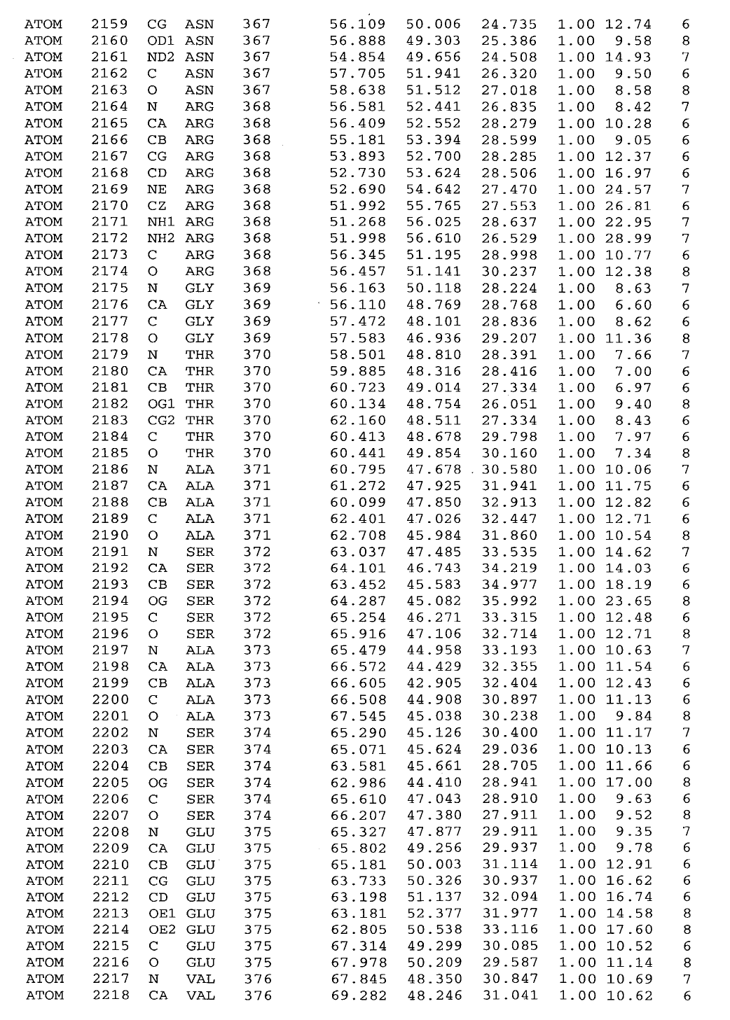

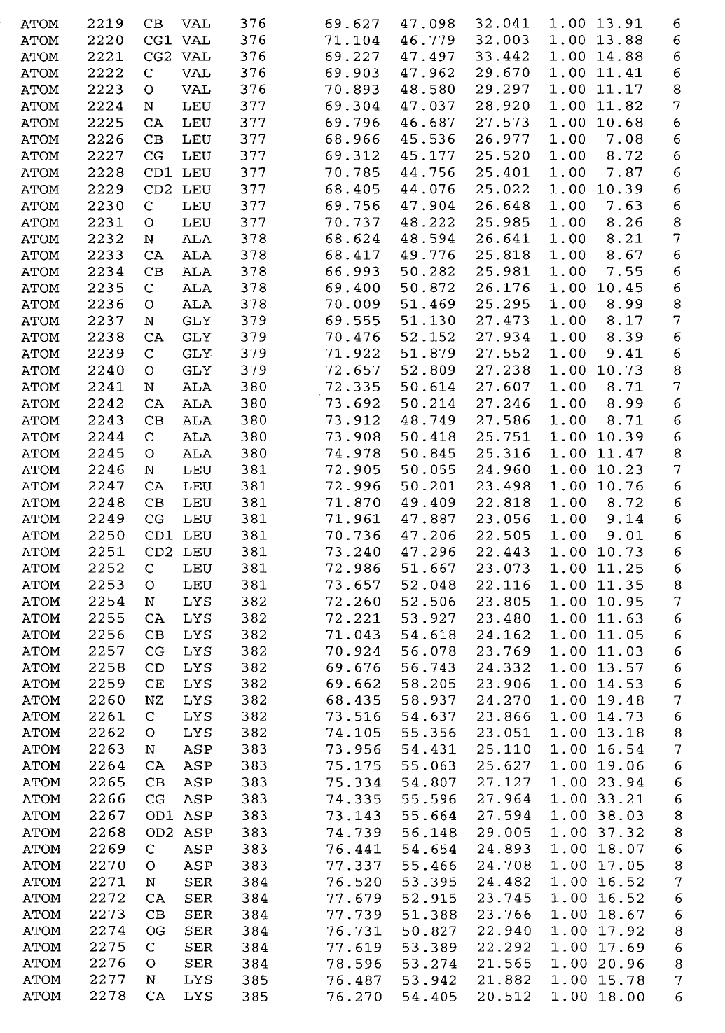

The present invention describes methods for expressing, mutating, refolding, purifying, crystallizing and solving to high resolution the X-ray crystal structure of the D1-C-terminal processing (D1) protease from Scenedesmus obliquus. The X-ray crystal structure describes the apoprotein. The three-dimensional structure (e.g., as provided on computer readable media of the present invention; FIG. 1) is useful for rational design of ligands of D1 protease. Such ligands can be synthesized and are useful as agronomic compounds for inhibiting the activity of D1 protease.

In this disclosure, a number of terms and abbreviations are used. The following definitions are provided.

“D1-C-terminal processing protease” is abbreviated D1 protease.

“Multiwavelength Anomalous Diffraction” is abbreviated MAD.

“Multiple isomorphous replacement” is abbreviated MIR.

“Polymerase chain reaction” is abbreviated PCR.

The term “D1 protease” refers to an enzyme responsible for the processing of the D1 pre-protein at the C-terminal end for the production of the mature D1 polypeptide.

The terms “D1 pre-protein”, “D1 pre-polypeptide”, and “pre-D1” refer to the D1 precursor protein that has been N-terminally processed but contains an additional 8 to 16 amino acid residues at the C-terminal portion of the protein which are cleaved off by D1 protease at the carboxy side of D1 -Ala344 to yield the mature D1 protein.

The terms “D1 protein”, “D1 polypeptide”, and “mature D1 protein or polypeptide” refer to an electron transport polypeptide that is both N- and C-terminally processed and a subunit of the PSII reaction center. This polypeptide is implicated in coordinating a tetranuclear manganese (Mn) cluster which is found in the PSII reaction center of all photosynthetic organisms and is responsible for the coordination of the primary photoreactants.

The term “enzyme substrate” means any compound or material that is capable of interacting with or binding to the active enzymatic site of D1 protease where that substrate is catalytically cleaved by the interaction with the active site. As used herein a suitable substrate for the D1 protease enzyme may be the D1 pre-protein, or a portion of that pre-protein comprising the D1 processing site.

The term “D1 processing site” refers to the region on the D1 pre-protein that is cleaved by the D1 protease enzyme. As used herein “D1 processing” refers to the cleavage of the D1 pre-protein by D1 protease.

The term “D1 active site” or “active site” refers to the portion of the D1 protease enzyme responsible for D1 processing. For the purposes of the present invention an “active site” will comprise any region of 41 contiguous amino acid residues, located within a polypeptide having D1 processing activity, where there exists at least 60% amino acid identity between region and the corresponding region beginning at residue 361 and ending at residue 402 of the D1 protease enzyme isolated from the Scenedesmus obliquus as set forth in SEQ ID NO: 1.

The term “ligand” means any compound capable of interacting with the active site of D1 protease or binding to any other domain or sub-domain of D1 protease. Ligands may include but are not limited to enzyme substrates.

The term “complex” as used herein refers to the association of a protein with other substances or molecules useful in determining the structure of the protein. Thus, a protein may be complexed with a ligand or substrate at the active site. A “binary complex” refers to the association of the protein with one other substance, such as for example the binding of the enzyme with a ligand or substrate.

The term “atomic coordinate/X-ray diffraction data” means that data generated from an X-ray diffraction procedure that will enable the determination of the structure of a protein.

The term “predicted atomic coordinate data” or “coordinate data” means that data generated from a computer modeling program that predicts atomic coordinate data that will enable the determination of the structure of a protein.

The term “computer model output data” refers to the data generated by modeling and compound docking software using atomic coordinate/X-ray diffraction coordinates.

As used herein the general term “molecular modeling” will refer to the use of a computer algorithm to generate a predicted model of a protein. “Molecular modeling” may encompass specific type of modeling applications, as for example homology modeling or molecular replacement modeling.

The term “molecular replacement” refers to a computer based method of determining the three dimensional structure of a protein of interest using the atomic coordinates for a reference protein and the X-ray diffraction data from the protein of interest.

The term “homology modeling” refers to a computer based method of determining the three dimensional structure of a protein of interest using a combination of the primary structure of the protein of interest and the crystal structure of at least one reference protein.

The term, “rational compound design” means the use of a set of atomic coordinate/X-ray diffraction data derived from a protein or protein complex, in conjunction with computer modeling software to determine compounds that will most likely bind to or interact with a specific site on the protein or protein complex.

As used herein where references to the positions of amino acids in D1 protease are mentioned (e.g., Lys397), they will always be relative to the amino acid sequence set forth in SEQ ID NO: 1, unless otherwise indicated.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

As used herein the terms “percent identity” and “percent homology” will be used interchangeably. The term “percent identity” is a relationship between two or more polypeptide sequences or two or more polypeptide or polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG Pileup program found in the GCG program package, using the Needleman and Wunsch algorithm with their standard default values of gap creation penalty=12 and gap extension penalty=4 (Devereux et al., Nucleic Acids Res. 12:387-395 (1984)), BLASTP, BLASTN, and FASTA (Pearson et al., Proc. Natl. Acad. Sci. USA 85:2444-2448 (1988). The BLASTX program is publicly available from NCBI and other sources (BLAST Manual, Altschul et al., Natl. Cent. Biotechnol. Inf., Natl. Library Med. (NCBI NLM) NIH, Bethesda, Md. 20894; Altschul et al., J. Mol. Biol. 215:403-410 (1990); Altschul et al., (Gapped BLAST and PSI-BLAST: a new generation of protein database search programs), Nucleic Acids Res. 25:3389-3402 (1997)). The method to determine percent identity preferred in the present invention is by the method of DNASTAR protein alignment protocol using the Jotun-Hein algorithm (Hein et al., Methods Enzymol. 183:626-645 (1990)). Default parameters used for the Jotun-Hein method for alignments are: for multiple alignments, gap penalty=11, gap length penalty=3; for pairwise alignments ktuple=2. As an illustration, for a polynucleotide having a nucleotide sequence with at least 95% identity to a reference nucleotide sequence, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, for a polypeptide having an amino acid sequence having at least 95% “identity” to a reference amino acid sequence, it is intended that the amino acid sequence of the polypeptide is identical to the reference sequence except that the polypeptide sequence may include up to five amino acid alterations per each 100 amino acids of the reference amino acid. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

The determined structure is made using the D1 protease amino acid sequence (SEQ ID NO: 1) and/or atomic coordinate/x-ray diffraction data, which are analyzed to provide atomic model output data corresponding to the three-dimensional structure, e.g., as provided on computer readable media. The computer analysis of the atomic coordinate/x-ray diffraction data and/or the amino acid sequence allows the calculation of the secondary and/or tertiary structures, domains, and/or subdomains of the protein. These domains are combined and refined by additional calculations using suitable computer subroutines to determine the most probable or actual three-dimensional structure of the D1 protease, including potential or actual active sites, binding sites or other structural or functional domains or subdomains of the protein. The resulting three-dimensional structure is represented as atomic model output data on the computer readable media.

Structure determination methods are also provided by the present invention for rational design of D1 protease ligands. Such design uses computer modeling programs that calculate different molecules expected to interact with the determined active sites, binding sites, or other structural or functional domains or subdomains of a D1 protease. These ligands can then be produced and screened for activity in modulating or binding to a D1 protease, according to methods and compositions of the present invention.

The actual D1 protease-ligand complexes can optionally be crystallized and analyzed using x-ray diffraction techniques. The diffraction patterns obtained are similarly used to calculate the three-dimensional interaction of the ligand and the D1 protease, to confirm that the ligand binds to, or changes the conformation of, particular domain(s) or subdomain(s) of the D1 protease. Such screening methods are selected from assays for at least one biological activity of a D1 protease. The resulting ligands, provided by methods of the present invention, modulate or bind at least one D1 protease and are useful as inhibitors of the D1 protease enzyme. Ligands of a particular D1 protease can similarly modulate other D1 proteases from other sources such as other plants.

A D1 protease is also provided as a crystallized protein suitable for x-ray diffraction analysis. The x-ray diffraction patterns obtained by the x-ray analysis are of moderate, to moderately high, to high resolution, e.g., equal to or better than 3.5 Å where about 1.8 Å to about 0.7 Å is preferred. It is well understood in the art of x-ray diffraction that the lower the resolution figure the more refined the resolution and the more useful the data obtained from such a pattern. These diffraction patterns are suitable and useful for three-dimensional structure determination of a D1 protease, domain or subdomain thereof.

The determination of the three-dimensional structure of a D1 protease has a broad-based utility. Significant sequence identity and conservation of important structural elements are expected to exist among different D1 proteases and other homologs, including Prc protease (Genbank D00674; Hara, et al., Journal of Bacteriology 173, 4799-4813(1991)). Therefore, the three-dimensional structure from one or a few D1 proteases can be used to identify ligands that have the ability to inhibit the D1 protease enzyme or D1 protease homologs having different amino acid sequences. More specifically, the three-dimensional structure from one or more D1 proteases can be used to identify ligands that are inhibitory in other D1 proteases with different amino acid sequences. Inhibitors to D1 protease are expected to have herbicidal activity.

Isolated D1 Protease Polypeptides

A D1 protease polypeptide can refer to any subset of a D1 protease as a domain, subdomain, fragment, consensus sequence or repeating unit thereof. A D1 protease polypeptide of the present invention can be prepared by any of the following methods:

(a) recombinant DNA methods;

(b) proteolytic digestion of the intact molecule or a domain, subdomain or fragment thereof;

(c) chemical peptide synthesis methods well-known in the art; and/or

(d) by any other method capable of producing a D1 protease polypeptide and having a conformation similar to a structural or functional subdomain of a D1 protease.

A biological activity of D1 protease can be screened according to known and patented screening assays (Trost et al., J. Biol. Chem. 272:20348-20356 (1997); U.S. Pat. No. 5,876,945). The minimum peptide sequence to have activity is based on the smallest unit containing or comprising a particular domain, subdomain, fragment, region, consensus sequence, or repeating unit thereof, having at least one biological activity of a D1 protease, such as enzyme activity.

A D1 protease polypeptide of the invention can have at least 60% homology or sequence identity, such as 60-100% overall homology or identity, with one or more corresponding D1 protease subdomains or fragments as described herein, such as the amino acids of SEQ ID NO: 1. As would be understood by one of ordinary skill in the art, the above configurations of subdomains are provided as part of a D1 protease polypeptide of the invention, when expressed in a suitable host cell, or otherwise synthesized, to provide at least one structural or functional feature of a native D1 protease, such as at least one D1 protease-related biological activity. The active site of the D1 protease is the region most likely to be the subject of such analysis. The active site, in most D1 protease enzymes, spans a distance of about 40 amino acid residues, as for example in the Scenedesmsus enzyme where the active site region comprises amino acids 361 to 402. Comparisons of the active sites of D1 protease enzymes in this active site region to the Scenedesmsus active site by BESTFIT (version 9.0-OpenVMS, Genetics Computer Group (GCG)), using default parameters are shown below:

| | % identity with Scenedesmsus D1 |
|---|---|
| D1 protease source | protease Active Site Region |
| Tobacco | 71% |
| Spinach | 74% |
| Wheat | 74% |
| Synechocystis CtpA | 74% |
| Synechocystis CtpC | 60% |

Thus, relevant D1 protease fragments, domains or sub-domains of D1 protease would have at least 60% amino acid identity to the D1 protease active site.

Such activities can be assayed using a suitable assay, to establish at least one D1 protease biological activity of one or more D1 protease of the invention. A D1 protease polypeptide of the invention is not naturally occurring or is naturally occurring but is in a purified isolated form which does not occur in nature. Assay methods for D1 protease are known. For example, Trost et al., (J. Biol. Chem. 272:20348-20356 (1997)) and U.S. Pat. No. 5,876,945 disclose a method of determining D1 protease activity. Alternatively, a suitable assay for D1 protease may be designed by the skilled person.

As previously noted, percent homology or identity can be determined, for example, by comparing sequence information using the GAP or BESTFIT computer programs (version 9.0-OpenVMS, Genetics Computer Group (GCG)). The GAP program utilizes the alignment method of Needleman and Wunsch (J. Mol. Biol. 48:443 (1970)) and performs the comparison across the entire length of the sequences. The BESTFIT program uses the local homology program of Smith and Waterman (Adv. Applied Mathematics 2:482-489 (1981)) to find the best segment of similarity between two sequences. The preferred default parameters for the GAP and BESTFIT programs are routinely used. Both programs define percent identity as the number of aligned symbols (i.e., nucleotides or amino acids) which are the same, in the respective aligned sequences, divided by the total number of symbols in the shorter of the two sequences.

Thus, one of ordinary skill in the art, given the teachings and guidance presented in the present specification, will know how to add, delete or substitute other amino acid residues in other positions of a D1 protease to obtain substituted, deletional or additional variants thereof.

Non-limiting examples of substitutions of D1 protease domains or polypeptides of the invention are those in which at least one amino acid residue in the protein molecule has been removed and a different residue added in its place. The types of substitutions which can be made in the protein or peptide molecule of the invention can be based on analysis of the frequencies of amino acid changes between a homologous protein of different species. Based on such an analysis, alternative substitutions are defined herein as exchanges within one of the following five groups:

1. Small aliphatic, nonpolar or slightly polar residues: Ala, Ser, Thr (Pro, Gly);

2. Polar, negatively charged residues and their amides: Asp, Asn, Glu, Gln;

3. Polar, positively charged residues: His, Arg, Lys;

4. Large aliphatic, nonpolar residues: Met, Leu, Ile, Val (Cys); and

5. Large aromatic residues: Phe, Tyr, Trp.

Most deletions and additions and substitutions according to the invention are those which do not produce radical changes in the characteristics of the protein or peptide molecule. “Characteristics” is defined in a non-inclusive manner to define both changes in secondary structure, e.g., α-helix or β-sheet, as well as changes in physiological activity, e.g., in biological activity assays. However, when the exact effect of the substitution, deletion, or addition is to be confirmed, one skilled in the art will appreciate that the effect of at least one substitution, addition or deletion will be evaluated by at least one D1 protease screening assay, such as, but not limited to, immunoassays or bioassays, to confirm at least one D1 protease biological activity.

Computer Related Embodiments

An amino acid sequence of a D1 protease (SEQ ID NO: 1) and/or atomic coordinate/x-ray diffraction data, useful for computer structure determination of a D1 protease or a portion thereof, can be “provided” in a variety of mediums to facilitate use thereof. As used herein, provided refers to a manufacture, which contains a D1 protease amino acid sequence and/or atomic coordinate/x-ray diffraction data of the present invention, e.g., the amino acid sequence provided in SEQ ID NO: 1, a representative fragment thereof, or an amino acid sequence having at least 60-100% overall identity of SEQ ID NO: 1, or at least 60% identity to the active site of the D1 protease enzyme. Such a medium provides the amino acid sequence and/or atomic coordinate/x-ray diffraction data in a form which allows a skilled artisan to analyze and determine the three-dimensional structure of a D1 protease or a subdomain thereof.

In one application of this embodiment, D1 protease, or at least one subdomain thereof, amino acid sequence and/or atomic coordinate/x-ray diffraction data of the present invention is recorded on computer readable media. As used herein, “computer readable media” refers to any medium which can be read and accessed directly by a computer. Such media include, but are not limited to: magnetic storage media, such as floppy discs, hard disc storage medium, and magnetic tape; optical storage media such as optical discs or CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon an amino acid sequence and/or atomic coordinate/x-ray diffraction data of the present invention.

As used herein, “recorded” refers to a process for storing information on computer readable medium. A skilled artisan can readily adopt any of the presently known methods for recording information on computer readable medium to generate manufactures comprising an amino acid sequence and/or atomic coordinate/x-ray diffraction data information of the present invention.

A variety of data storage structures are available to a skilled artisan for creating a computer readable medium having recorded thereon an amino acid sequence and/or atomic coordinate/x-ray diffraction data of the present invention. The choice of the data storage structure will generally be based on the means chosen to access the stored information. In addition, a variety of data processor programs and formats can be used to store the amino acid sequence and/or atomic coordinate/x-ray diffraction data of the present invention on computer readable medium. The amino acid sequence information can be represented in a word processing text file, formatted in commercially-available, word processing software, or represented in the form of an ASCII file, or stored in a database application. A skilled artisan can readily adapt any number of data-processor structuring formats (e.g., text file or database) in order to obtain computer readable medium having recorded thereon the information of the present invention.

By providing on computer readable media having stored therein a D1 protease sequence and/or atomic coordinates derived from x-ray diffraction data, a skilled artisan can routinely access the sequence and atomic coordinates or x-ray diffraction data to model a three dimensional structure of D1 protease, a subdomain thereof, or a ligand thereof. Computer algorithms are publicly and commercially available which allow a skilled artisan to access this data provided on a computer readable medium and analyze it for structure determination and/or rational inhibitor design. See, e.g., Biotechnology Software Directory, Mary Ann Liebert Publ., New York (1995).

The present invention further provides systems, particularly computer-based systems, which contain the amino acid sequence and/or atomic coordinate/x-ray diffraction described herein. Such systems are designed to do structure determination and rational design for a D1 protease or at least one subdomain thereof. Non-limiting examples are microcomputer workstations available from Silicon Graphics Incorporated and Sun Microsystems running Unix based, Windows NT or IBM OS/2 operating systems.

As used herein, “a computer-based system” refers to the hardware means, software means, and data storage means used to analyze the amino acid sequence and/or atomic coordinate/x-ray diffraction of the present invention. The minimum hardware means of the computer-based systems of the present invention comprises a central processing unit (CPU), input means, output means, and data storage means. A skilled artisan can readily appreciate which of the currently available computer-based systems are suitable for use in the present invention. A monitor is optionally provided to visualize structure data.

As stated above, the computer-based systems of the present invention comprise a data storage means having stored therein a D1 protease or fragment amino acid sequence and/or atomic coordinate/x-ray diffraction data of the present invention and the necessary hardware means and software means for supporting and implementing an analysis means. As used herein, “data storage means” refers to memory which can store amino acid sequence or atomic coordinate/x-ray diffraction data of the present invention, or a memory access means which can access manufactures having recorded thereon the amino acid sequence or atomic coordinate/x-ray diffraction data of the present invention.

As used herein, “search means” or “analysis means” refers to one or more programs which are implemented on the computer-based system to compare a target sequence or target structural motif with the amino acid sequence or atomic coordinate/x-ray diffraction data stored within the data storage means. Search means are used to identify fragments or regions of a D1 protease which match a particular target sequence or target motif. A variety of known algorithms are disclosed publicly and a variety of commercially available software for conducting search means are and can be used in the computer-based systems of the present invention. A skilled artisan can readily recognize that any one of the available algorithms or implementing software packages for conducting computer analyses that can be adapted for use in the present computer-based systems.

As used herein, “a target structural motif,” or “target motif,” refers to any rationally selected sequence or combination of sequences in which the sequence(s) are chosen based on a three-dimensional configuration or electron density map which is formed upon the folding of the target motif. There are a variety of target motifs known in the art. Protein target motifs include, but are not limited to, enzymatic active sites, structural subdomains, epitopes, functional domains and signal sequences. A variety of structural formats for the input and output means can be used to input and output the information in the computer-based systems of the present invention.

A variety of comparing means can be used to compare a target sequence or target motif with the data storage means to identify structural motifs or interpret electron density maps derived in part from the atomic coordinate/x-ray diffraction data. A skilled artisan can readily recognize that any one of the publicly available computer modeling programs can be used as the search means for the computer-based systems of the present invention.

Structure Determination

Crystallization of the instant D1 protease enzyme may be accomplished by a variety of means. For example crystals of the present D1 protease or D1 protease bound to a suitable ligand can be grown by, vapor diffusion (either by sitting drop or hanging drop) and by microdialysis. Seeding of the crystals in some instances is required to obtain x-ray quality crystals. Standard micro and/or macro seeding of crystals may therefore be used.

Of course, the specific D1 protease of the present invention provided herein serves only as an example, since the crystallization process can tolerate a range of lengths of the flexible portion of the protein. Similarly, the crystallization process will also tolerate a limited removal of amino acids in the globular portion (e.g., less than ten amino acids). Therefore, any person with skill in the art of protein crystallization having the present teachings and without undue experimentation could construct a variety of alternative forms of the D1 protease which could be crystallized.

Once a crystal of the present invention is grown, x-ray diffraction data can be collected using a synchrotron source such as Cornell High Energy Synchrotron source (CHESS), under standard cryogenic conditions. A variety of methods are available. For example the skilled person could characterize crystals by using x-rays produced in a conventional source (such as a sealed tube or a rotating anode) or using a synchrotron source. Methods of characterization include, but are not limited to, precision photography, oscillation photography and diffractometer data collection. Se-Met multiwavelength anomalous dispersion (MAD) data (Hendrickson, Science 254:51-58 (1991)) can be collected using reverse-beam geometry to record Friedel pairs at four x-ray wavelengths, corresponding to two remote points above and below the Se absorption edge and the K-absorption edge inflection point and peak. Data can be processed using readily available software such as DENZO and SCALEPACK (Szebenyi et al., AIP Conf: Proc. 417(Synchrotron Radiation Instrumentation):187-191 (1997)), for example.

Alternatively, it is possible to define the three dimensional structure of a protein using computer based methods such as molecular replacement and homology modeling. The method of molecular replacement combines the atomic coordinates for a reference protein and the x-ray diffraction data from the protein of interest to determine the three dimensional protein structure. The object in molecular replacement is to use this combined set of data to determine the relative positions of atoms within the crystal. The method may be accomplished using commercially available software such as AmoRe, fully described by Navaza et al., Methods Enzymol. (1997), 276(Macromolecular Crystallography, Part A), 581-594). Within the context of the present invention molecular replacement methods may be used to generate three dimensional structures for plant D1 protease enzymes using the method of molecular replacement and employing coordinates generated from the Scenedesmus obliquus enzyme and x-ray diffraction data from the plant enzyme.

The process of homology modeling uses a combination of the primary structure of the protein of interest and the crystal structure of at least one reference protein. The 3-dimensional model is generated based on the protein's amino acid sequence. The model may be constructed by first aligning the amino acid sequence of the protein of interest with the sequence of the reference protein. In regions where the homology between the two proteins is low, information gleaned from secondary structure and site directed mutageneis may be useful. Next, structurally conserved regions of the protein of interest are determined based on the alignment and then the coordinates for these regions are copied from the crystal structure data of the reference protein. The model is then refined using computerized methods. Homology modeling is a technique well known in the art and has been used to determine the three-dimensional structure of a variety of proteins (see for example Grazyna et al., Life Sciences, 61, 2507, (1997) describing the use of homology modeling for the determination of the three-dimensional structure of cytochrome p-450). The present invention provides a method for the determination of the three-dimensional structure of plant D1 protease enzymes using the crystal structure of the Scenedesmus enzyme and the amino acid sequence of the plant enzyme of interest.

The Fold of the Structure

D1 protease is an elongated shape monomeric molecule about 77.5 Å long with the widest cross section measured 47.1 Å×27.6 Å located in the middle section of the molecule. It contains three folding domains: (i) the A domain (amino acid residues 78-147, 401-415) containing a three-helix bundle followed by a short beta strand and a two turn helix; (ii) the B domain (residues 160-249) [which is a PDZ domain, as described in Ponting, Protein Science 6, 464 (1997)] containing a severely twisted five-stranded anti-parallel β-sheet with a two turn helix sitting on top, and; (iii) the C-domain (residues 254-400, 416-463) containing two β-sheets. Within the C domain one β-sheet is a six-stranded mixed β-sheet twisted about 100 degrees and with three helices packed against one side of the sheet and the C-terminal helix on the other side. The other β-sheet is a small three stranded anti-parallel β-sheet which has some contact with the three helices on the other sheet. The fifth strand on the large sheet and the first strand on the small sheet extend to the A domain and together with the beta strand in that domain form a three-stranded anti-parallel sheet. This part of the two beta strands (residue 401-415) is an integral part of the A domain. The linkers between domain A and domain B, as well as between domain B and domain C, have weaker density, indicating that the structure in these regions is more flexible than the rest of the structure. The B domain has very few interactions with the other two domains and therefore it is possible that the conformation observed in this structure may be affected by crystal packing. This domain may have the ability to adjust its orientation upon the binding of different substrates or inhibitors, or maybe even during the course of reaction. Superposition of the C2 I form and R32 form structure shows small but detectable domain movement.

Analysis of the Active Site

Unlike the classical serine proteases, D1 protease does not have a steep active site cleft. Instead, its active site region is rather opened, similar to the one in HCV protease (PDB ID code 1A1R. J. L. Kim et. al., Cell Vol. 87 page 343, 1996). The active site is formed by all three domains with the C domain on one side and the A and B domains on the other. This shallow cleft runs across the entire cross section of 47.1 Å in the molecule. The opening of the cleft is about 15 Å throughout the cleft. Both the active site Lys397 and Ser372 are located on the large C domain. They are located in the middle of the cross section and at the bottom of the cleft. The Lys397 is in the middle of the fifth strand of the large β-sheet, one of the two strands that extends to the A domain. Ser372 is at the N-terminal of the 3^{rd} alpha-helix. The distance between the two main chains' CA's of these two residues is 5.1 Å. The NE of the Lys397 is hydrogen bonded to the OG of Thr168 and the OG of the serine side-chain which interacts with two water molecules in form C2 I. In form R32 the side-chain of the serine shows two conformations. The first interacting with a water molecule and the second interacting with the main chain carbonyl of Lys397. These observations show that, without the bound substrate, the active site residues can have more than one conformation in solution. In both cases, the two side chains are not within hydrogen bonding distance. However, computer modeling shows that they can be brought to form a hydrogen bond for catalysis by adjusting their side chain torsion angles. No density of the inhibitor phenylboronic acid, which was co-crystallized with the enzyme, can be found in the immediate vicinity of these two resides. In the active site cleft, there is a large and open hydrophobic pocket formed by the A and C domains with residues 320, 324, 337, 339, 347, 349, 376, 399, 400, and 419 on one side of the active site. This pocket is large enough to accommodate three or four hydrophobic or neutral side-chains. It is the likely binding site for the P side of the substrates bordering the scissile bond in which the sequences of the first four residues are absolutely conserved. There is a smaller hydrophobic patch, formed by residues 140, 152, 212, 213, and 403, on the other side of the active site. The patch is located on the bottom of the cleft between domains A and B. This part of the cleft is slightly deeper, however. This is likely the potential binding pocket for the p¹ side of the substrate, in which only the P1 and the P2′ residues of the substrate are also hydrophobic.

Analysis of the Surface Properties

The natural substrate of D1 protease is the C-terminal extention of the D1 polypeptide of the PS II reaction center, an integral membrane protein. It is likely that the D1 protease interacts with the membrane to facilitate the binding of substrate. However, electrostatic calculations, using the program MOLMOL (Koradi, R., Billeter, M., and Wüthrich, K., J. Mol. Graphics 14:51-55 (1996)), show no extensive positively charged areas on the protein surface that can be used for interaction with the membrane surface. It also has no large hydrophobic patch outside the active site cleft that can be used as a membrane binding site. This suggests that if the protease interacts with the membrane, the interacting area should be small and local. One possible candidate is a small cluster of four conserved Arg/Lys residues (residues 90, 94, 108 and 110) in the A domain near the putative hydrophobic binding pocket for the P side of the substrate.

Two conserved cysteine residues Cys260 and Cys451 are on the surface of the protein, and adjacent to each other. These two are the only cysteine residues in the Scenedesmus obliquus enzyme. They are also the only conserved cysteine residues among all known eukaryotic D1 proteases. They are remote from the active site cleft, and they form a disulfide bond in the native structure. In the Se-Met mutant structure, the disulfide bond is reduced, since the protein was prepared in the presence of 10 mM of reducing agent DTT. The breakage of this disulfide bond does not affect the enzymatic activity nor does it substantially change the structure of the Scenedesmus enzyme.

Predictive Methods for Ligand Design

The coordinates shown in FIG. 1 define the hydrogen bonding network for the D1 protease Scenedesmus enzyme. This model can be used for visualizing the orientations and interactions of amino acids within the active site for the purpose of designing novel ligands and substrates of the enzyme through the use of computer modeling using a docking program such as GRAM, DOCK, or AUTODOCK (Dunbrack et al., 1997, supra), to identify potential ligands and/or antagonists for D1 protease. This procedure can include computer fitting of potential ligands to the ligand binding site to ascertain how well the shape and the chemical structure of the potential ligand will complement the binding site (Bugg et al., Scientific American December: 92-98 (1993); West et al., TIPS 16:67-74 (1995)). Computer programs can also be employed to estimate the attraction, repulsion, and steric hindrance of the two binding partners (i.e., the ligand-binding site and the potential ligand). Generally the tighter the fit, the lower the steric hindrances, and the greater the attractive forces, the more potent the potential ligand or inhibitor since these properties are consistent with a tighter binding constant. Furthermore, the greater the specificity in the design of a potential ligand the more likely that the ligand will not interact as well with other proteins. This will minimize potential side-effects due to unwanted interactions with other proteins.

Initially potential ligands and/or agonists can be selected for their structural similarity to a known ligand, such as the tetrapeptide chloromethylketone (Z-LDLA-CMK) [SEQ ID NO: 11], where Z=carbobenzoxy, and CMK=chloromethylketone, and LDLA represent the tetrapeptide Leu-Asp-Leu-Ala. The structural analog can then be systematically modified by computer modeling programs until one or more promising potential ligands are identified. Alternatively a potential ligand could be obtained by initially screening a random peptide library produced by recombinant bacteriophage for example, (Scott and Smith, Science, 249:386-390 (1990); Cwirla et al., Proc. Natl. Acad. Sci., 87:6378-6382 (1990); Devlin et al., Science 249:404-406 (1990)). Preferred for use in the present invention is the program Sybyl® (TRIPOS).

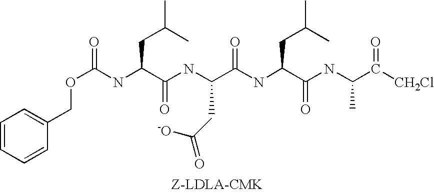

Within the computer program Sybyl® (TRIPOS) ligand molecules may be visualized by using the Build/Edit algorithms to make and break bonds and to add or delete atoms to aid in the design of novel ligands and substrates. The models allow for the visualization of designed or other inhibitors in three dimensions within the active site (after removal of the ligand structures from the models) by using the docking routine within Sybyl® or other such programs to manually position such inhibitors within the active site. After manually docking the ligands the D1 protease-ligand structures may be minimized by using the minimization procedures within Sybyl® in order to improve the models. After deleting the ligand, computer programs such as DOCK® (written by Paul McCloskey, University of California; a WWW site for the DOCK® program may be found at the URL http://www.cmpharm.ucsf.edu/kuntz/dock.html) or UNITY® (TRIPOS) may be used for computer automated dockings of three dimensional libraries of compounds as described in Kuntz, I. D. et al., Acc. Chem. Res. 27:117-123 (1994)) and Kuntz, I. D., Science 257:1078-1082 (1992) which aid in the discovery of novel ligands and substrates. Such programs apply constraints imposed by the enzyme active site and other constraints imposed by the user for computer generation of three dimensional sub-structures which are useful for searching through three dimensional data bases. The models lacking ligands using coordinates as displayed in FIG. 1 (for example) may be applied to computer programs such as Leapfrog® (TRIPOS) for building virtual molecules within the active site from small three dimensional molecular fragments for the purpose of discovering new ligands and substrates of the enzyme. Sybyl®, DOCK®, UNITY®, Leapfrog® and other such computer programs can calculate an approximate binding energy for each of the molecules docked thus allowing the user to select favorable molecules for synthesis and substrate analysis against the activity of the enzyme. Useful ligands of D1 protease discovered by these enablements may be evaluated for their ability to inhibit the enzyme.

### EXAMPLES

### General Methods

Standard recombinant DNA and molecular cloning techniques used here are well known in the art and are described by Sambrook et al. (1989), J., Fritsch, E. F. and Maniatis, T. Molecular Cloning: A Laboratory Manual, Cold Spring Harbor Laboratory Press, Cold Spring Harbor, 1989 (hereinafter “Maniatis”); and by T. J. Silhavy, M. L. Bennan, and L. W. Enquist, Experiments with Gene Fusions, Cold Spring Harbor Laboratory Press, Cold Spring, N.Y. (1984) and by Ausubel et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiolog, Second Edition, Sinauer Associates, Inc., Sunderland, Mass. (1989). All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

Manipulations of genetic sequences were accomplished using the suite of programs available from the Genetics Computer Group Inc. (Wisconsin Package Version 9.0, Genetics Computer Group (GCG), Madison, Wis.). Where the GCG program “Pileup” was used the gap creation default value of 12, and the gap extension default value of 4 were used. Where the CGC “Gap” or “Bestfit” programs were used the default gap creation penalty of 50 and the default gap extension penalty of 3 were used. In any case where GCG program parameters were not prompted for, in these or any other GCG program, default values were used.

The meaning of abbreviations is as follows: “sec” means second(s), “min” means minute(s), “h” means hour(s), “d” means day(s), “μL” means microliter(s), “mL” means milliliter(s), “L” means liter(s), “mM” means millimolar, “M” means molar, “mmol” means millimole(s).

Plasmids and Bacterial Strains:

Plasmids: Scenedesmus obliquus D1P insert in PET-32a expression vector

Bacteria host strain: BL21(DE3)plysS

Media and Buffers:

Media:

LB medium

M9 complete medium:

2×M9 salts

2 mm MgSO₄

25 μg/ml FeSO₄-7H₂O

0.4% glucose

40 μg/ml Amino Acid mix I

40 μg/ml Amino Acid mix II

1 μg/ml vitamin mix

2-20 μg/ml uracil

40 μg/ml L-methionine or L-seleno-methionine

pH˜7.0

Stock solutions for preparing M9 complete medium 20×M9 salts:

10 g NH₄Cl

30 g KH₂PO₄

68 g Na₂HPO₄ or 128 g Na₂HPO₄-7H₂O

add H₂O to 500 mL

Amino Acid mix I:

16 amino acids, each at 4 mg/mL

excluding Met, Tyr, Trp, Phe

Amino Acid mix II:

3 amino acids, each at 4 mg/mL

Tyr, Trp, Phe

Tyr is hard to dissolve, add last

final solution may still be turbid, resuspend well before use

L-methionine or L-seleno-methionine: 10 mg/mL

Uracil: 2 mg/mL, dissolve in 65° C. H₂O

Glucose: 20%

MgSO₄: 1M

FeSO₄-7H₂O: 12.5 mg/mL

Vitamin mix: each at 1 mg/mL, store at −20° C. riboflavin, niacinamide, pyridoxine monohydrochloride, thiamine riboflavin may not dissolve completely, filter the mix

Buffers:

Lysis buffer:

20 mM HEPES pH 7.2

1 mM EDTA

5 mM MgCl₂

0.1% Triton X-100

0.1 mg/mL lysozyme

0.01 mg/mL RNAse

0.05 mg/mL DNAse

Denaturing buffer:

8 M guanidinine hydrocloride

20 mM HEPES pH 7.2

1 mM EDTA

5 mM DTT, freshly added

100 μM PMSF, freshly added

Inclusion body wash buffer:

20 mM HEPES pH 7.2

1 mM EDTA

0.1% Triton X-100

0.3 M NaCl

Refolding Buffer:

20 mM MES pH 6.0

10% Glycerol

10 mM CHAPS

1 mM EDTA

1 mM GSSG

1 mM GSH

100 μM PMSF

Wash Buffer:

20 mM pH 6.5

10% Glycerol

10 mM CHAPS

1 mM EDTA

100 μM PMSF

MonoQ Buffer:

Buffer A:

20 mM MES pH 6.5

10% Glycerol

10 mM CHAPS

1 mM EDTA

100 μM PMSF

Buffer B:

20 mM MES pH 6.5

10% Glycerol

10 mM CHAPS

1 mM EDTA

100 μM PMSF

1 M NaCl

TSK Buffer:

20 mM HEPES pH 7.2

10% Glycerol

10 mM CHAPS

1 mM EDTA

100 μM PMSF

Buffer modifications for L-seleno-methionine labeled protein:

10 mM DTT was added into all buffers

### Example 1

Cloning Scenedesmus obliquus D1 Protease Gene for Expression

The polymerase chain reaction (PCR) was used to amplify the coding region for the mature D1 protease, by simultaneously using as template the overlapping 5′ Race and 3′ Race PCR products described in Trost et al. (J. Biol. Chem. 272:20348-20356 (1997)). The 5′ primer sequence was ATG ACC ATG GTG ACA AGC GAG CAG CTG CTG TT (SEQ ID NO: 2) and contained an Nco1 site, while the 3′ primer sequence was AGC TGA TGC GGA TCC TTA CCC AAA CAG CCG CGG CGC A (SEQ ID NO: 3) and contained a BamH1 site. The resulting 1.2 kb product was initially ligated into the pGEM-t vector (Promega, Madison Wis.) and transformed into Escherichia coli, which was plated on LB ampicillin. Plasmid DNA was recovered from selected colonies using the Promega Wizard miniprep kit, and then digested with Nco1 and BamH1 restriction enzymes to excise the D1 protease gene fragment. This fragment was ligated into the expression vector pET-32a (Novagen). It should be noted that cloning into the pET-32a vector resulted in the expression of a fusion protein consisting of thioredoxin plus two affinity tags linked to mature D1 protease. Cleavage of the fusion by enterokinase results in a mature D1 protease (D1 protease (+AM)) that is longer by two amino acids (alanine+methionine) than the native mature protein (SEQ ID NO: 10). Nucleotide sequencing was used to confirm the wild type sequence.

### Example 2

Site-Directed Mutagenesis

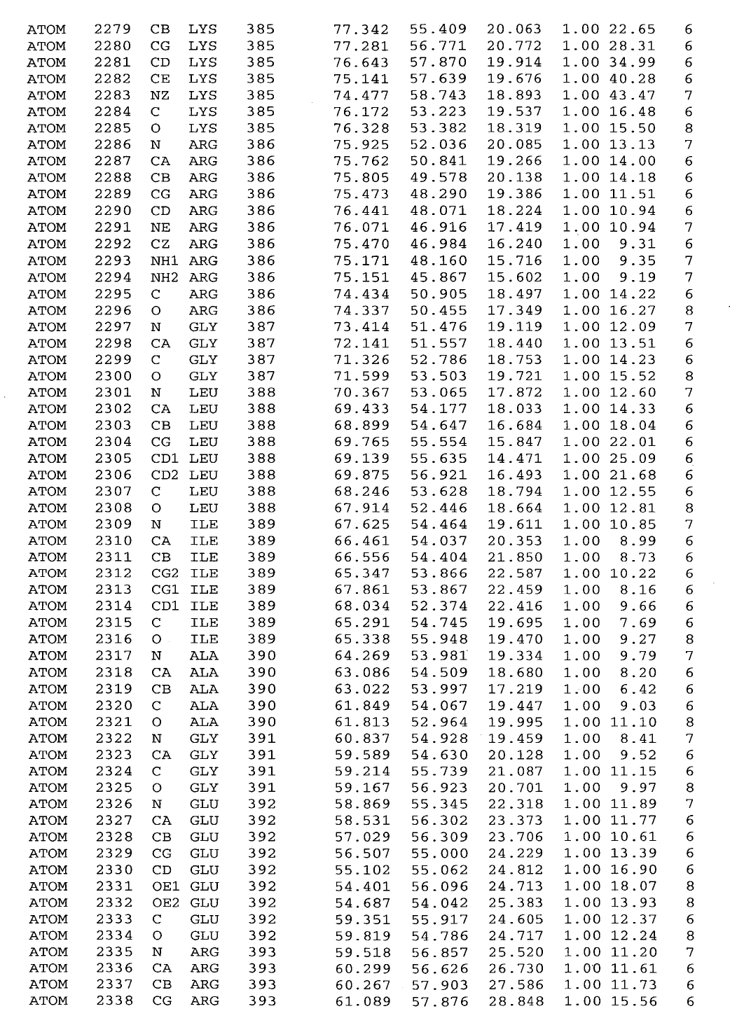

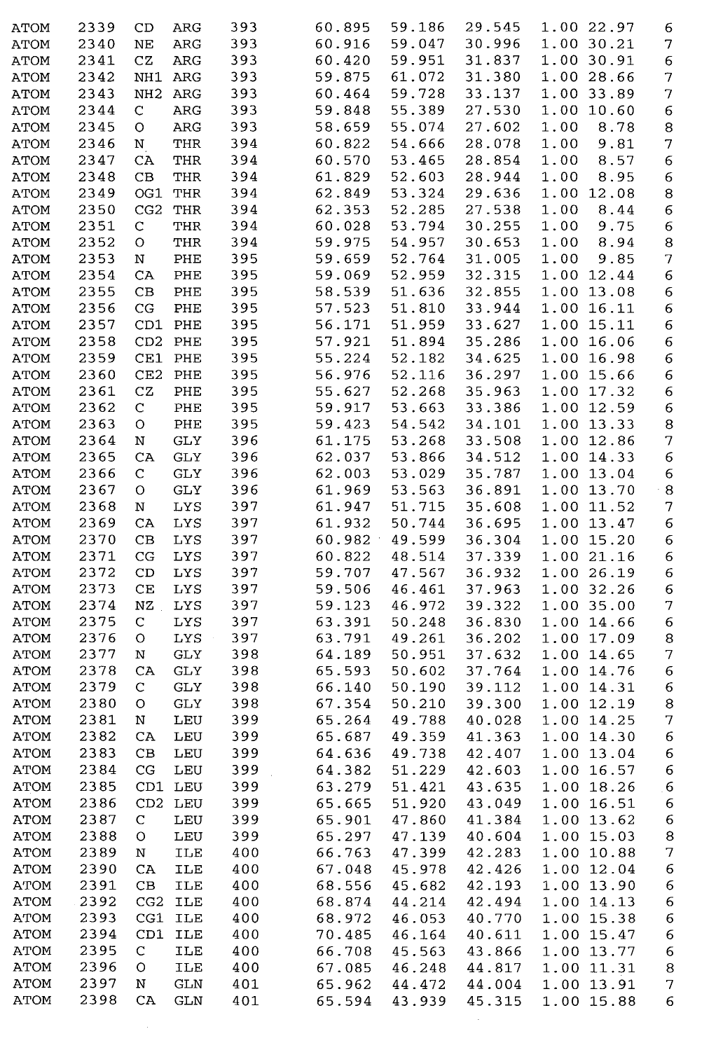

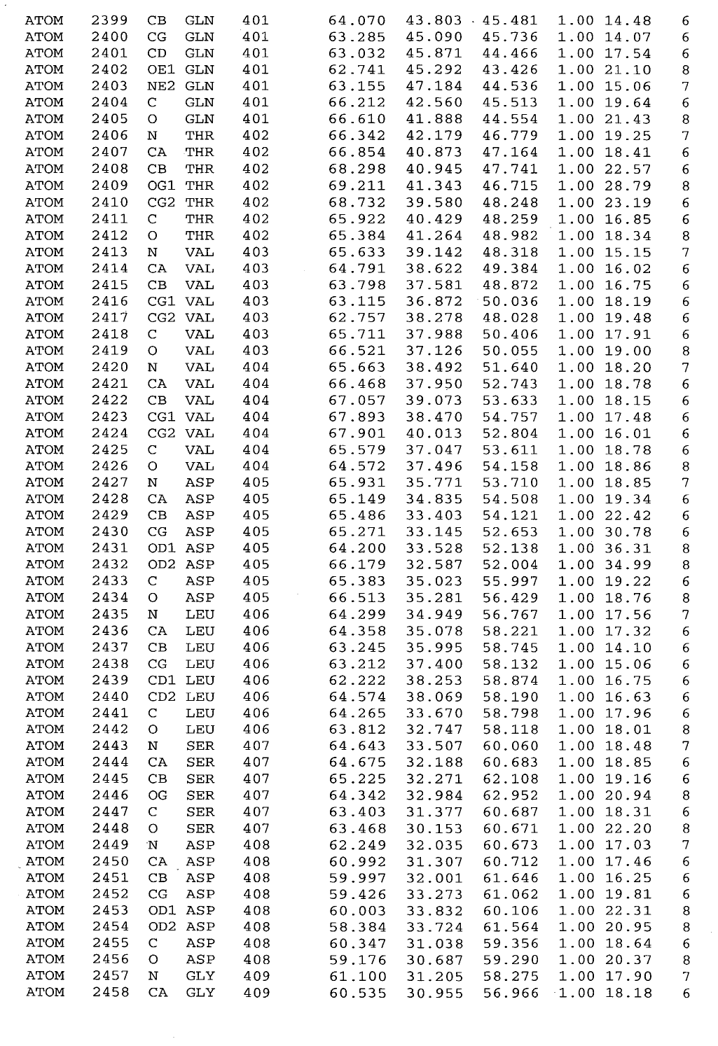

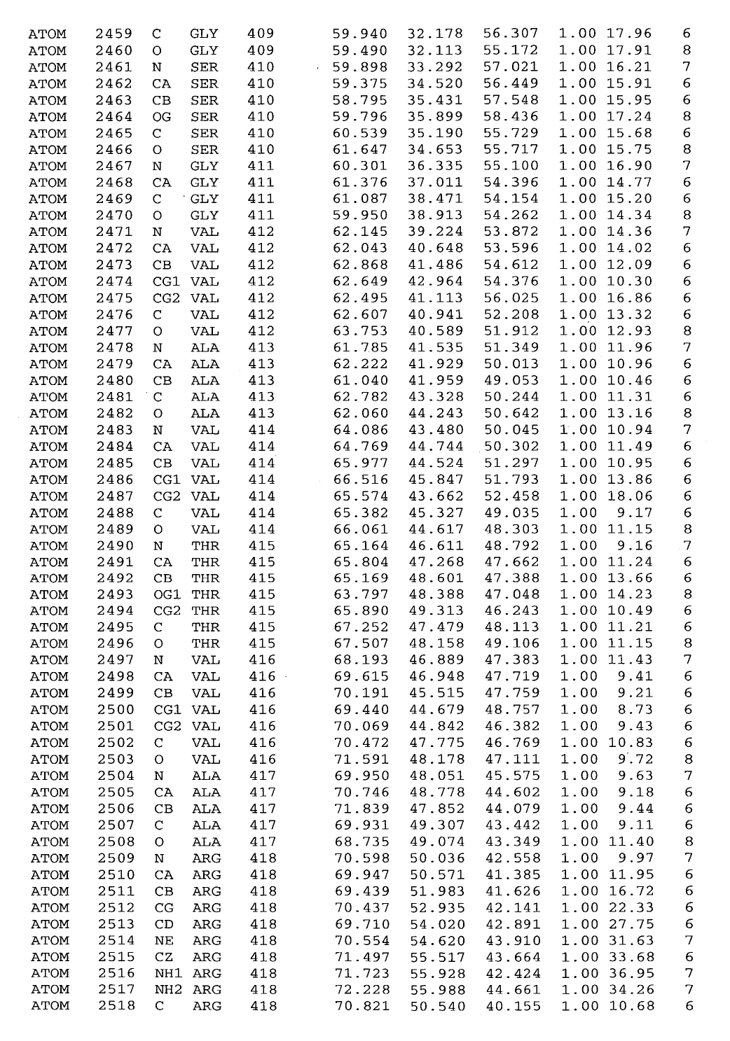

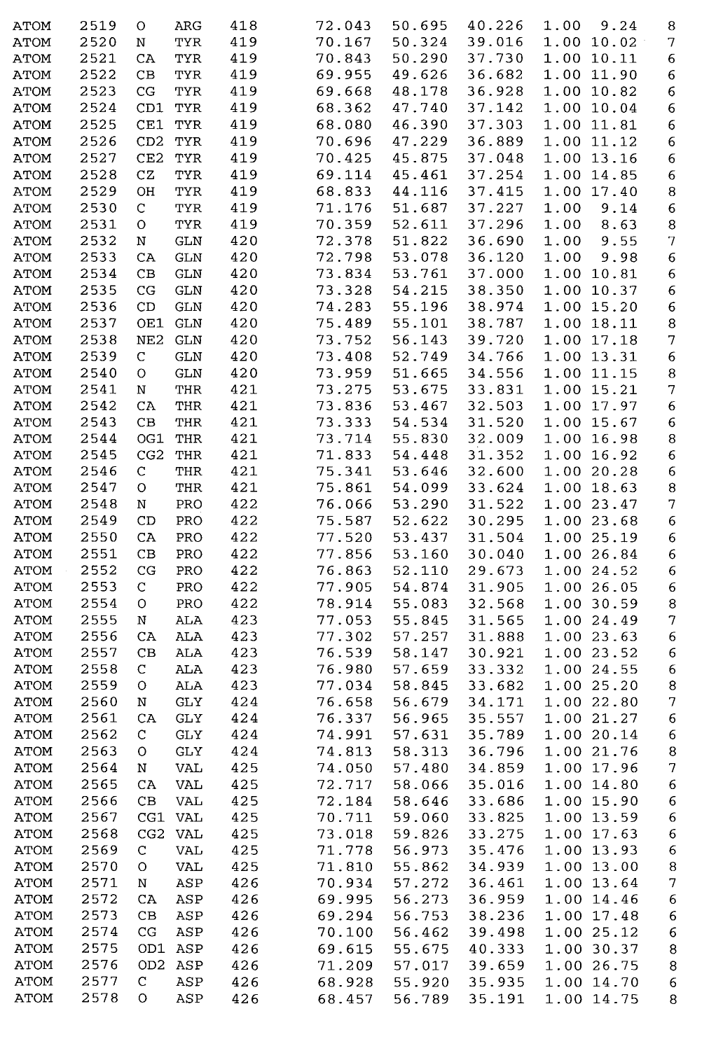

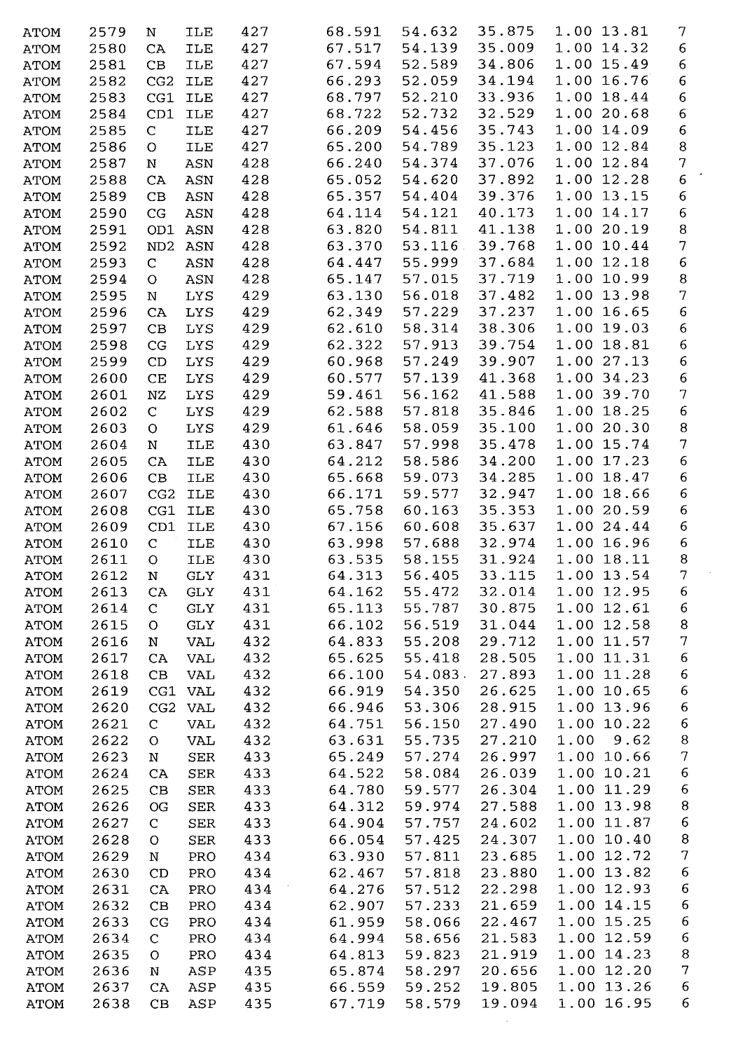

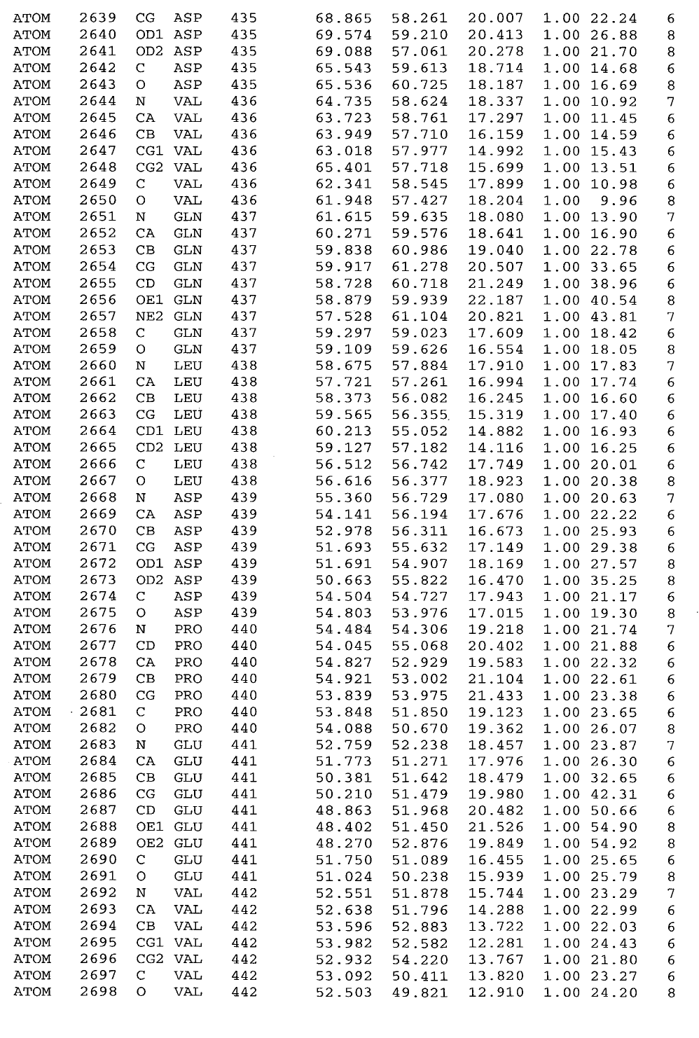

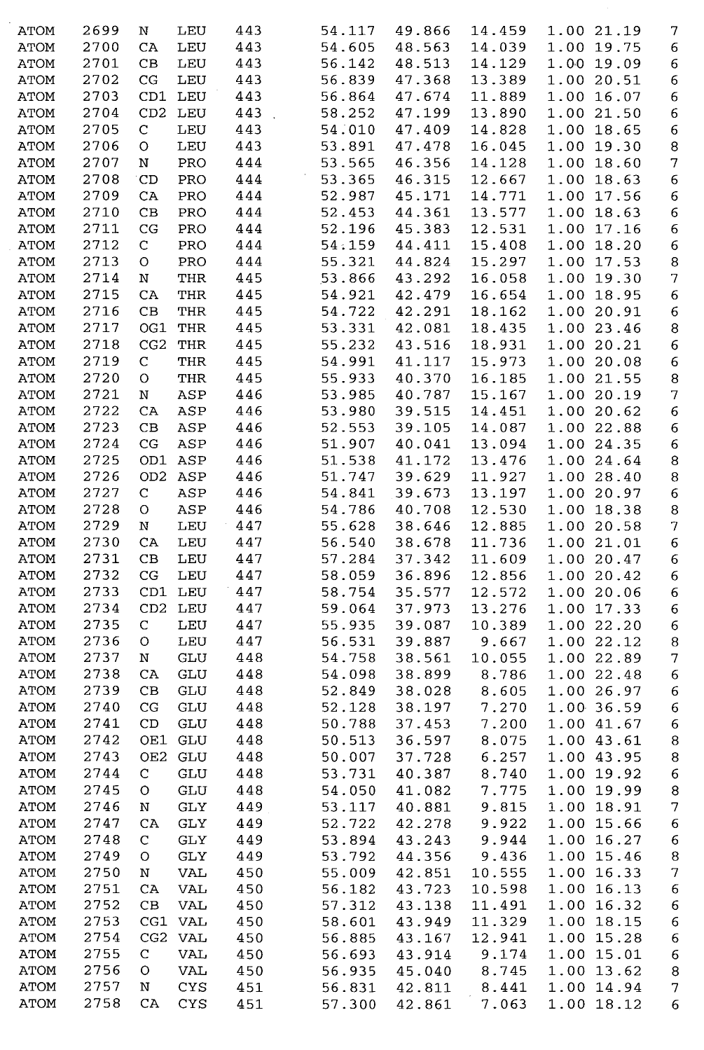

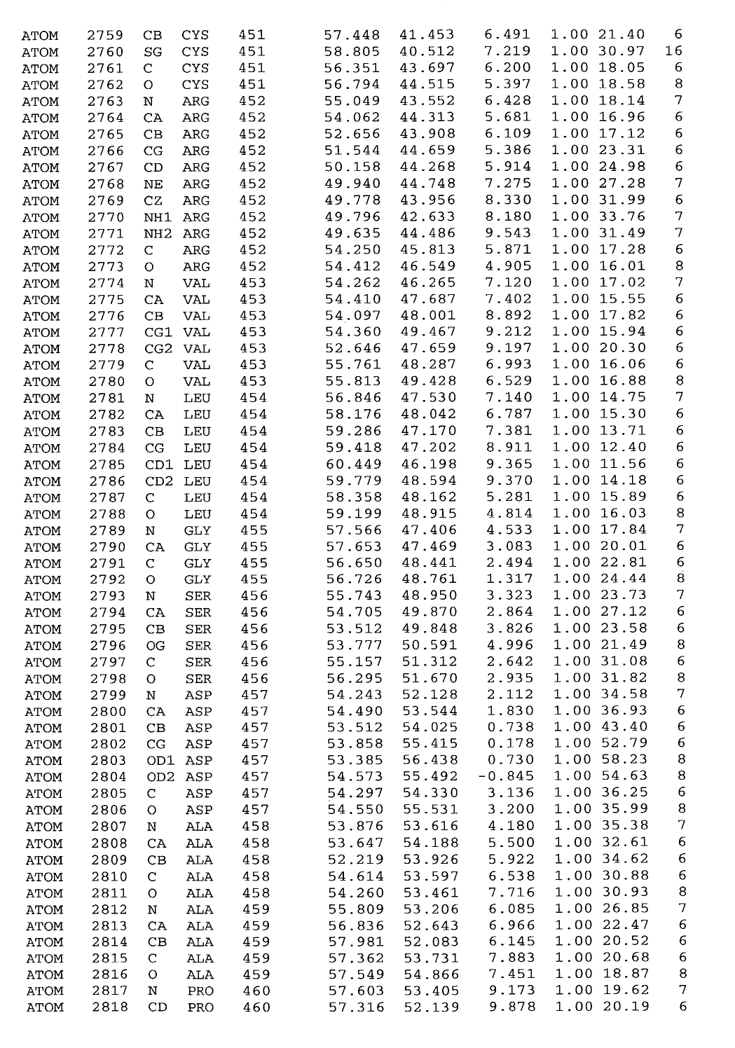

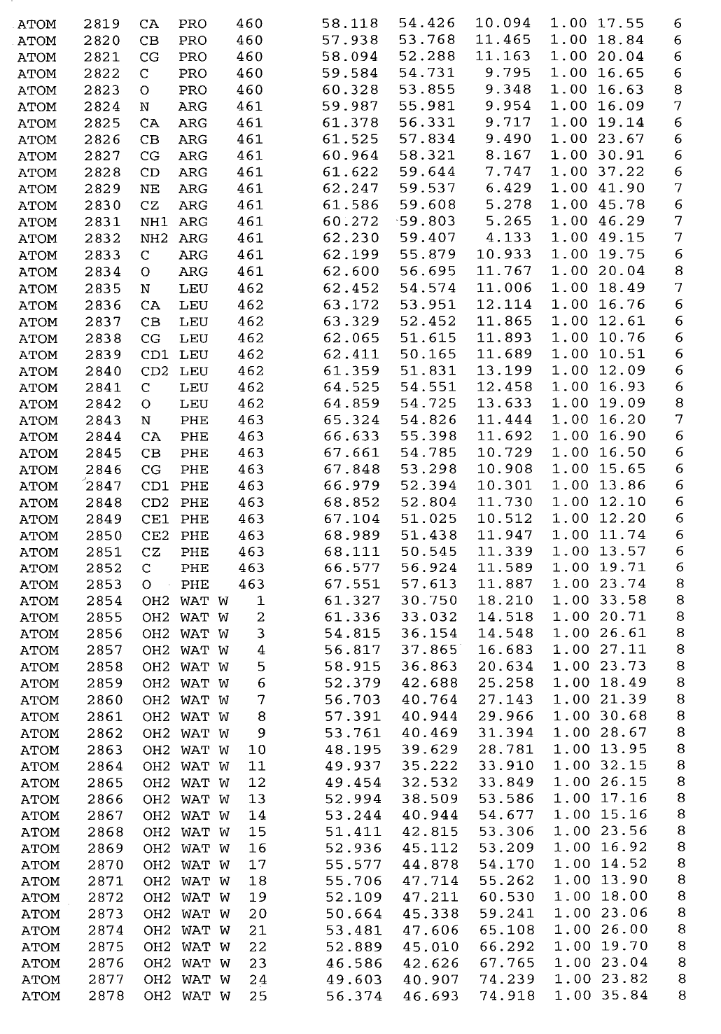

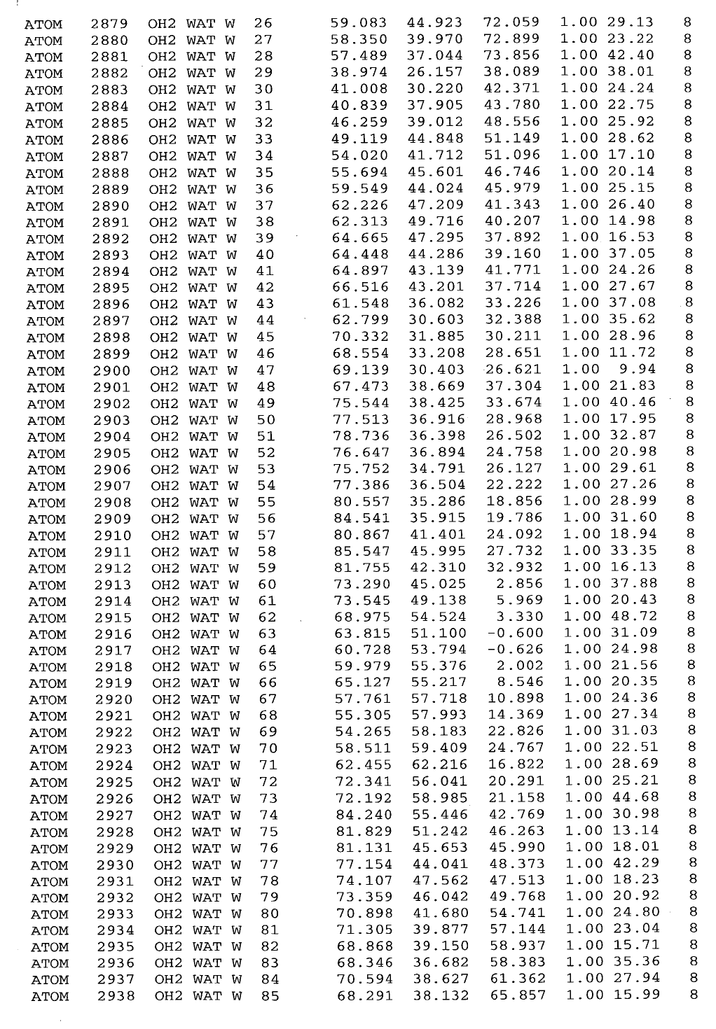

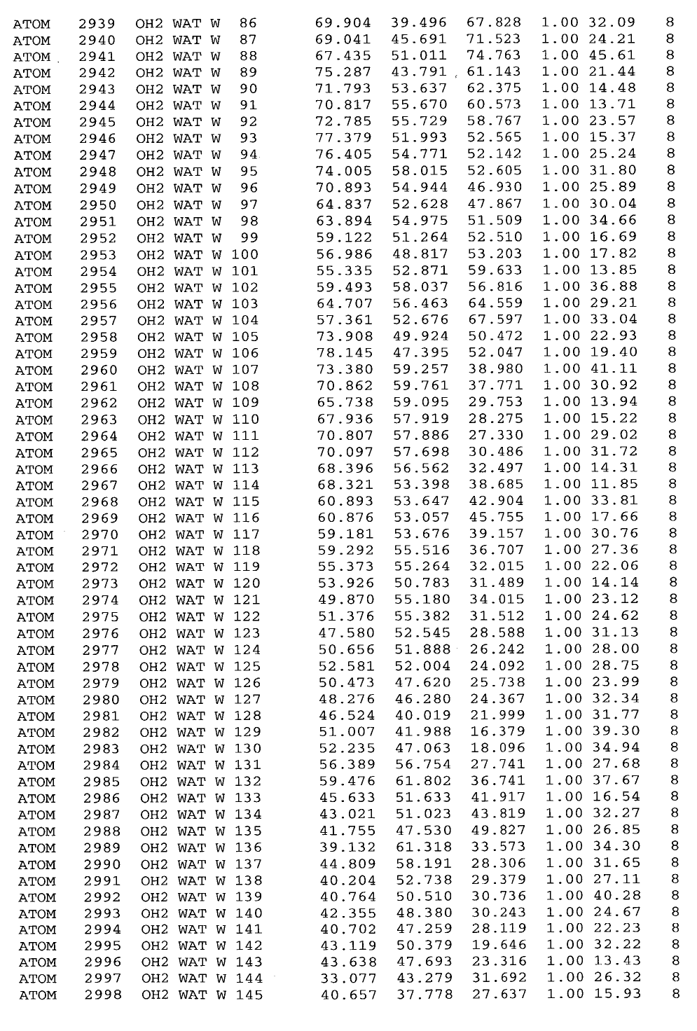

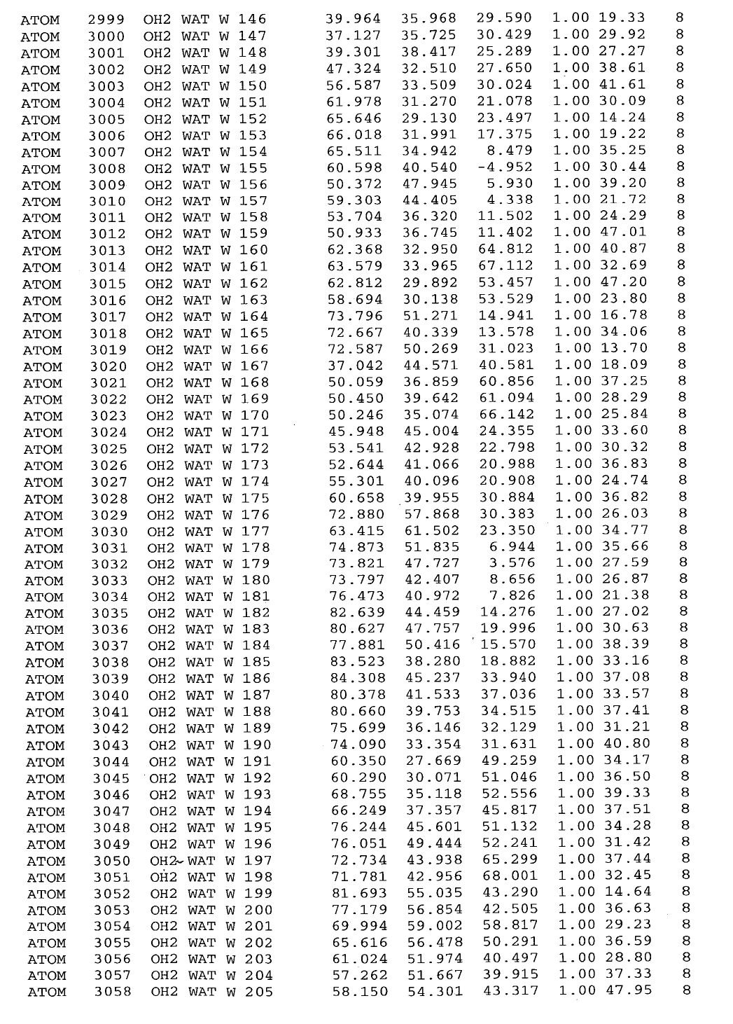

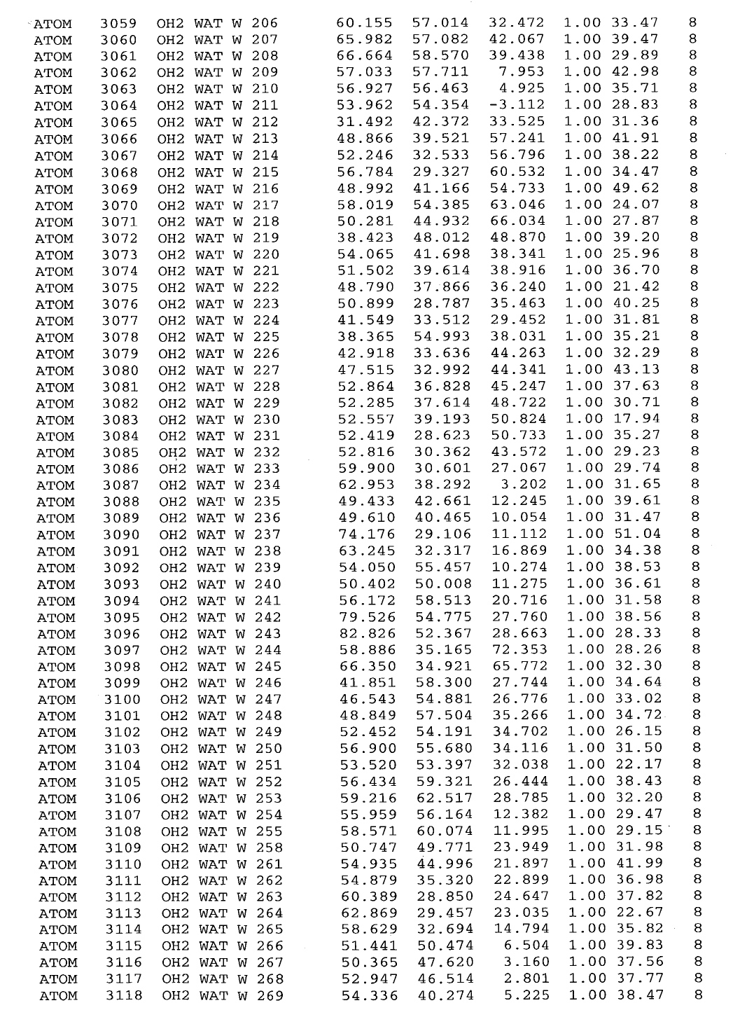

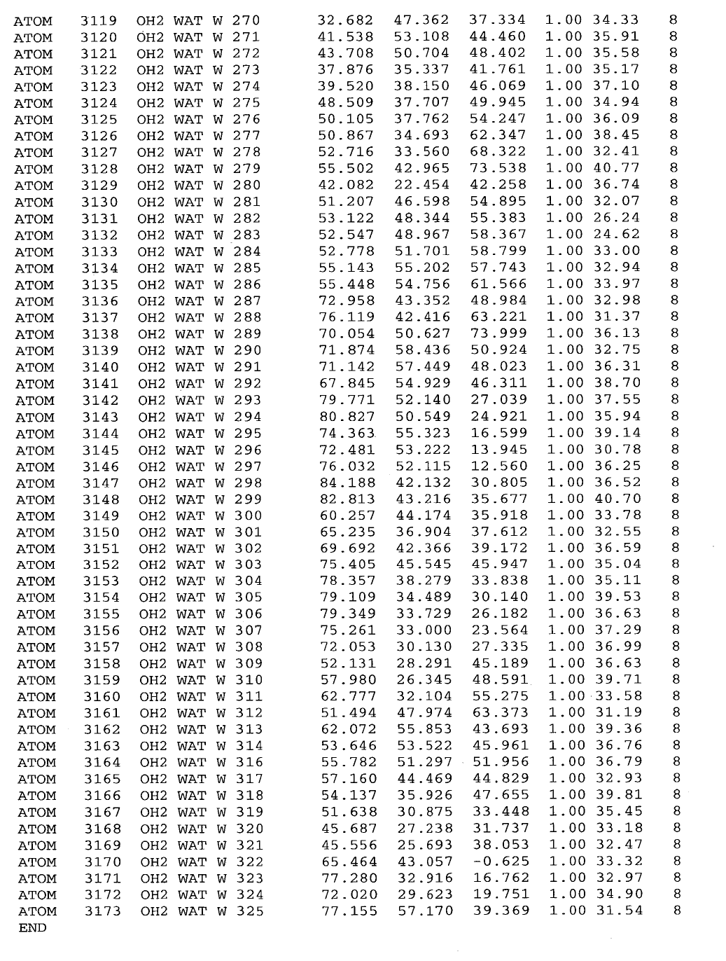

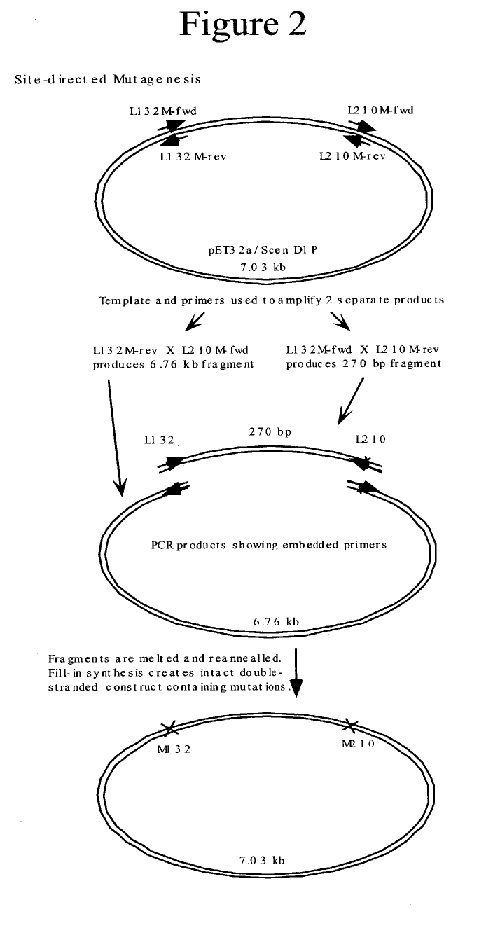

MAD (Multiwavelength Anomalous Diffraction), using the selenium K-edge, was used for solving the crystallographic phase problem. Ideally, MAD phasing requires the presence of at least one seleno-methionine per 10 kDa of protein mass. As the wild type D1 protease (+AM) contains only three methionines, it was decided to add two additional ones to the protein (SEQ ID NO: 10). Site-directed mutagenesis was used to replace codons Leu57 (corresponding to Leu132 of SEQ ID NO: 1) and Leu135 (corresponding to Leu210 of SEQ ID NO: 1) with methionine codons, giving the polypeptide as set forth in SEQ ID NO: 4. These leucines were chosen because there are methionines located in these positions in higher plant versions of the D1 protease (e.g. spinach, wheat and tobacco). The mutated protease would then contain five methionines per 40.8 kDa, suitable for MAD phasing using seleno-methionine. The mutations were simultaneously introduced using a procedure involving PCR, reannealing, and fill-in synthesis (FIG. 2). The primers GAT GCC ATC CGC AAG ATG CTG GCG GTG CTG GAC (L132M-fwd; SEQ ID NO: 5) and GTC CAG CAC CGC CAG CAT CTT GCG GAT GGC ATC (L132M-rev; SEQ ID NO: 6) were used to modify L132, while the primers ACG GCT GTG AAG GGG ATG TCG CTG TAT GAC GTG (L210M-fwd; SEQ ID NO: 7) and CAC GTC ATA CAG CGA CAT CCC CTT CAC AGC CGT (L210M-rev; SEQ ID NO: 8) were used to modify L210. The mutagenic PCR was done in two separate reactions, using as template the pET-32a-D1P(+AM) protease expression construct described above. Oligonucleotide primers, L132M-fwd (SEQ ID NO: 5) plus L210M-rev (SEQ ID NO: 8), produced a 270 bp fragment. Oligonucleotide primers, L132M-rev (SEQ ID NO: 6) and L210M-fwd (SEQ ID NO: 7) produced a 6.76 kb fragment, which included the vector sequence. The two fragments were combined, melted, and annealed so as to prime each other for synthesis of a complete 7.03 kb construct. The synthesis reaction contained 7.5 units Pfu polymerase, 1×reaction buffer (Stratagene) and 5 μL 10 mM nucleotide stock (Stratagene) in a volume of 50 μL. The reaction mix was held at 72° C. for 30 min to allow for polishing of 3′ extensions, then cycled once at 94° C. for 1 min, 60° C. for 30 sec and 68° C. for 20 min. Ten μL of the synthesis reaction was used to transform XL1-blue host cells which were plated on LB ampicillin. Six colonies were picked for sequence verification. All contained the desired mutations.

### Example 3

Expression of Scenedesmus obliquus D1 Protease

The Escherichia coli host expression strain BL21(DE3)plysS (Novagen) was transformed using plasmid pET-32(a)-D1P(+AM) according to standard protocols (Novagen). The transformed cells were plated on solid LB medium containing 150 μg/mL ampicillin and incubated overnight at 37° C. A single colony containing the mature wild-type Scenedesmus obliquus D1 protease expression clone (+AM) was inoculated into 250 mL LB medium plus carbanecillin (100 μg/mL) and incubated at 37° C. overnight on a rotary shaker. The overnight culture was used to inoculate 9.75 L fresh LB medium plus carbanecillin in a 10-L fermentor. Once the optical density reached 0.4-0.5 at 600 nm, 1 mM IPTG (isopropyl-β-D-thiogalactopyranoside) was added to induce expression. After 2.5-3 h of induction at 37° C., the cells were harvested by centrifugation at 8000 rpm using a GSA rotor (Sorvall), frozen in liquid nitrogen and stored at −75° C. The 10-L culture yielded about 25 g of wet cell paste.

To obtain L-seleno-methionine labeled protein, a single colony of BL21(DE3)plysS(met⁻), bearing expression vector with mutated (Leu132 and 210 replaced by Met) mature Scenedesmus obliquus D1 protease (+AM), was inoculated into 20 mL M9 complete medium containing L-methionine (40 μg/mL) plus 100 μg/mL carbanecillin. The culture was incubated at 37° C. overnight on a rotary shaker. The bacteria were then collected, washed and resuspended in 20 mL M9 complete medium without L-methionine. Two liters of M9 complete medium containing L-seleno-methionine (40 μg/mL) and 100 μg/mL carbanecillin were inoculated with the washed bacteria. The two liters were distributed equally among four 6-L flasks. The cells were grown at 37° C. until the OD₆₀₀ reached 0.6. Protein expression was then induced with 1 mM IPTG at 37° C. and allowed to continue overnight. The cells were harvested by centrifugation at 8000 rpm using a GSA rotor (Sorvall). Approximately 5 g wet weight bacteria paste/2 L culture was collected.

### Example 4

Inclusion Body Isolation

Bacterial cell paste was resupended in Lysis buffer (1 g wet weight cells/2 mL Lysis buffer) and incubated on ice for 15 min. The lysate was sonicated (Branson Sonifier cell disruptor 185) for 1 min on ice to ensure complete lysis. Following sonication, the lysate was incubated on ice for another 30 min with occasional mixing, and centrifuged at 20,000×g for 20 min. The pellet containing inclusion bodies was collected and washed with Inclusion body wash buffer for at least 5 times before the pellet was solubilized with Denaturing buffer.

### Example 5

Refolding of Solubilized Fusion Protein

Fifty mL of fusion protein, solubilized in Denaturing buffer (OD₂₈₀=1), was added while stirring to 1 L of Refolding buffer at a rate of 0.1 mL/min at 4° C. The Refolding buffer+protein was then left to stir overnight at 4° C.

### Example 6

Sample Preparation and Chromatography Purifications

The Refolding buffer+protein was concentrated to 50 mL and washed with MonoQ buffer A to lower the guanidinium hydrochloride concentration to less than 10 mM. The concentrated and washed fusion protein was loaded onto an HR10/10 MonoQ column (Pharmacia) preequilibrated with MonoQ buffer A. The protein was eluted using a 0-1 M NaCl linear gradient elution. The active fusion protein peak eluting at 90 mM NaCl was pooled, concentrated and digested with recombinant enterokinase (Novagen) at a concentration of 1 unit/300 μg fusion protein to release the mature Scenedesmus obliquus D1 protease (+AM). The recombinant protease (D1 protease (+AM)) contains two additional amino acids (Ala and Met) at its N-terminus as compared to the natural mature D1 protease. The extra residues have no effect on enzyme activity. The products of the overnight digestion were then desalted on a BioRad Econo-Pac 10DG column and loaded onto a MonoQ HR10/10 column preequilibrated with the MonoQ Buffer A. Gradient elution proceeded as with the fusion protein except that the mature polypeptide eluted at 78 mM NaCl. The active fractions were pooled and concentrated to less than 500 μL for size exclusion chromatography on a G-2000SW TSK-gel column (TosoHaas). The active mature Scenedesmus obliquus D1 protease (+AM) fractions were pooled, concentrated to 3.5 mg/mL in an Amicon concentrator cell (YM30 membrane), frozen in liquid nitrogen and stored at minus 75° C.

### Example 7

Preparation of D1 Protease for Crystallography

The concentrated Scenedesmus obliquus D1 protease (+AM) protease was diluted 40-fold into 20 mM HEPES-NaOH, pH 7.5 plus 1 mM phenylboronic acid and concentrated back to 50 μL using a Centricon 30 concentrator (Millipore). This enzyme was then used as is for crystallization trials.

### Example 8

Crystallization of D1 Protease from Scenedesmus obliquus

Single crystals of D1 protease from Scenedesmus obliquus were obtained at room temperature (˜20° C.) by vapor diffusion in hanging drops. The hanging drop experiments were set up on Q plate II multi-well trays from Hampton Research. The crystallization drops consist of 1 μL of 3.5 mg/mL protein in 20 mM HEPES pH 7.5 and 1 mM phenylboronic acid, and 1.0 μL of reservoir solution. Each drop was mixed on a siliconized glass cover slip. The cover slip was inverted and placed over a reservoir containing 0.5 or 1.0 mL of reservoir solution. The crystallization tray was then sealed with clear tape. Crystals were obtained from two different conditions. The reservoir solution in condition number one contains 17-18% PEG 4K, 10% isopropanol and 0.1 M HEPES pH 7.5. The reservoir solution in condition number two contains a mixture of 30-40% saturated ammonium sulfate and 10-20% of 2 M lithium sulfate. Two crystal forms with the same space group C2 and slightly different cell dimensions were obtained from condition number one. Form C2 has the cell dimensions of a=110.9 Å b=64.05 Å c=63.4 Å and β=122.0°; form C2 II has the dimensions of a=108.6 Å b=63.12 Å c=60.68 Å and β=119.8°. The diffraction limit for both of them is 1.8 Å. These crystals were transferred to stabilizing solution containing 20% PEG4000 10% isopropanol, 0.1 M HEPES pH 7.5 and 20% glycerol prior to data collection at cryo-temperature. The crystals were either fresh frozen in liquid propane or in a minus 170° C. cryo-stream. The crystals obtained from condition number two have the space group of R32 and cell dimensions of a=b=148.7 Å, c=100.31 Å using hexagonal indexing. The crystals were quickly washed in the solution containing 45% saturated ammonium sulfate, 10% of 2 M lithium sulfate and 20% glycerol right before being put in the minus 160 or the minus 170° C. cold nitrogen stream for data collection.

### Example 9

Data Collection and Structure Determination of L132M/L210M Mutant of Scenedesmus obliquus D1 Protease

The structure of L132M/L210M mutant of Scenedesmus obliquus D1 protease has been solved to 2.2 Å resolution by selenomethionine mutliwavelength anomalous diffraction (MAD) method (Hendrickson, W. A., Horton J. R., LeMaster D. M., EMBO J. 9:1665-1672 (1990)). The native enzyme has only three methionines, including one at the N-terminus. The double mutant was designed and created to generate additional selenium sites in order to augment the MAD signal for structure determination. The Se-Met mutant was crystallized in conditions close to those of the native enzyme, in the presence of 0-0.5% percent BME or 0-5 mM DTT. MAD data sets were collected at the APS 5-ID beam line. The exact anomalous absorption edge of the Se-Met protein crystal used for data collection was determined by X-ray fluorescence measurement using an AMPTEK detector. A four-wavelength MAD data set at the wavelengths of the inflection point (0.97891 Å), the peak (0.97876 Å), high remote (0.96369 Å) and low remote (0.99462 Å) of the anomalous absorption spectrum was collected at a temperature of minus 160° C., using a MAR CCD detector. The entire four-wavelength data set was collected from one C2 I form crystal. A data set of 100% completeness at a resolution of 1.8 Å was collected for each wavelength. These data were processed with the program DENZO/SCALEPACK (Otwinowski, Z., “Oscillation Data Reduction Program,” in, Data Collection and Processing, Sawyer, L., Isaccs, N. and Bailey S., eds, pp. 56-62 (1993), SECR Daresbury Laboratory, Warrington, UK). The data set of each wavelength was processed twice, one with each Friedel pair merged and one with each Friedel pair as two independent reflections. The crystal used for this data collection was form C2 I.

The locations of four of the selenium sites were solved by direct method with the program SHELX 97 (Sheldrick, G. M., “Location of Heavy Atoms by Automated Patterson Interpretation,” in, Direct Methods for Solving Macromolecular Structures, Fortier, S., ed., pp. 131-141 (1998), Dordrecht: Kluwer Academic Publishers). The phase problem was solved using the program PHASES (Furey, W. and Swaminathan, S., Am. Crystl. Assoc. Mtg. Abstr. PA33:18:73 (1990)) by treating the MAD data as a special case of multiple isomorphous replacement (MIR) (Ramakrishnan, V. and Biou, V. Methods Enzymol. 276:538-557 (1997)) problem. The dispersion component of the difference in anomalous scattering was isolated by calculating the difference amplitude of the same reflection measured at different wavelengths. Data used for this calculation were processed with the Friedel pair merged. The differences in the dispersion between the wavelengths were used as isomorphous differences in the phase refinement and calculation. The absorption component was isolated by measuring the difference between the two reflections of the Friedel pair in a data set with each Friedel pair treated as two independent reflections. These were used as the anomalous differences in the phase refinement and calculation. The data set of low-remote wavelength showed no anomalous scattering signal, dispersion or absorption, and was used as native. Local scaling implemented in the program PHASES was used for scaling data sets of other wavelengths to the native for isomorphous phase refinement. The positions, isomorphous occupancies, anomalous occupancies and B factor of the four selenium sites were refined using maximum likelihood refinement. A set of protein phases were derived from these refined parameters. The resulting Fourier map was then modified by solvent flatting, histogram matching and Sayer's equation, using program DM (Cowtan K., Joint CCP4 (1994) and ESF-EACBM Newsletter on Protein Crystallography 31:34-38) in the CCP4 package (Collaborative Computational Project Number 4, “The CCP4 Suite: Programs for Protein Crystallography”, Acta. Crystallogr. D50:760-763 (1994)). The modified map was of superior quality and allowed one to build the main-chains and side-chains with great confidence. Densities corresponded to a large number of water molecules can also be seen in this map. The map was displayed and the three dimensional model was constructed using the computer graphics program O (Jones et al., Acta. Crystallogr. A47:110-119 (1991)) on a Silicon Graphics R10000 computer.

### Example 10

Refinement of L132M/L210M Mutant of Scenedesmus obliquus D1 Protease

The initial structure was refined with X-PLOR (Brunger, et al. Science (1987) 235:458-460), using 90% of the data between 10.0 and 1.8 Å for which F>2 σ |F|. A free R factor was calculated for the remaining 10% of the data at each refinement cycle. A total of four cycles of refinement was carried out. Each cycle consists of simulated annealing using the slow-cooling protocol of X-PLOR, restrained B-factor refinement and manual model adjustment using program O (Jones et al., Acta Crystallogr. A47:110-119 (1991)). Water molecules were incorporated into the model at cycles 2-4 by inspecting the Fo-Fc map contoured at 3.5 σ after each cycle. At the last cycle of refinement only the data between 6.0 and 1.8 Å were used. The final data set is shown in FIG. 1.

The current model contains 385 residues, out of the total of 389 and 325 water molecules. Only three residues in the N-terminal and one in C-terminal are missing from the model. The working R factor for this model is 18.6% and the free R factor is 24.5% for 34125 reflections used for the refinement. The rms deviations from ideal values for bond lengths and bond angles are 0.009 Å and 1.486 degrees.

### Example 11

Structure of Native Scenedesmus obliquus D1 Protease Structure (Crystal Forms C2 I)

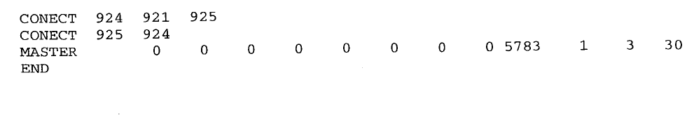

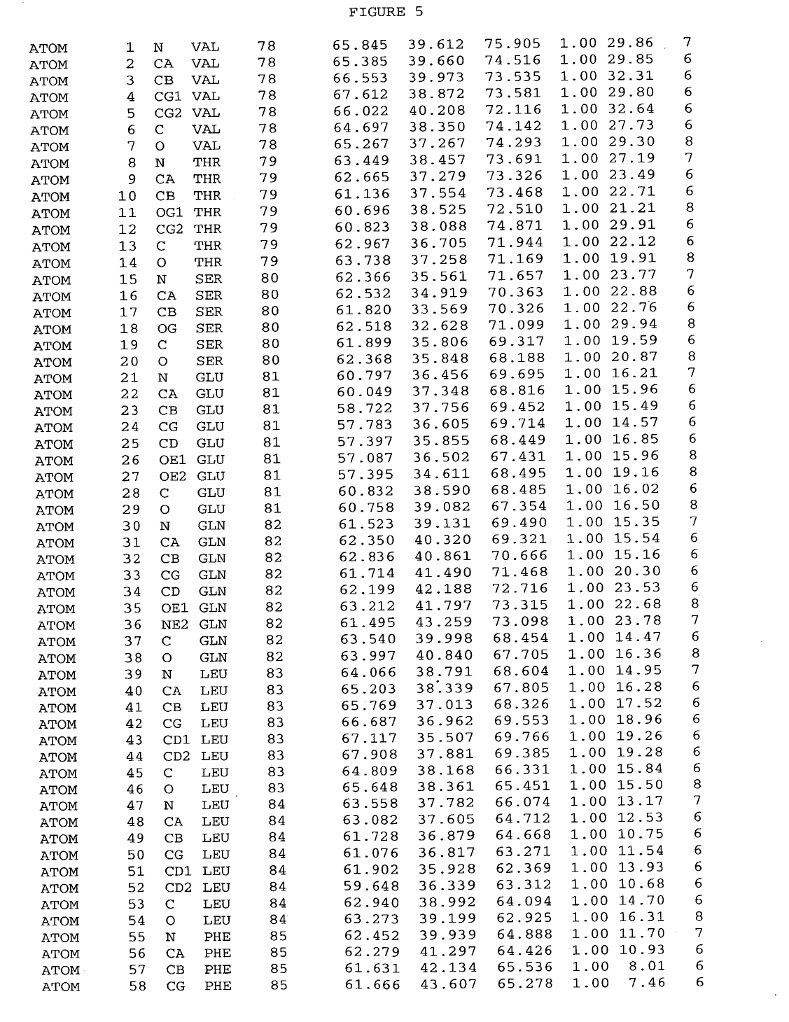

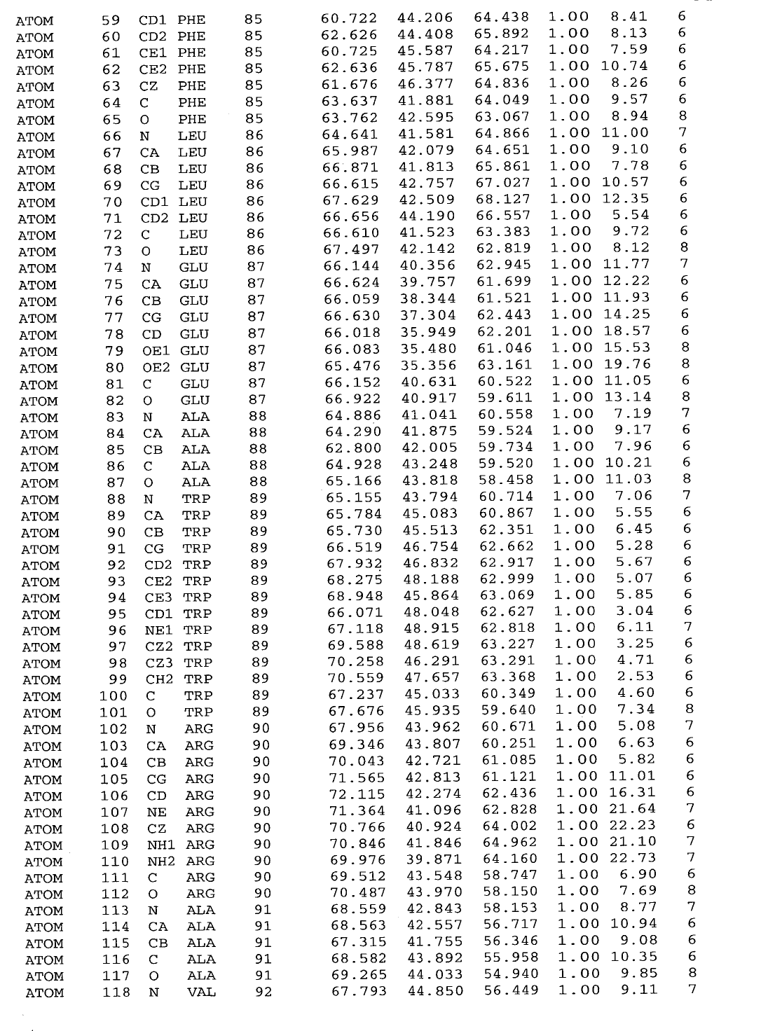

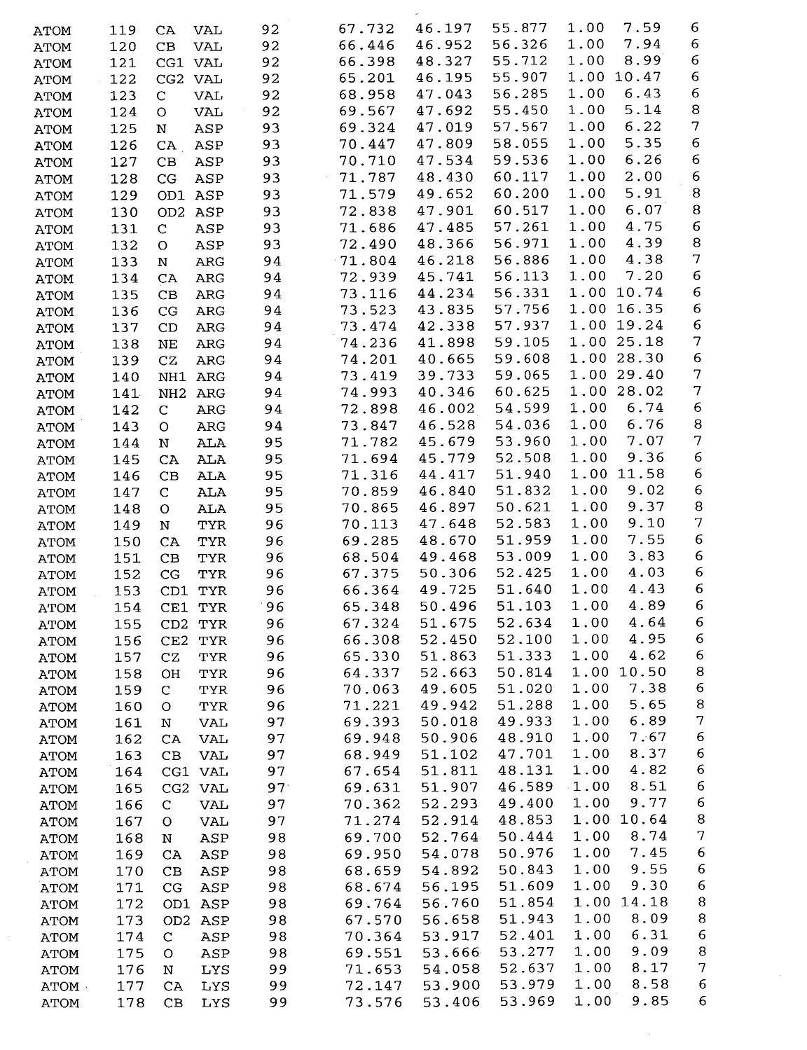

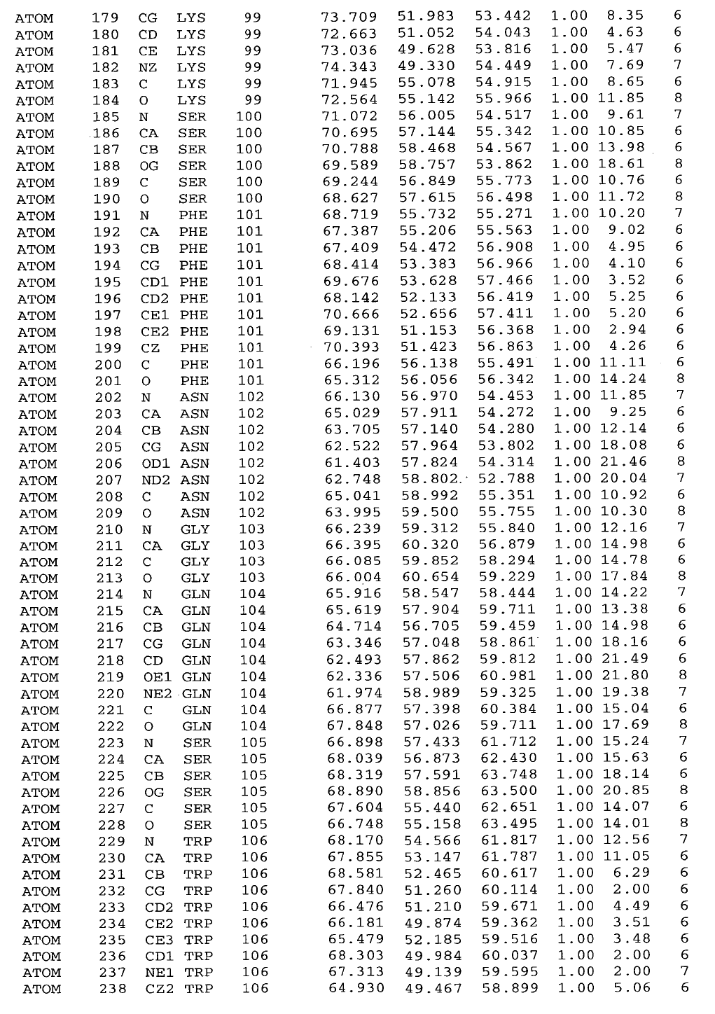

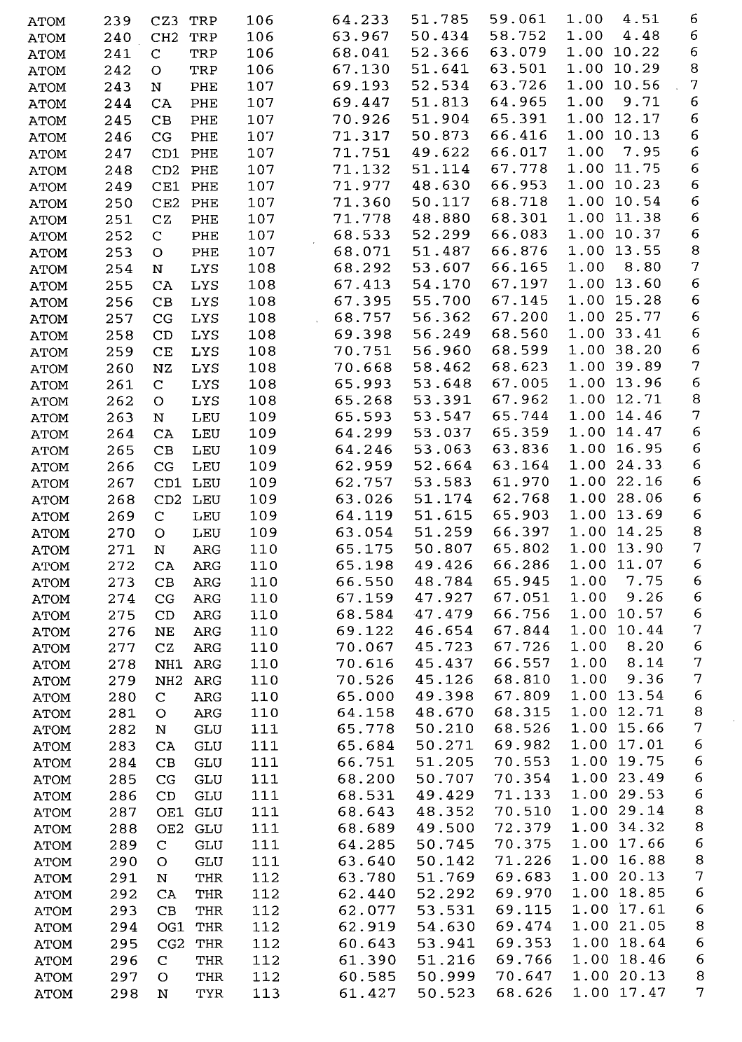

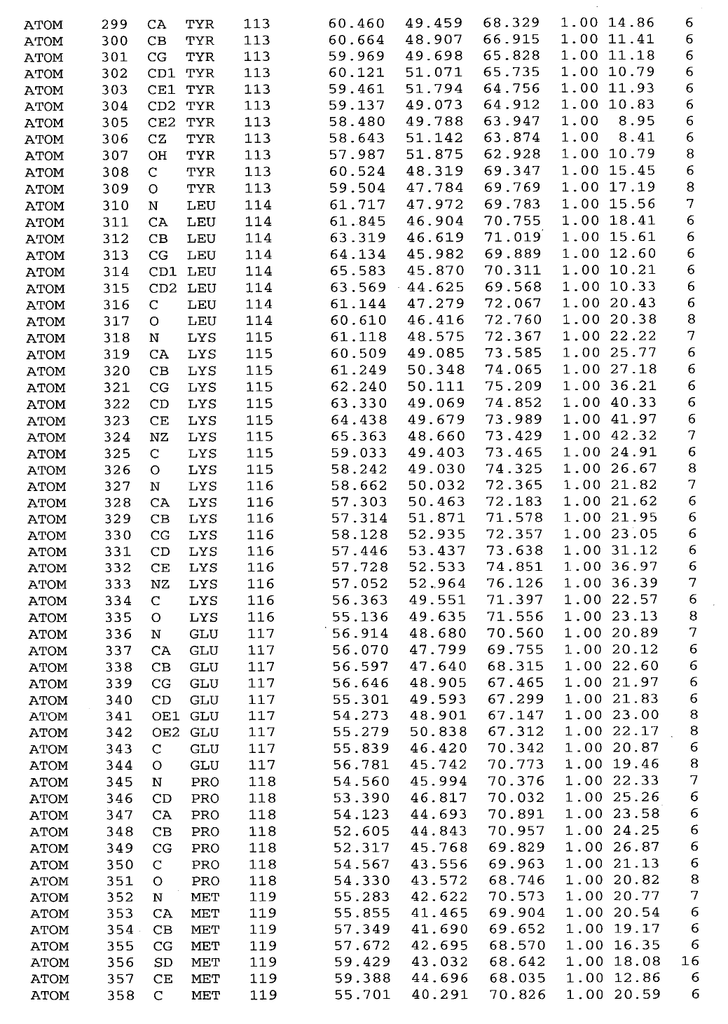

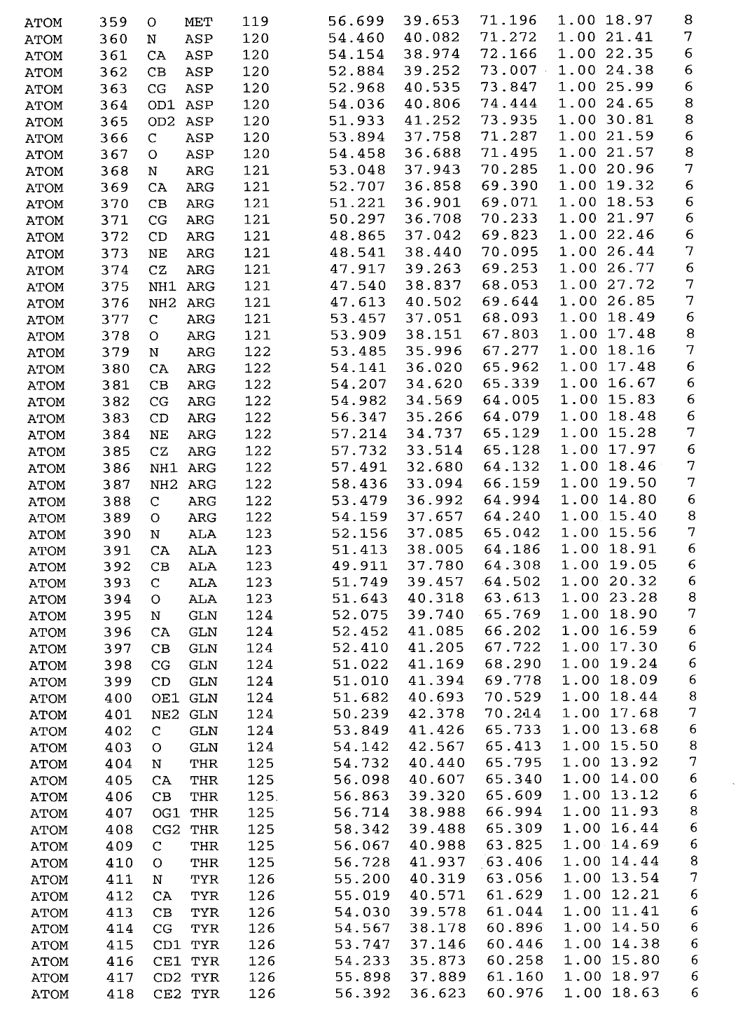

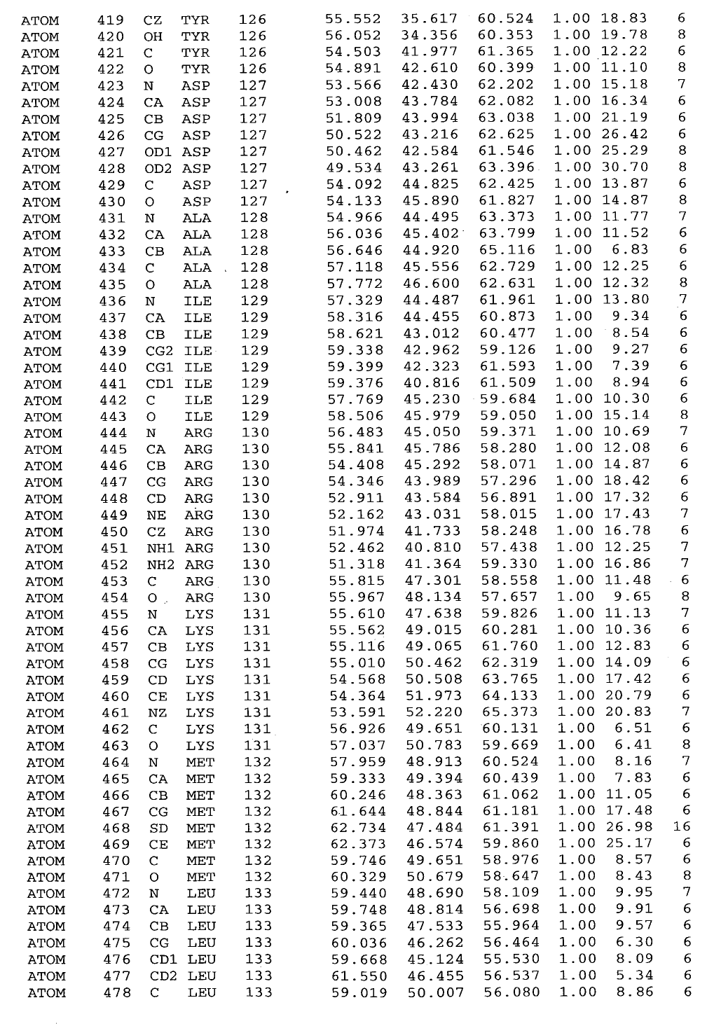

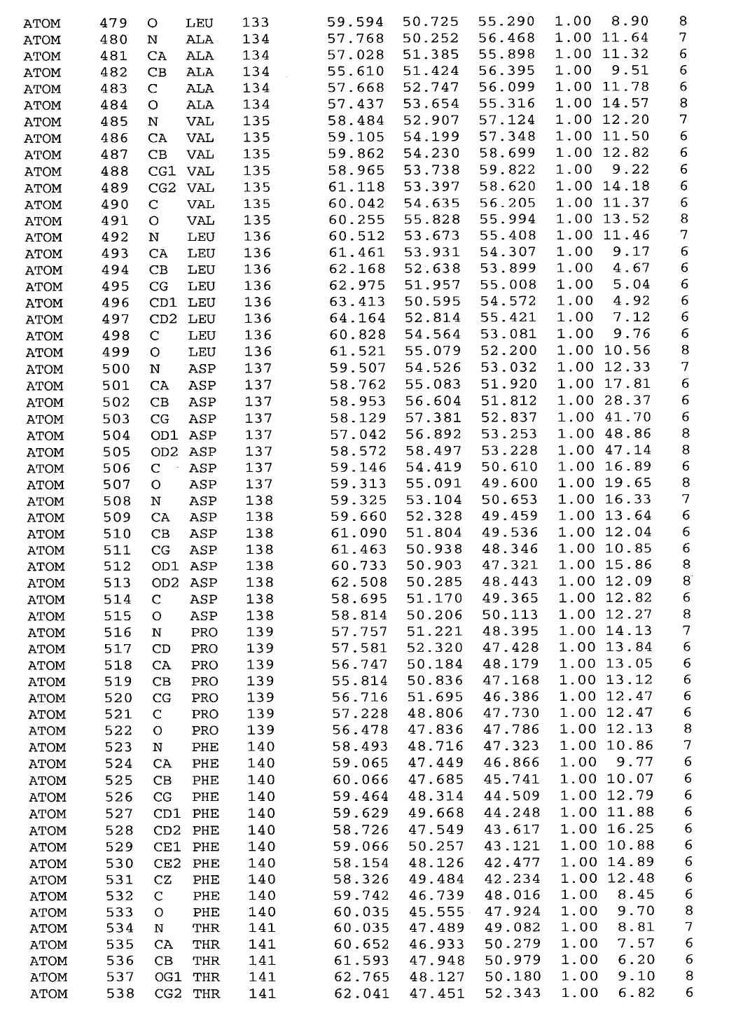

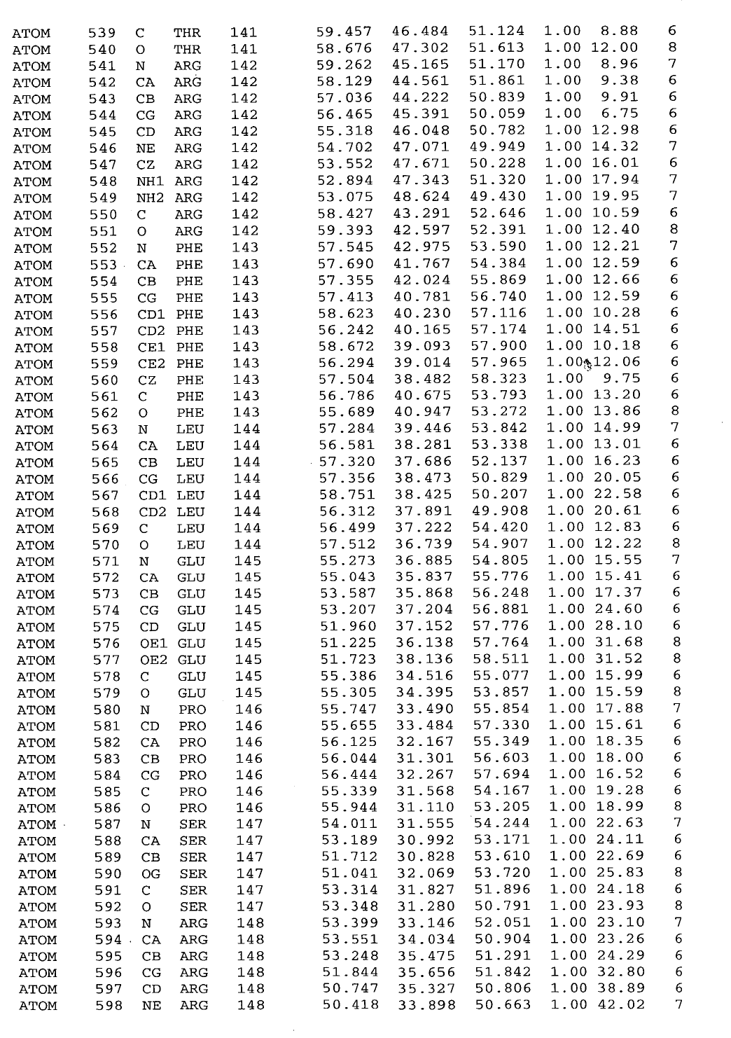

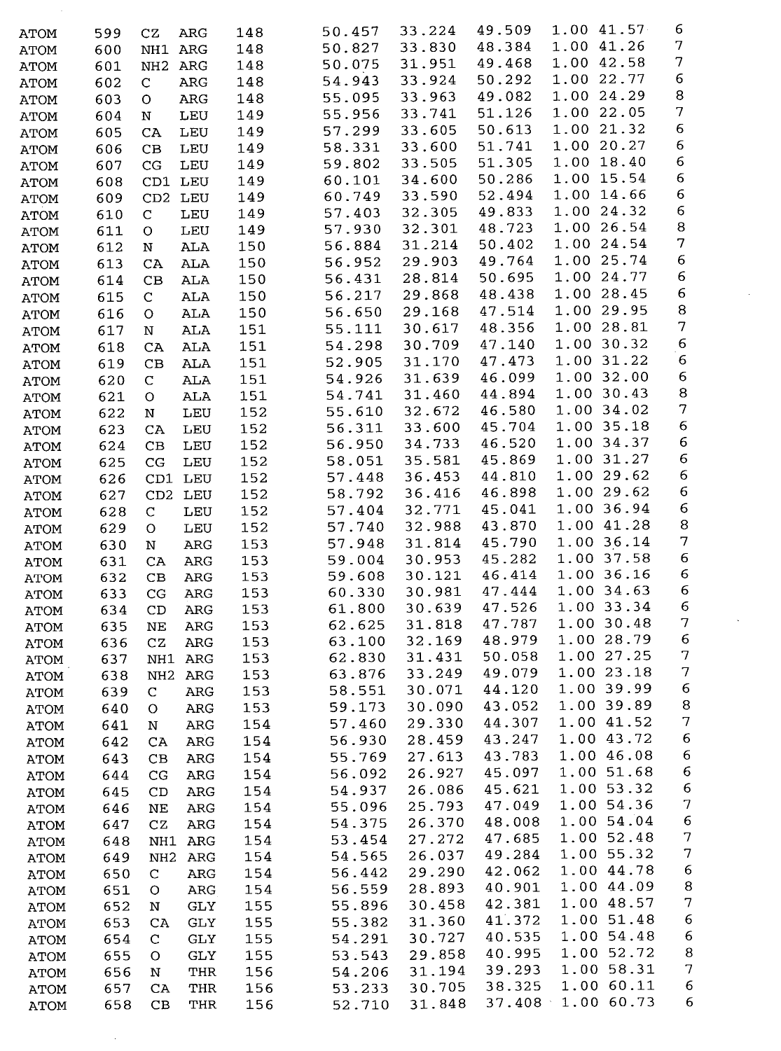

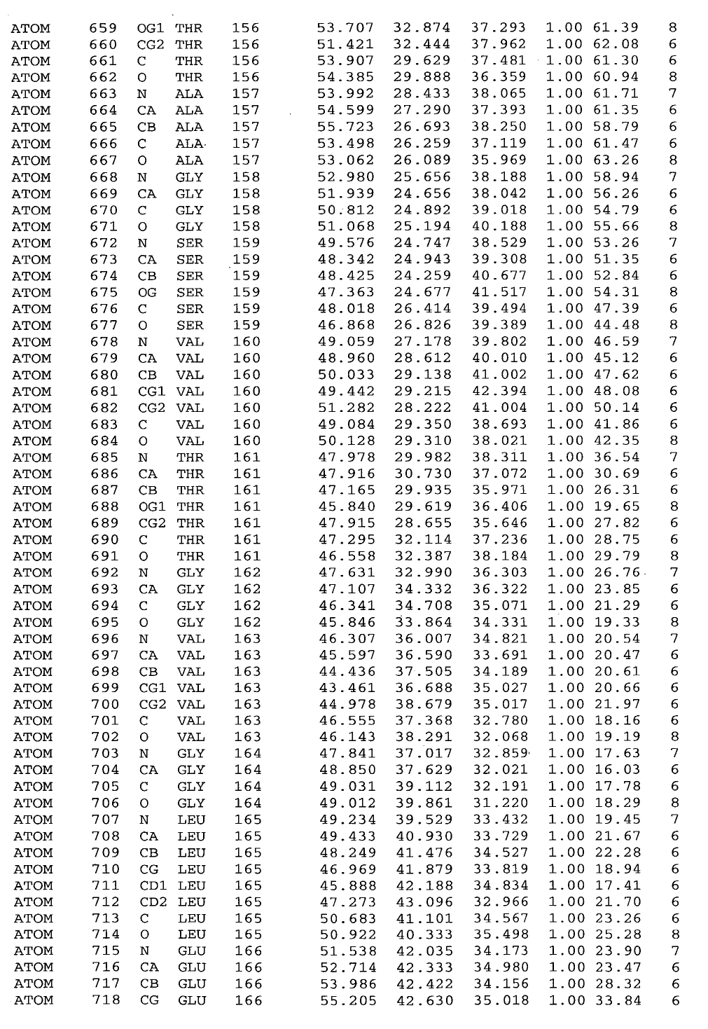

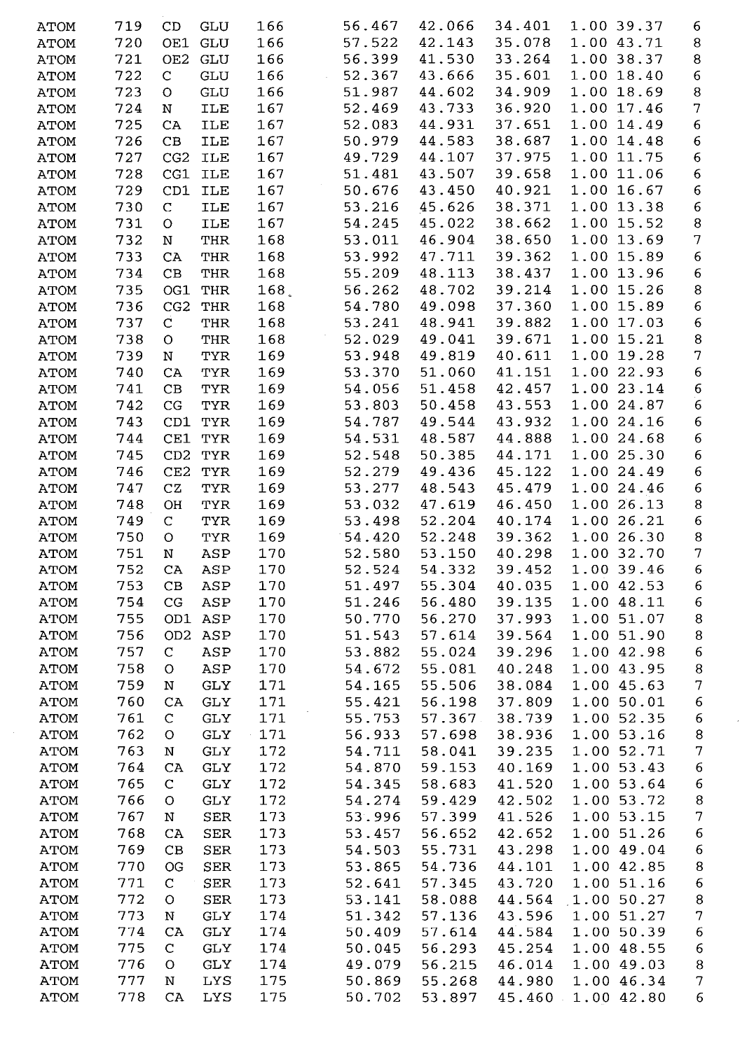

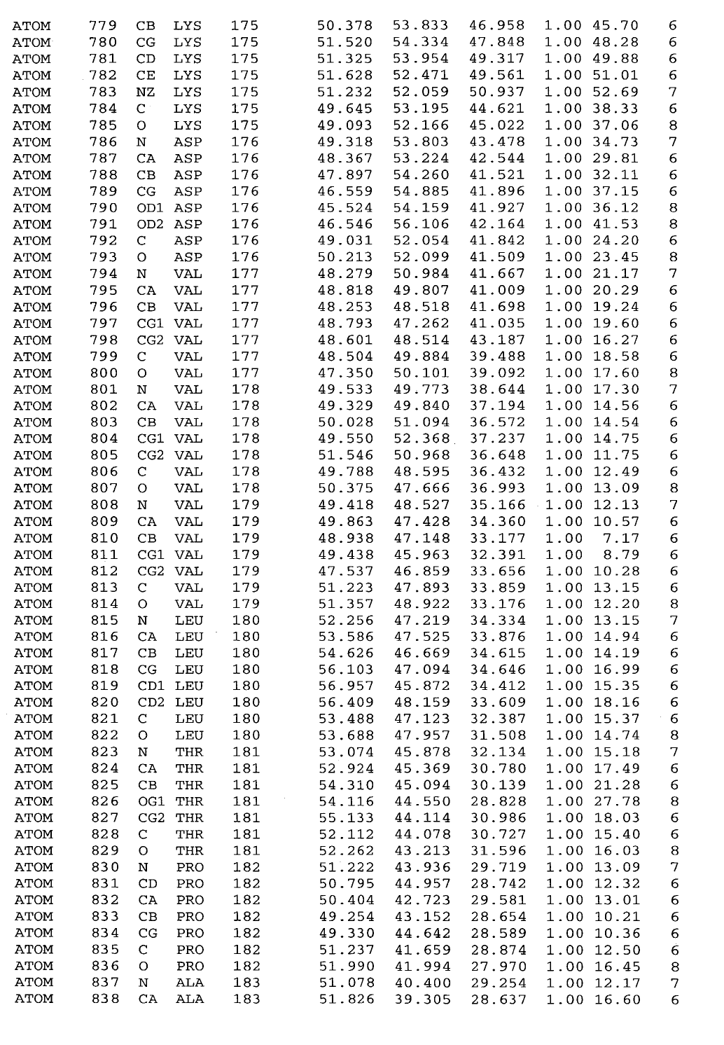

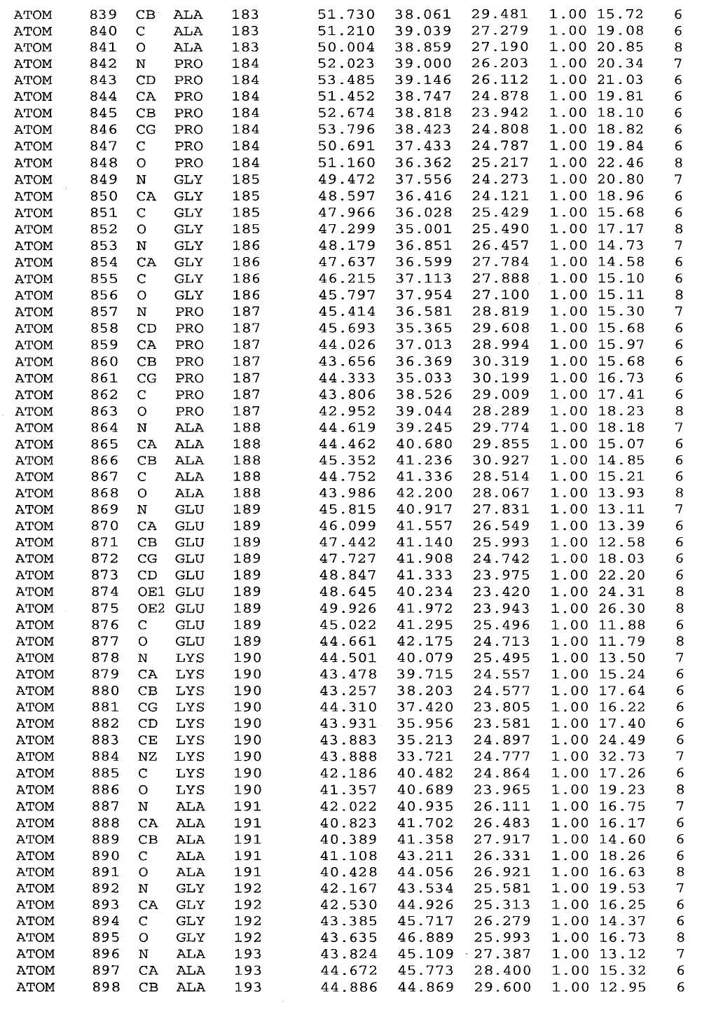

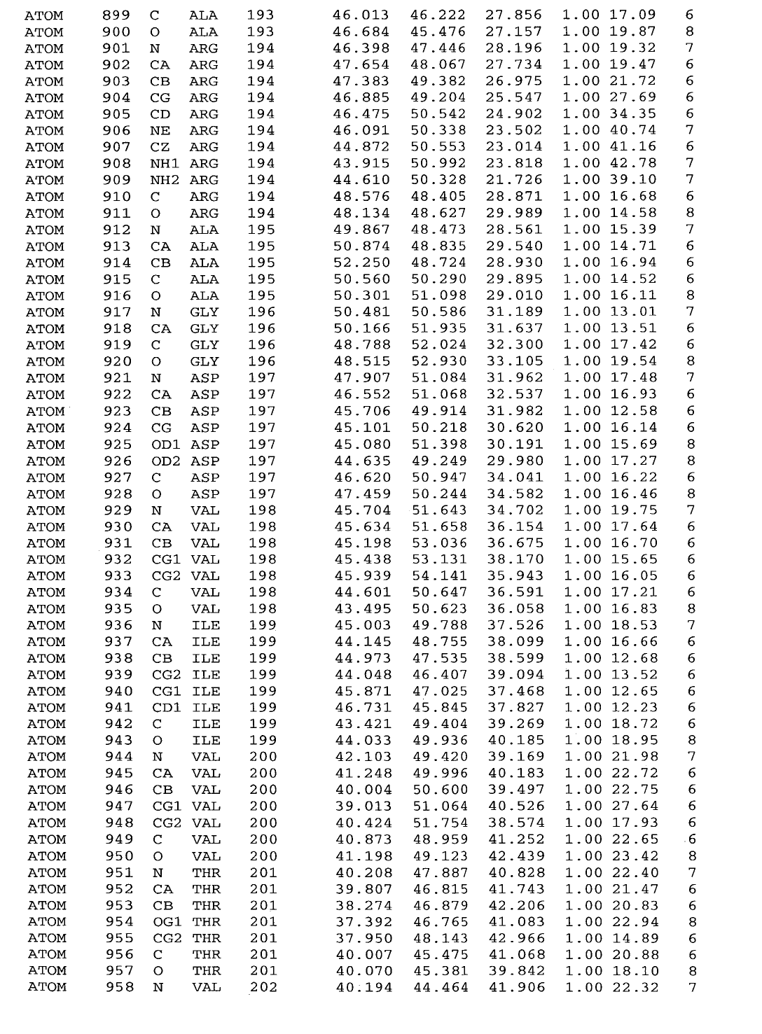

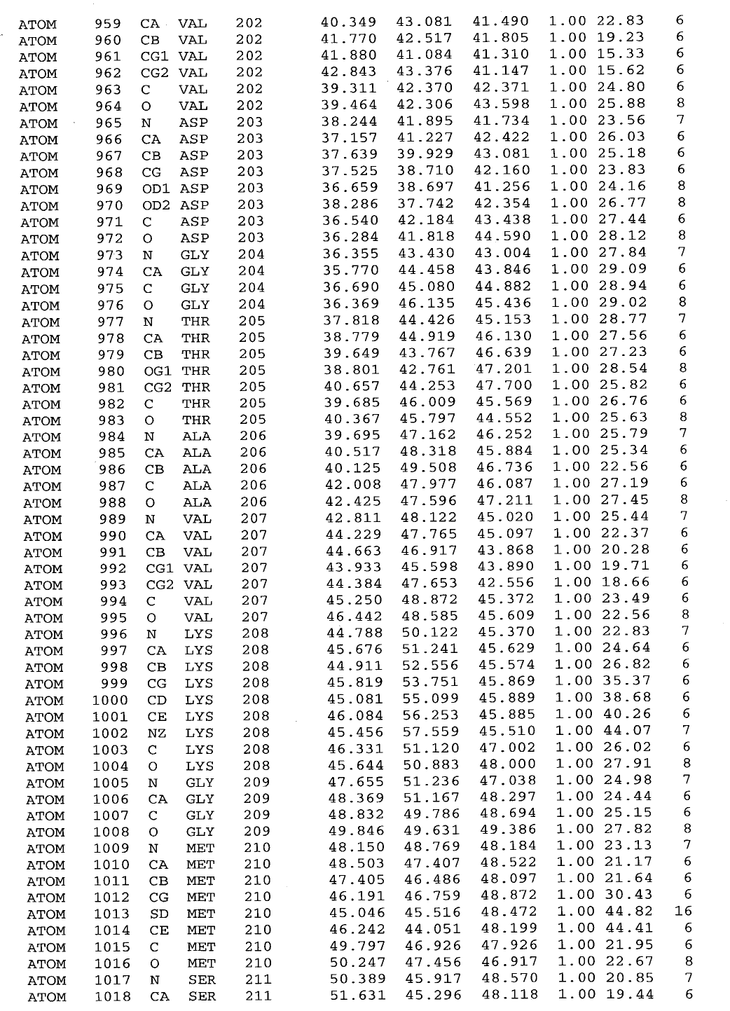

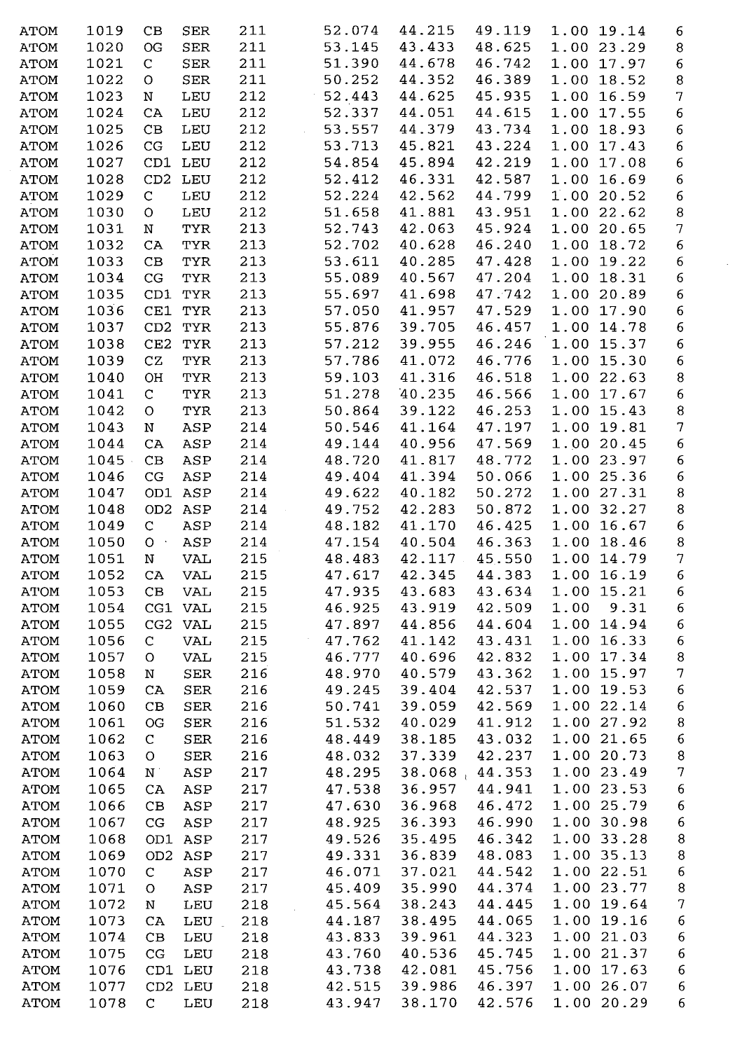

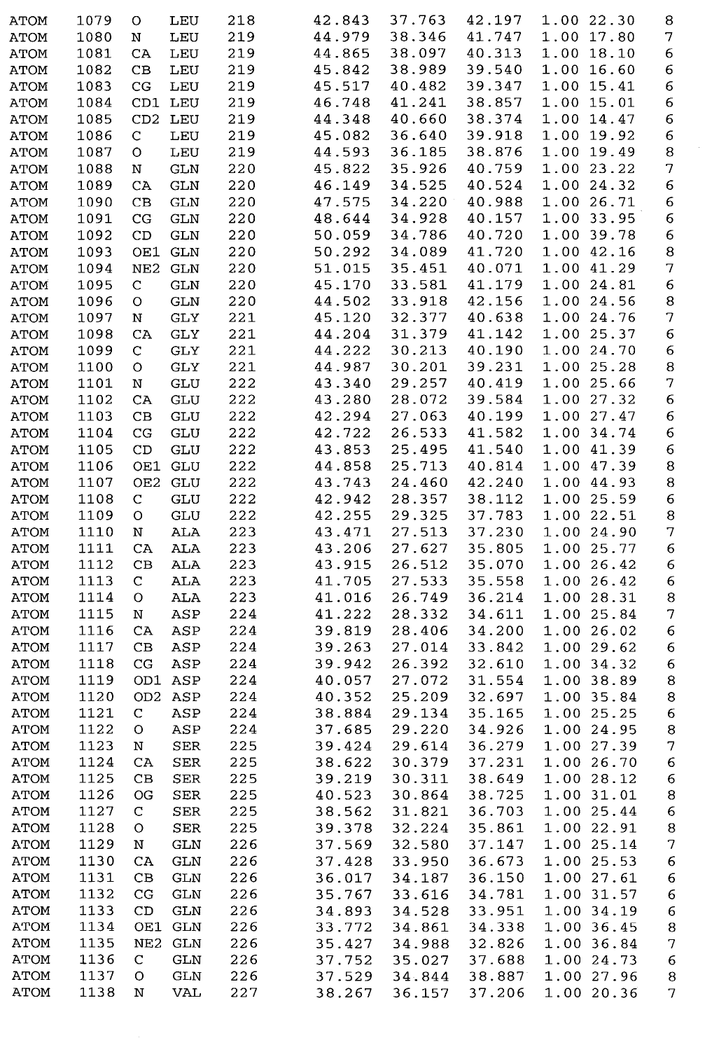

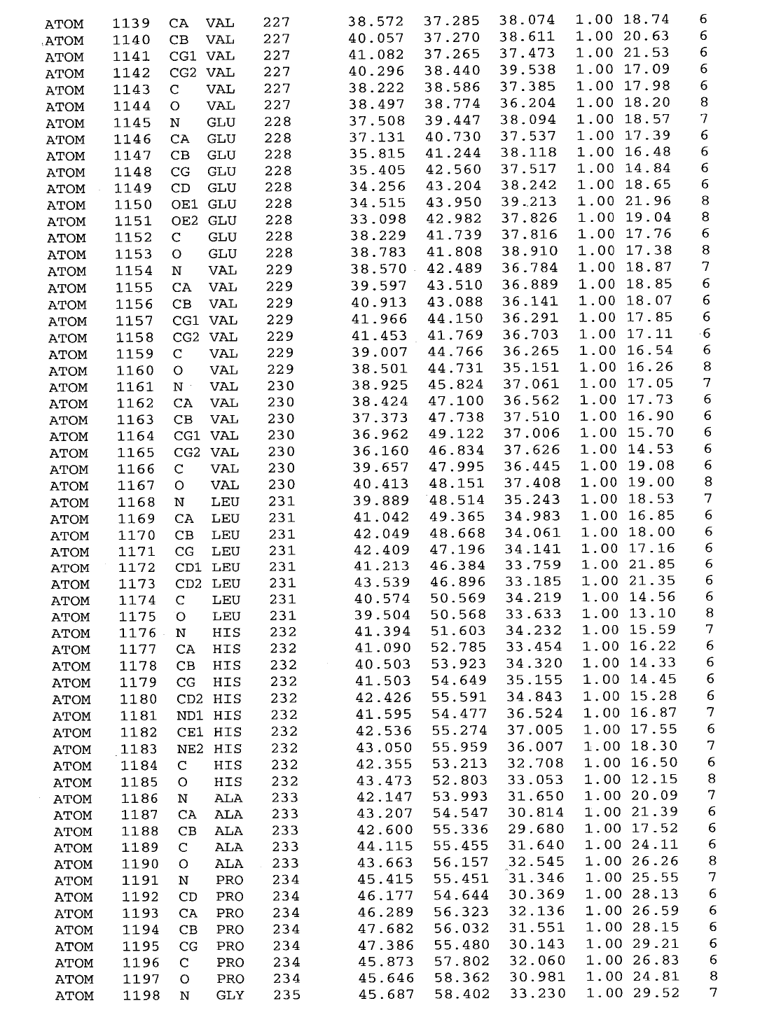

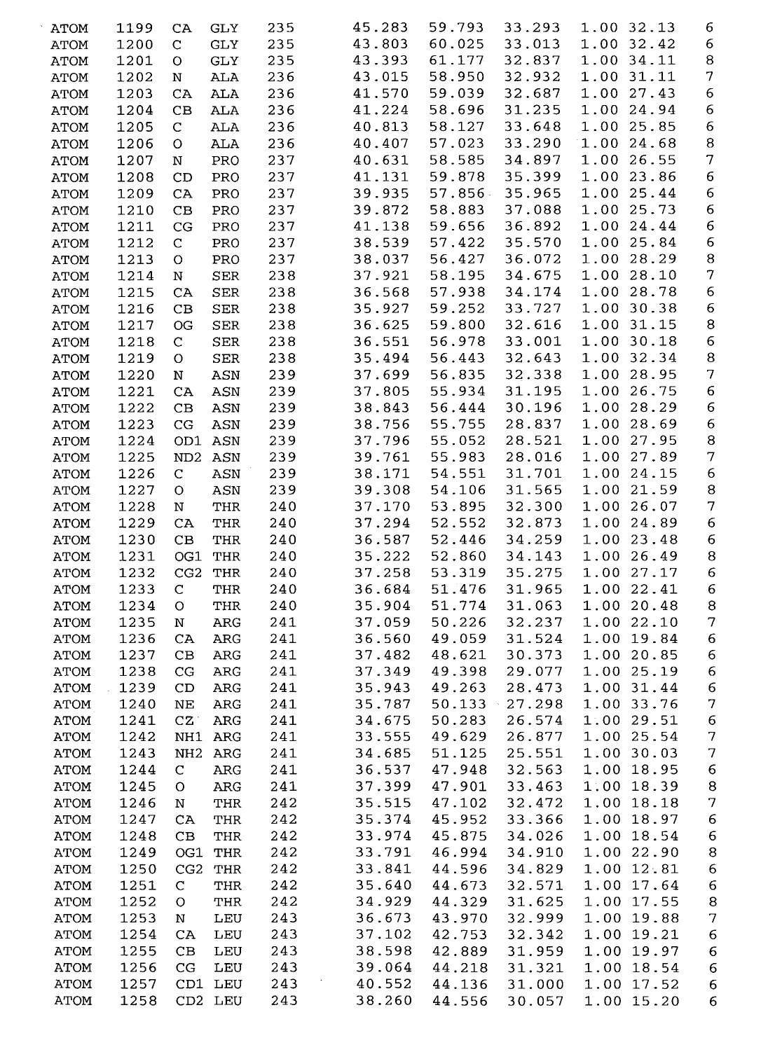

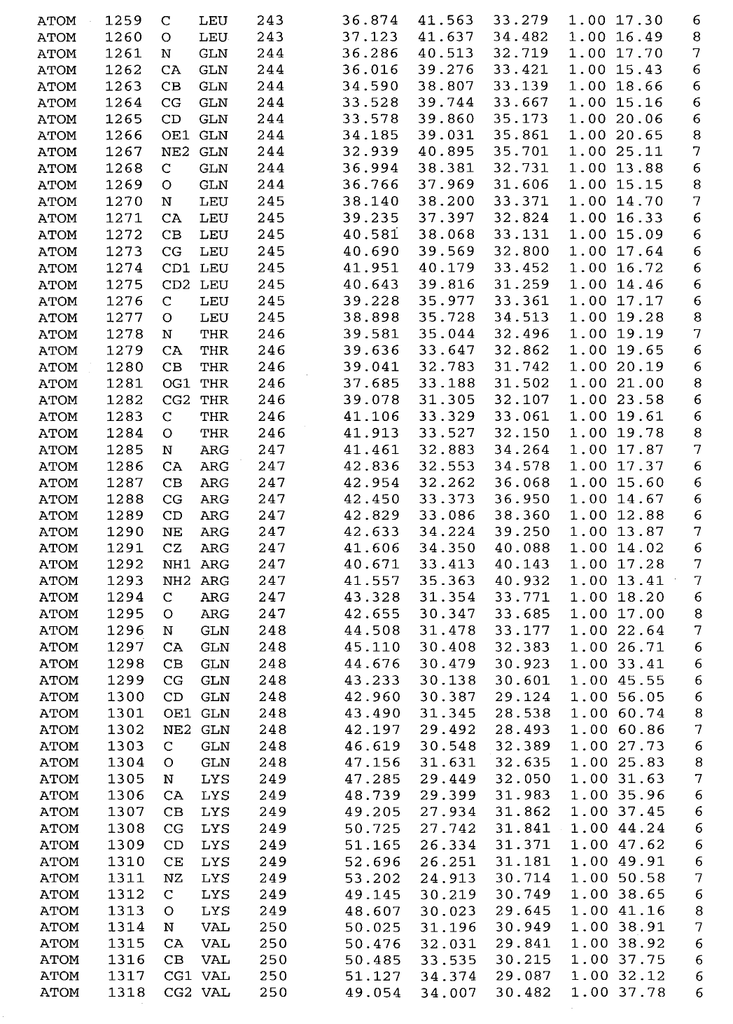

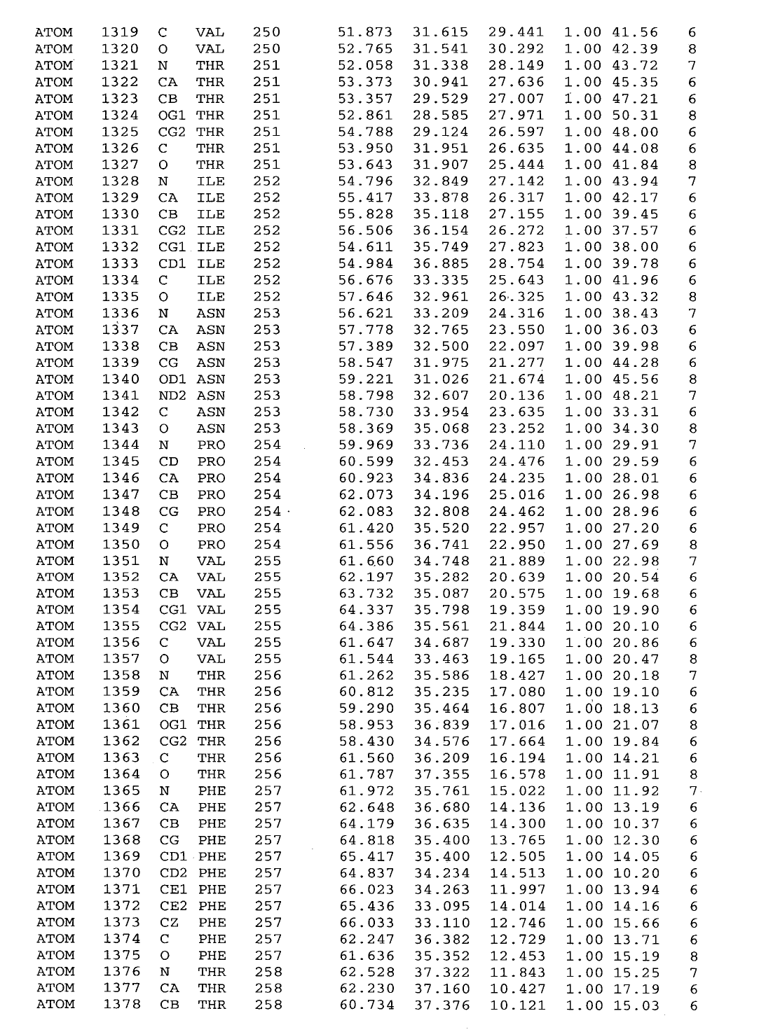

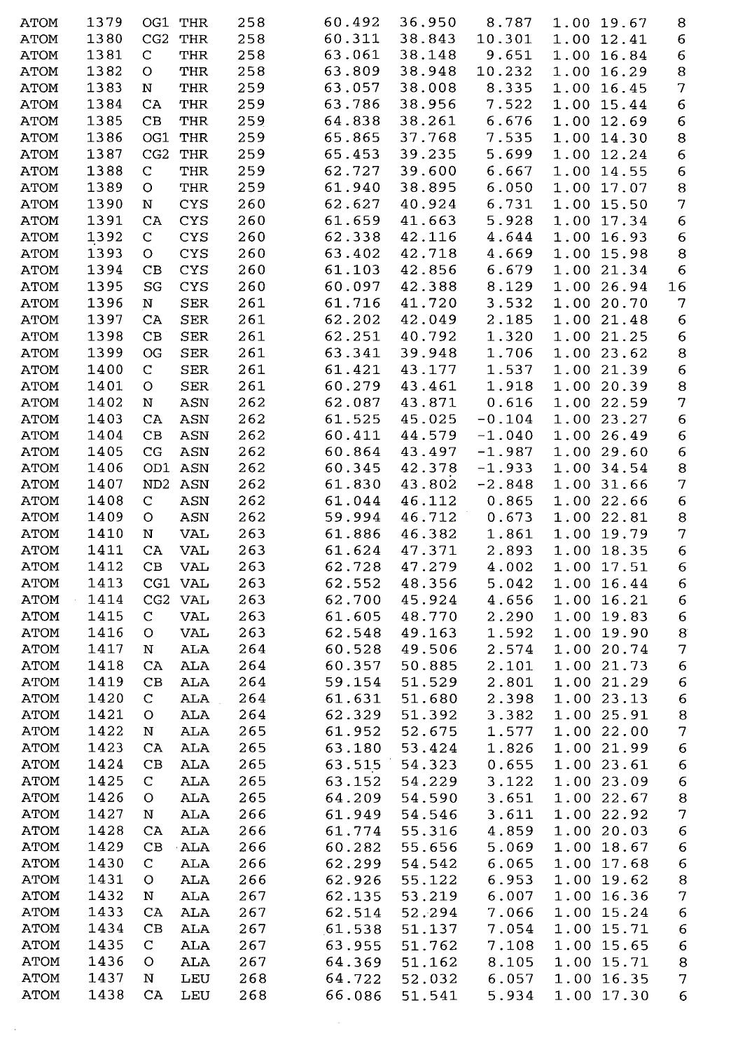

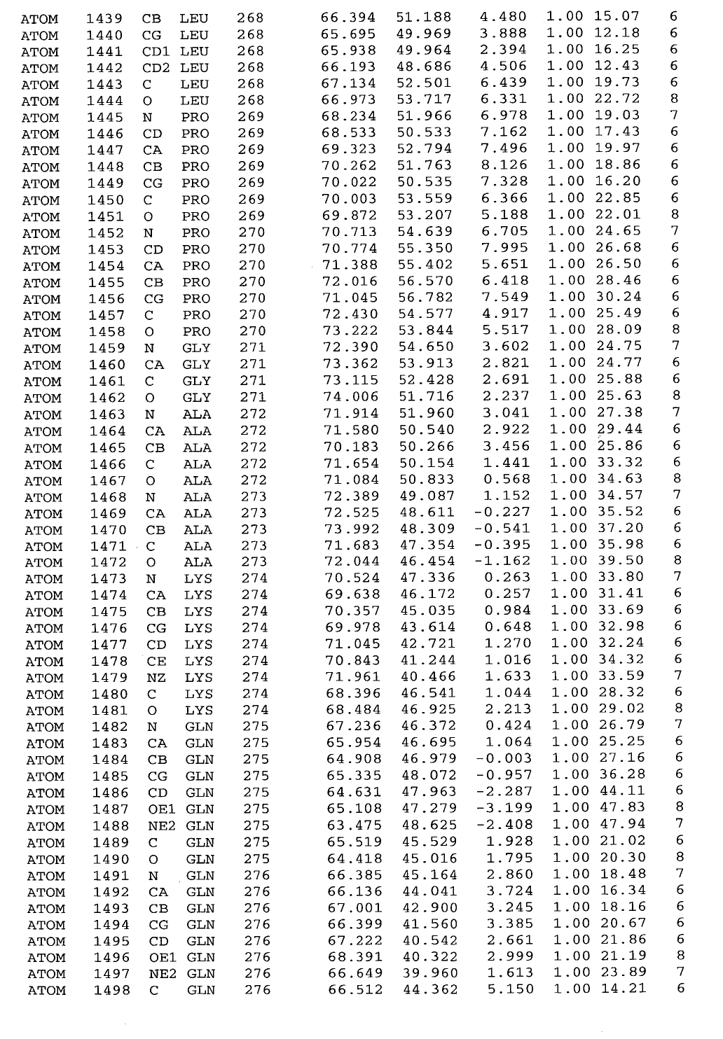

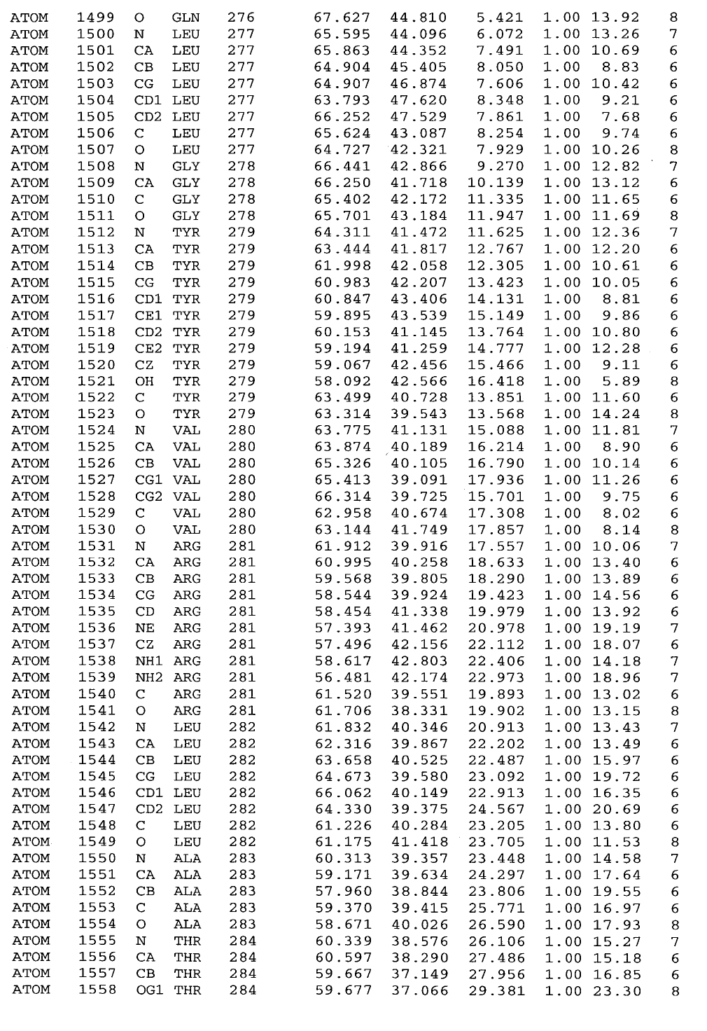

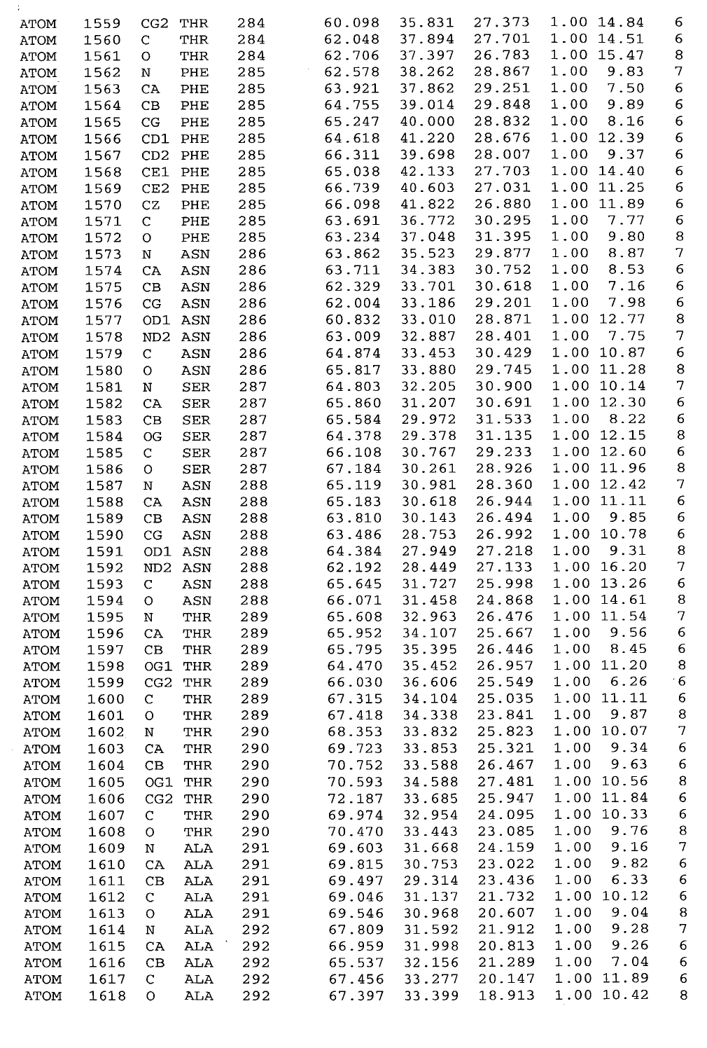

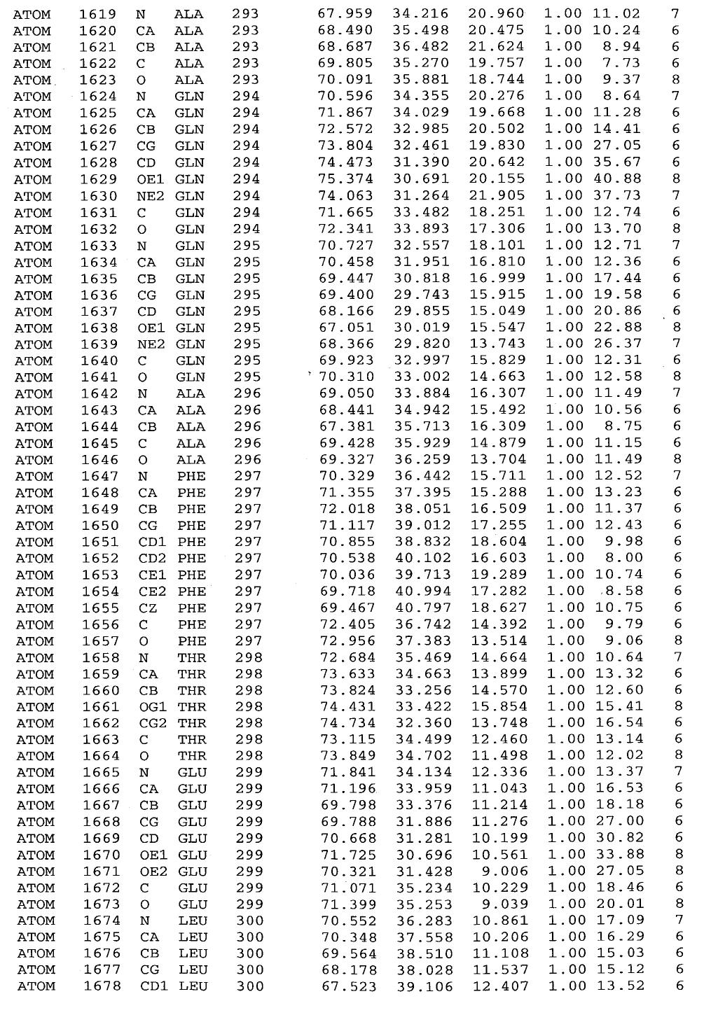

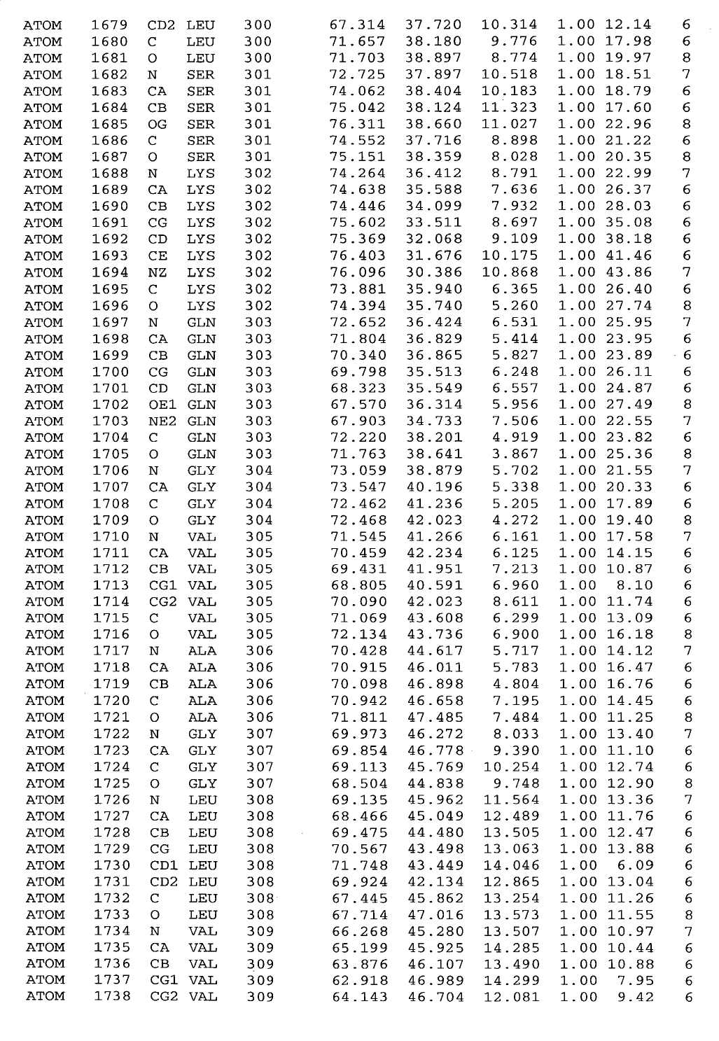

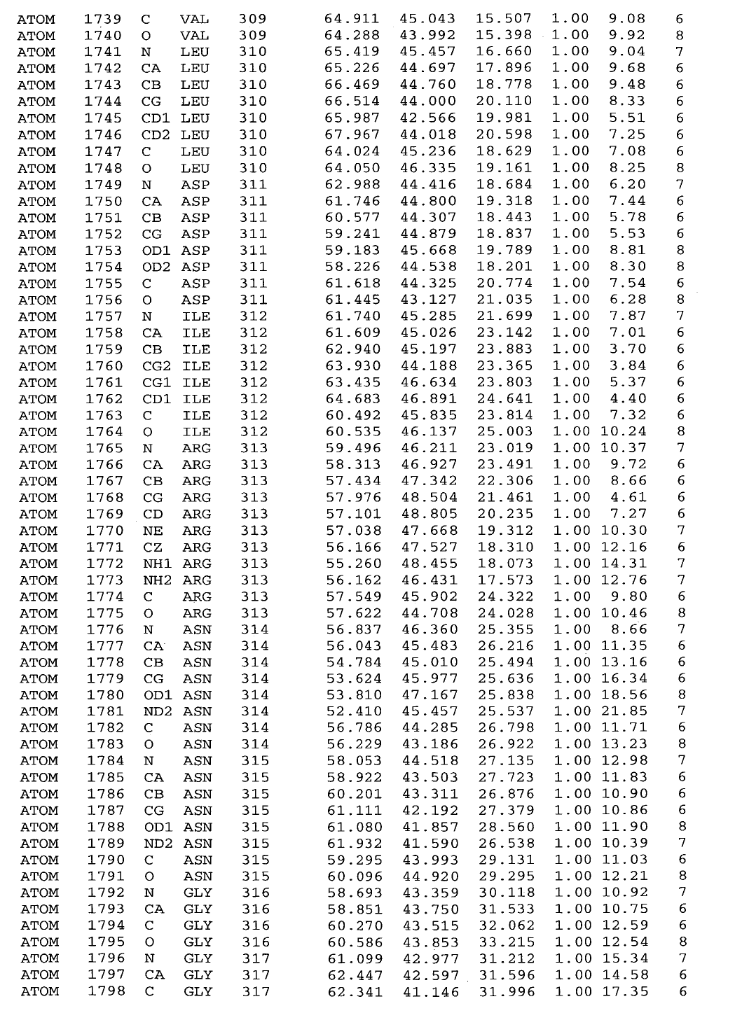

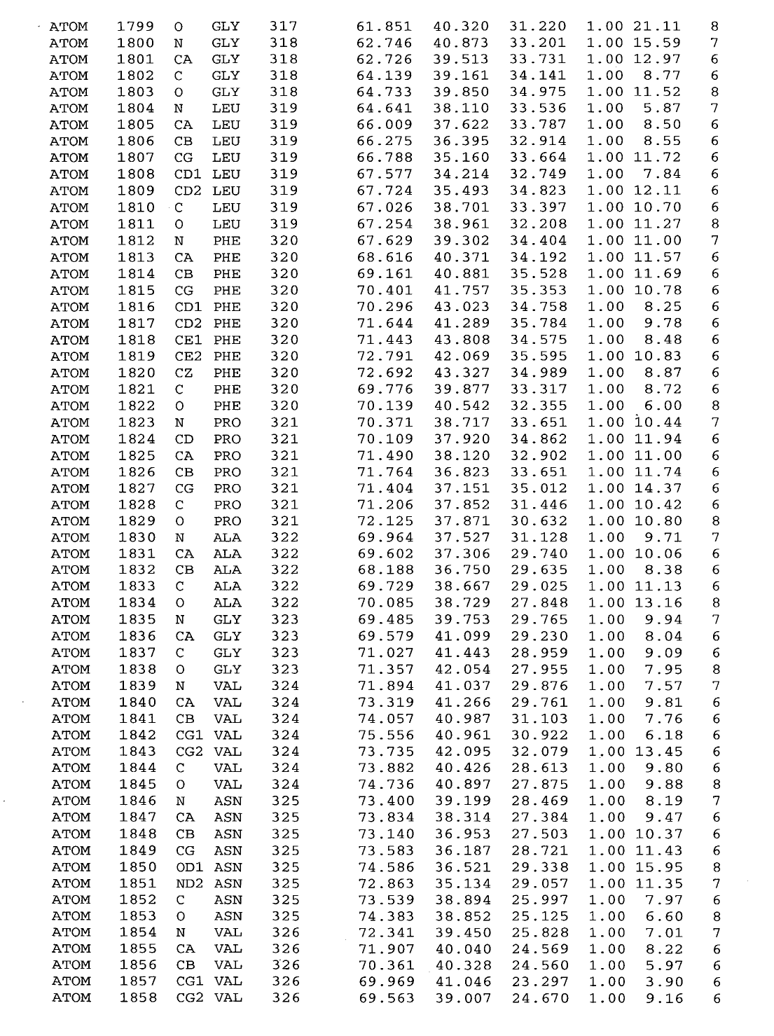

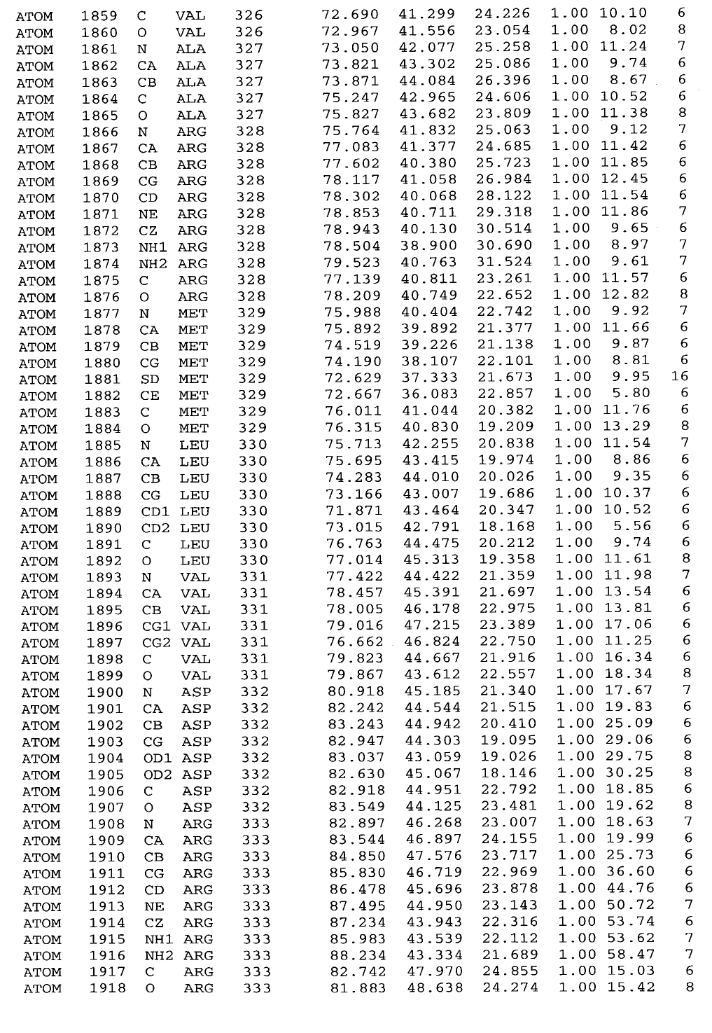

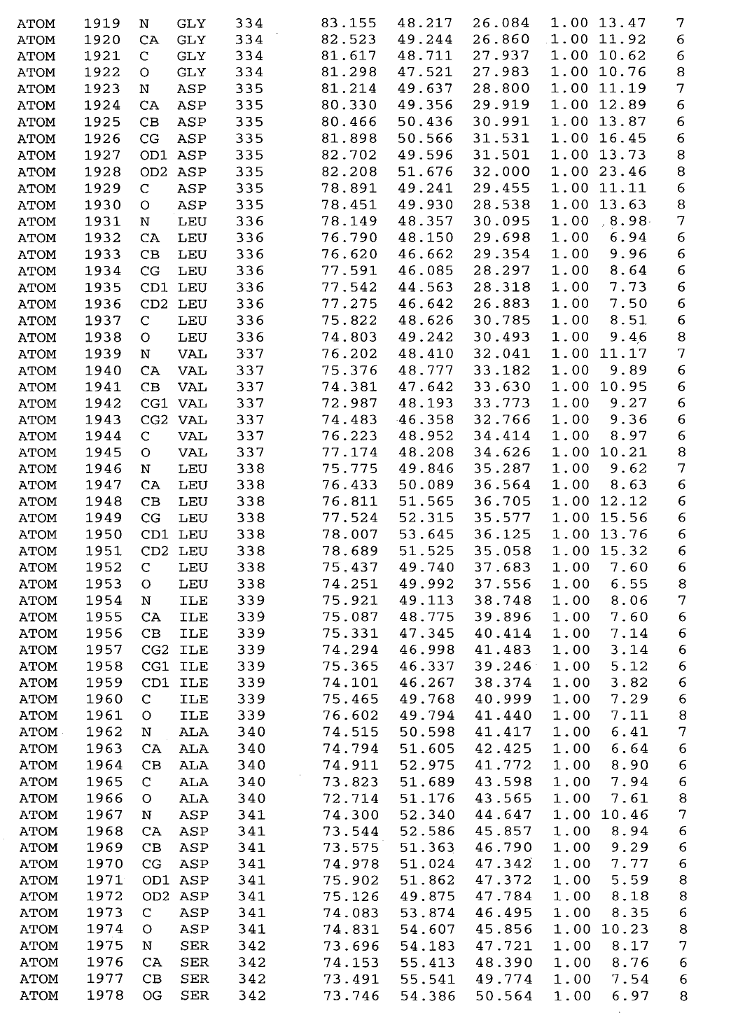

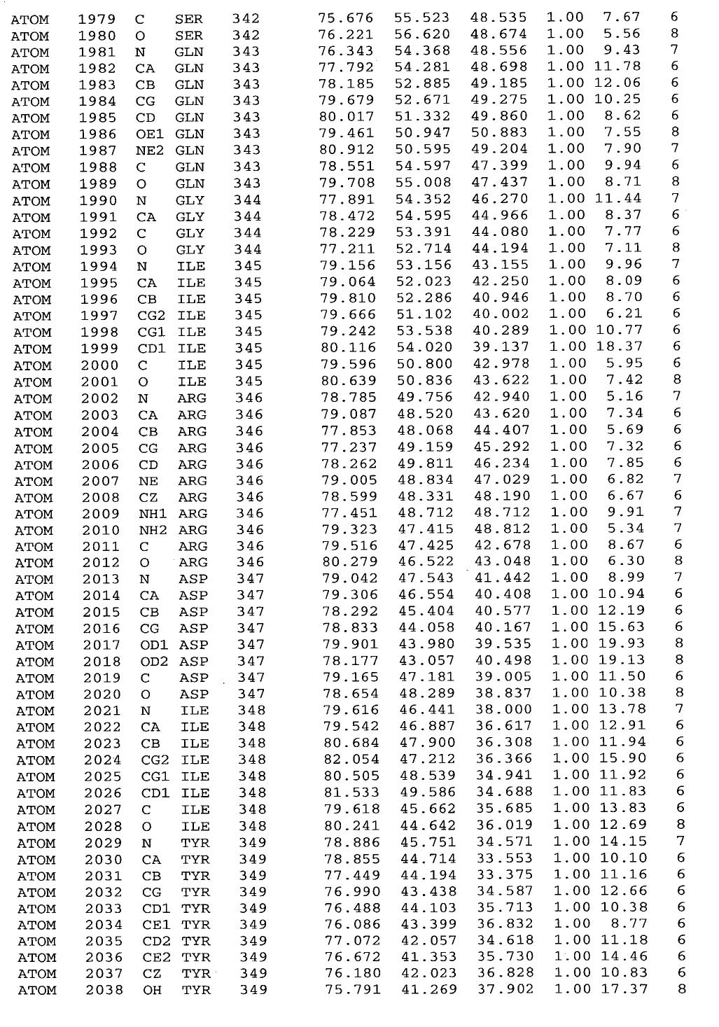

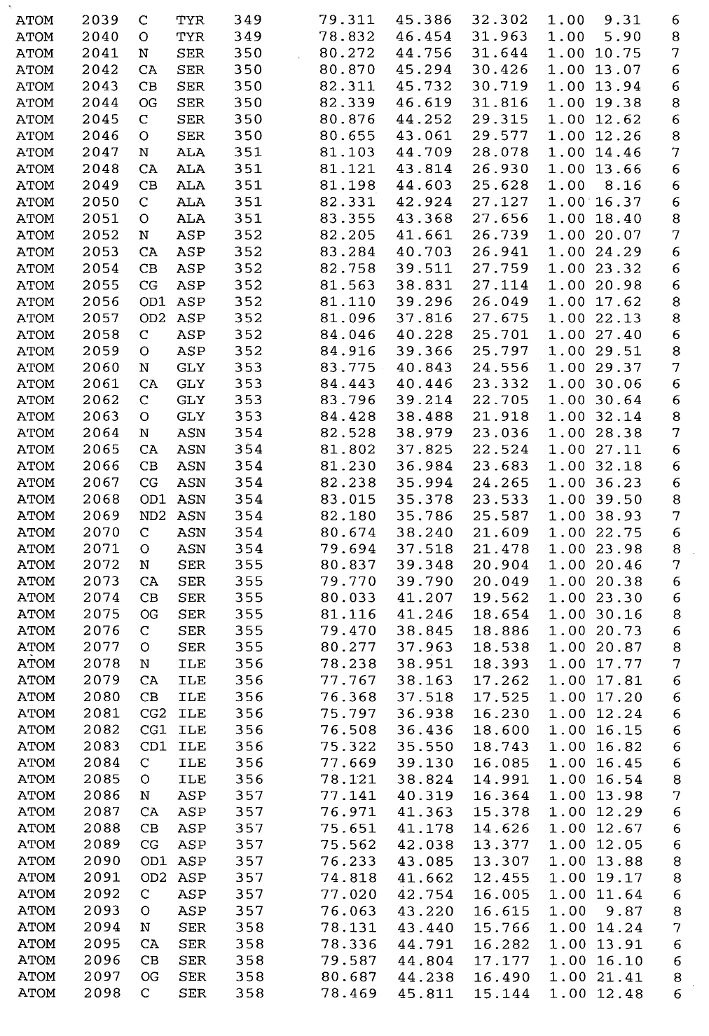

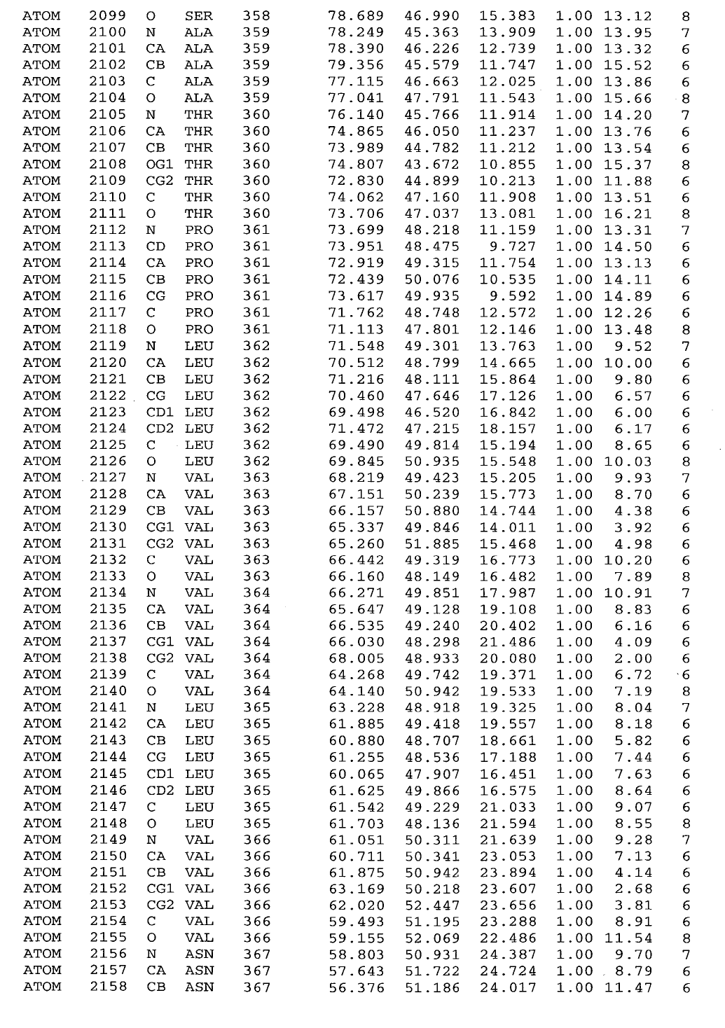

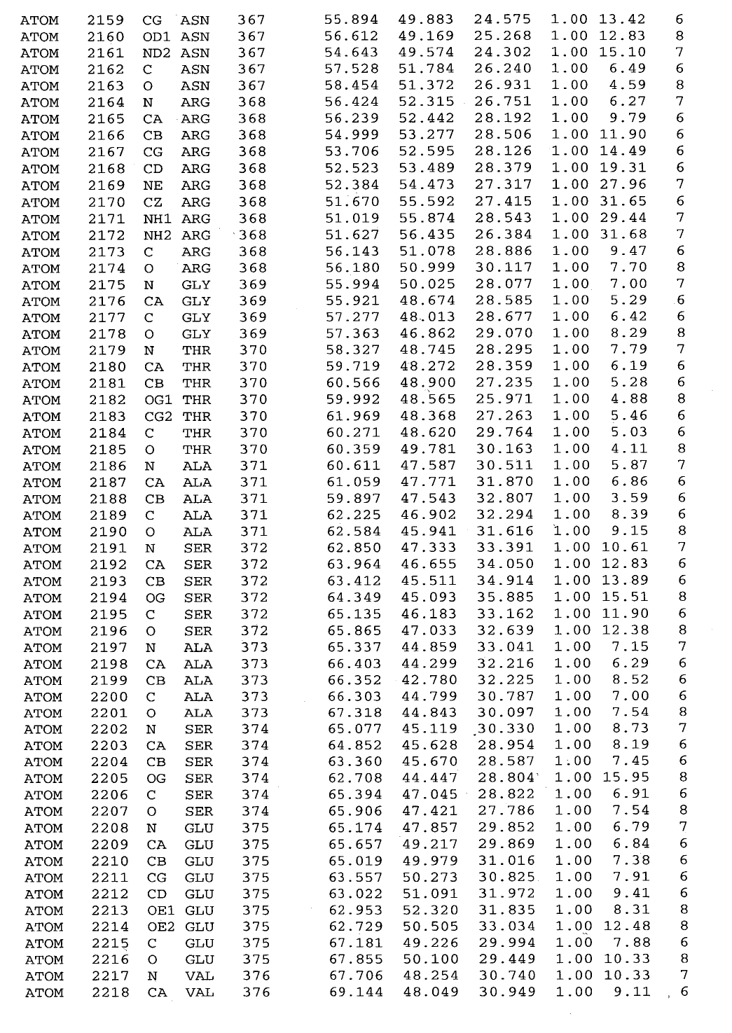

The refined Se-Met mutant model with water molecules removed was used to refine the native C2 I form 1.9 Å data set. The data set was collected at minus 170° C. on an Raxis IV imaging plate using X-ray generated by Kigaku rotating anode x-ray generator. X-PLOR was used for the refinement. The working R factor is 28.1% and the free R factor is 32.0% after one cycle of rigid body refinement, using the entire molecule as a group, one cycle of positional refinement and one cycle of restrained B-factor refinement. This indicates that the mutations and Se-Met substitution did not cause significant distortion in the structure. This data set is shown in FIG. 5.

### Example 12

Structure Determination of Native Scenedesmus obliquus D1 Protease Structure (Crystal Form R32)

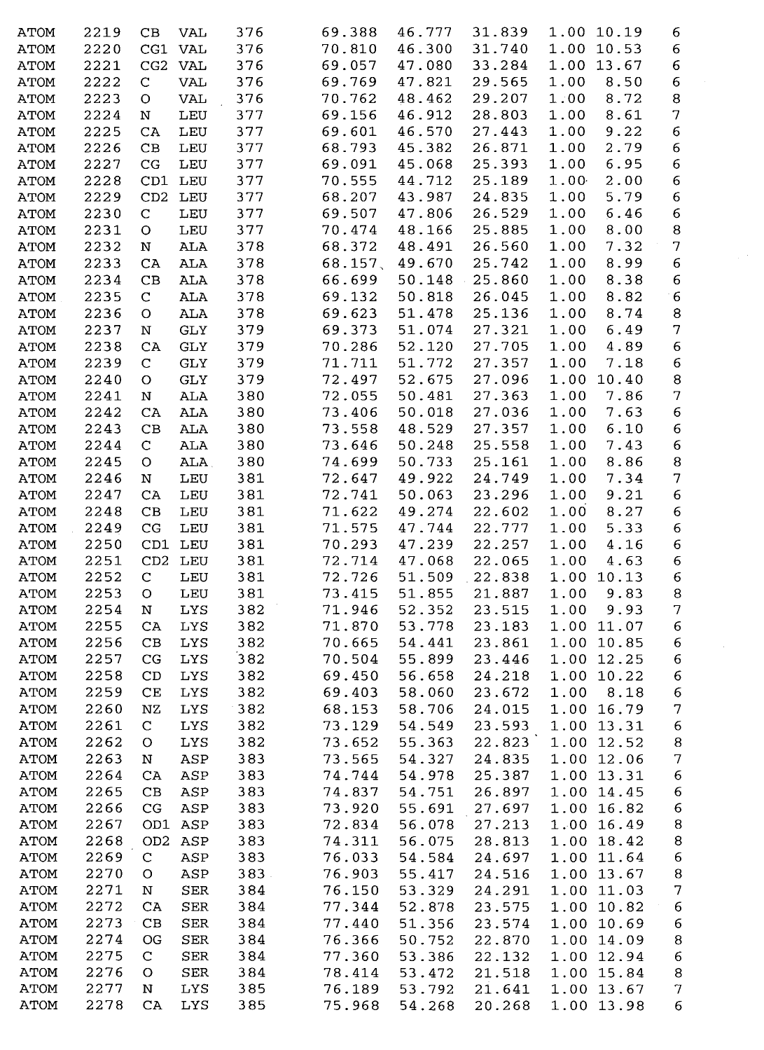

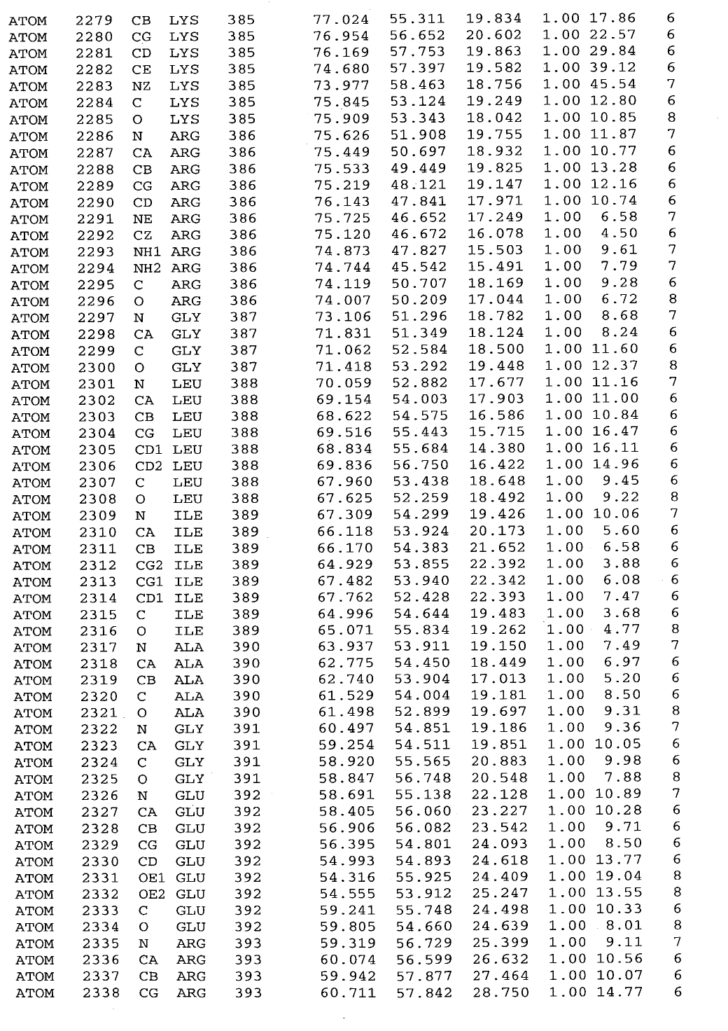

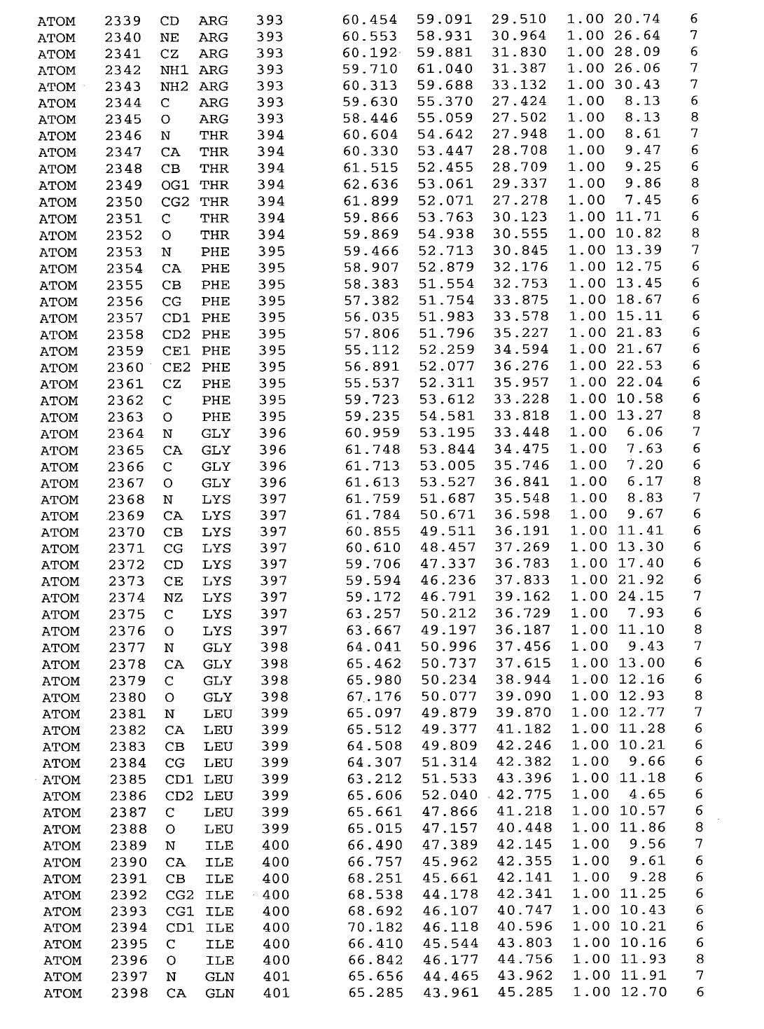

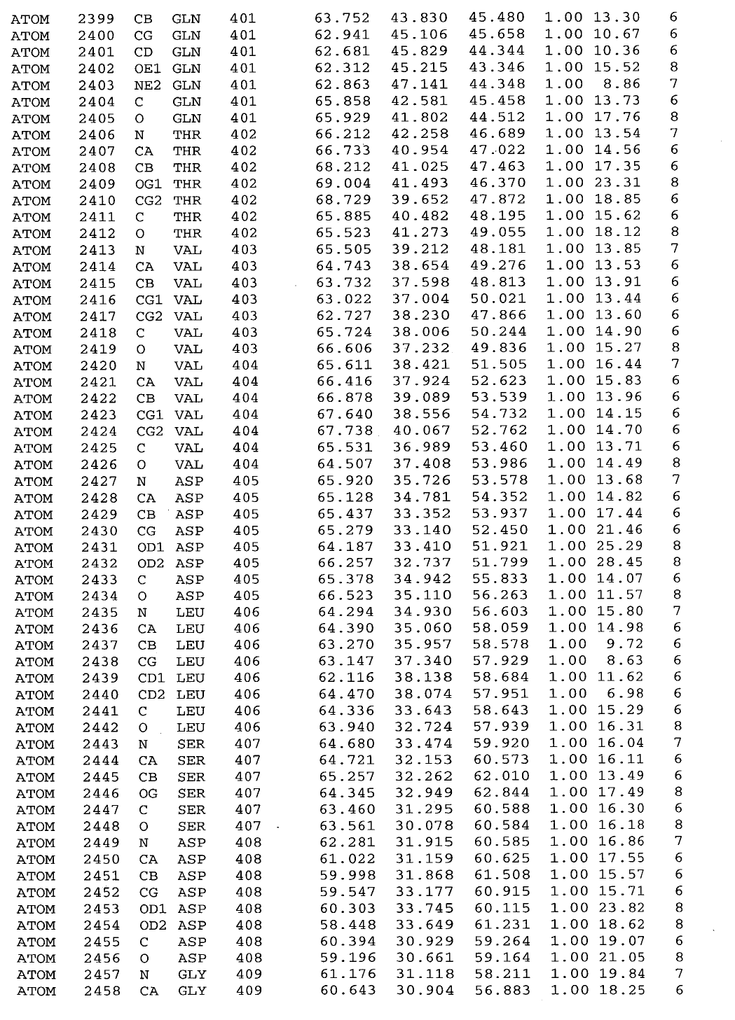

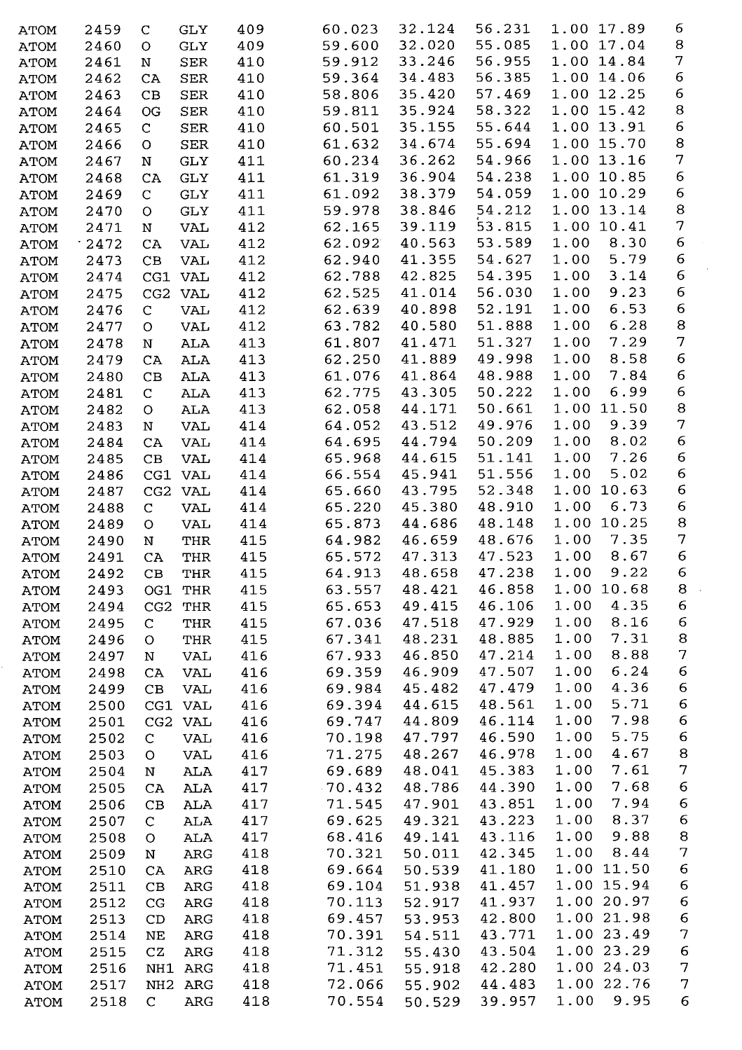

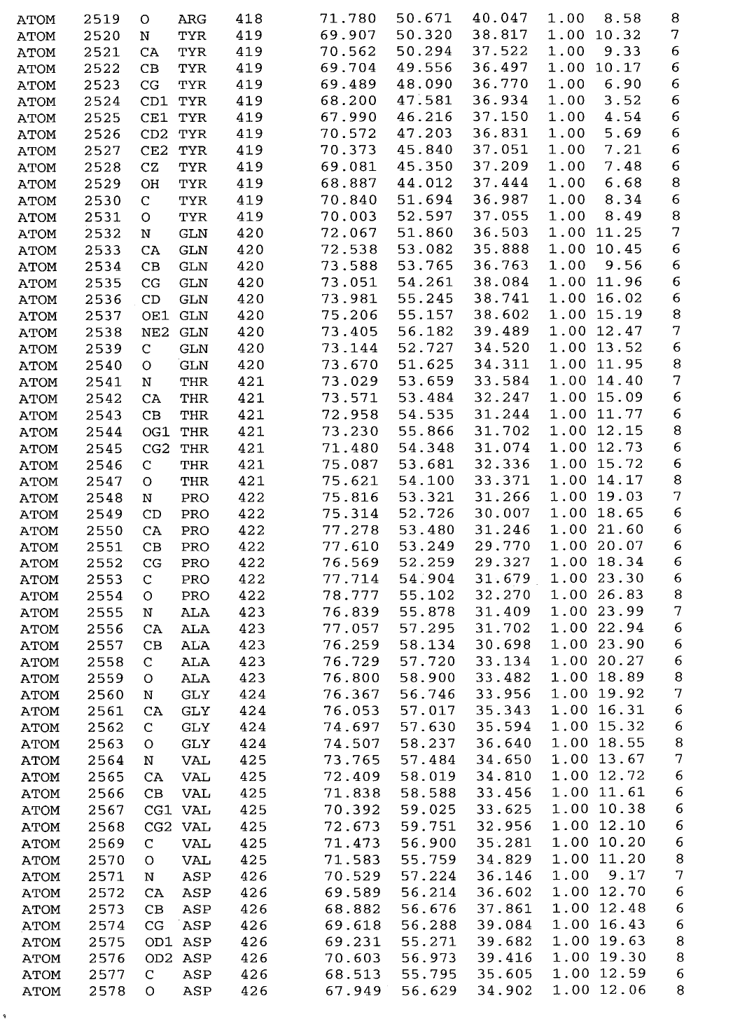

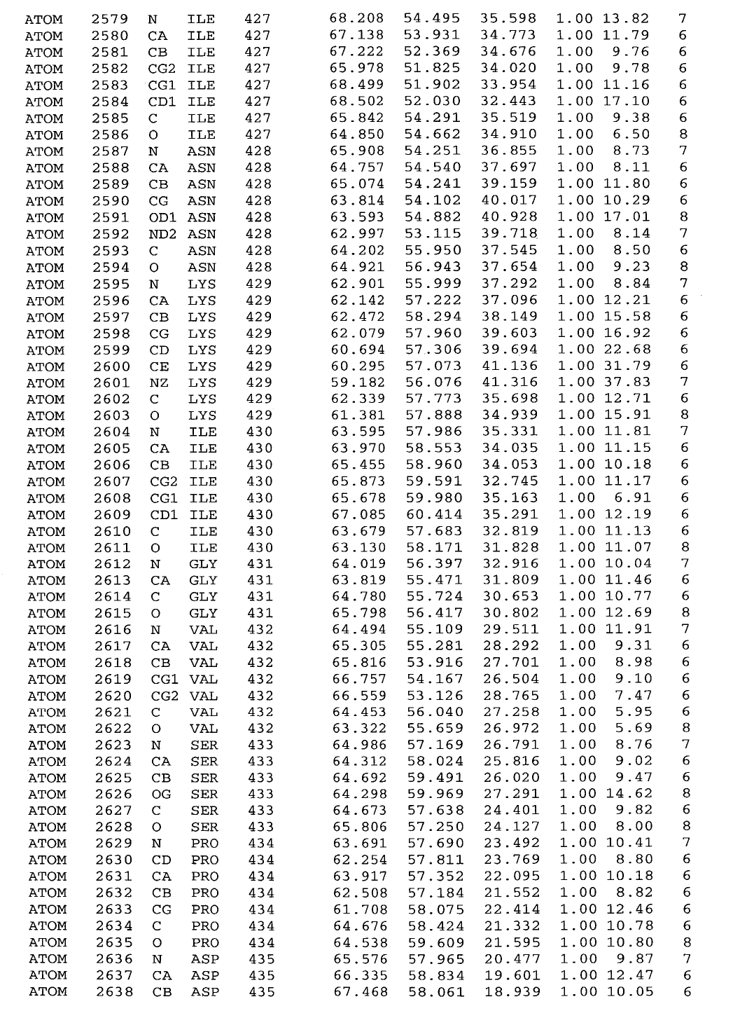

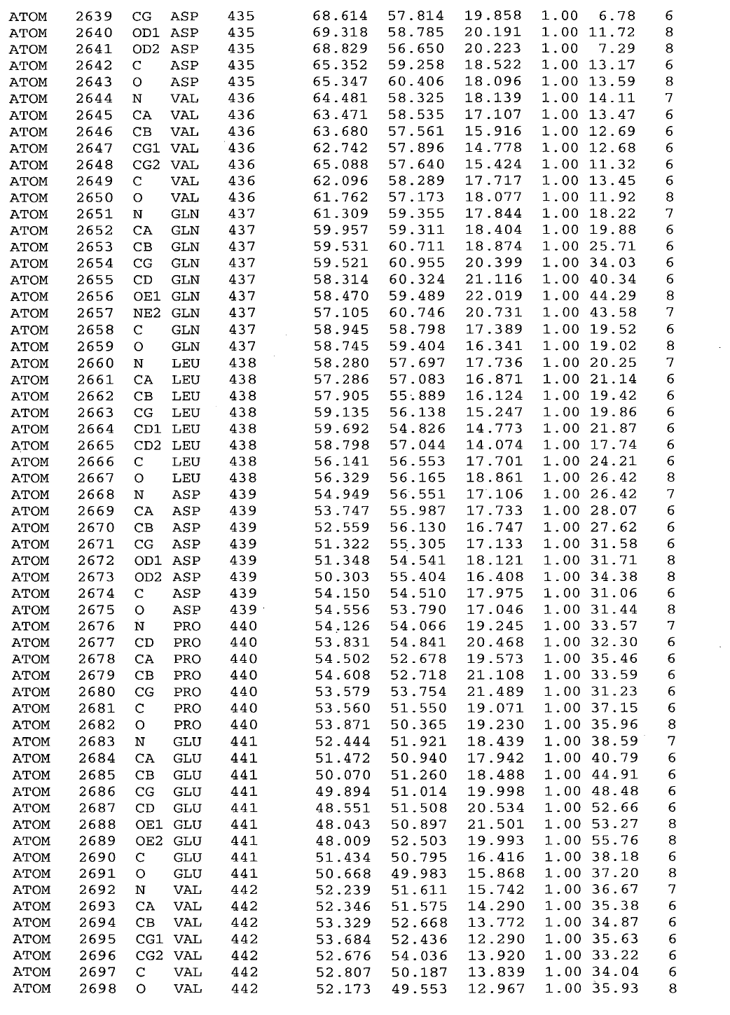

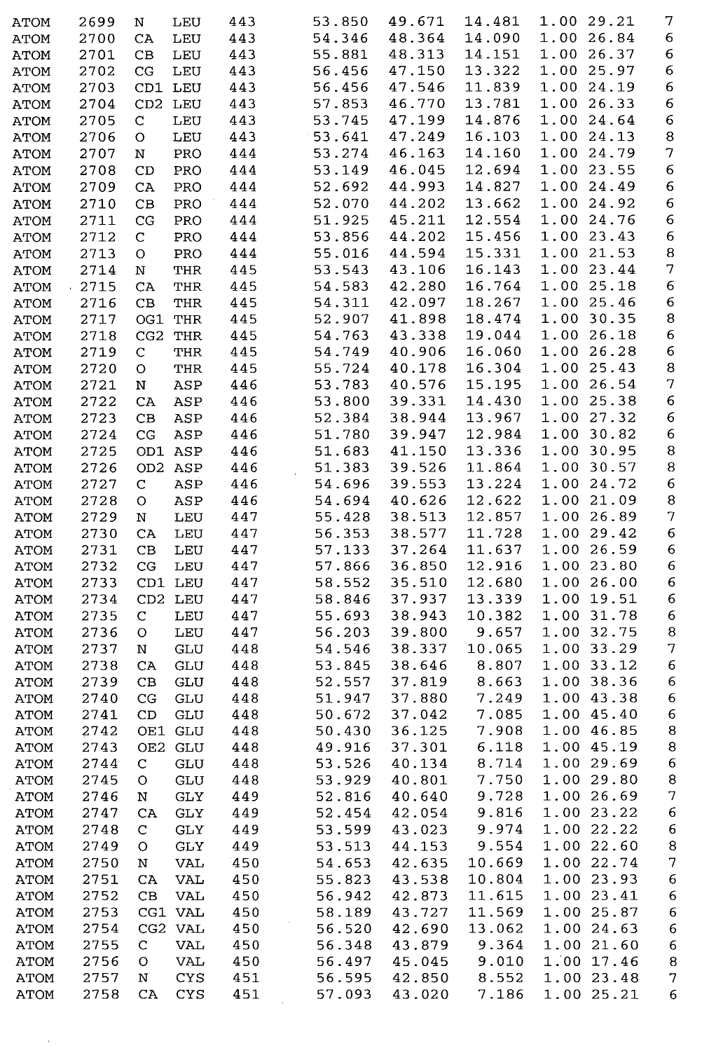

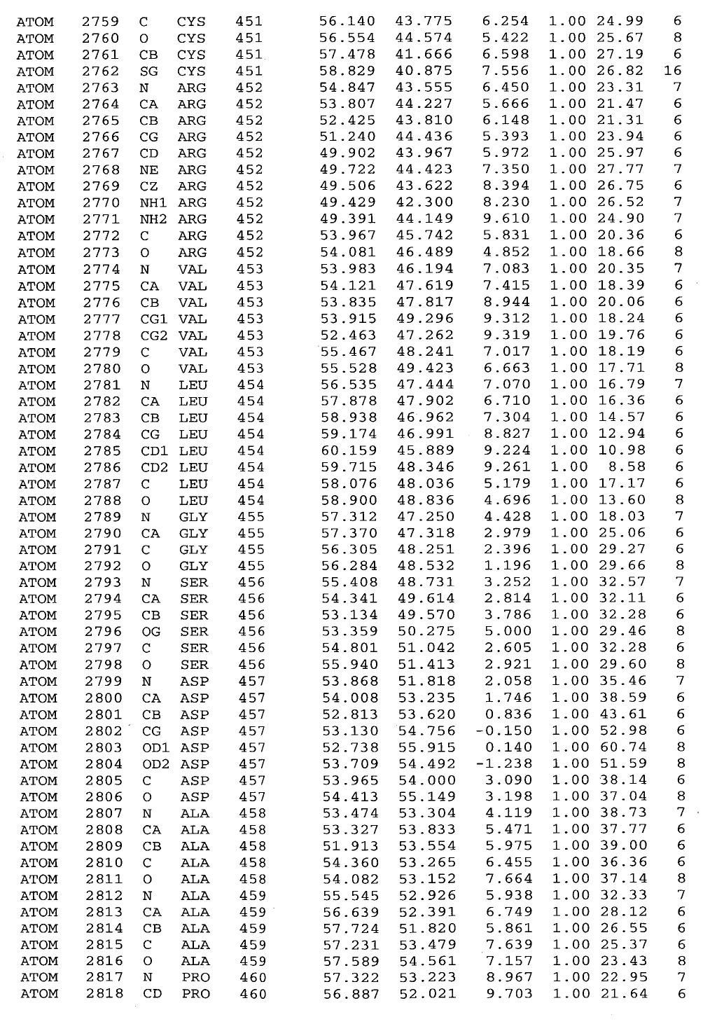

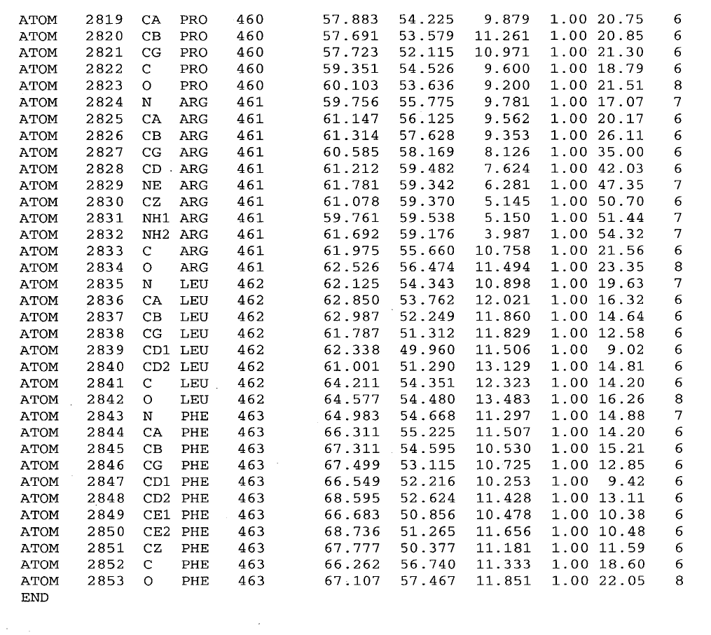

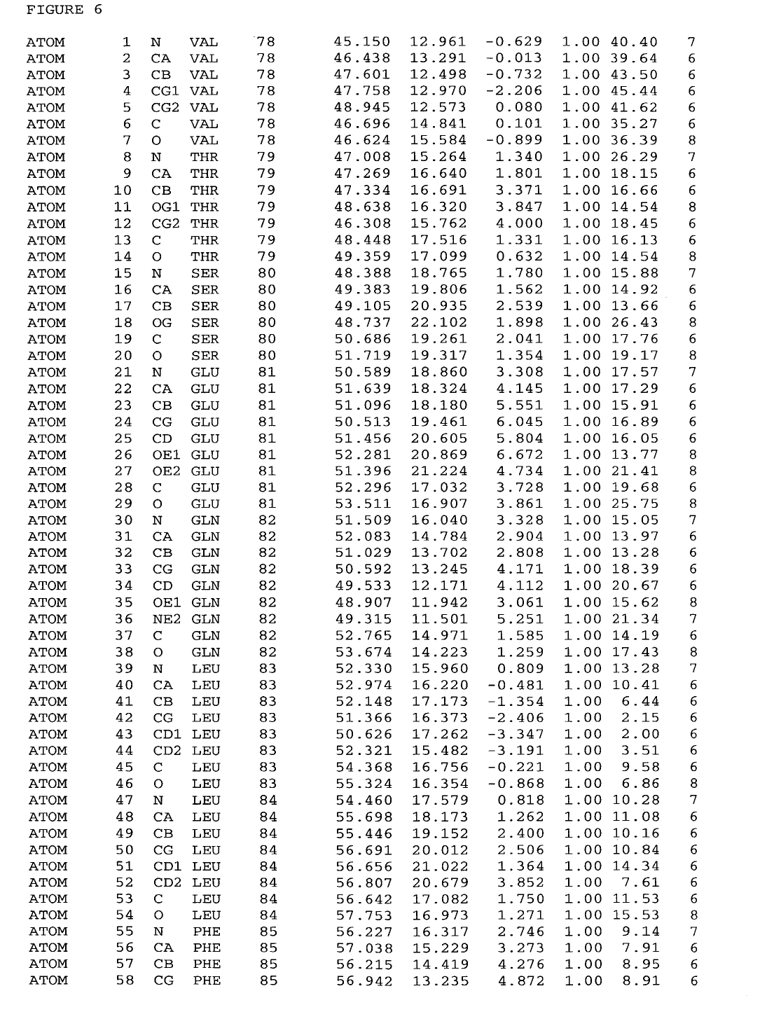

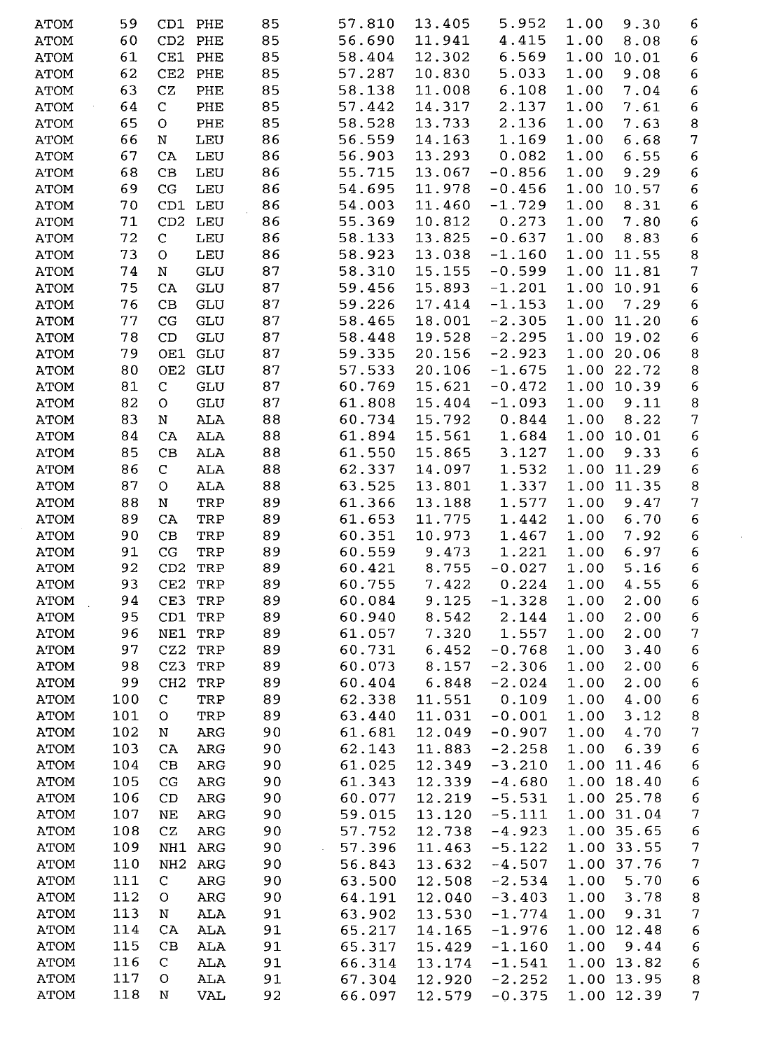

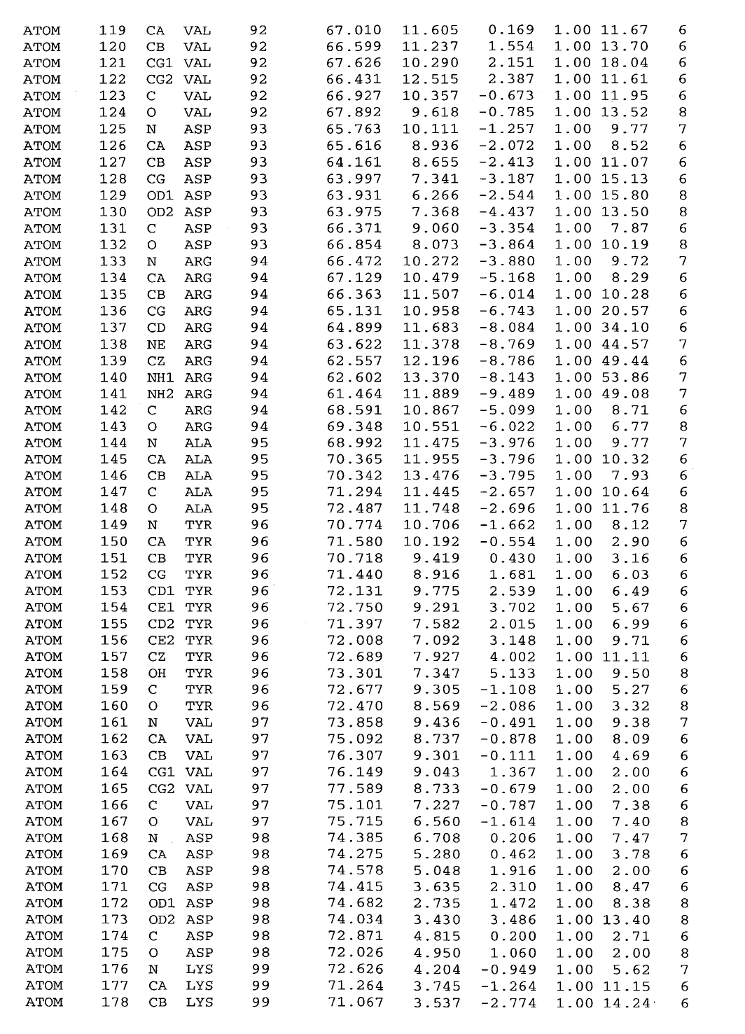

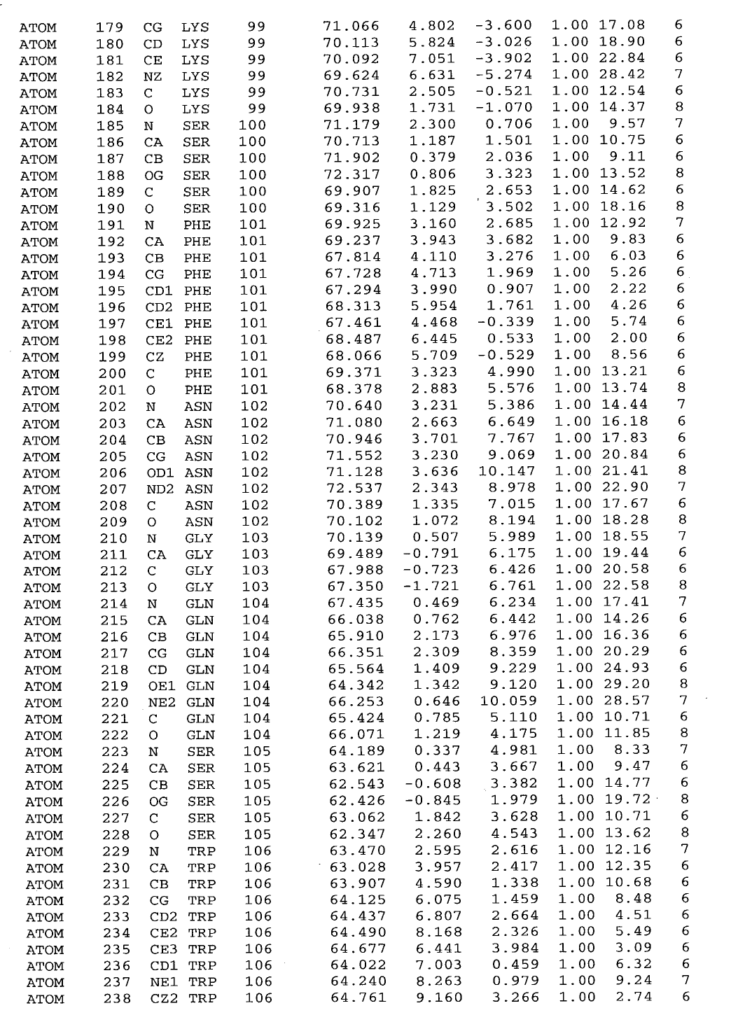

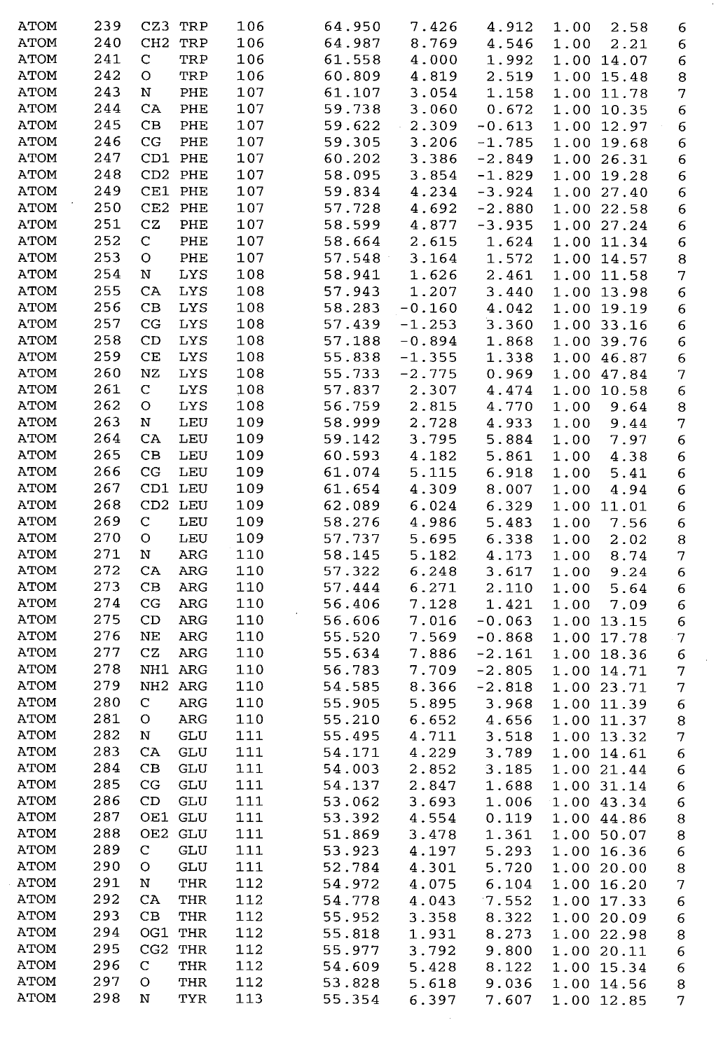

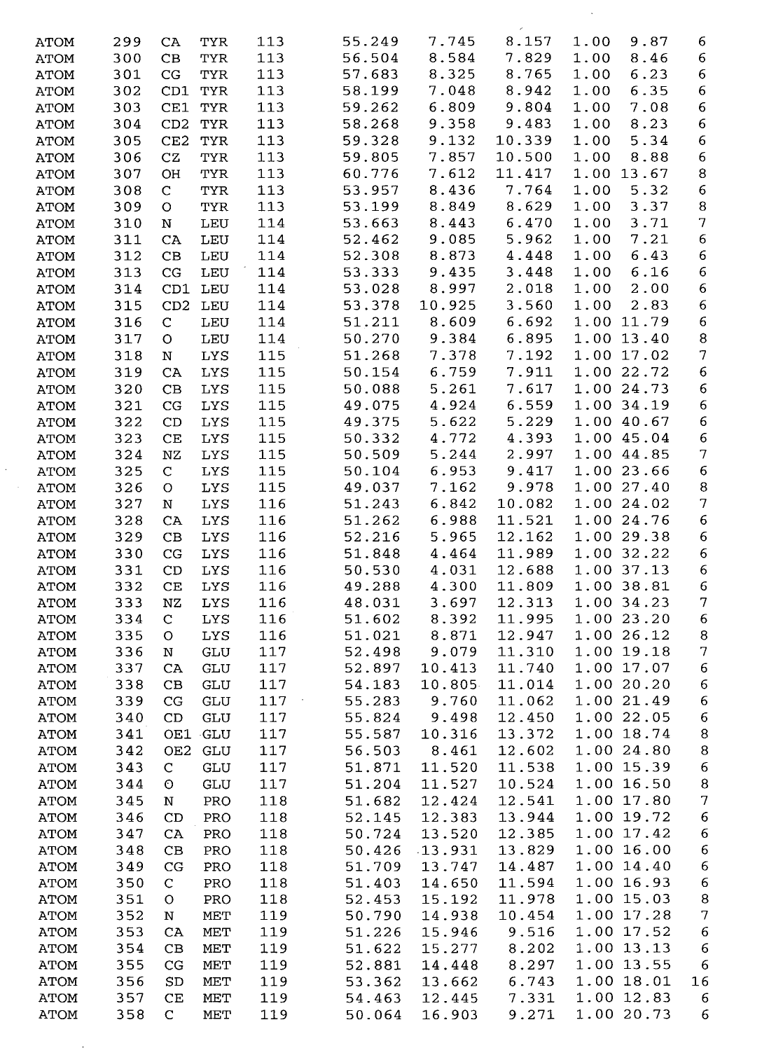

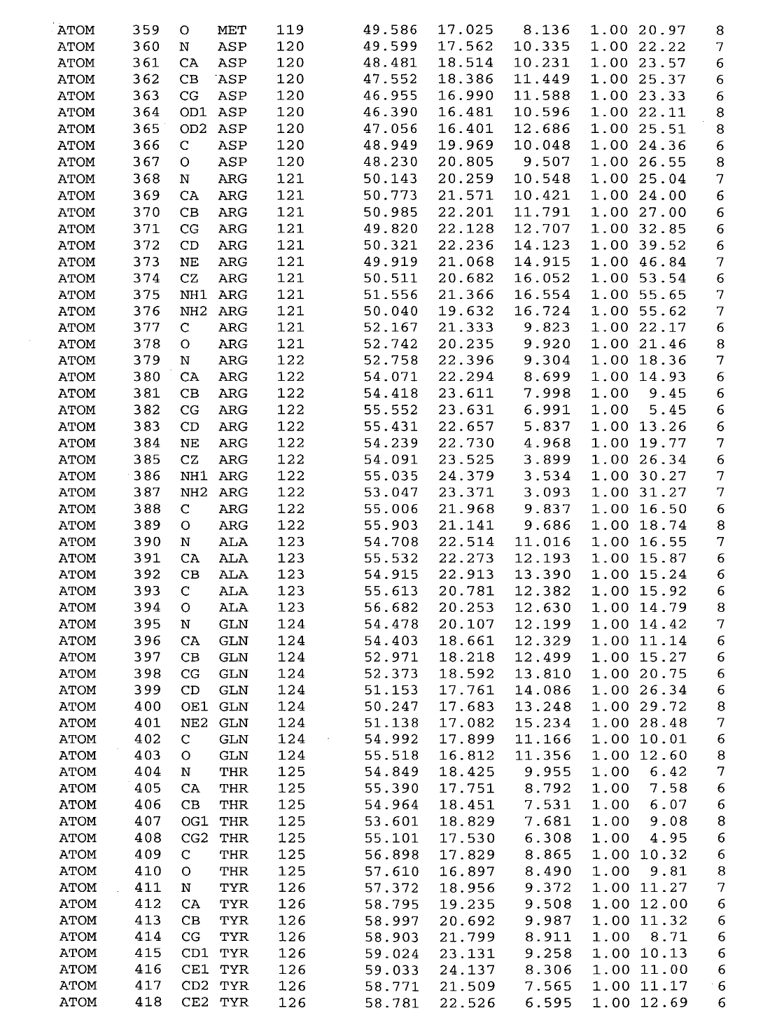

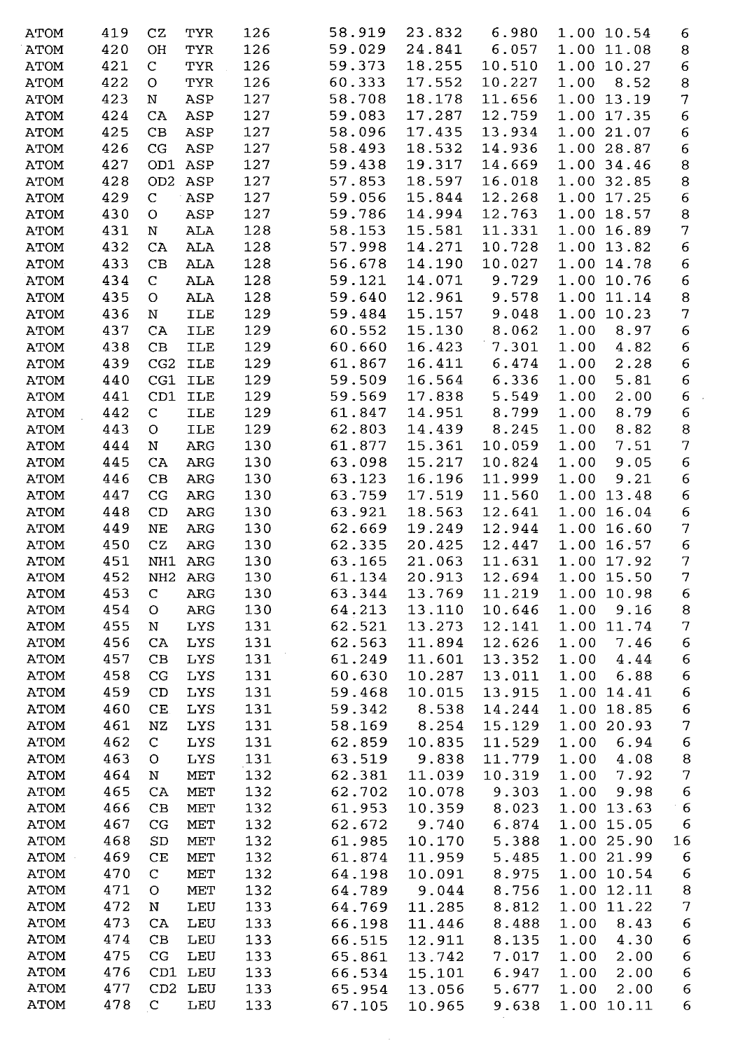

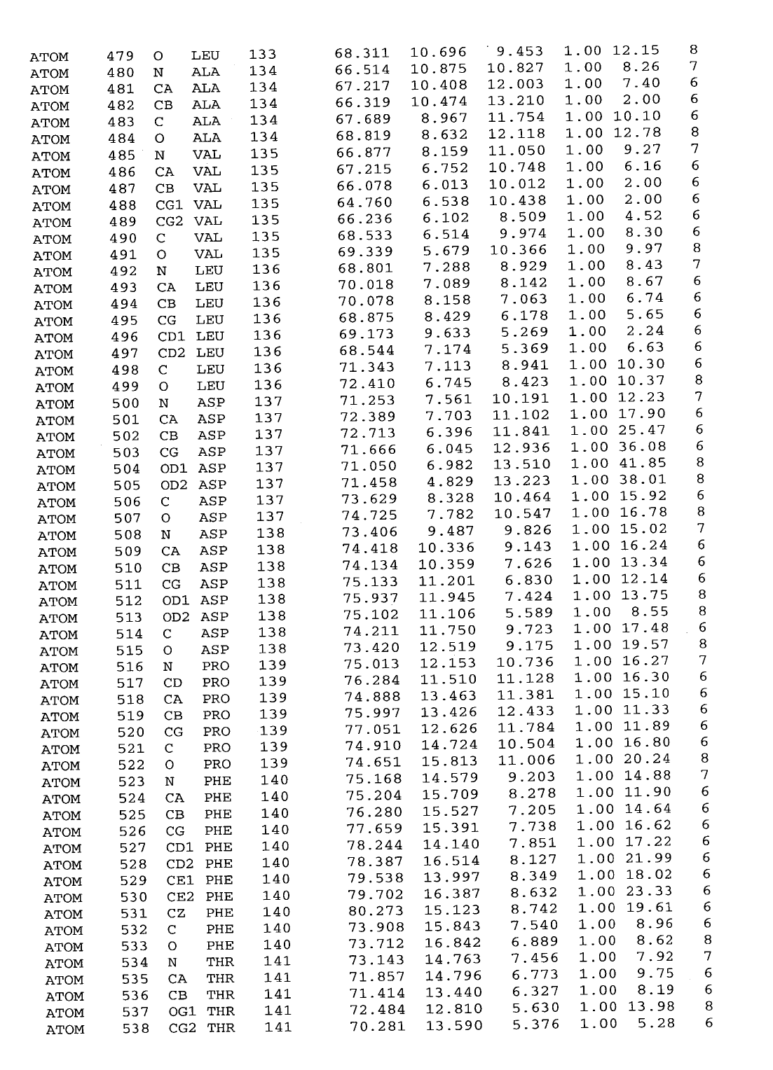

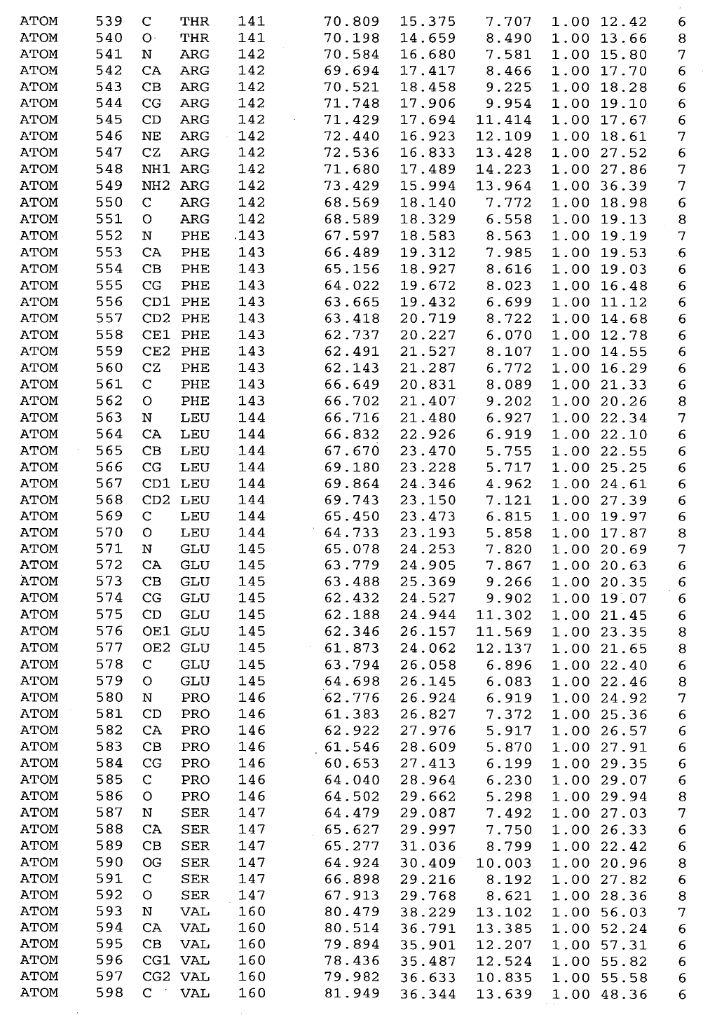

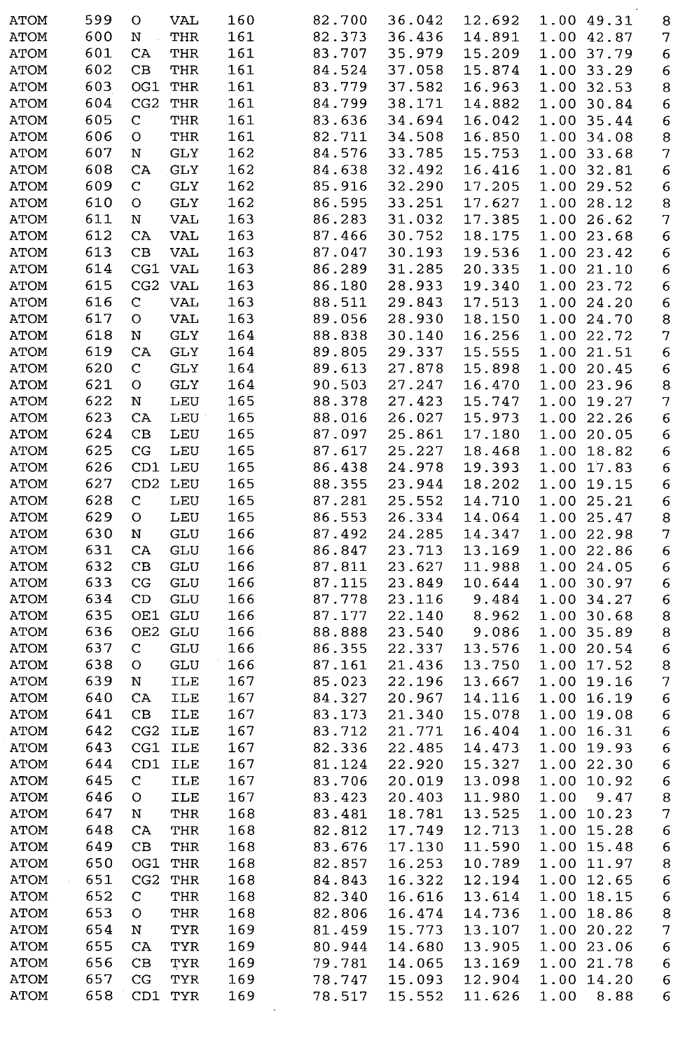

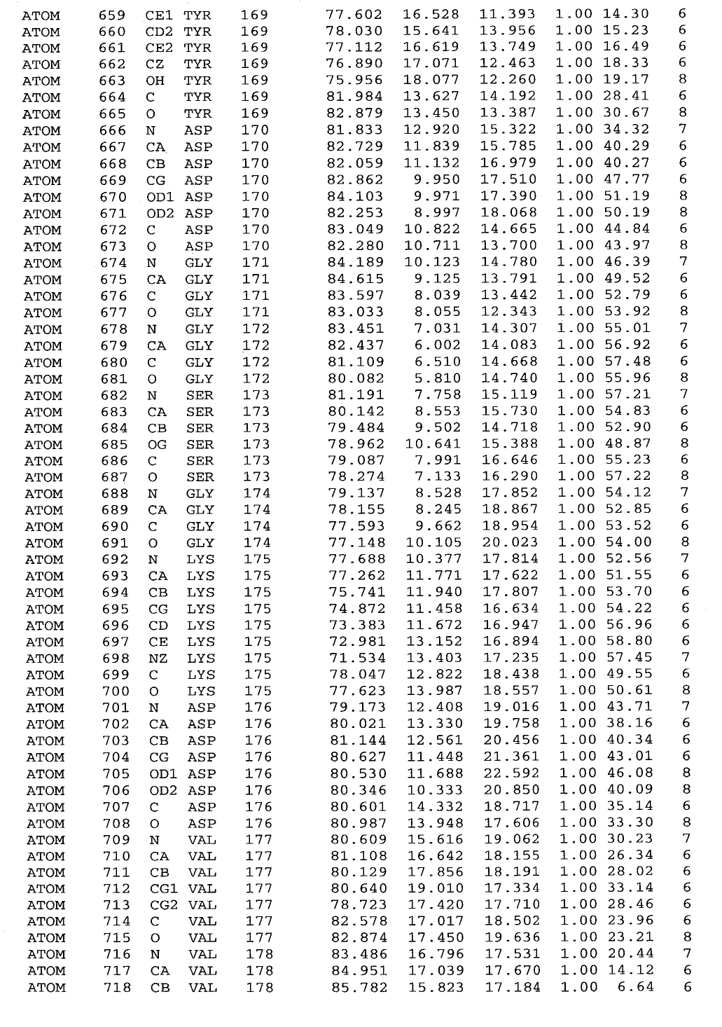

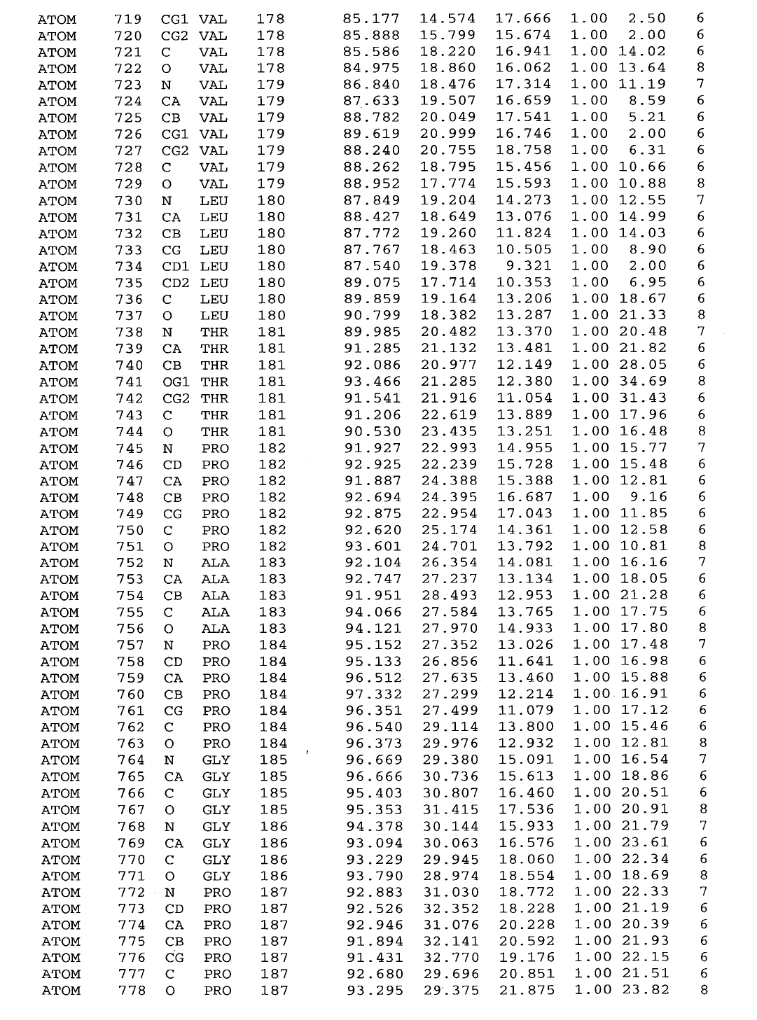

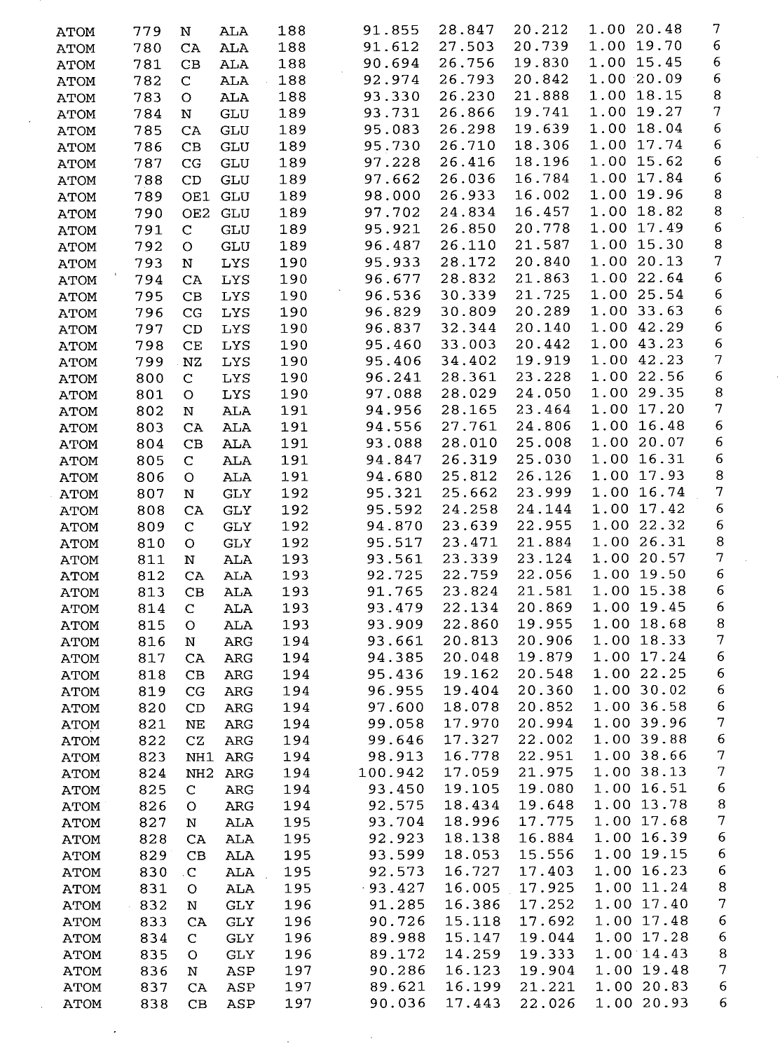

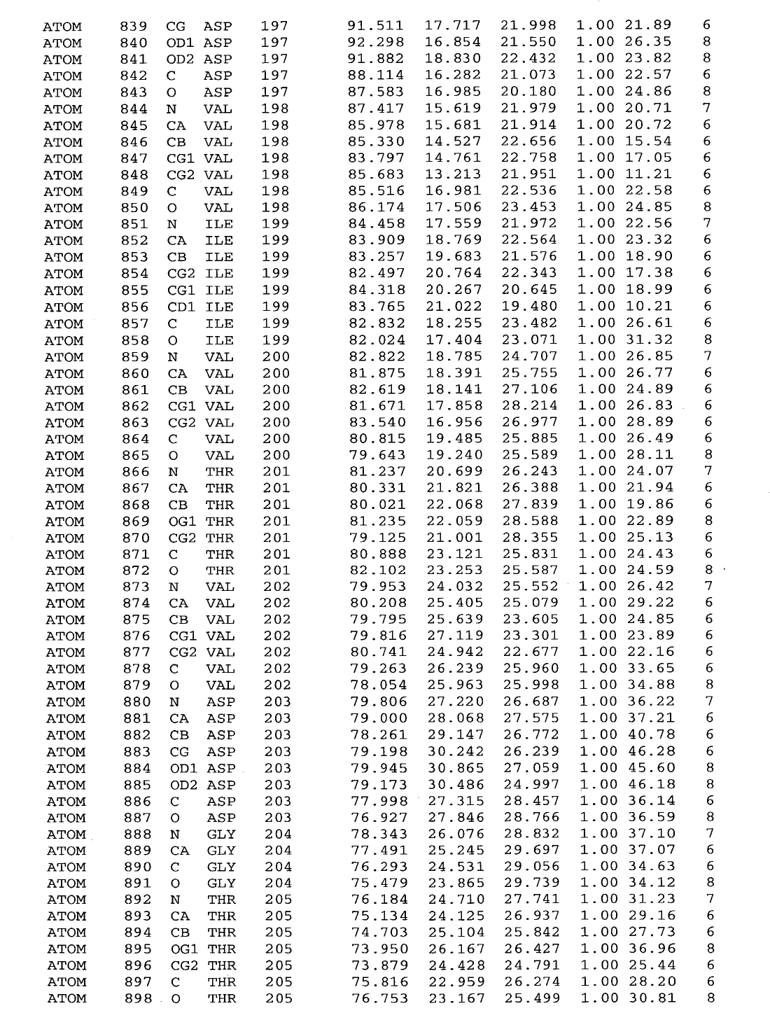

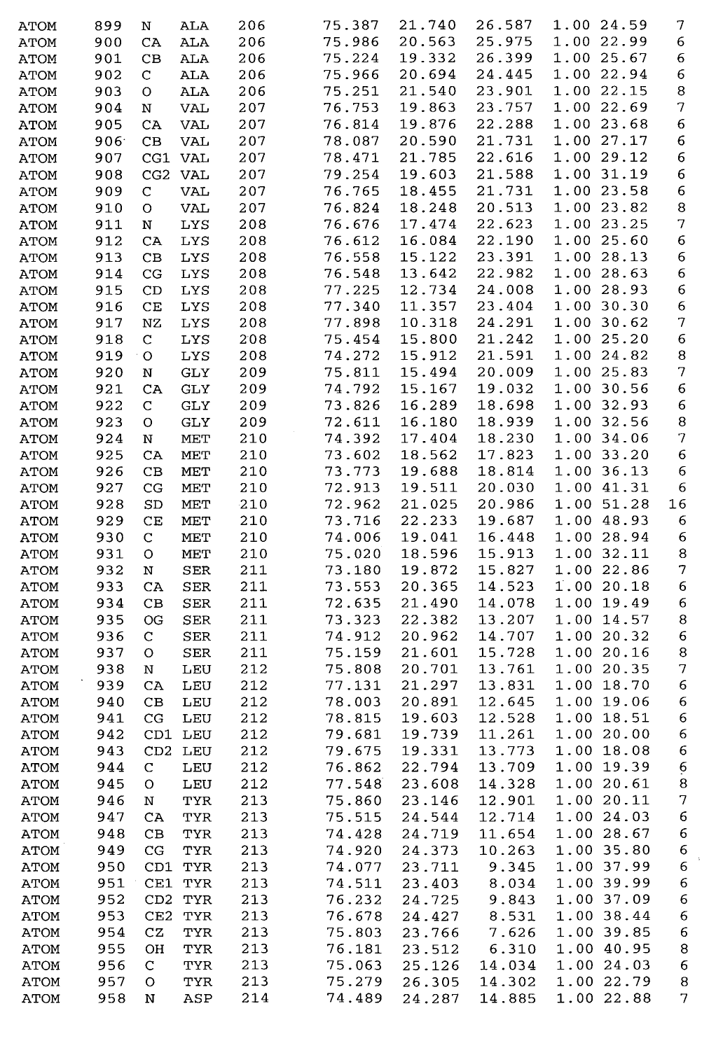

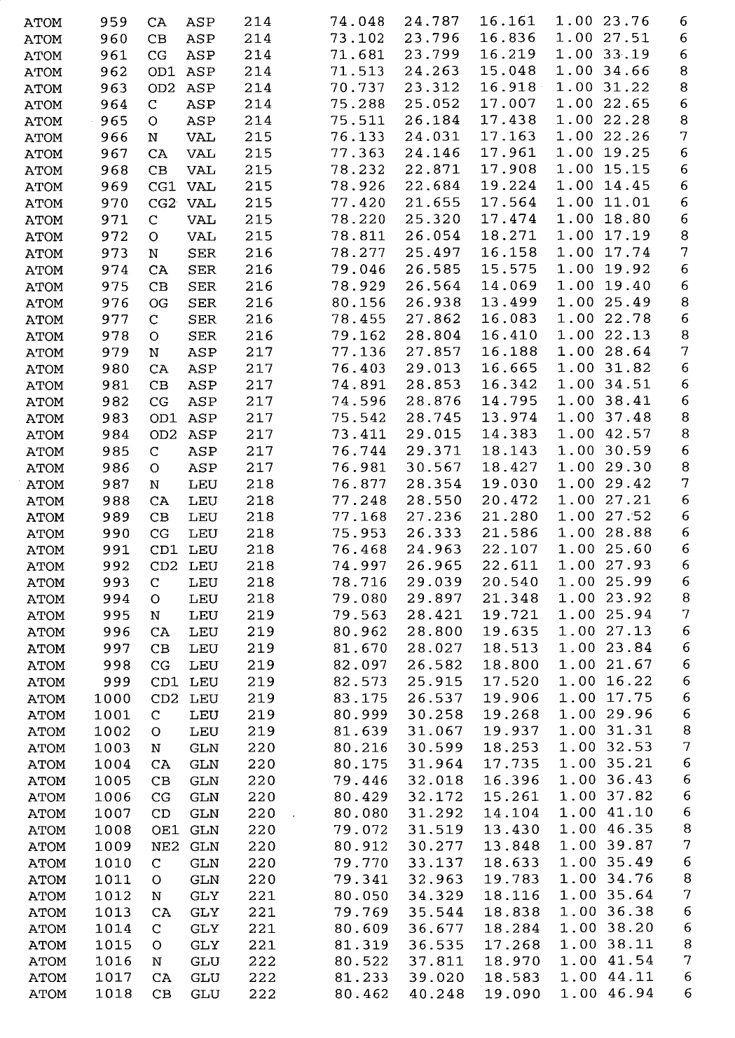

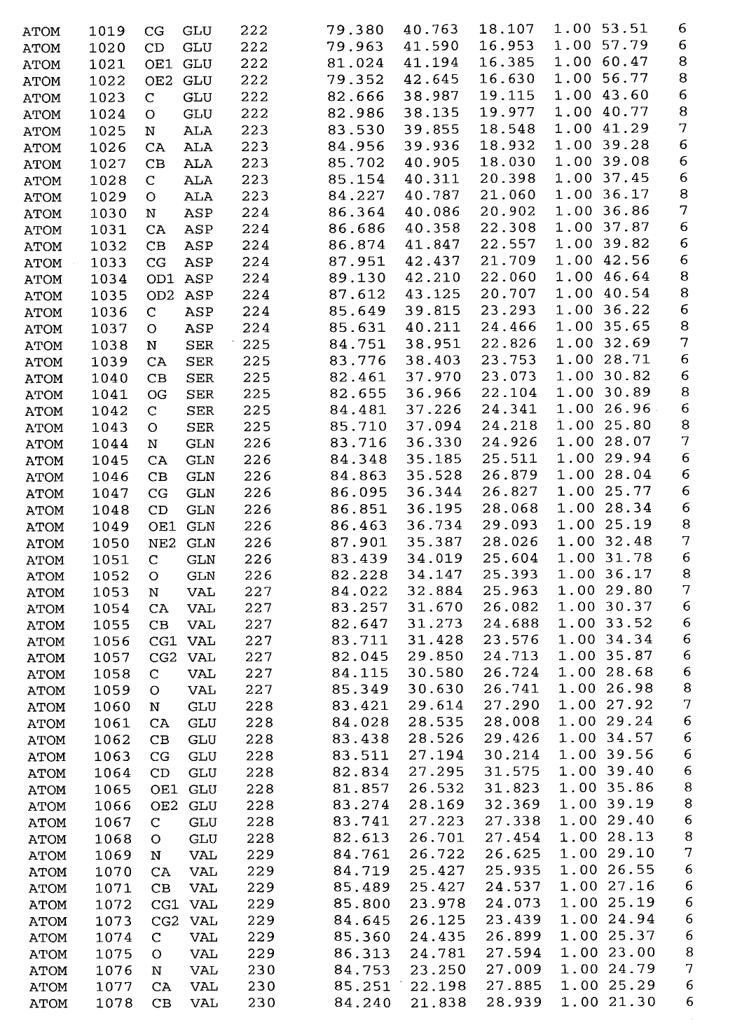

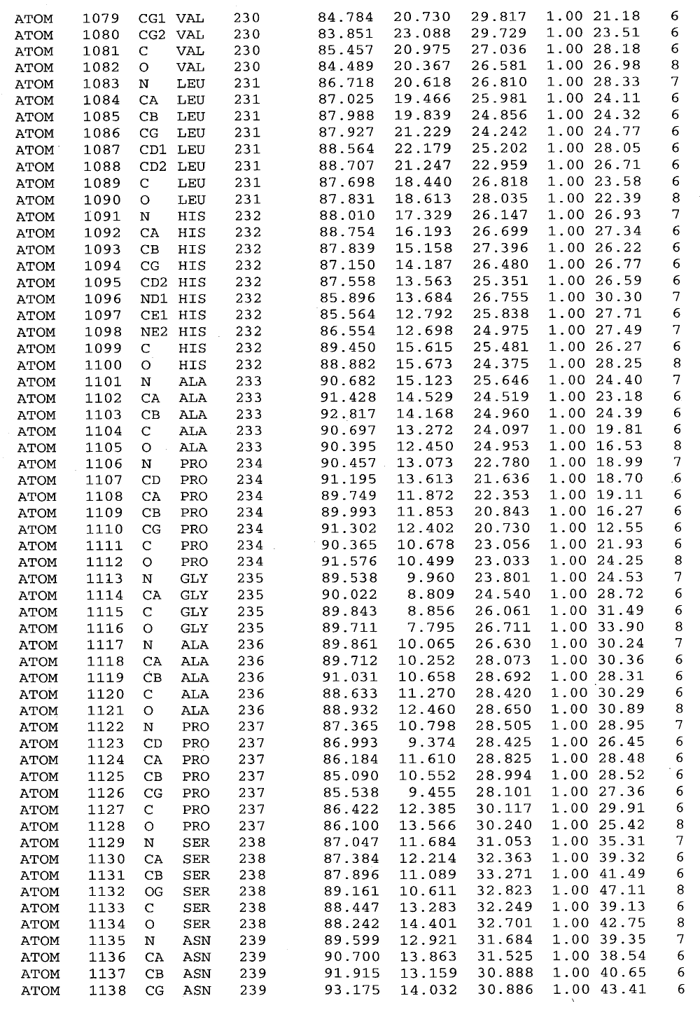

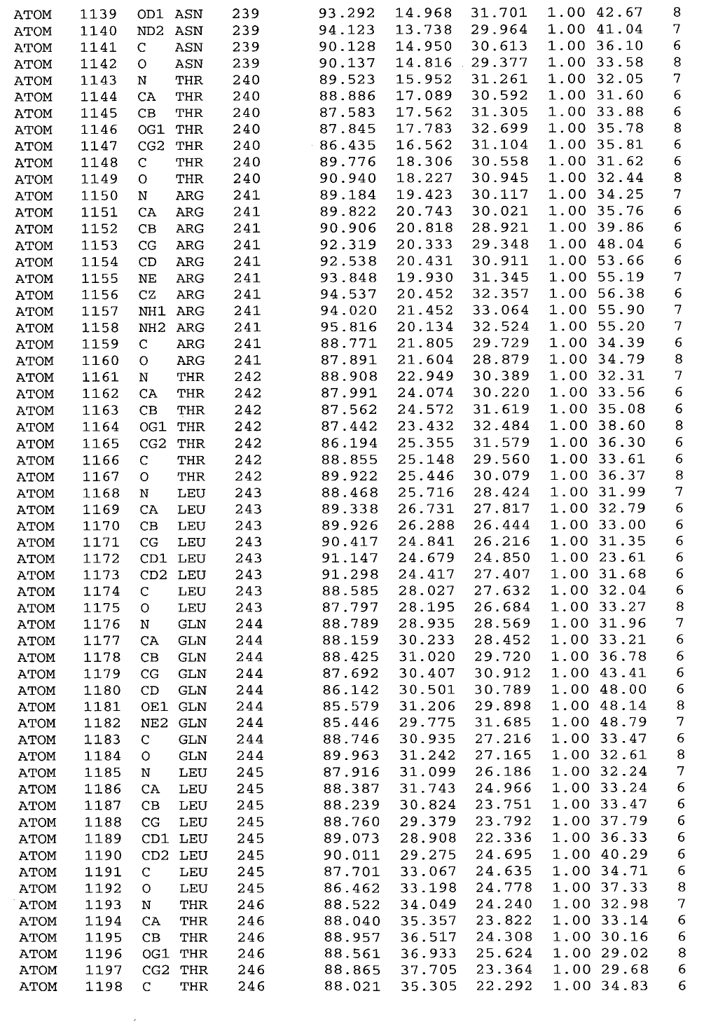

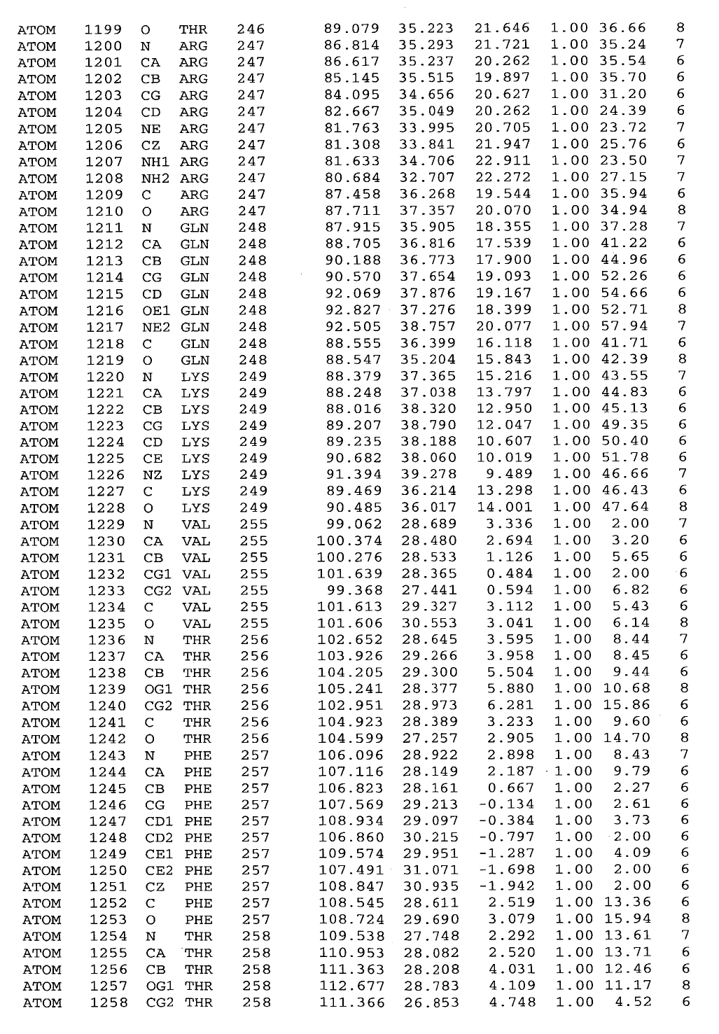

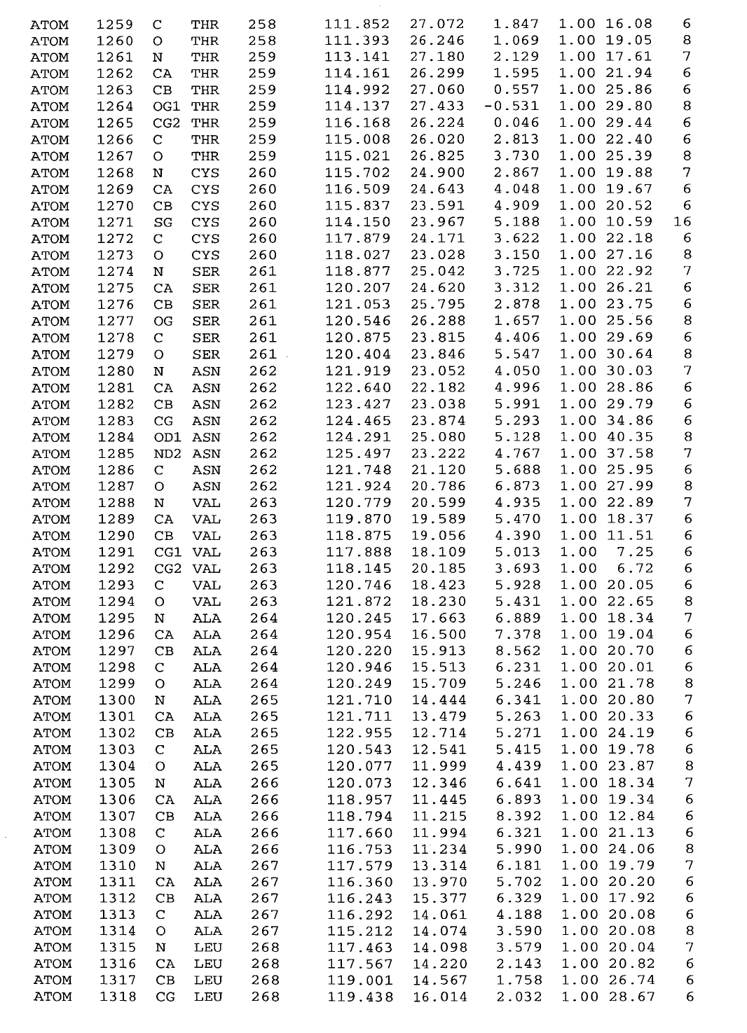

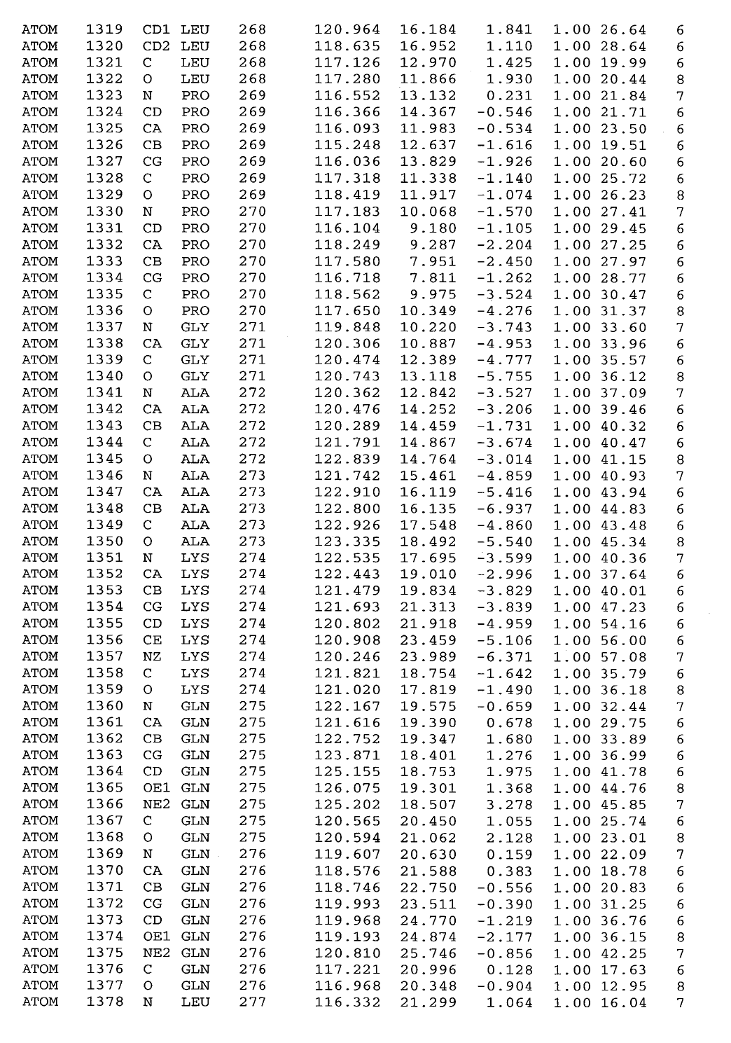

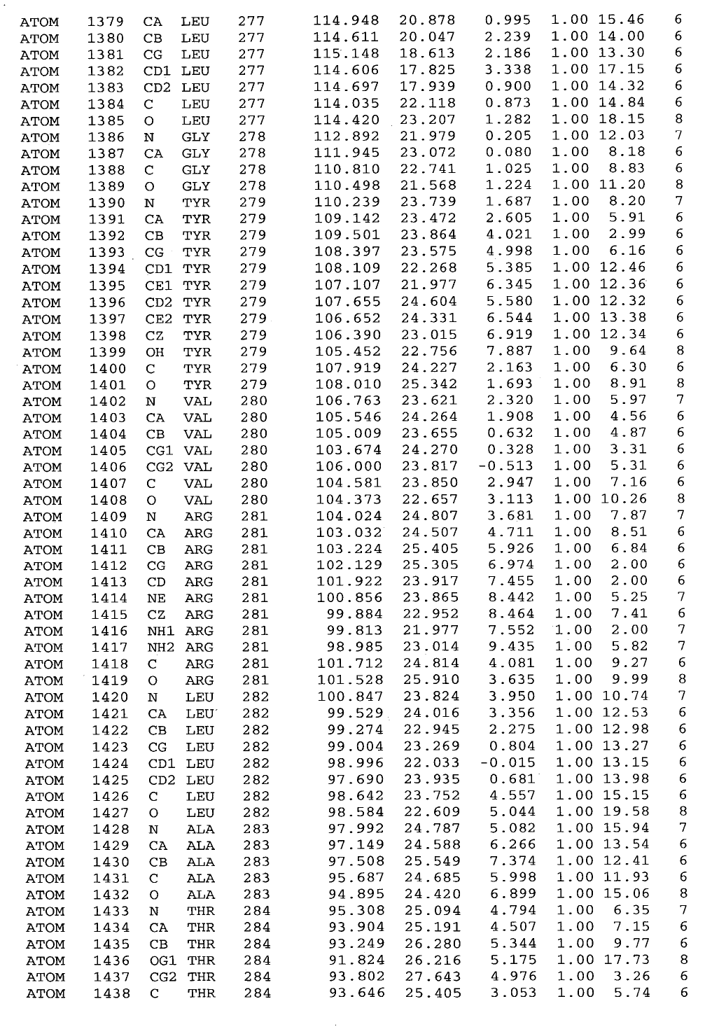

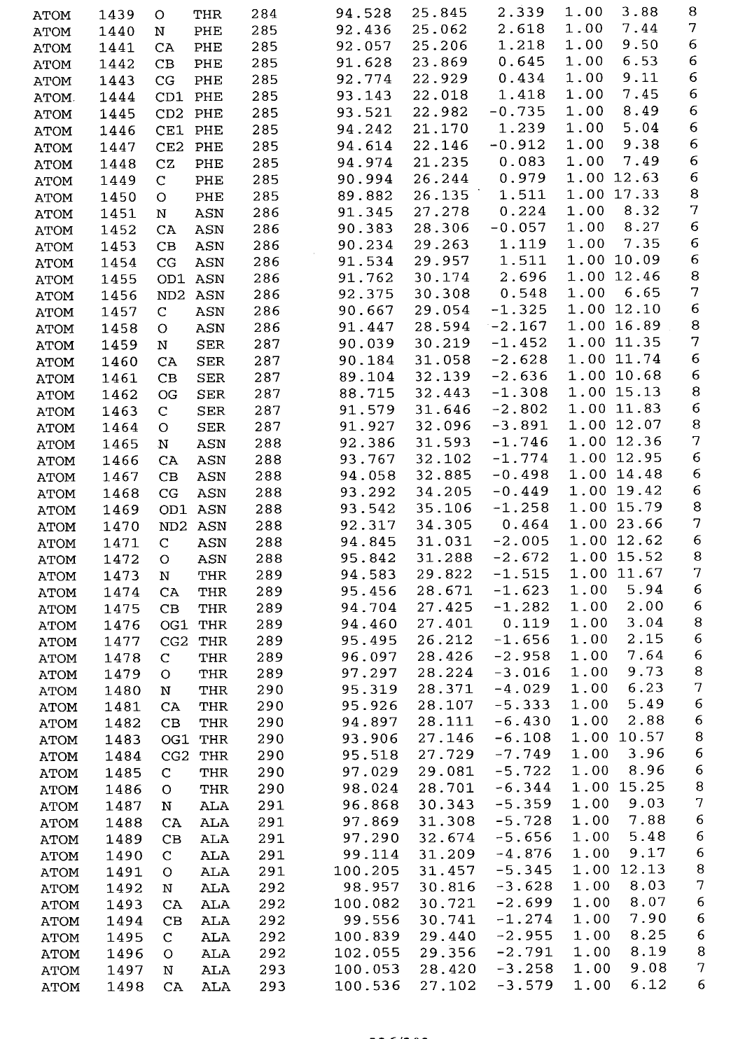

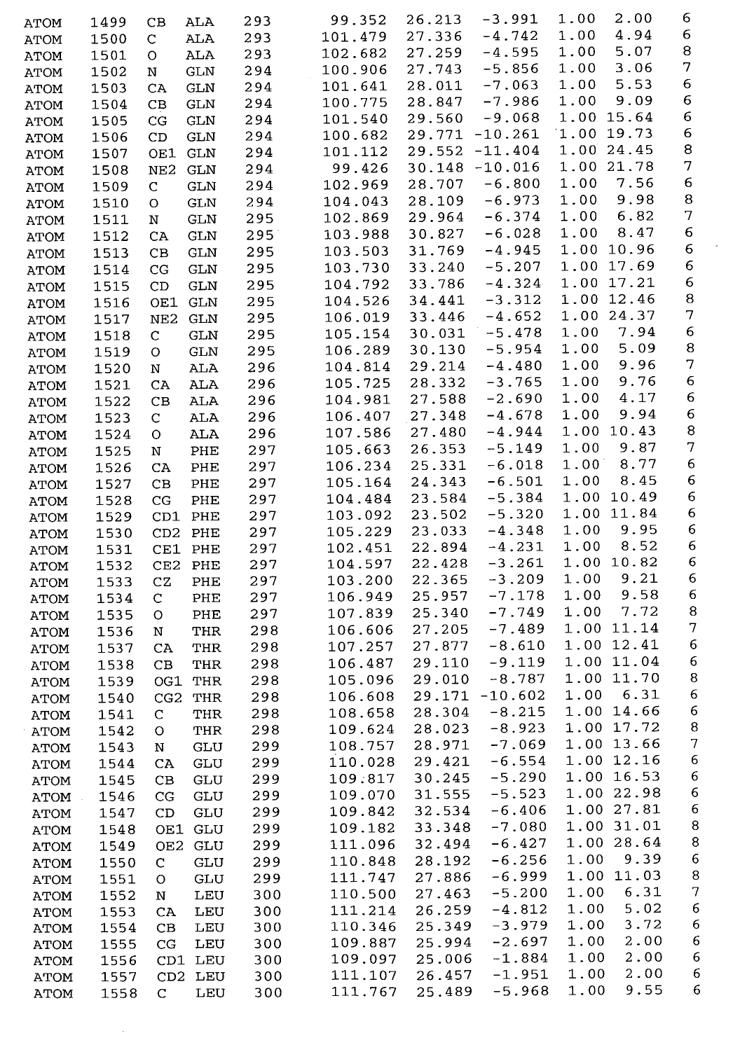

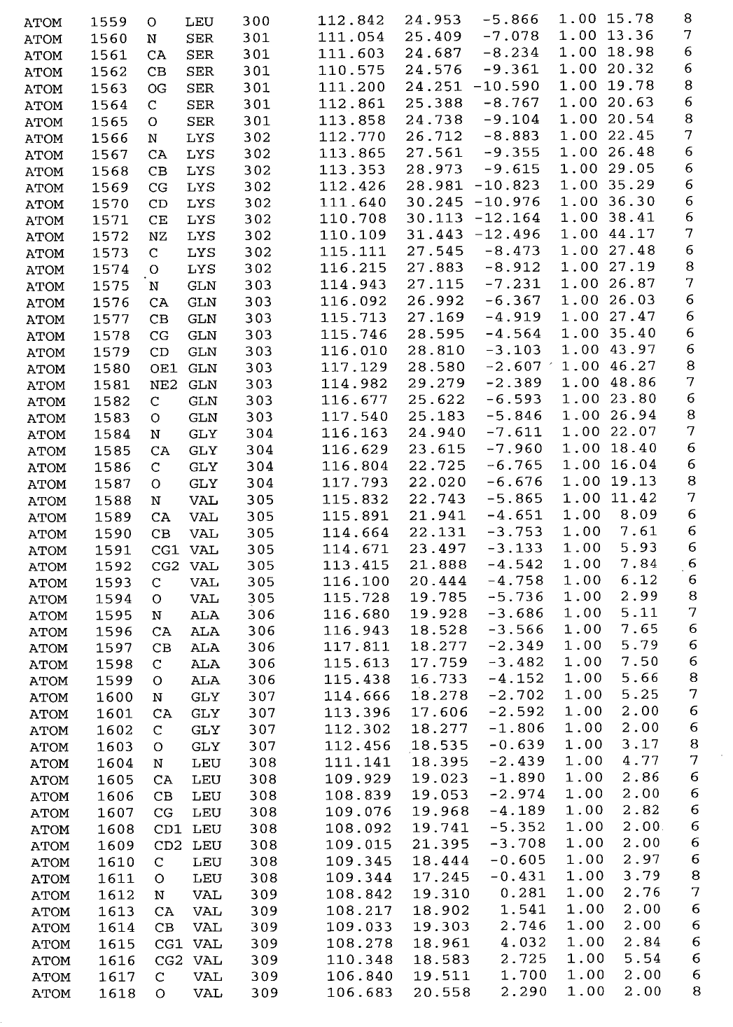

The data set of the native R32 form was collected in the same manner as native C2 I. Molecular replacement using the refined Se-Met mutant structure with water removed as the search model, was done using the program AMoRe (Navaza, J., Acta Crystallogr. A50:157-163 (1994)). Program X-PLOR was used for the refinement. Rigidbody refinement was done by breaking up the model into three folding domains and allowing each domain to move independently. After one cycle of positional refinement and one cycle of B-factor refinement, the working R factor is 27.0% and the free R factor is 37.0%. This data set is shown in FIG. 6.

### Example 13

Building a Homology Model of Wheat D1 Protease Based on the Coordinates of Scenedesmus obliquus D1 Protease

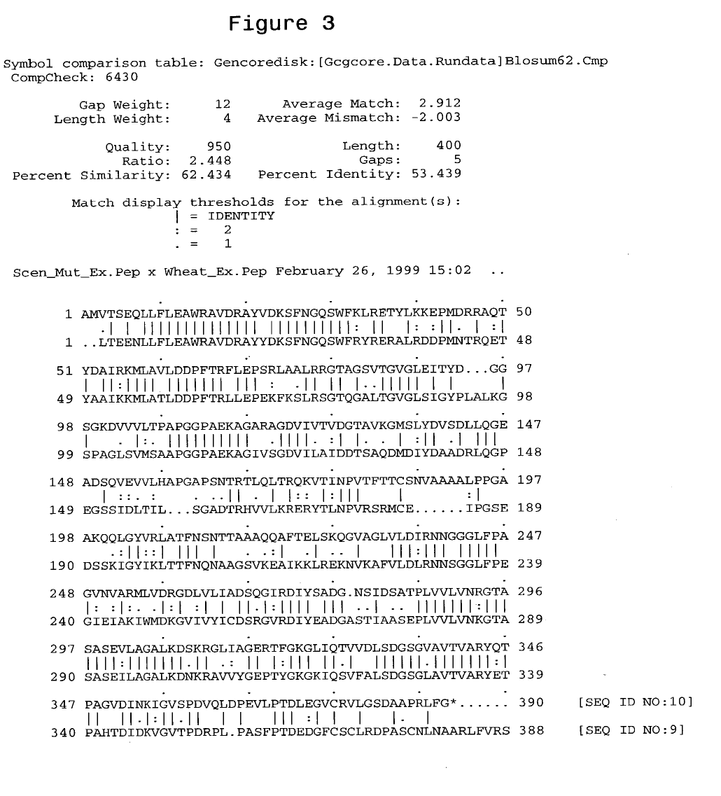

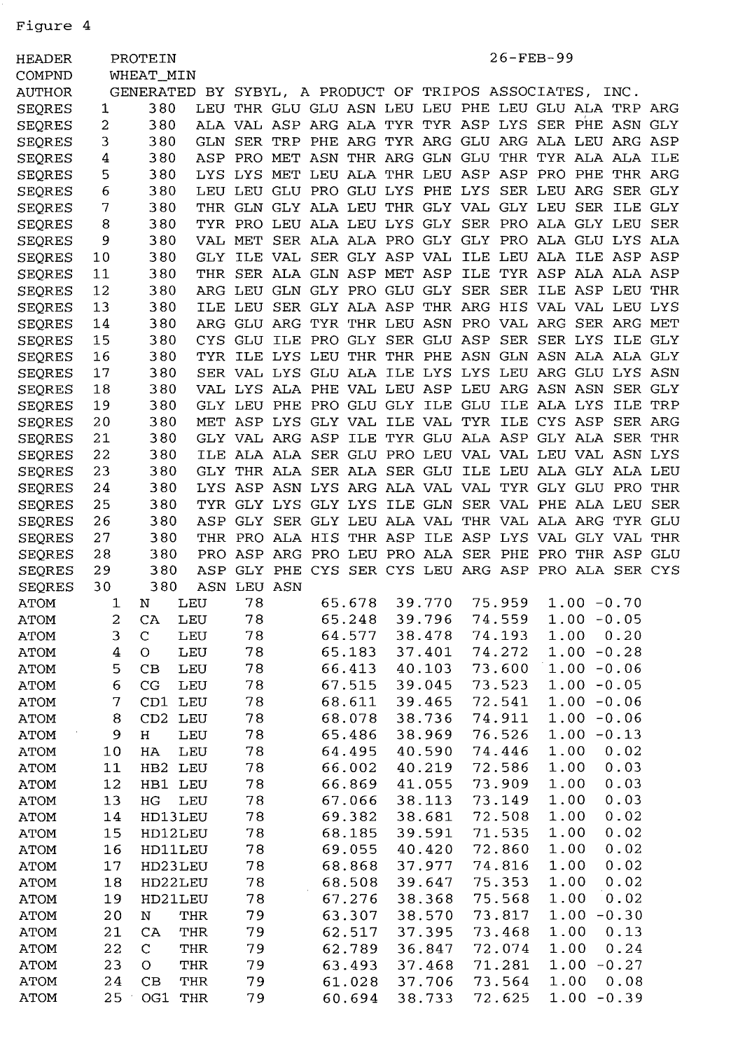

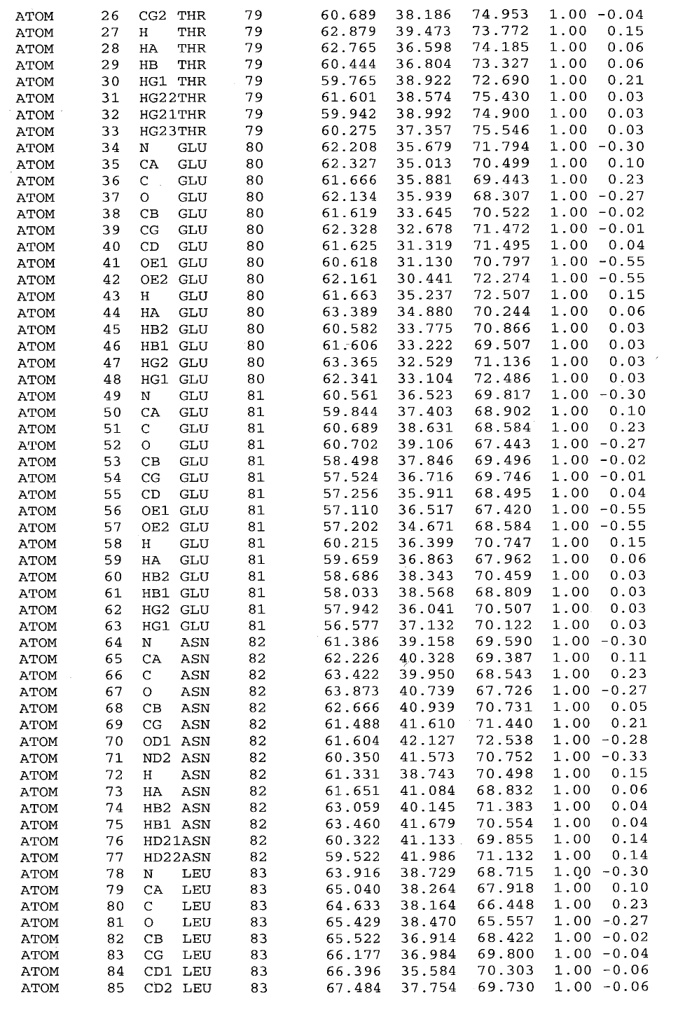

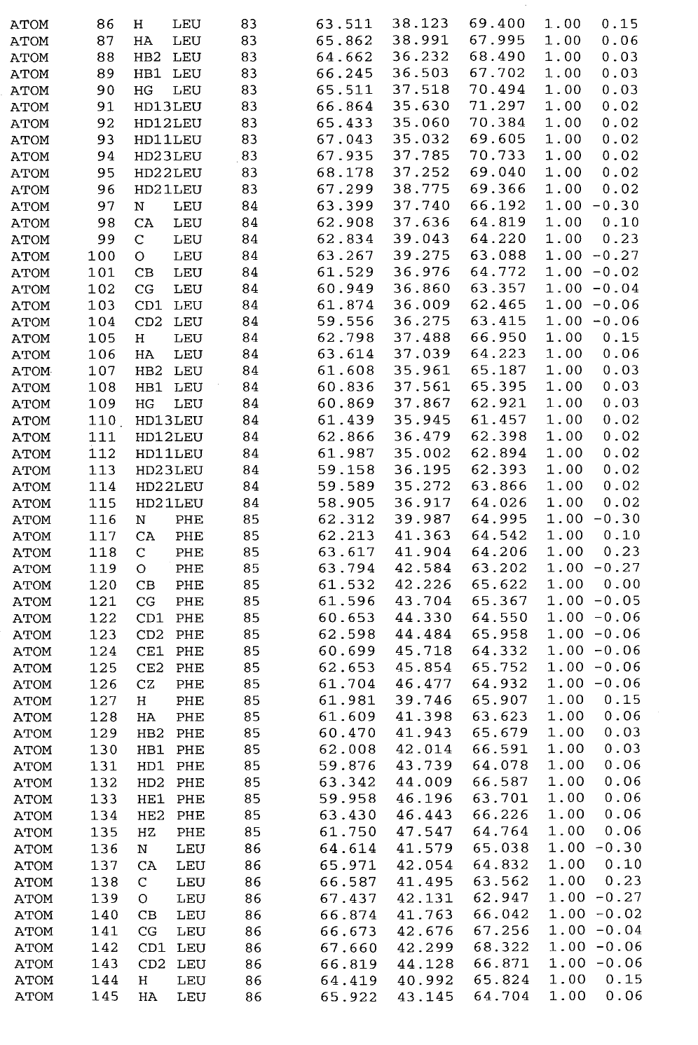

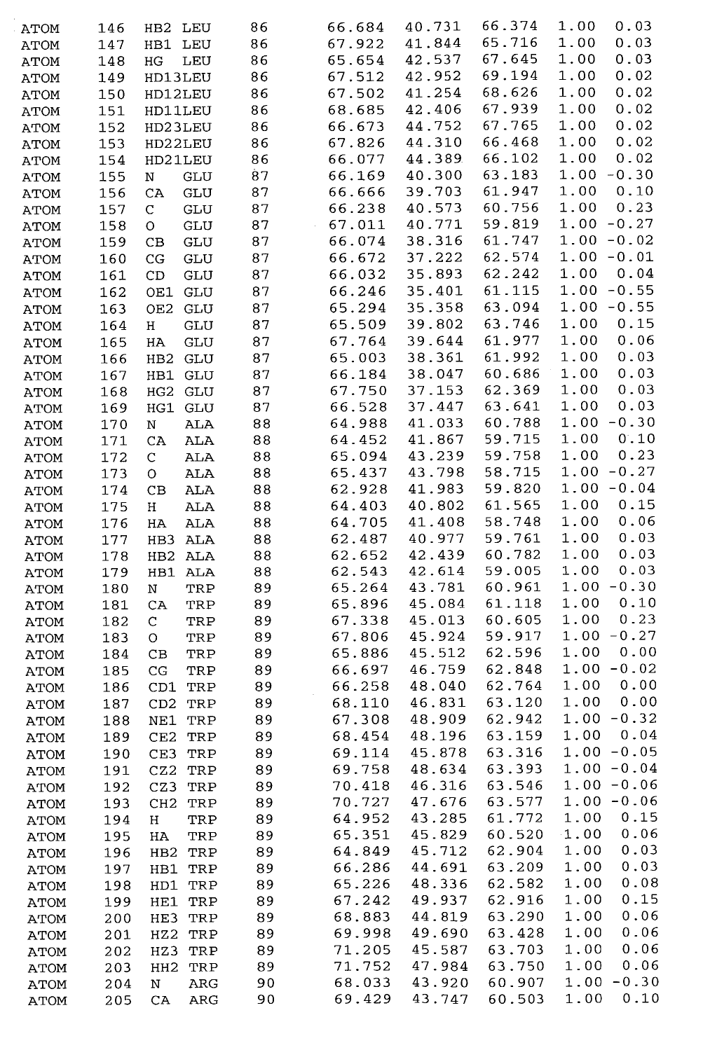

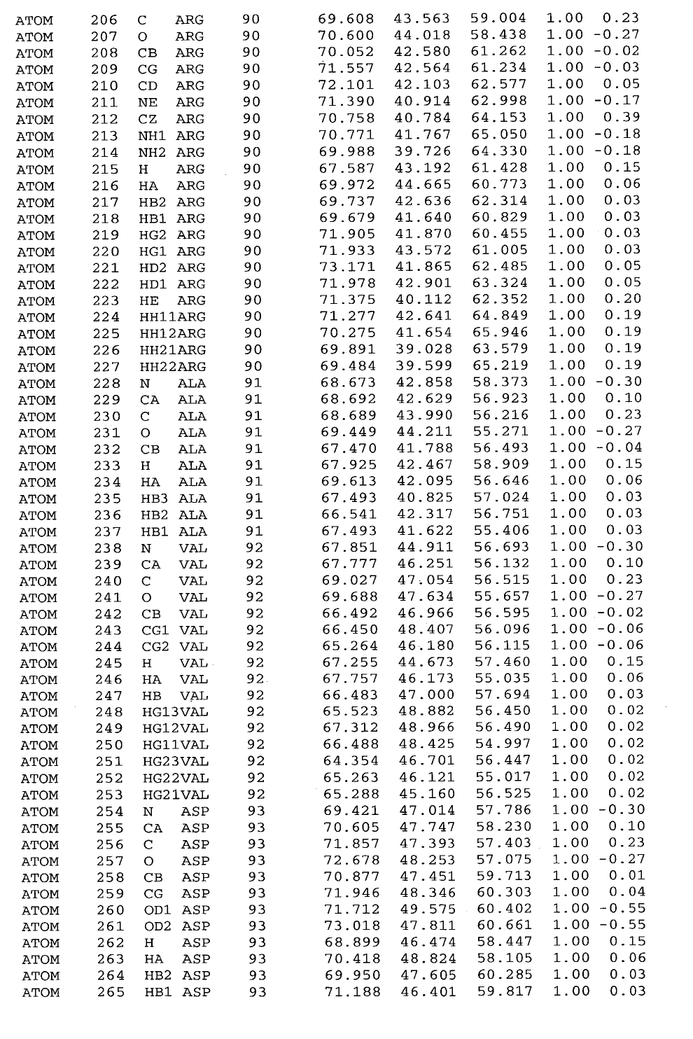

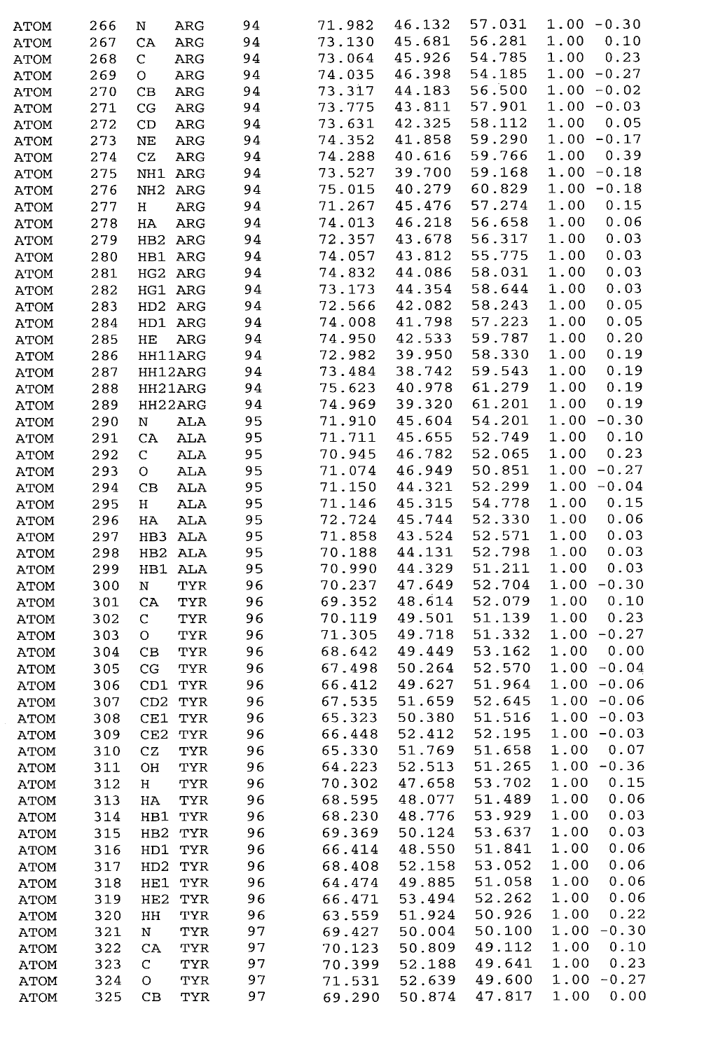

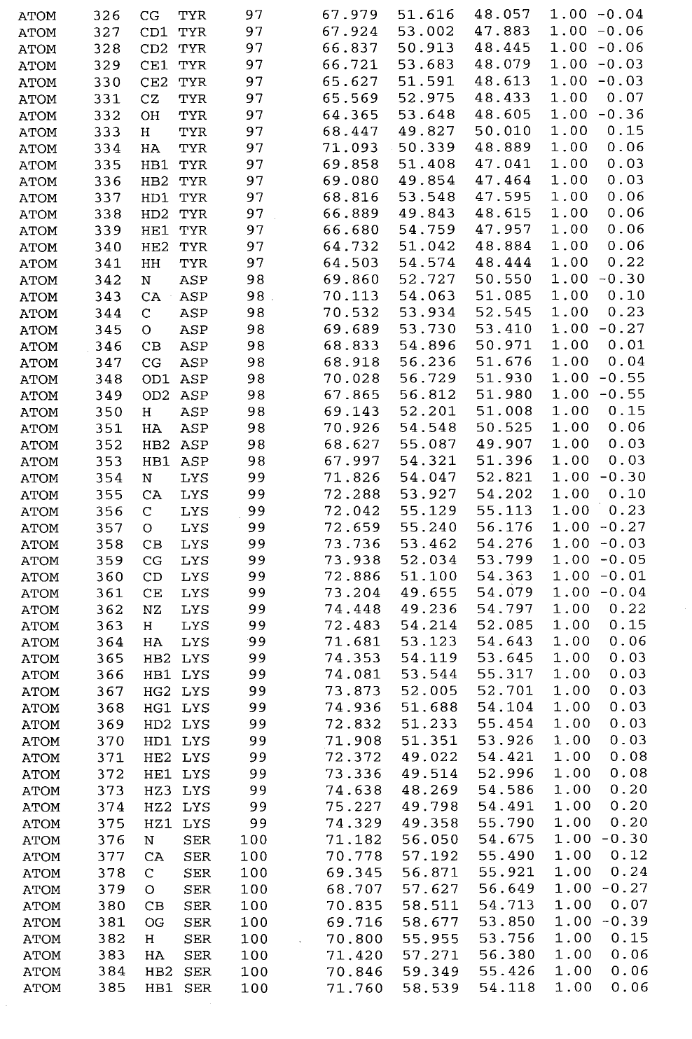

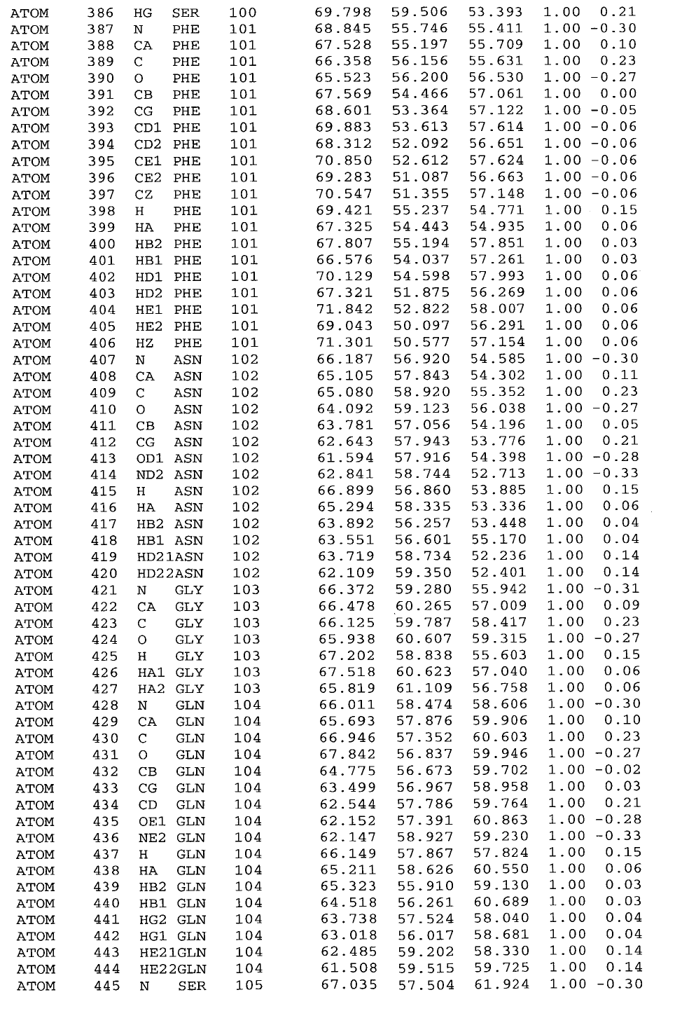

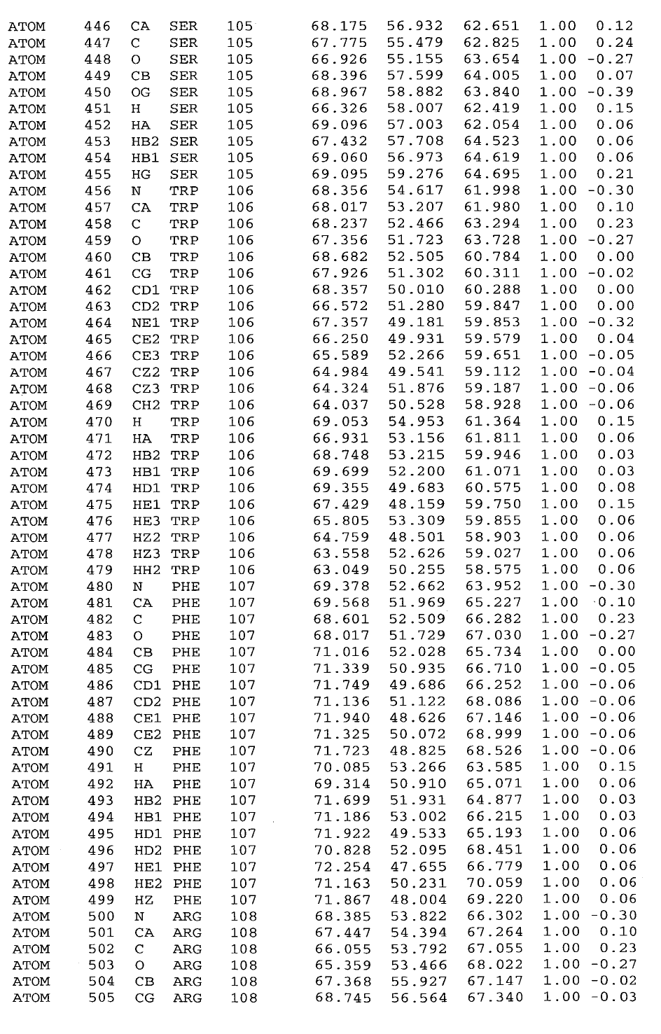

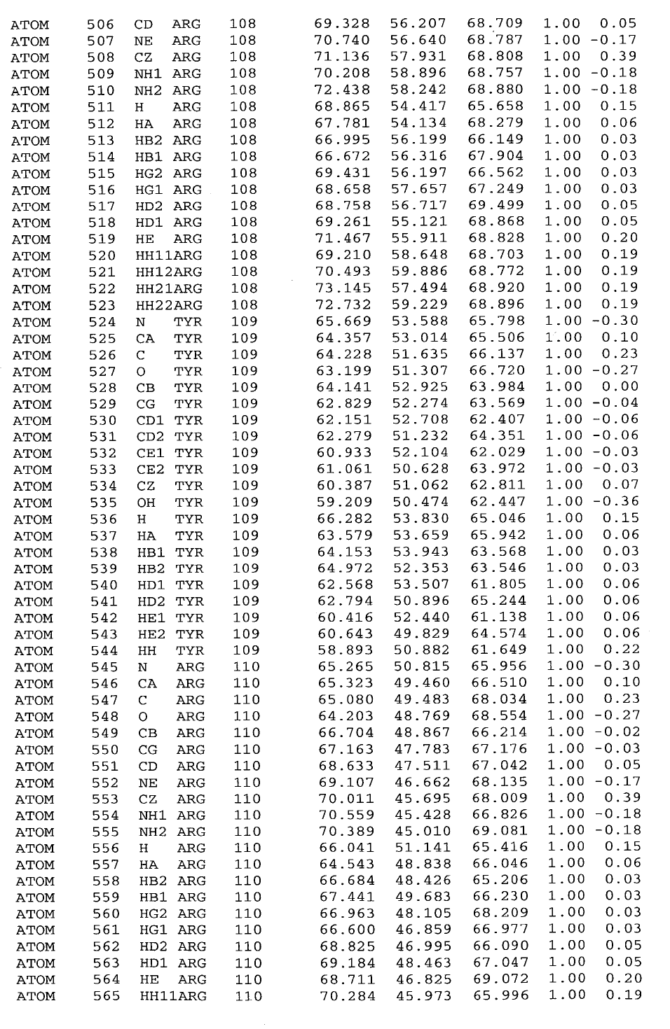

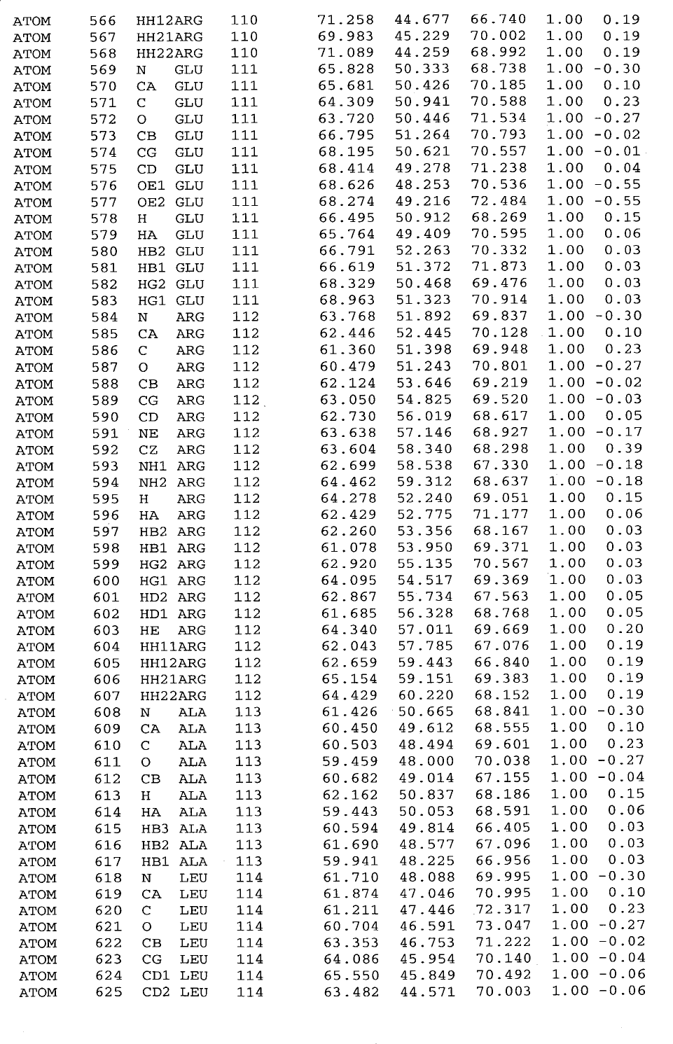

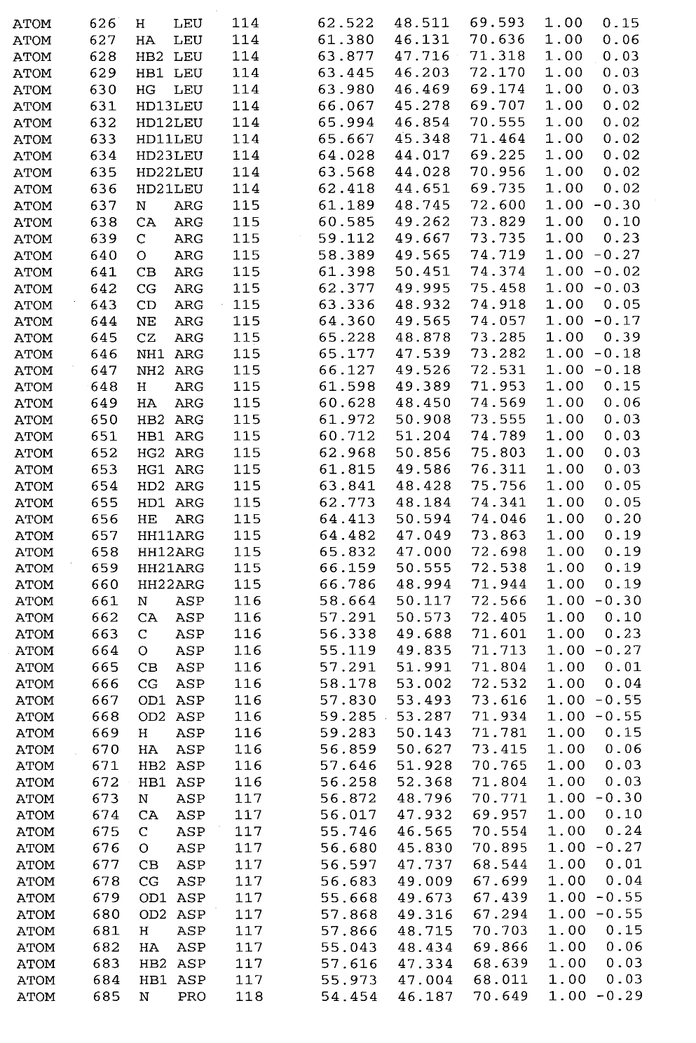

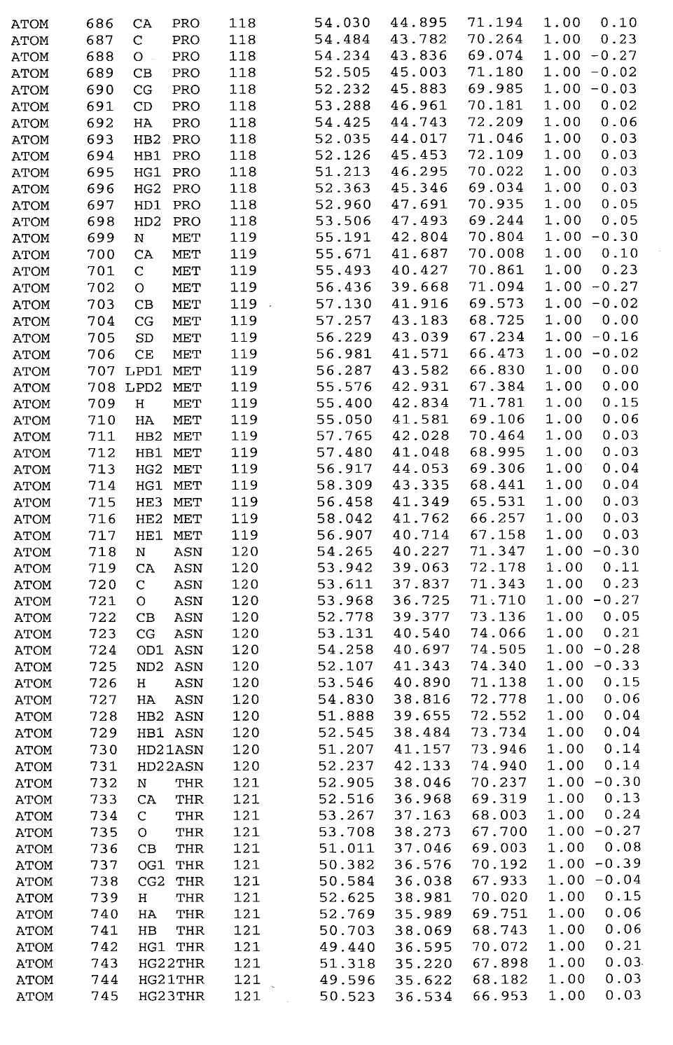

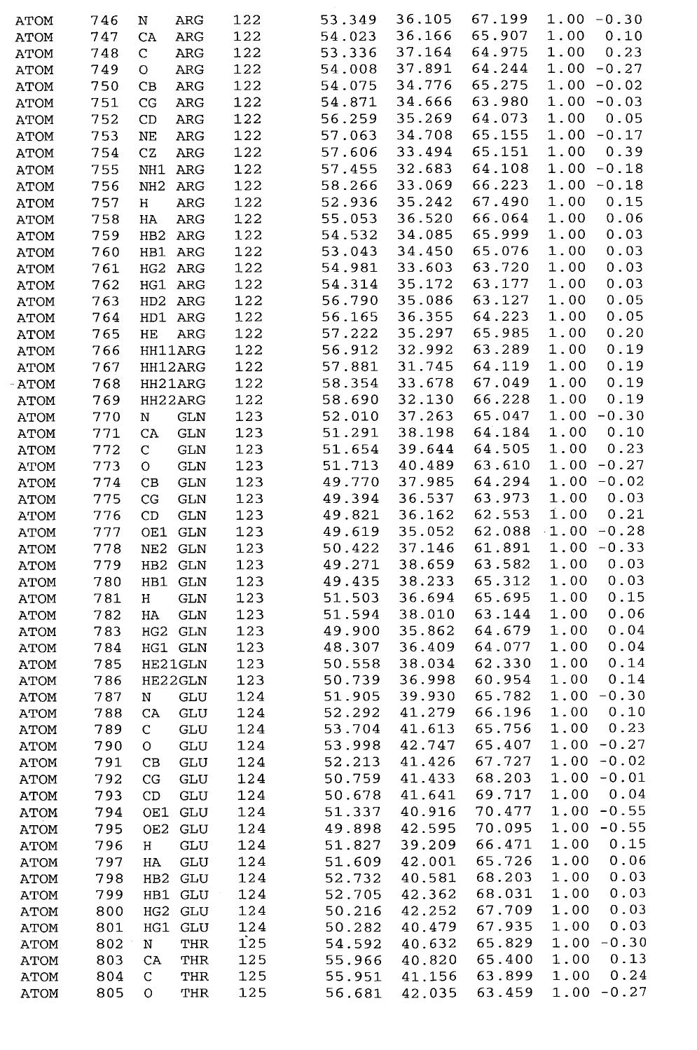

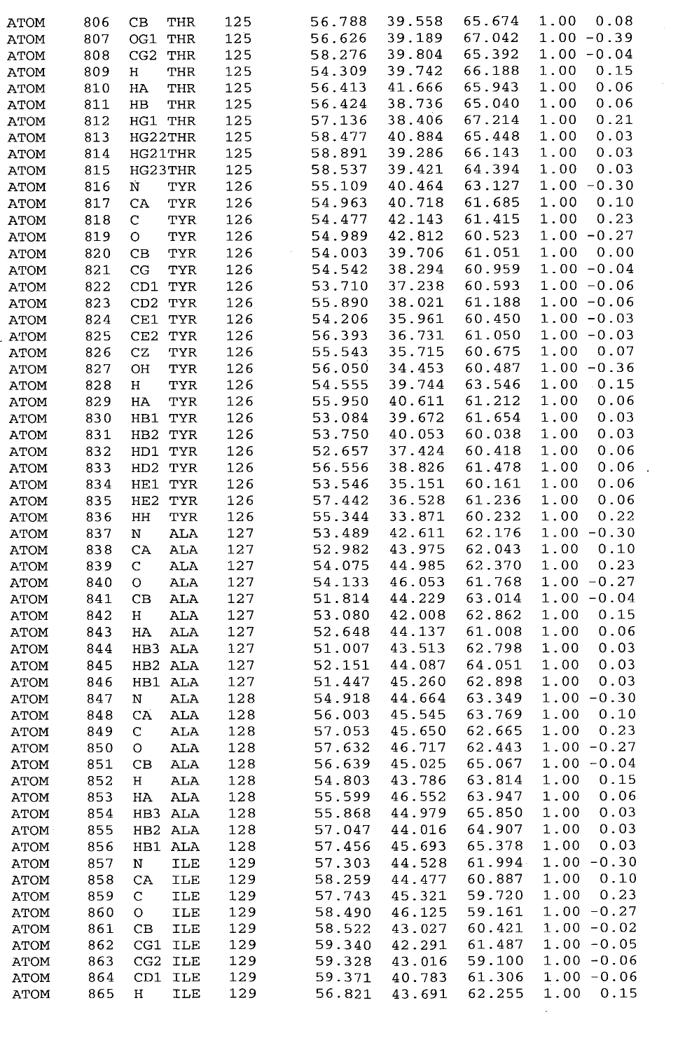

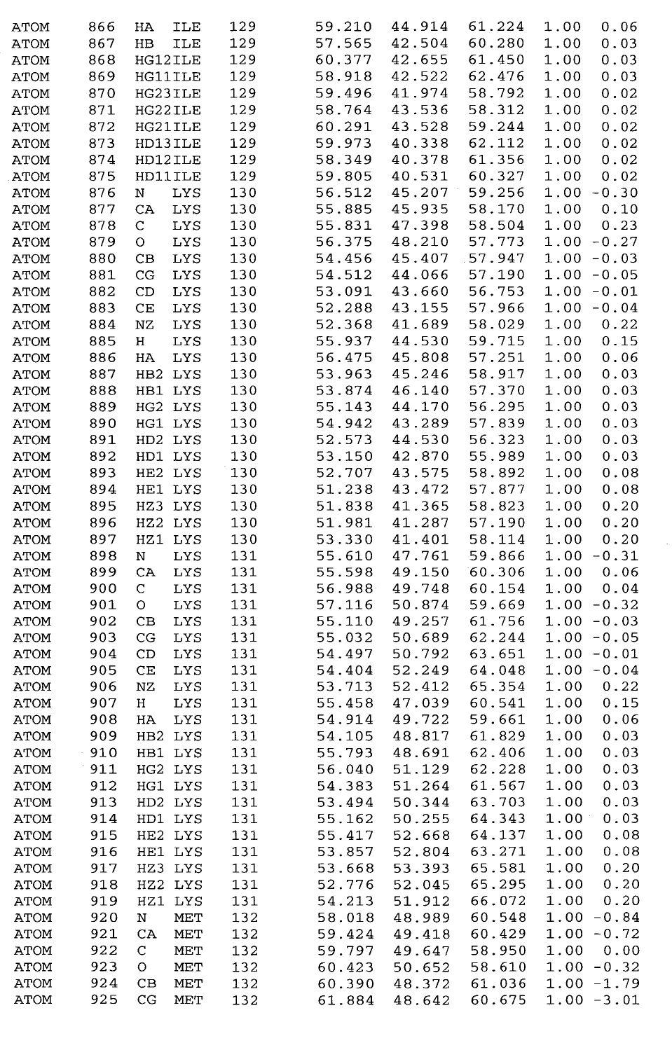

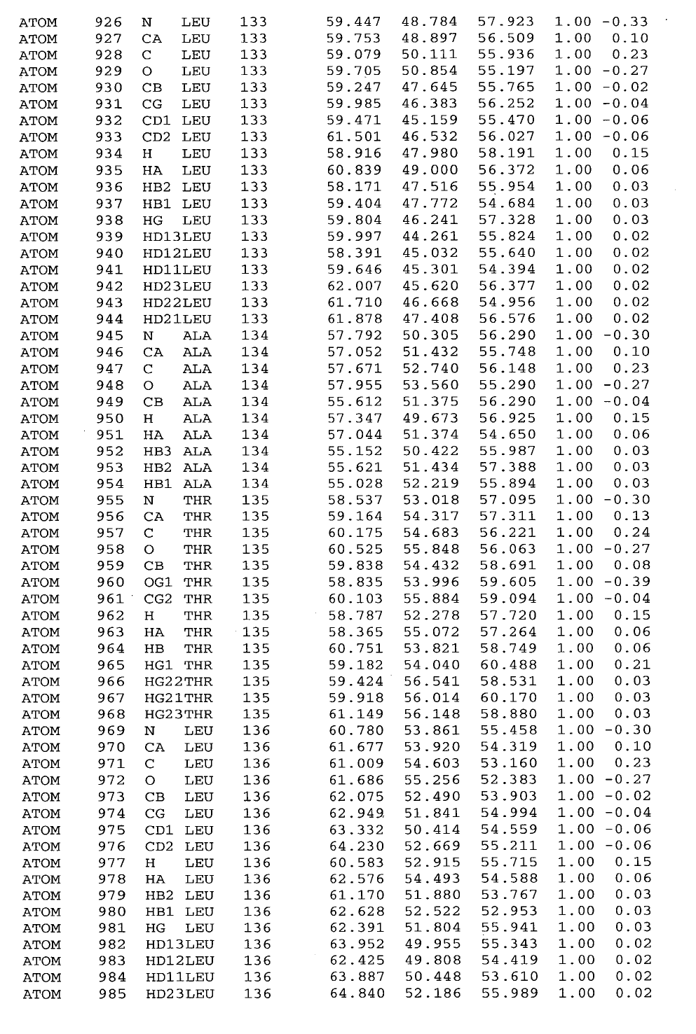

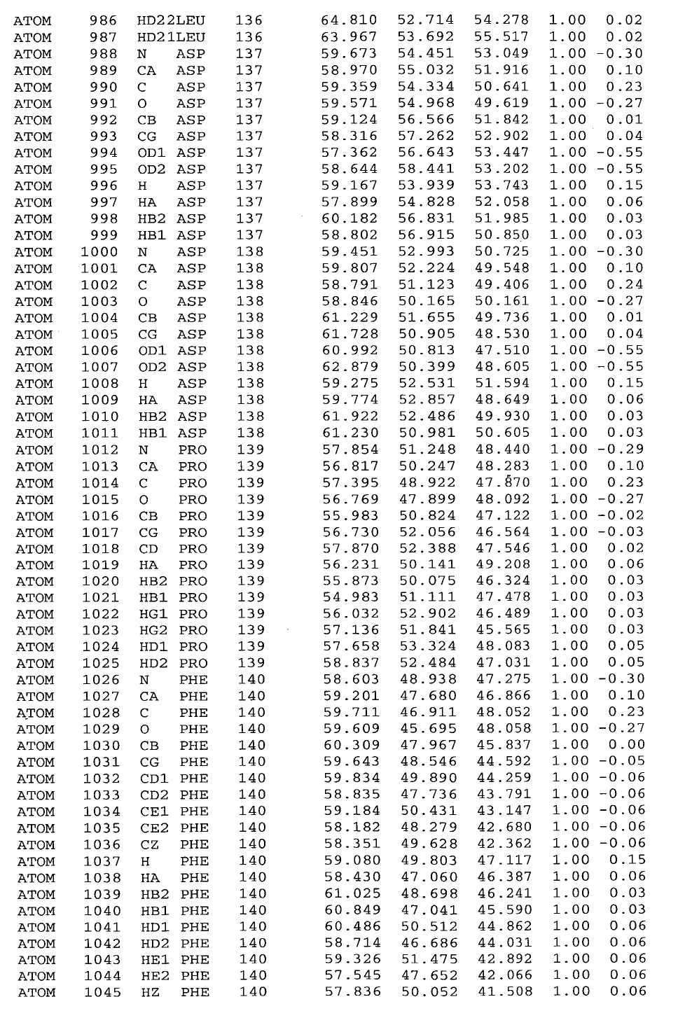

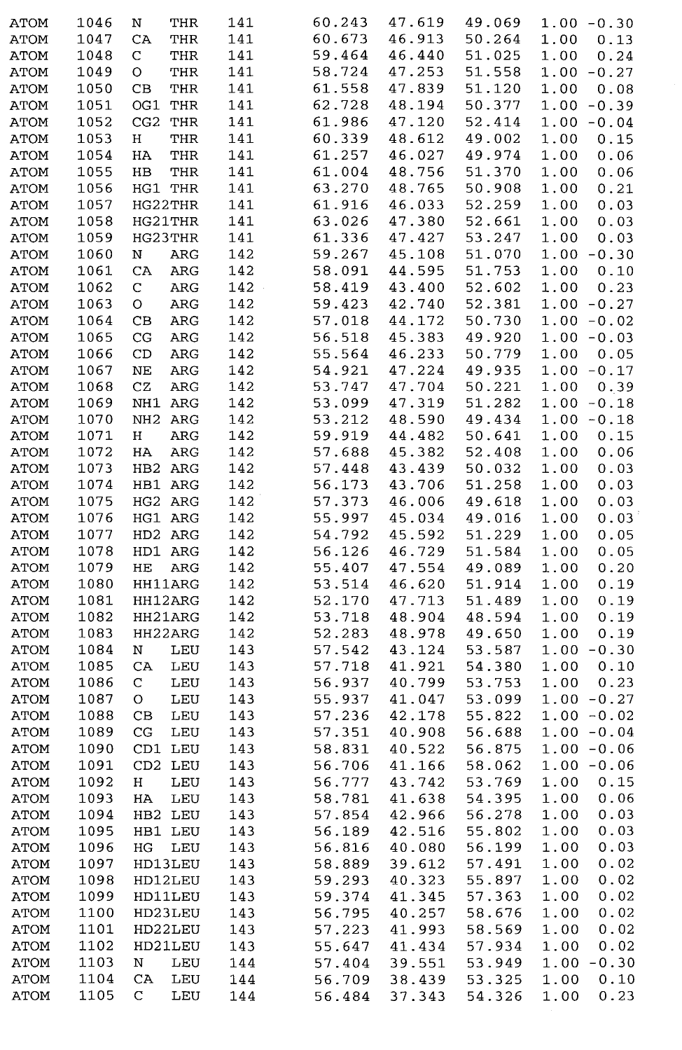

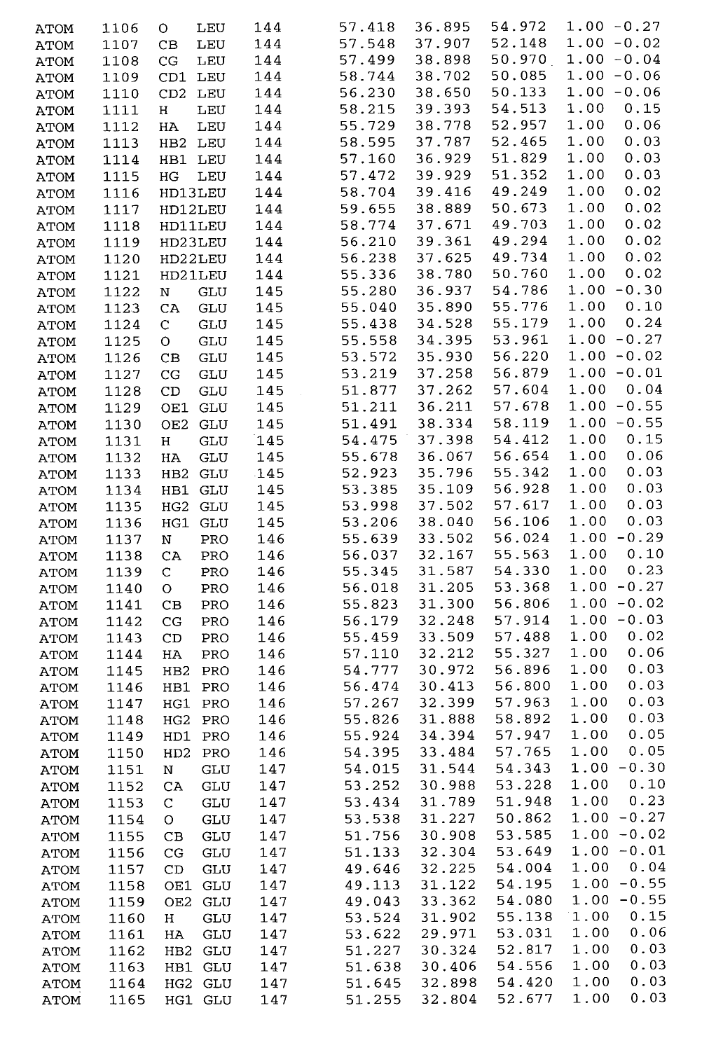

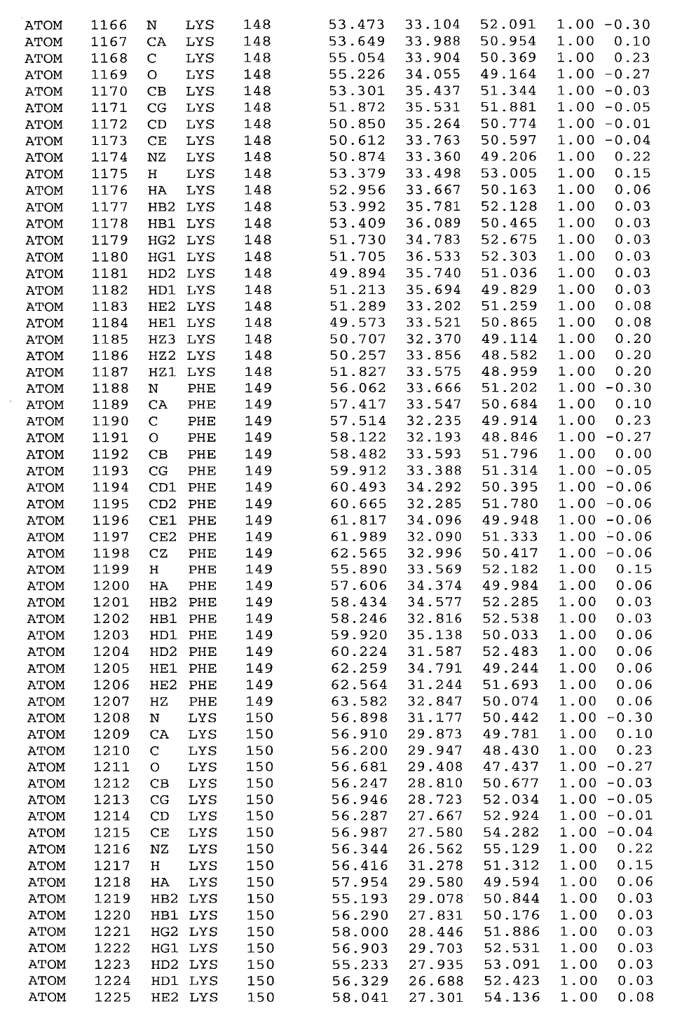

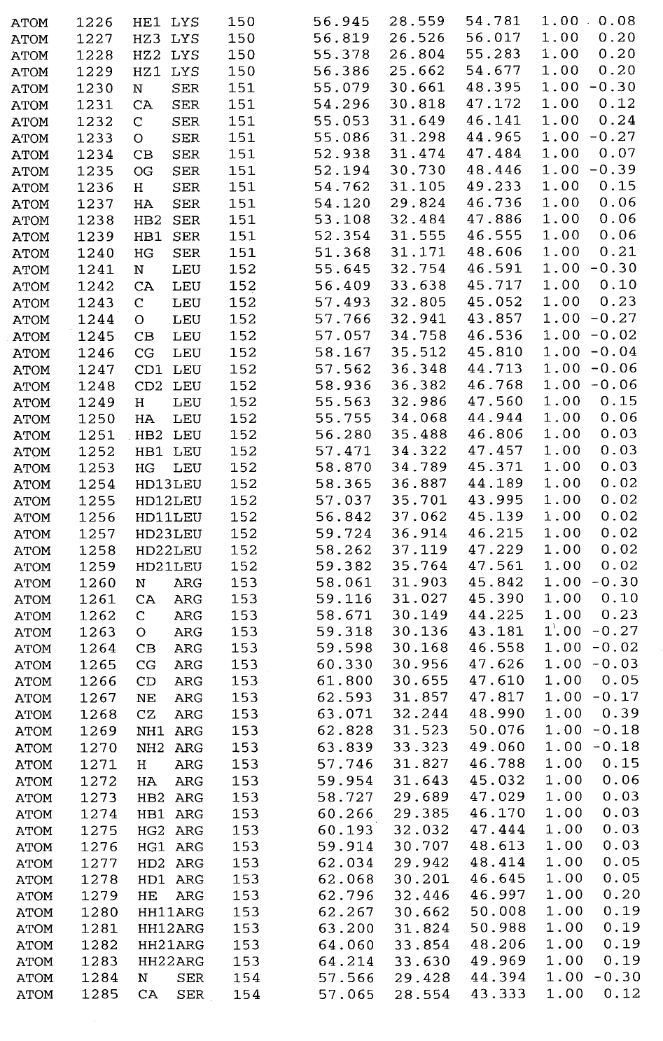

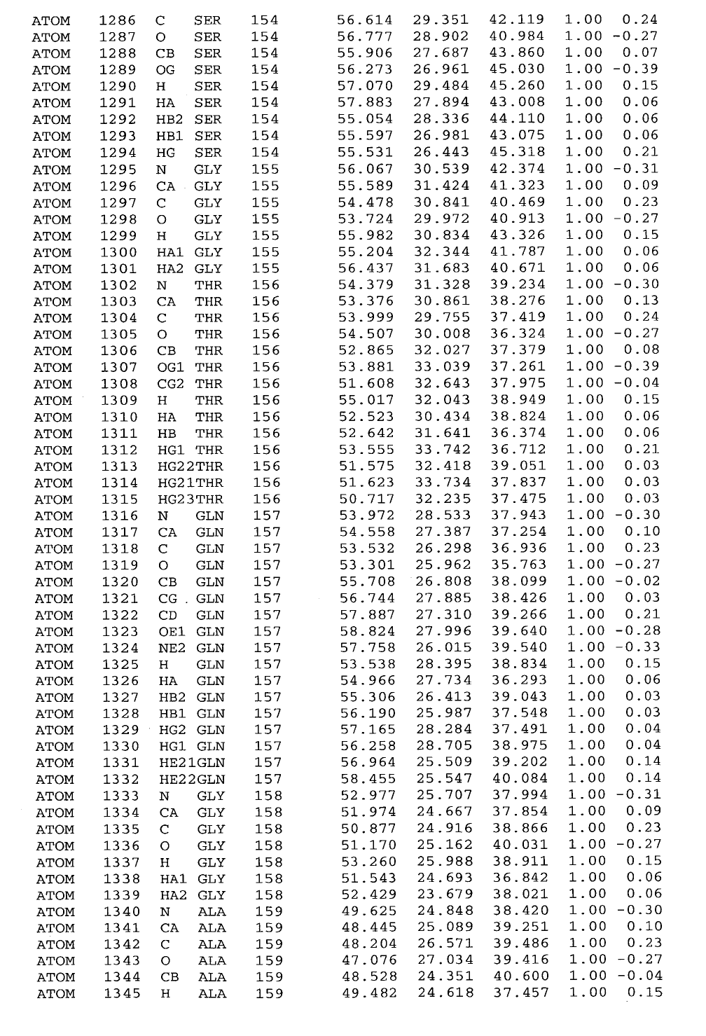

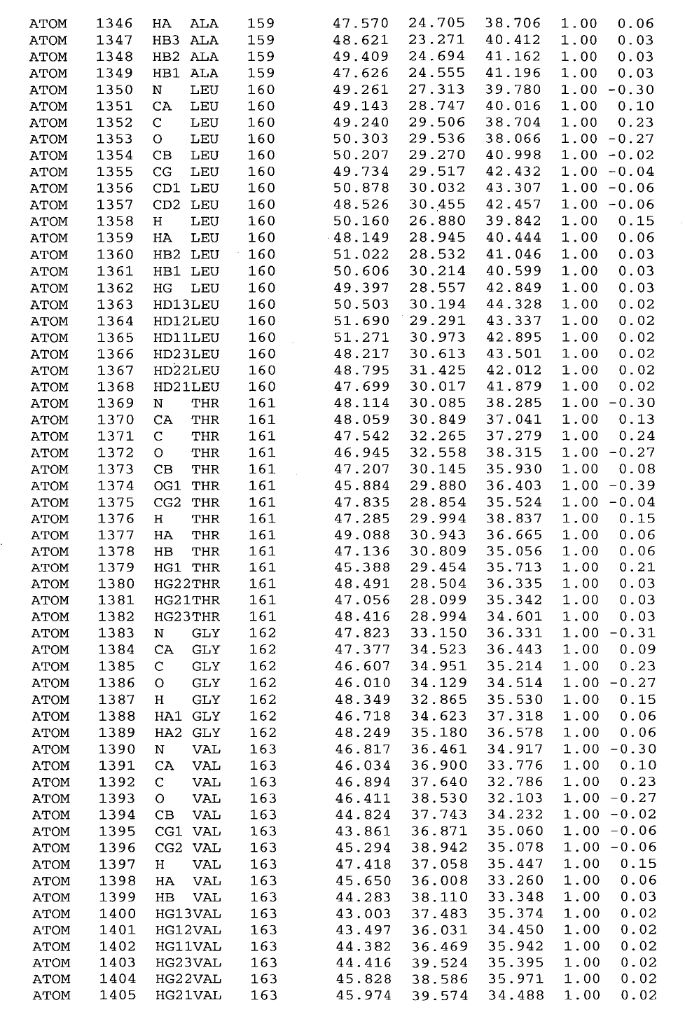

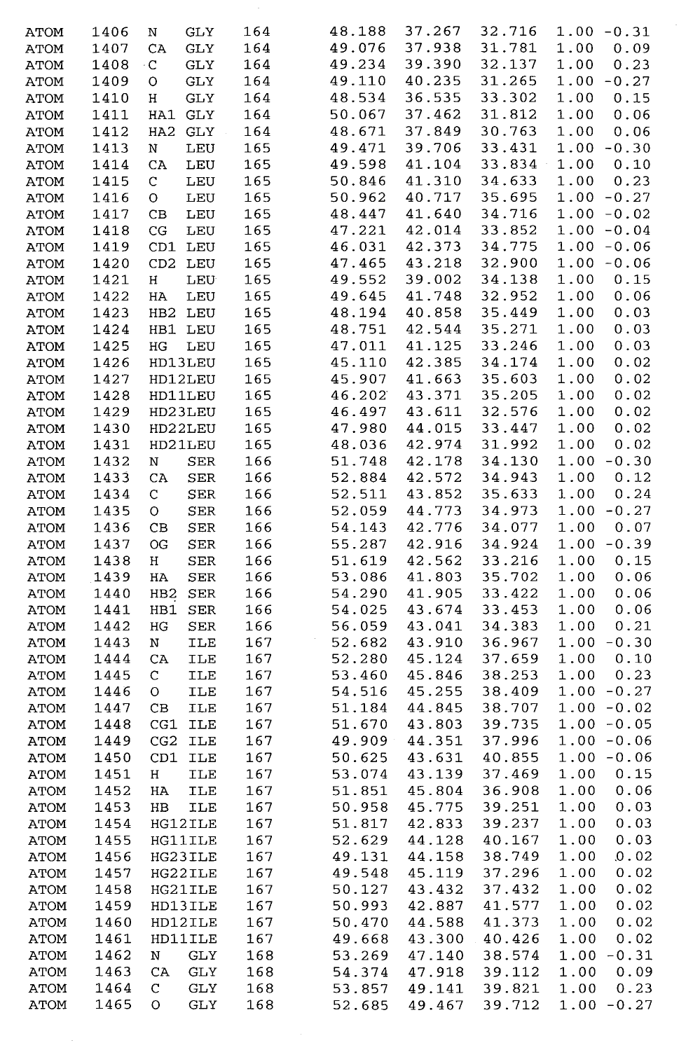

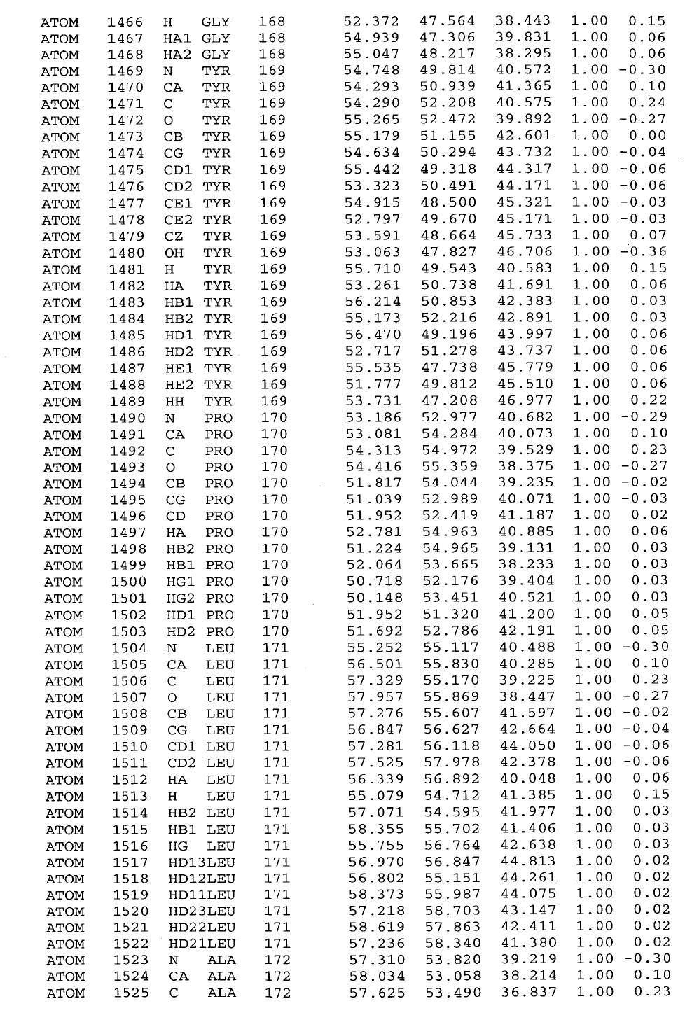

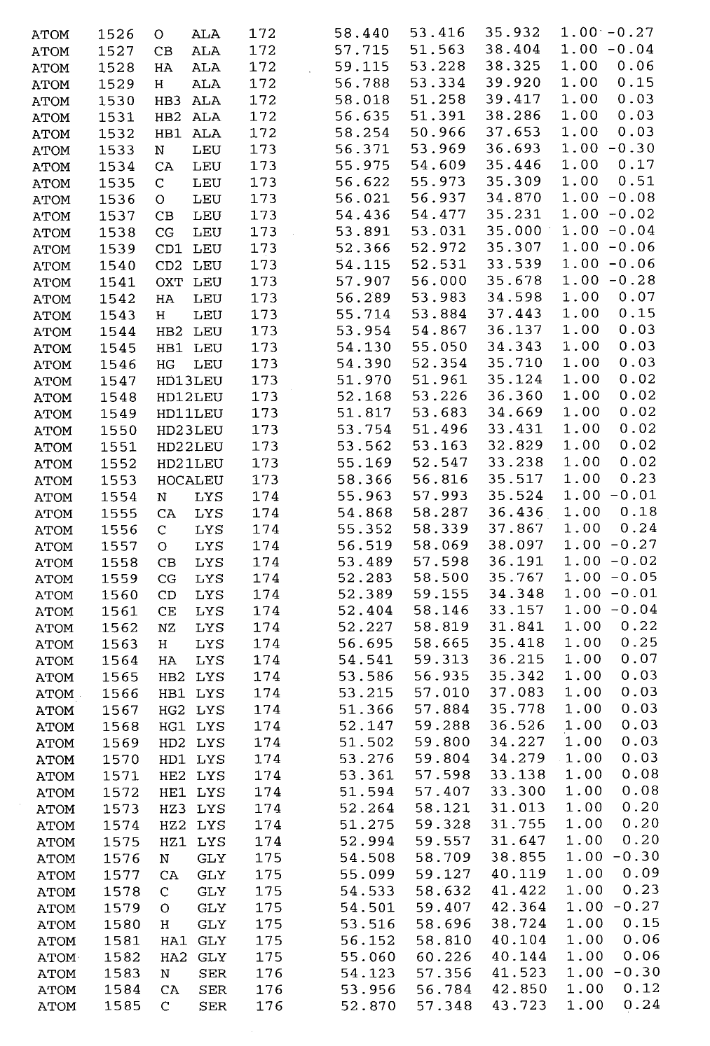

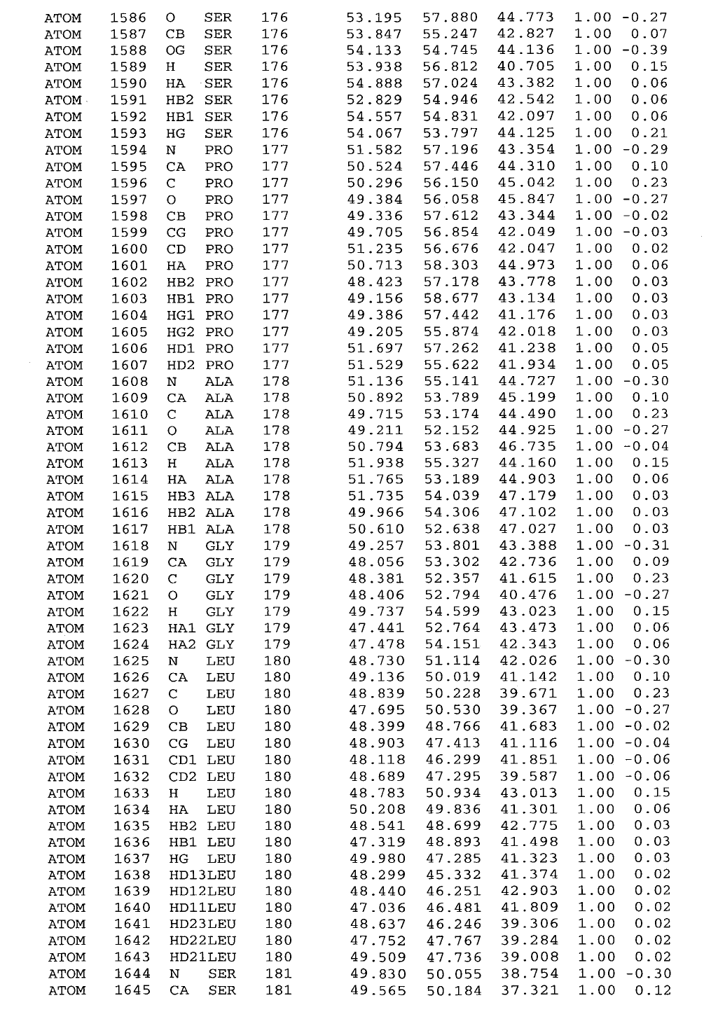

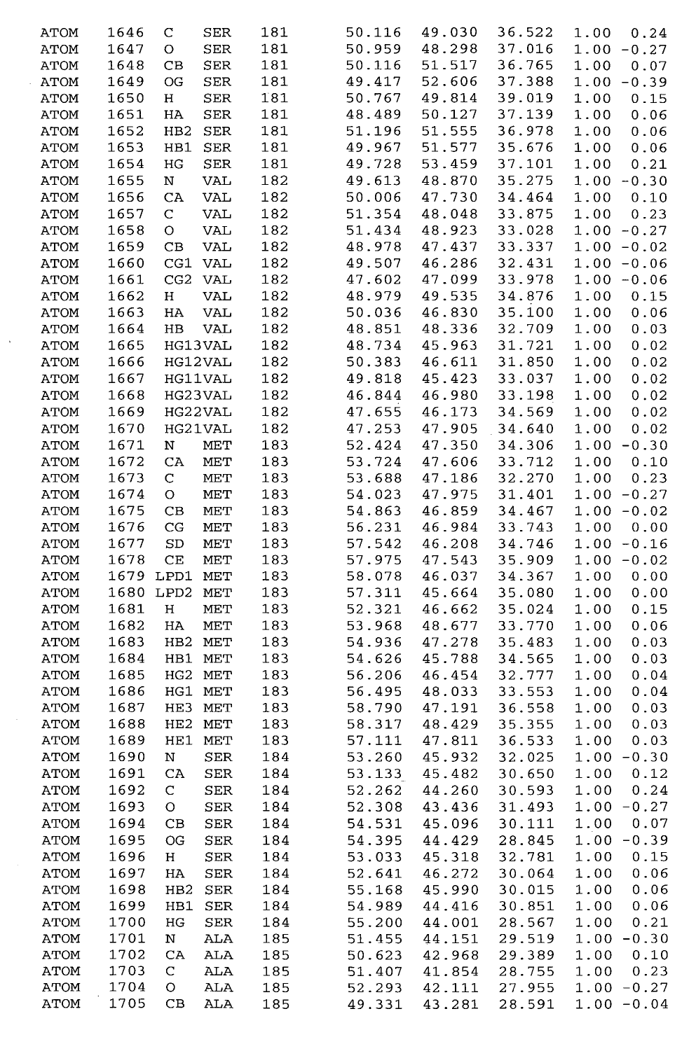

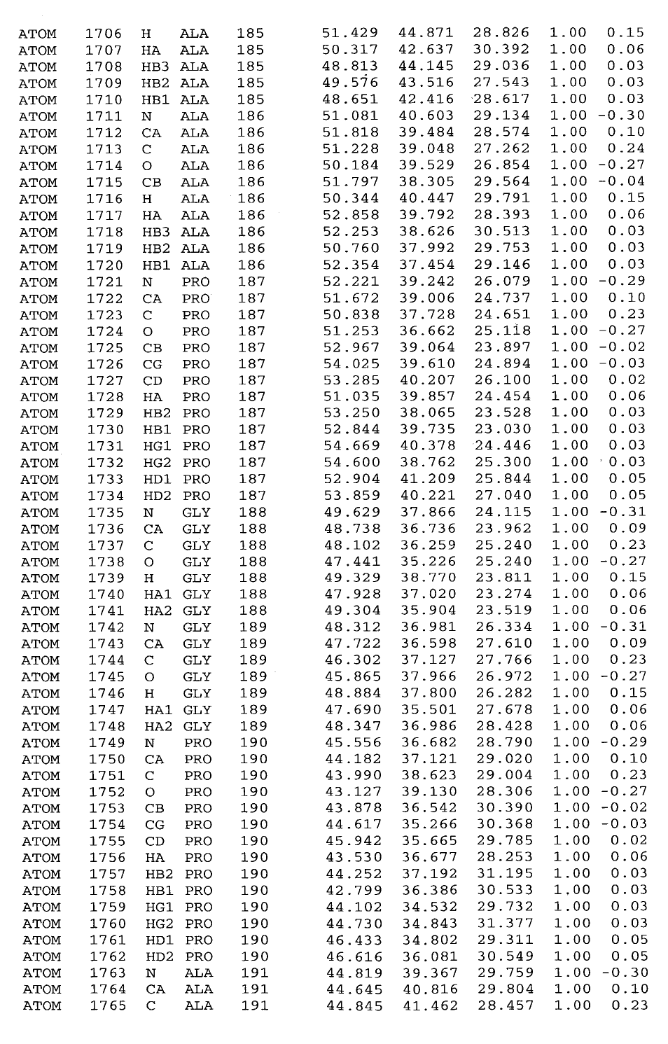

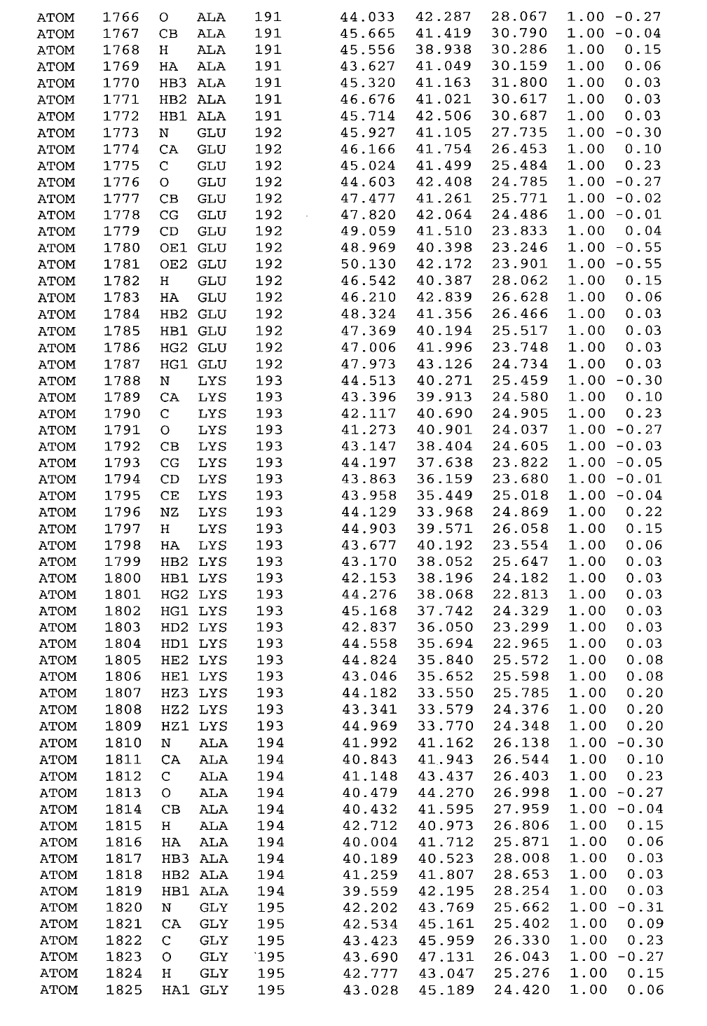

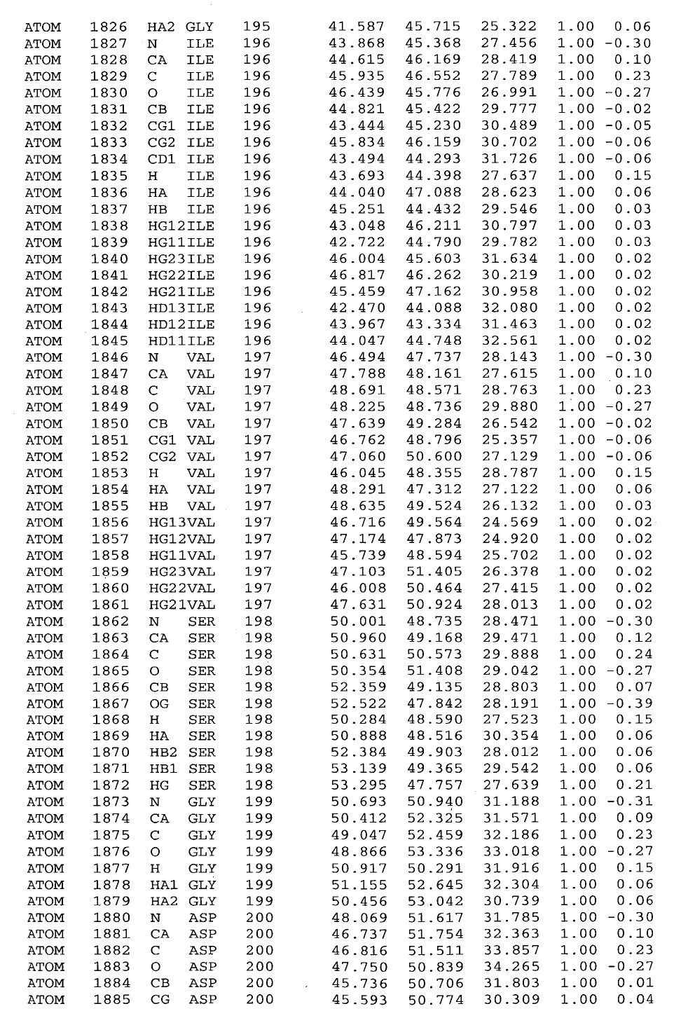

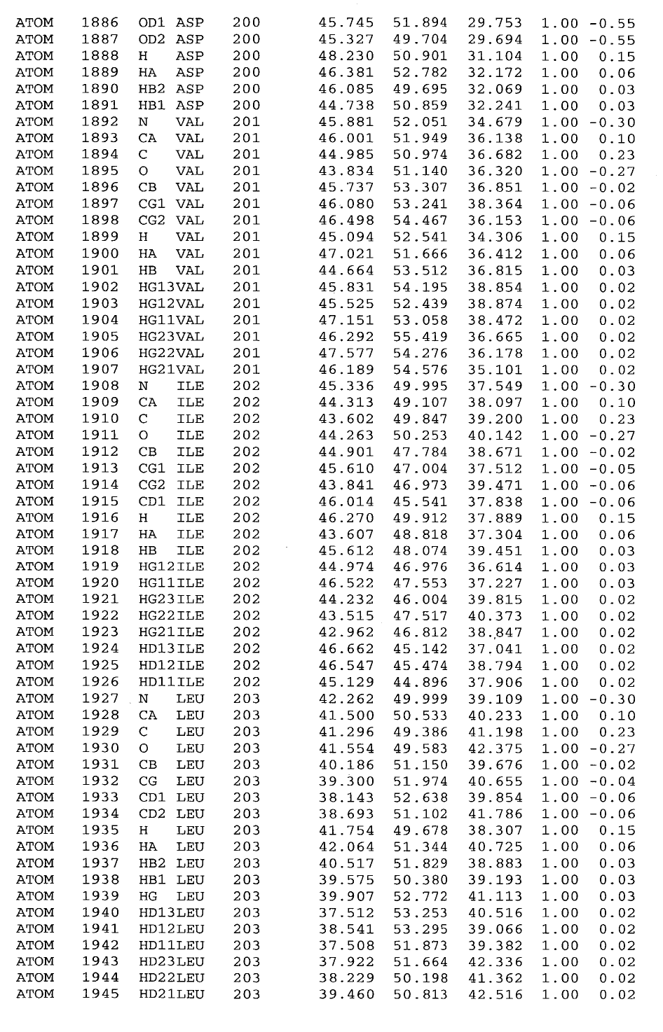

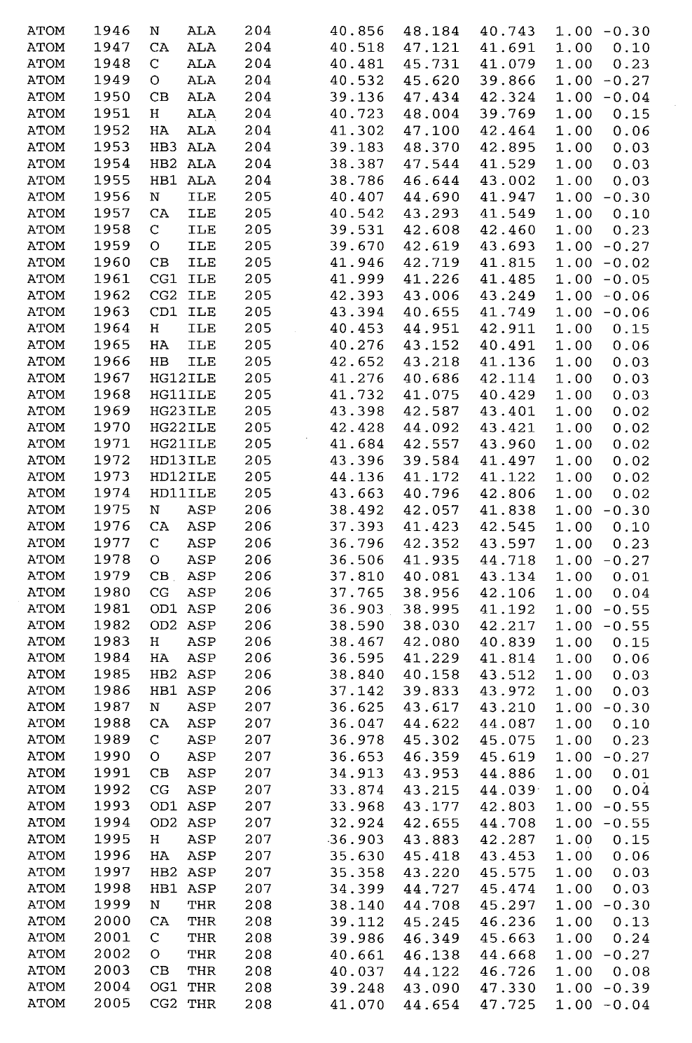

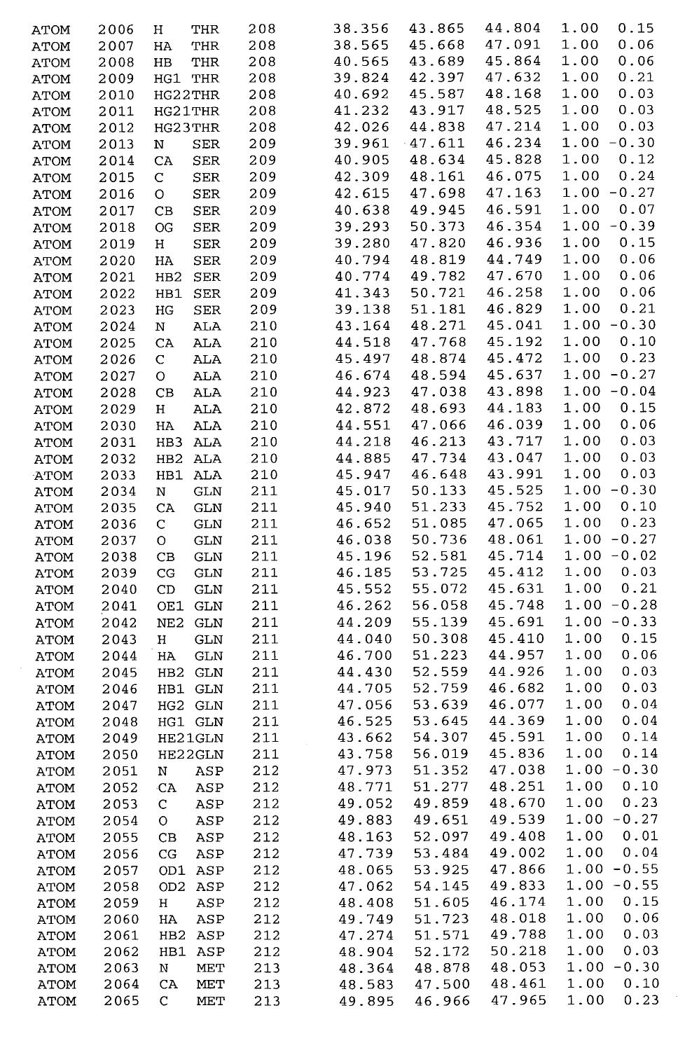

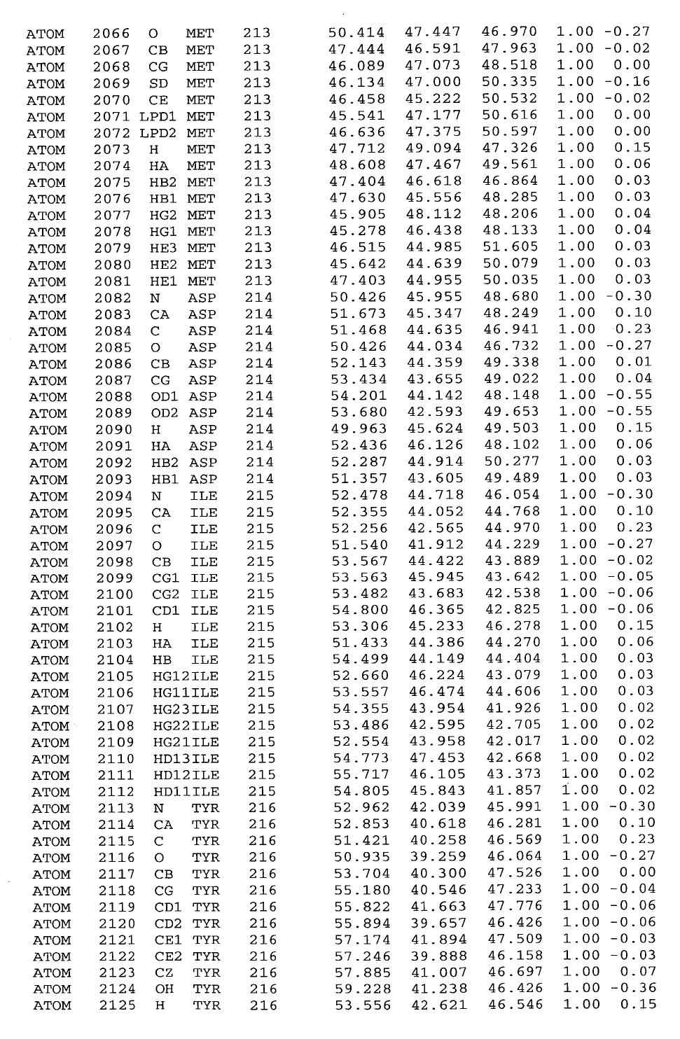

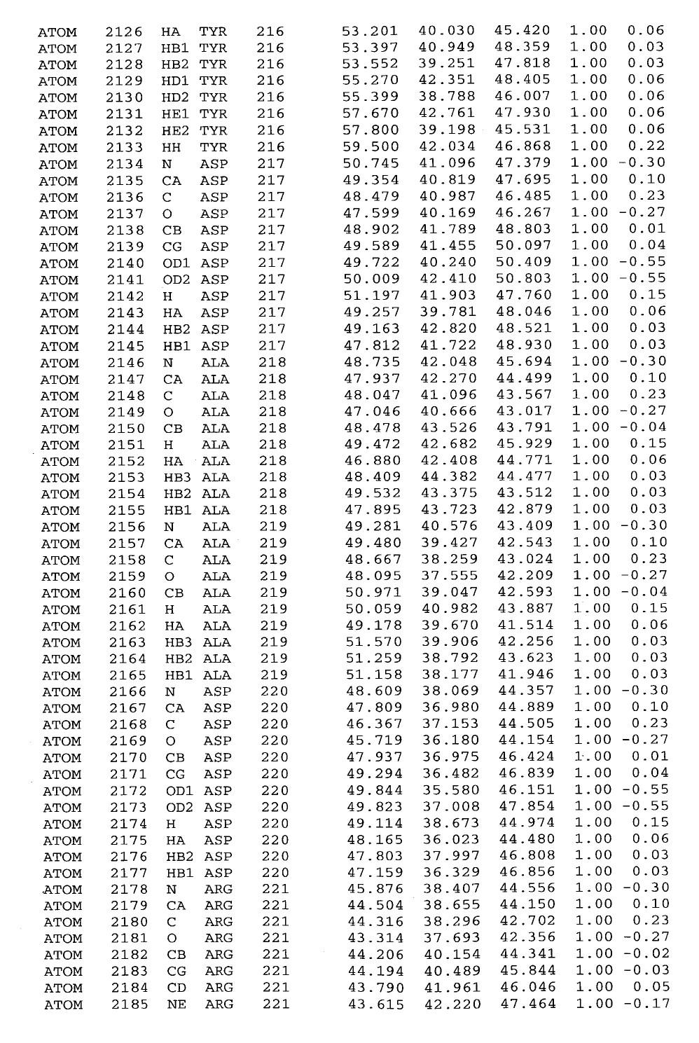

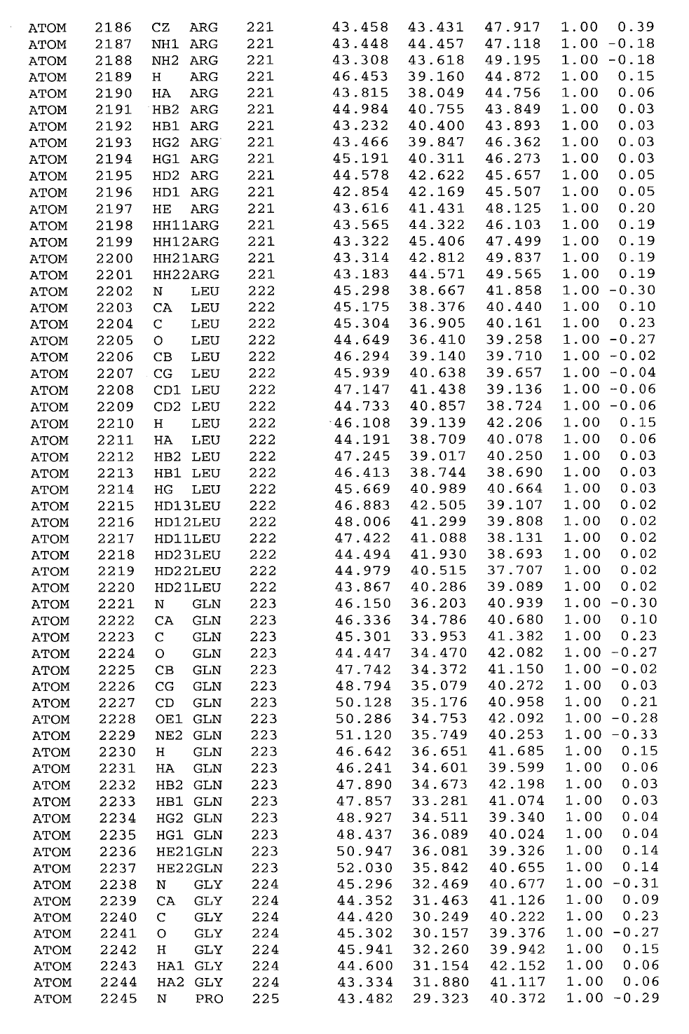

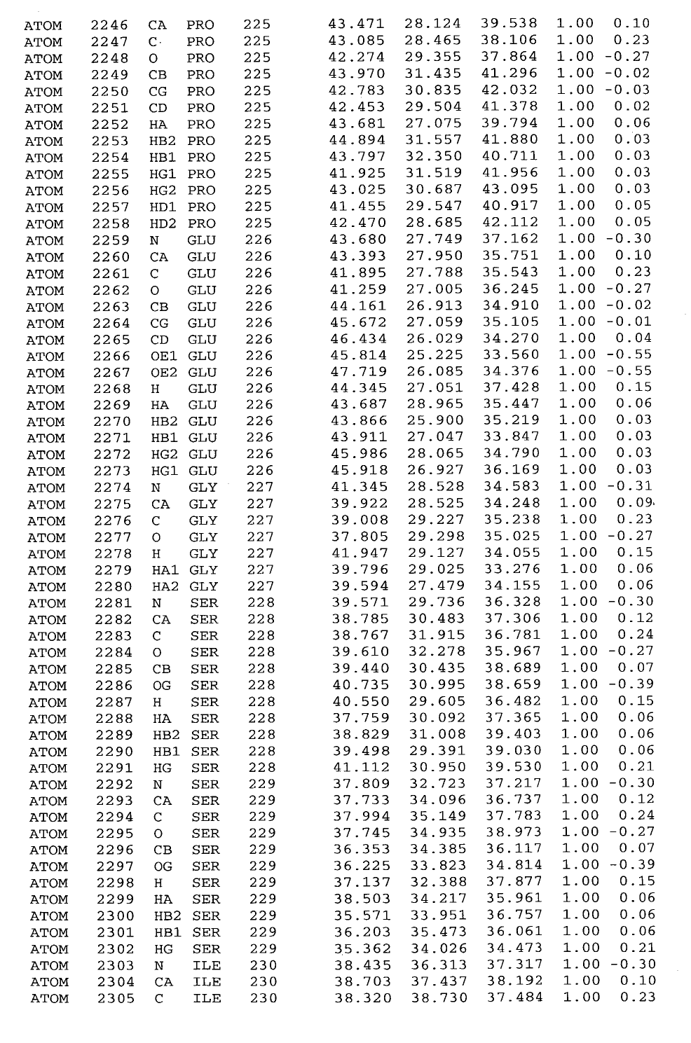

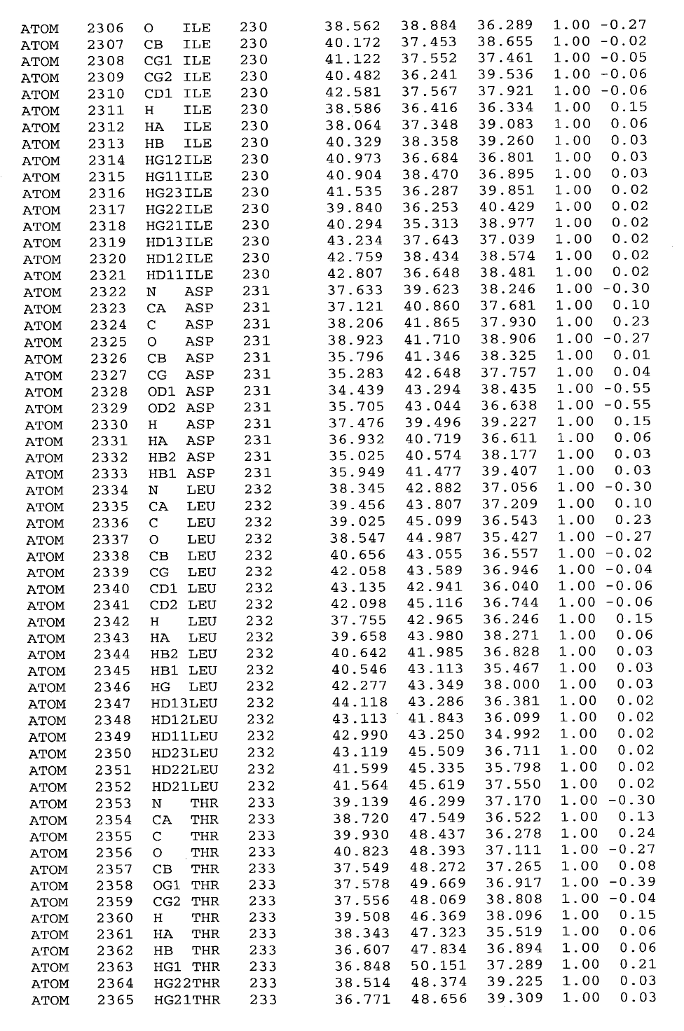

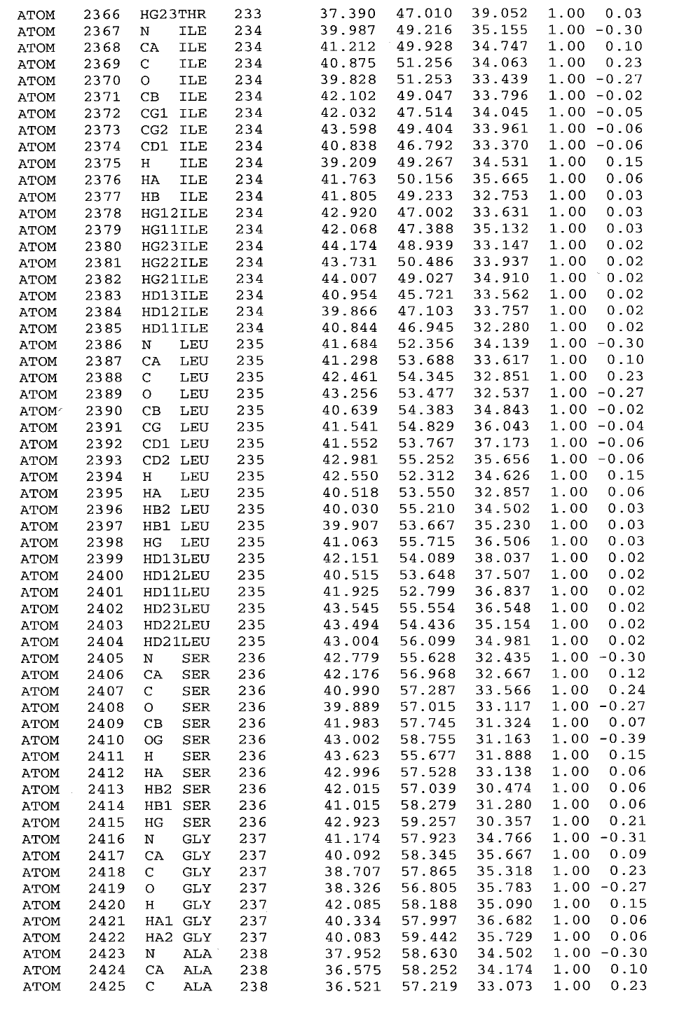

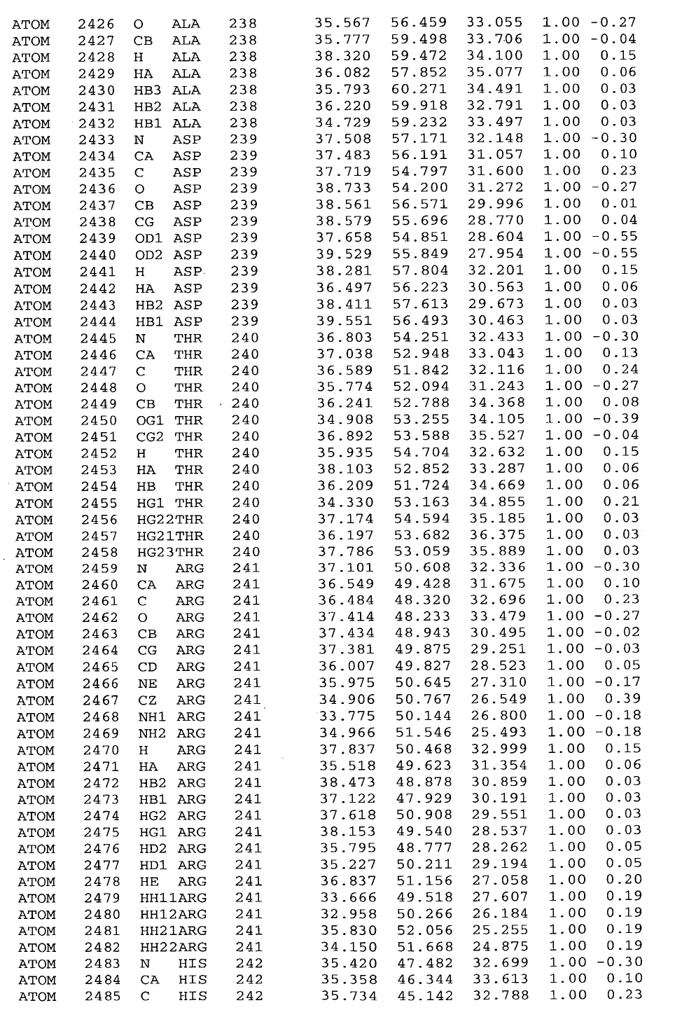

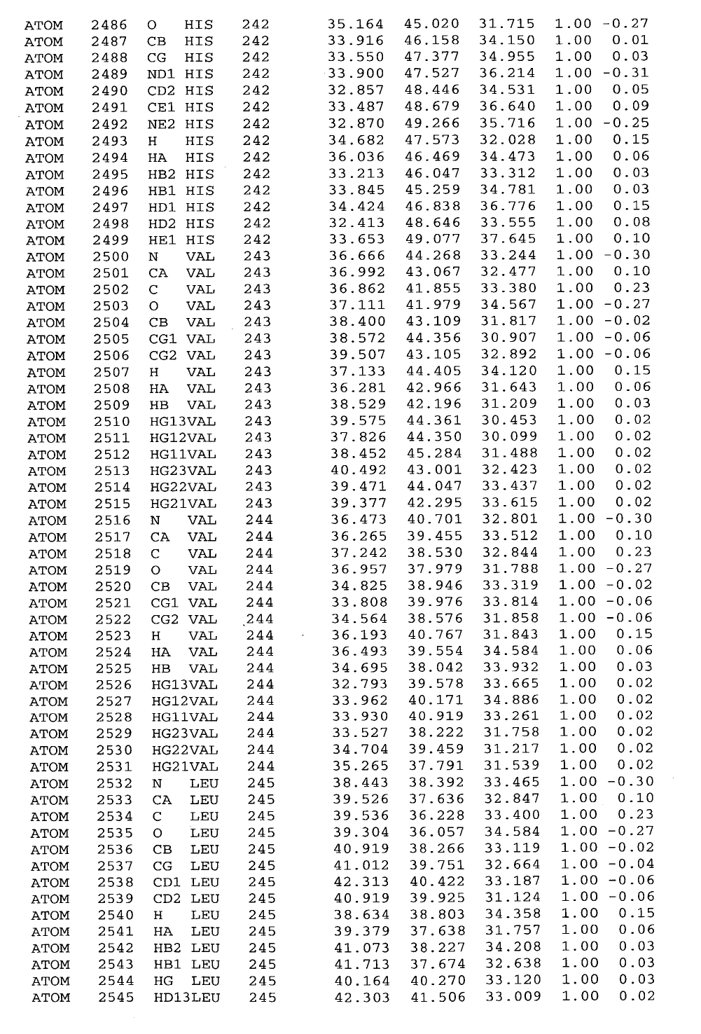

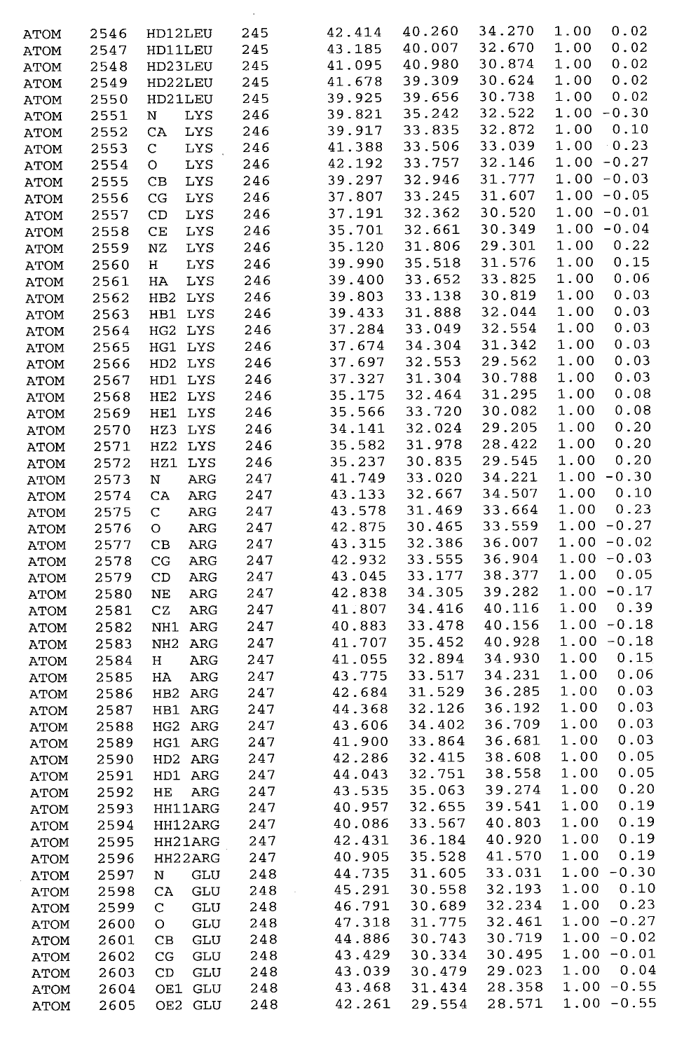

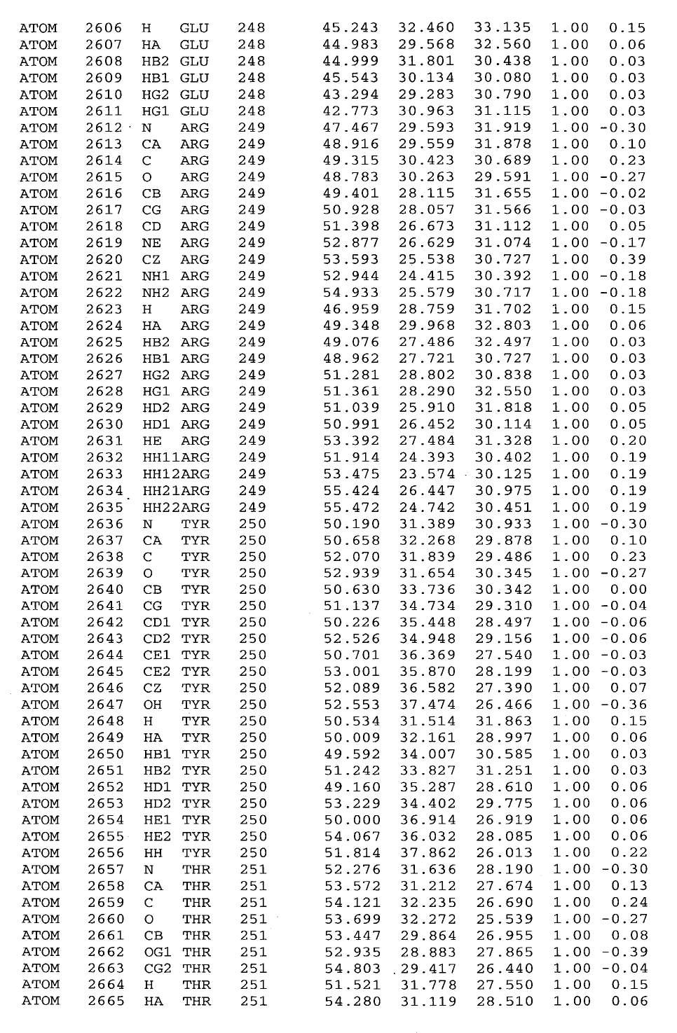

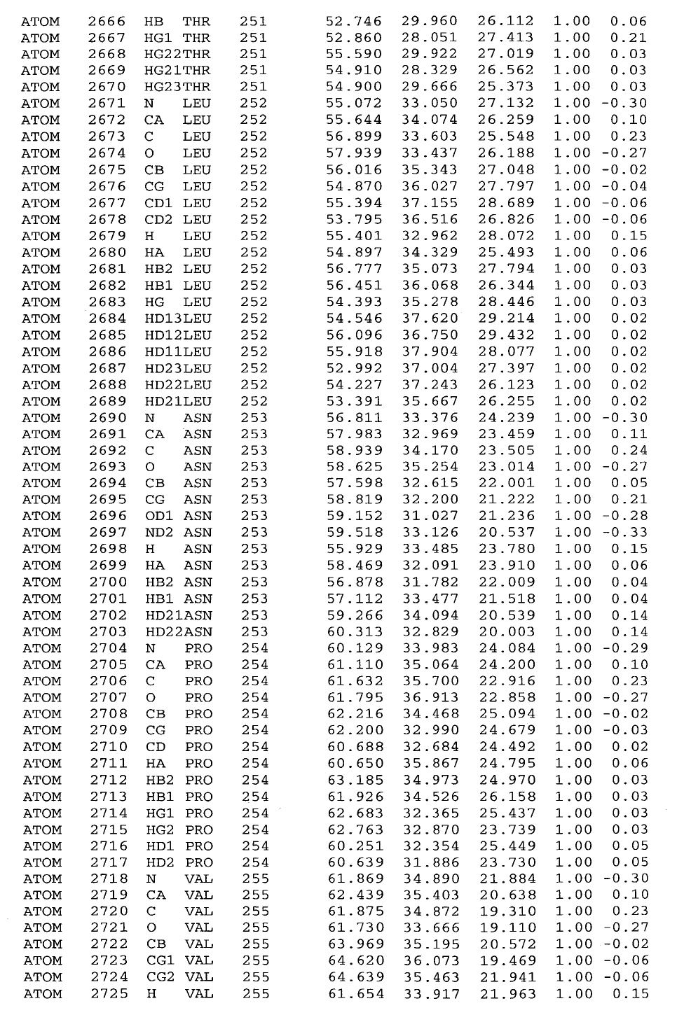

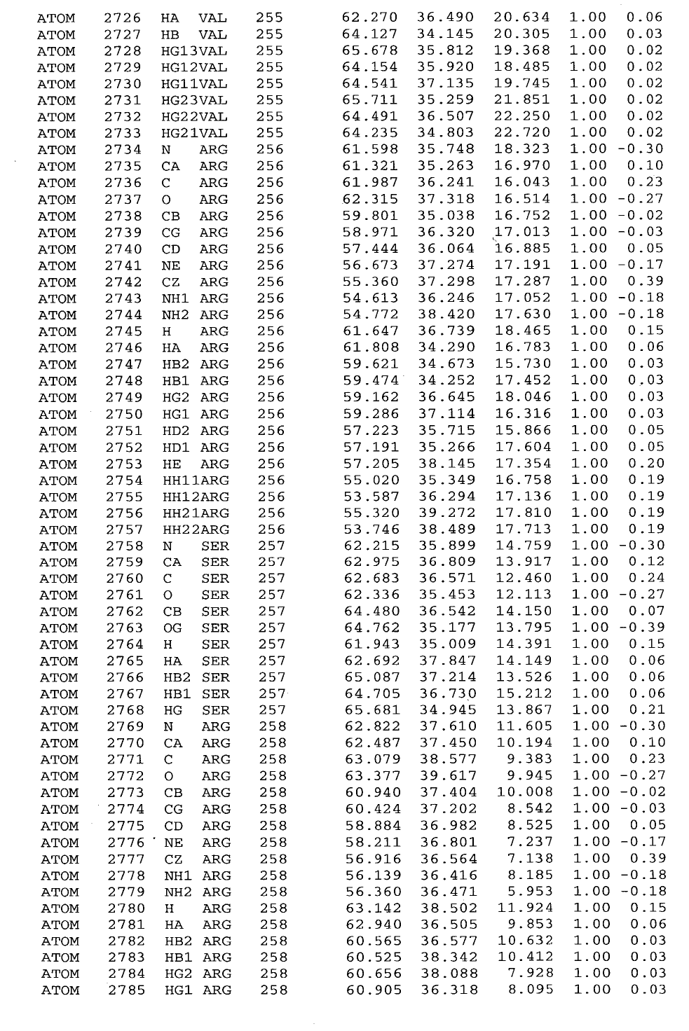

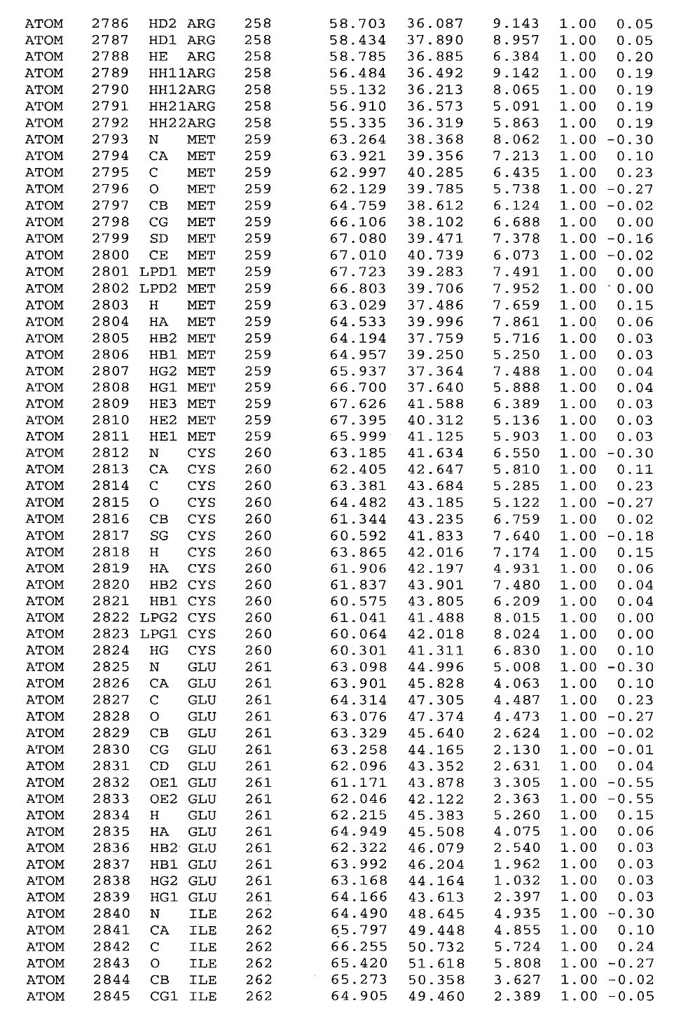

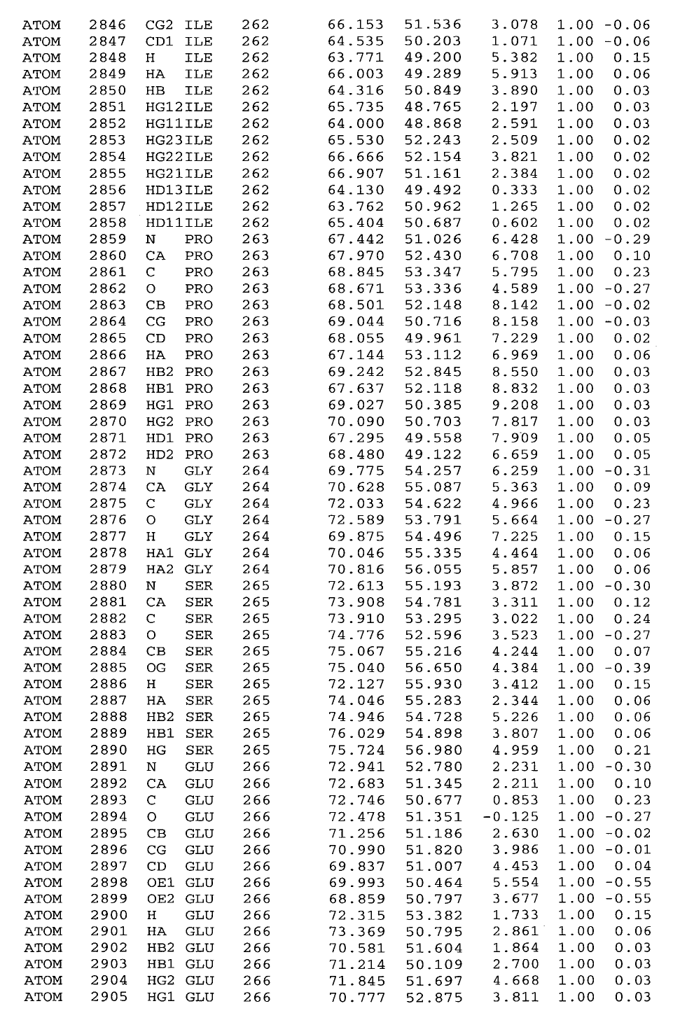

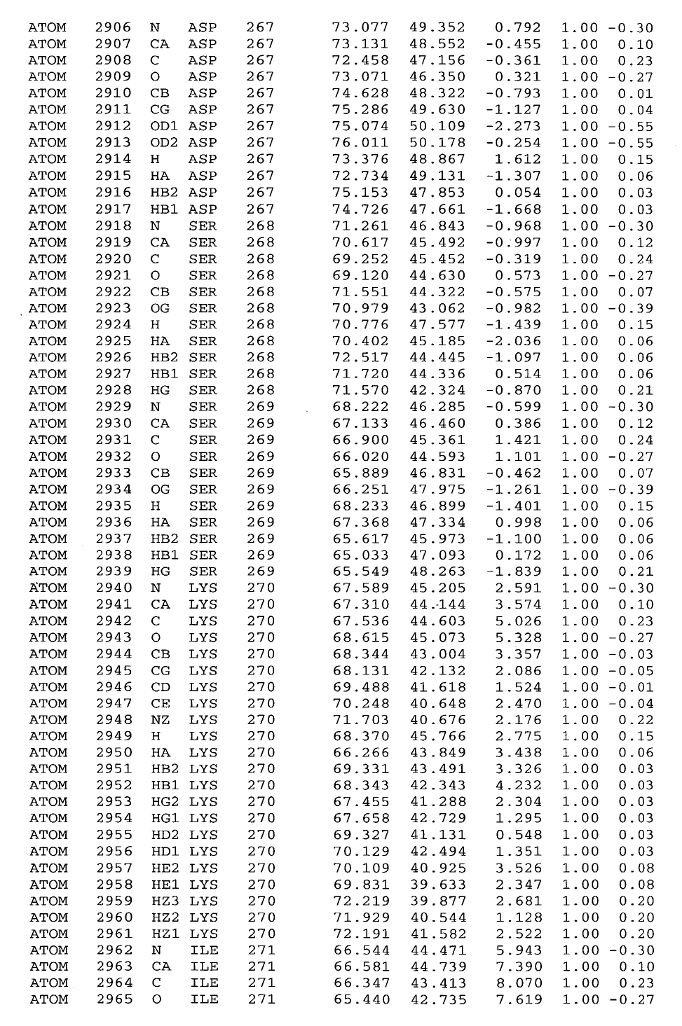

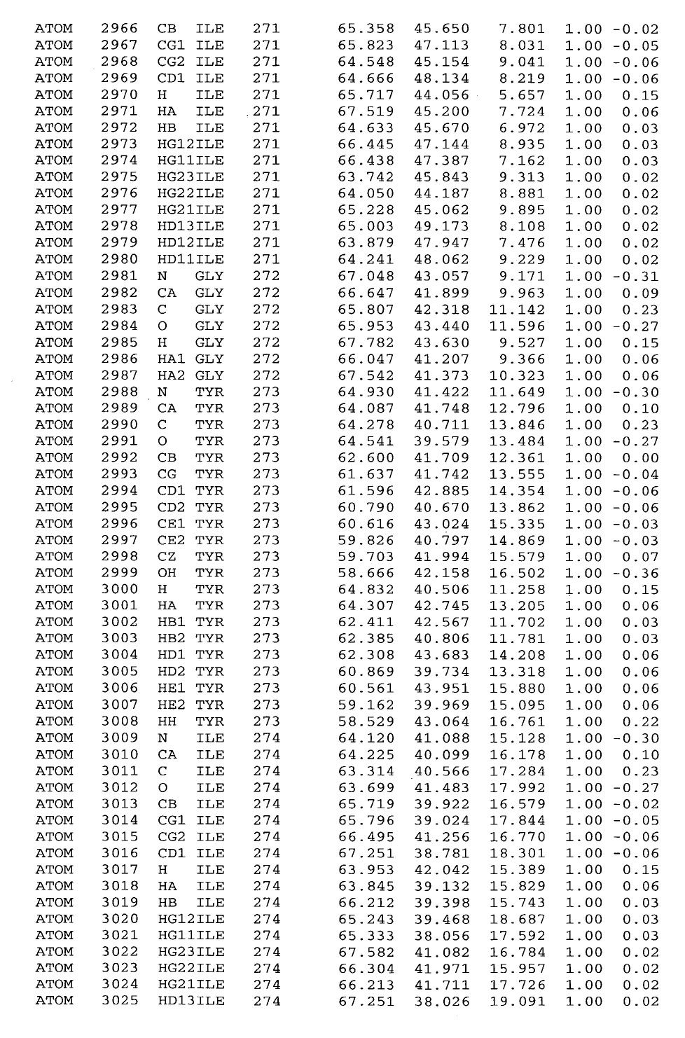

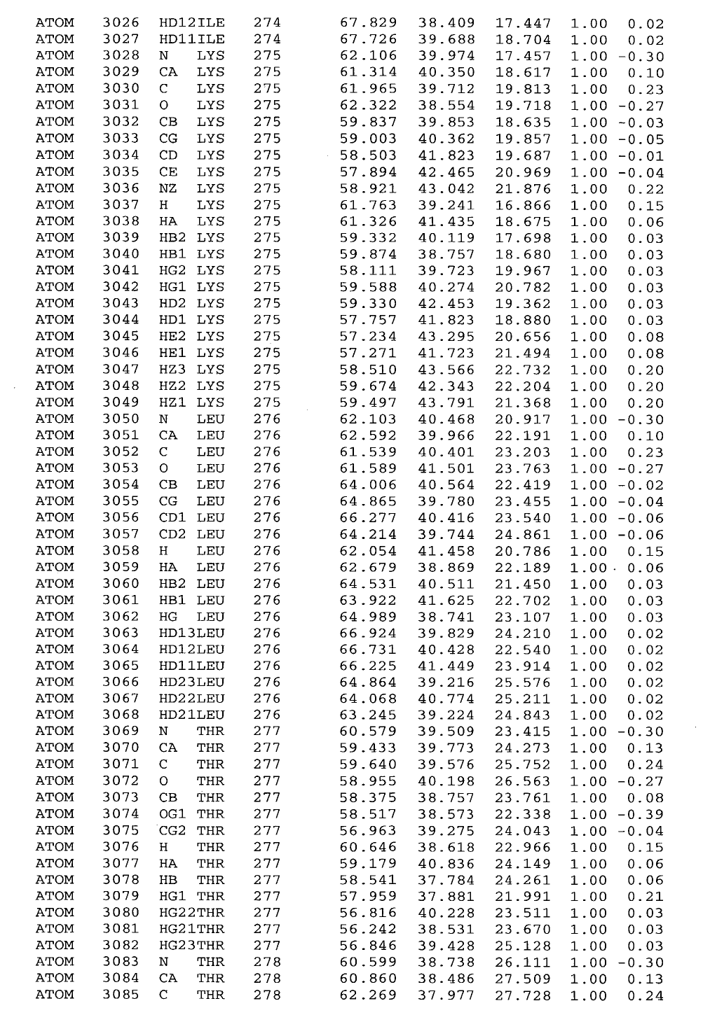

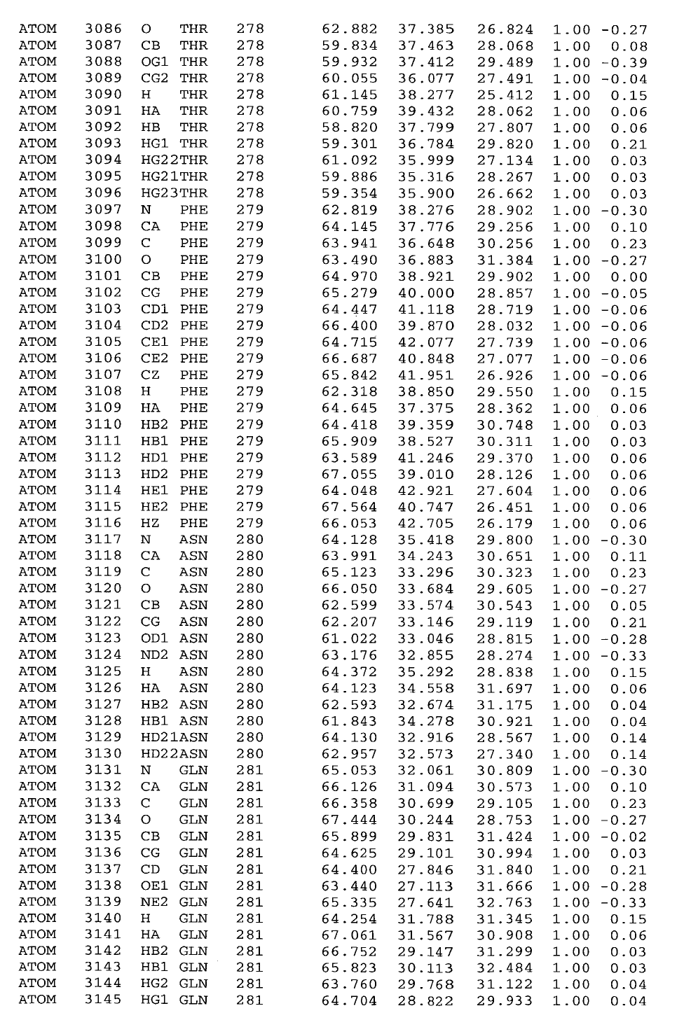

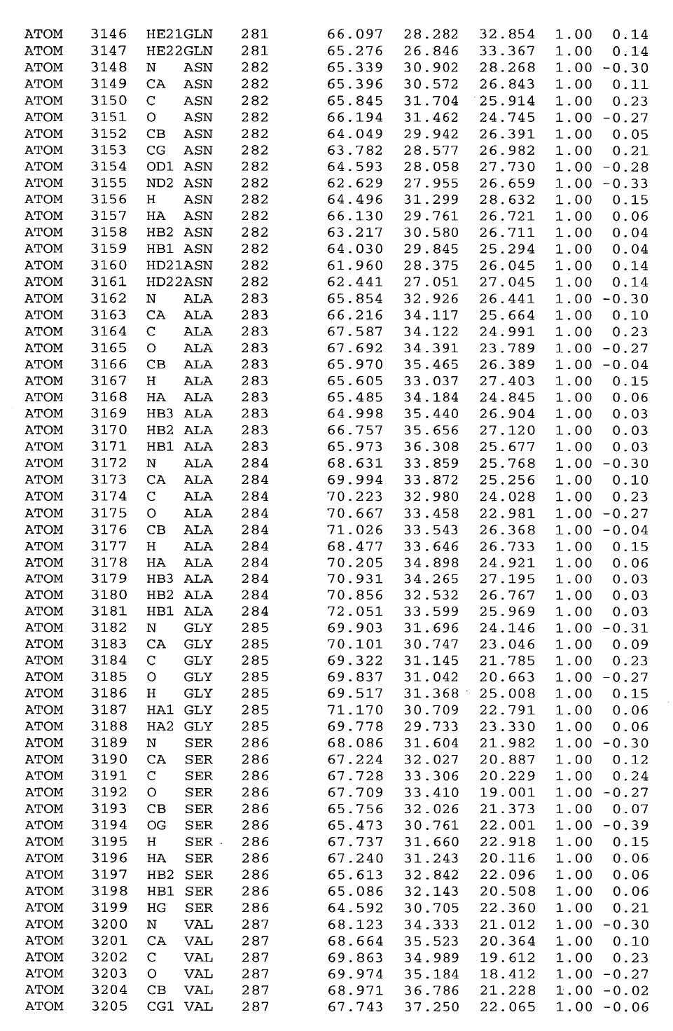

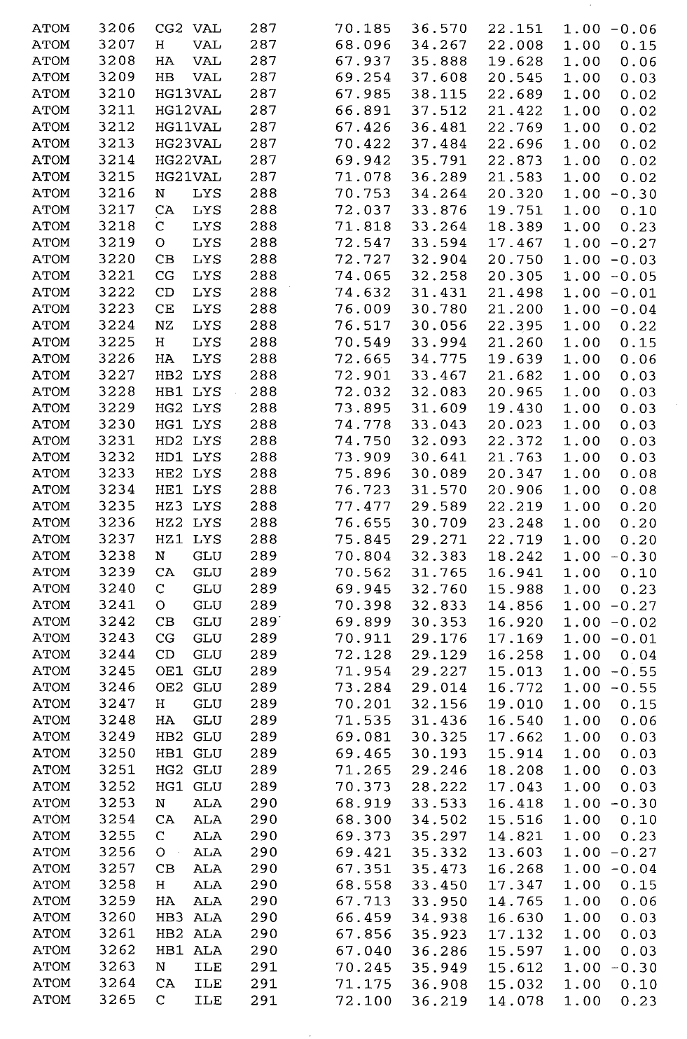

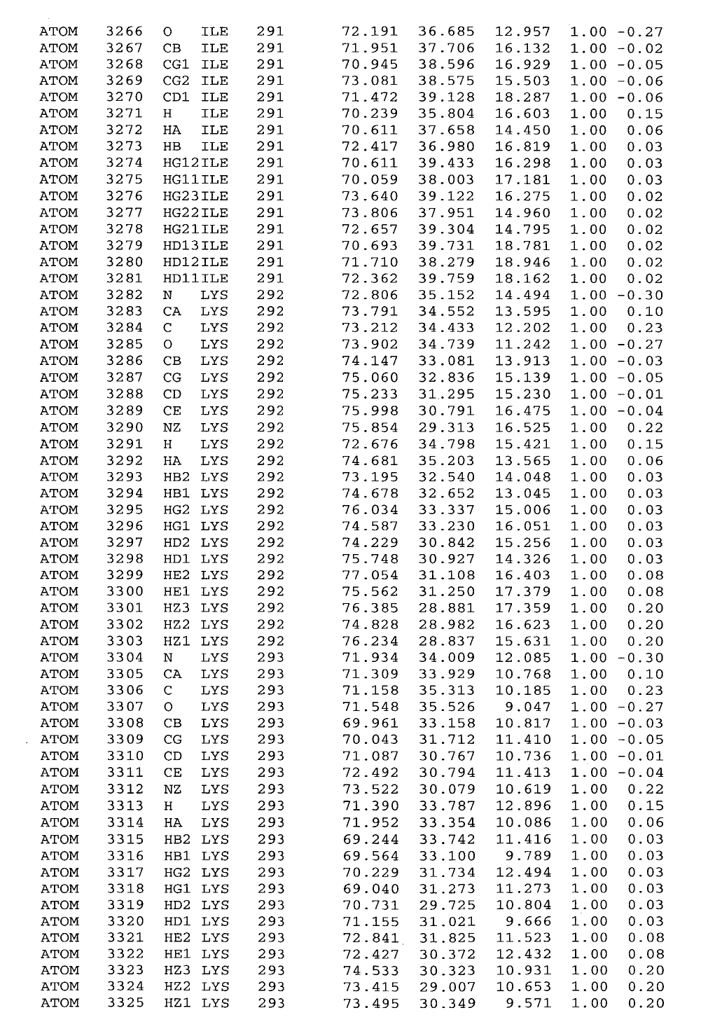

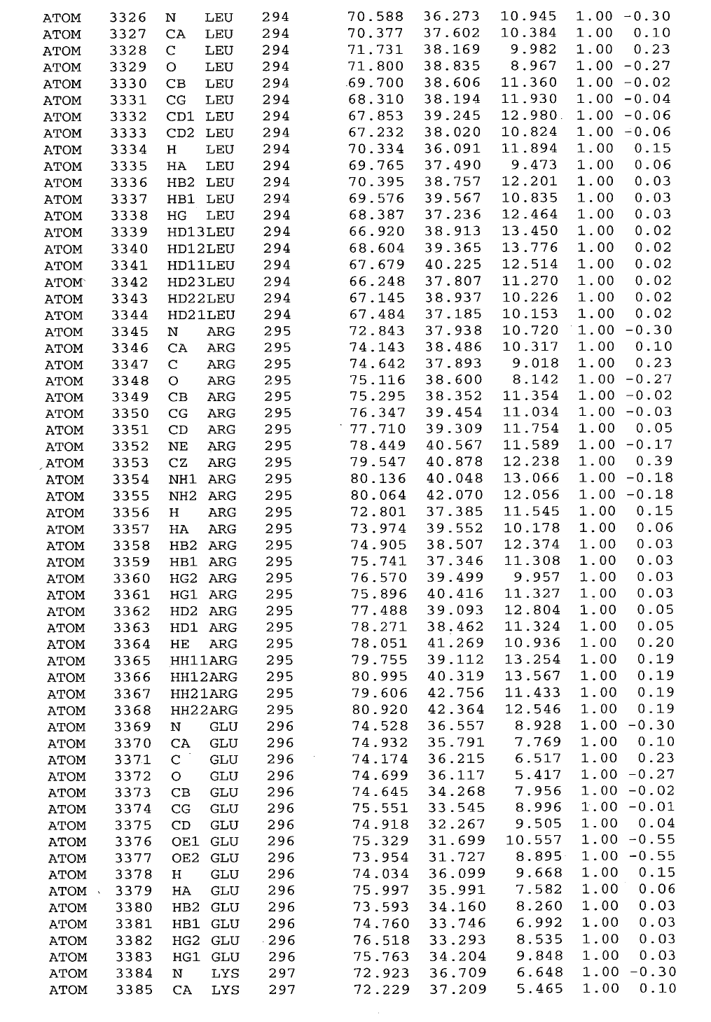

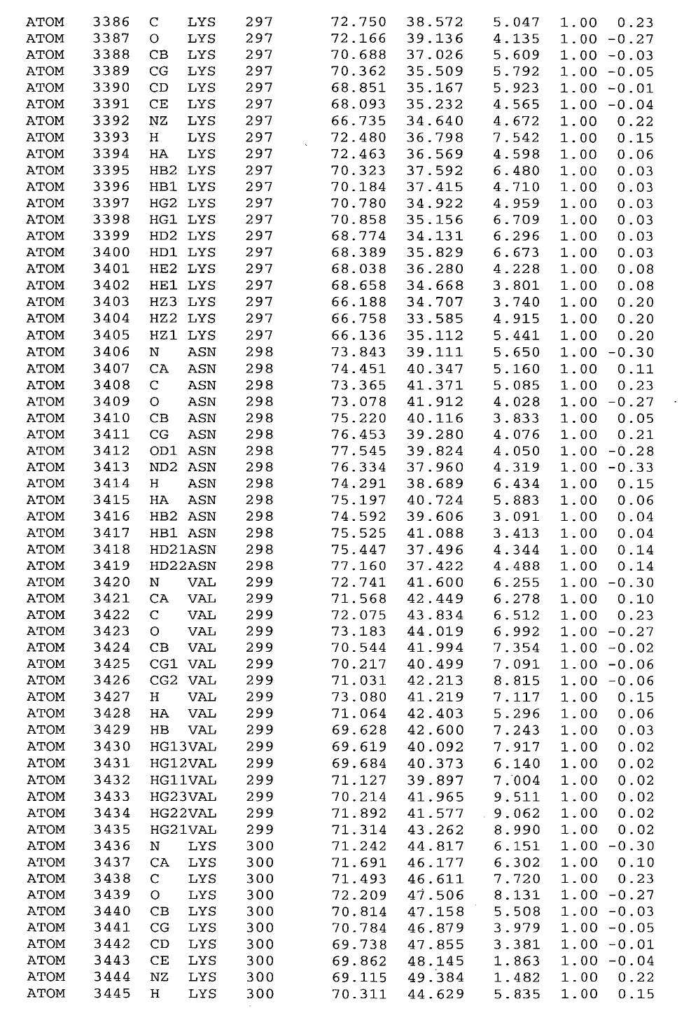

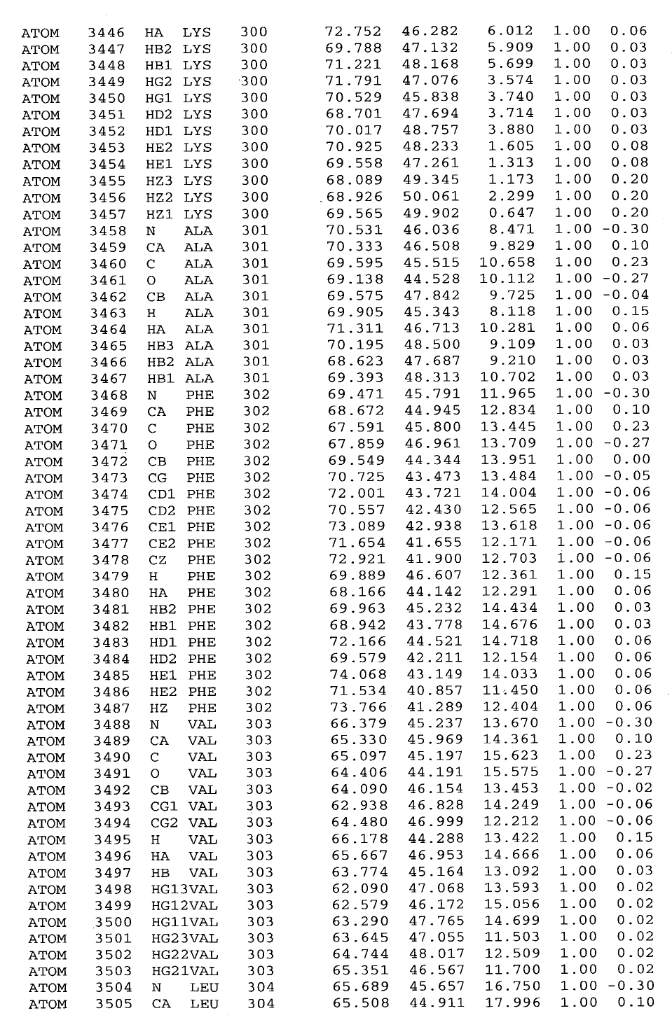

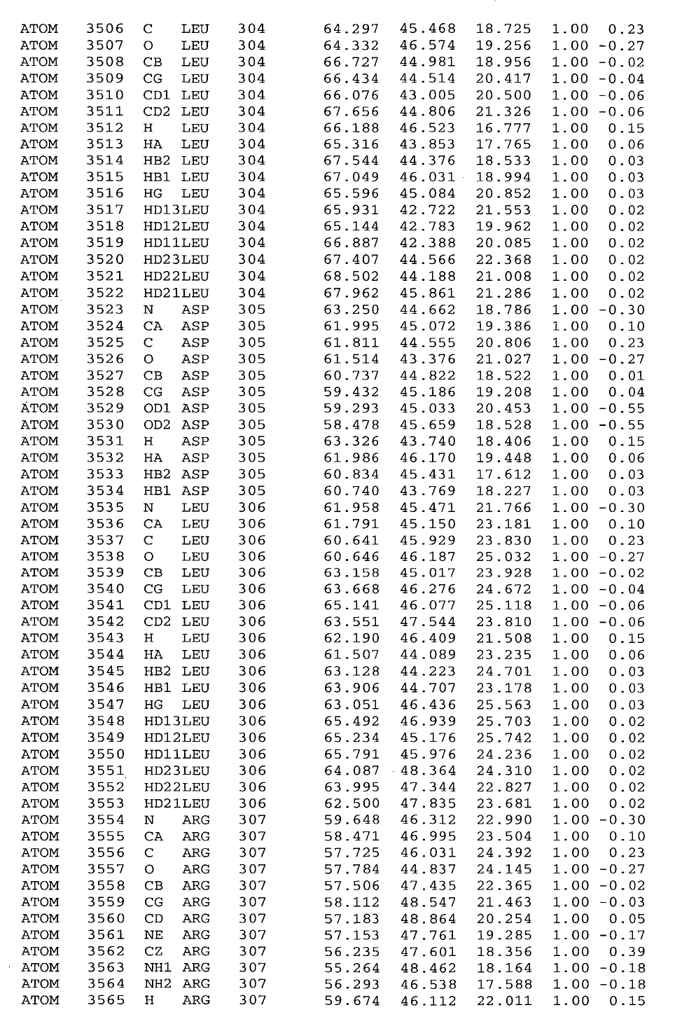

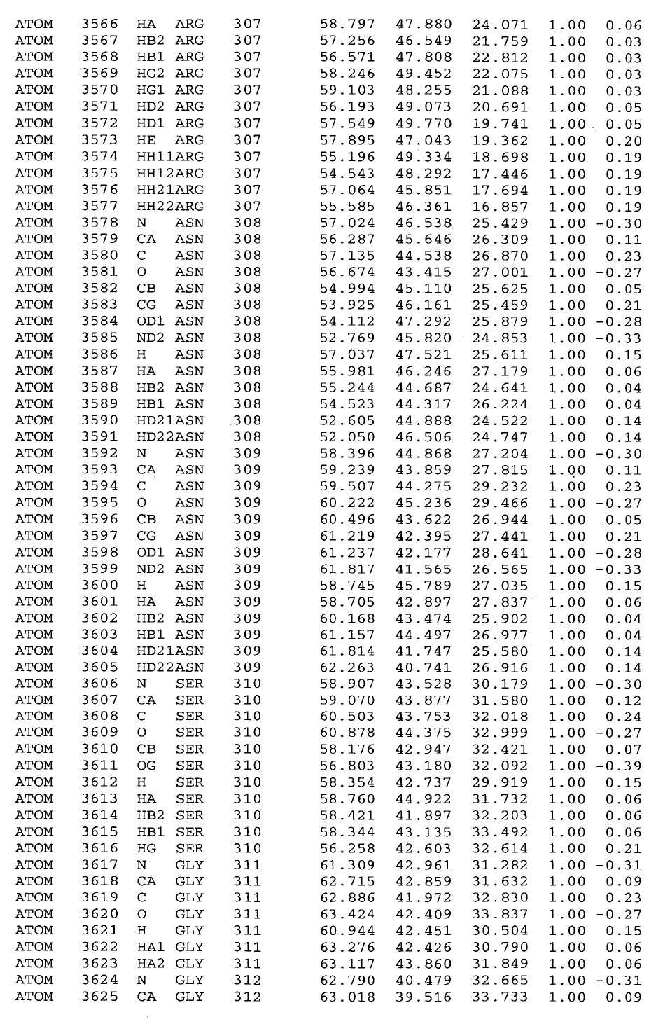

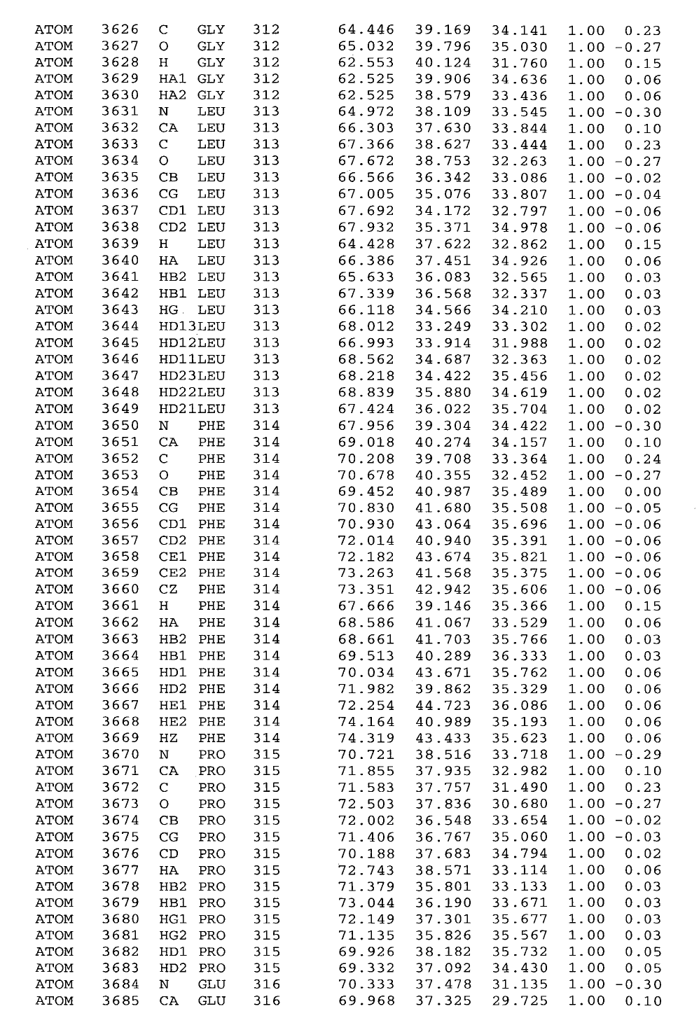

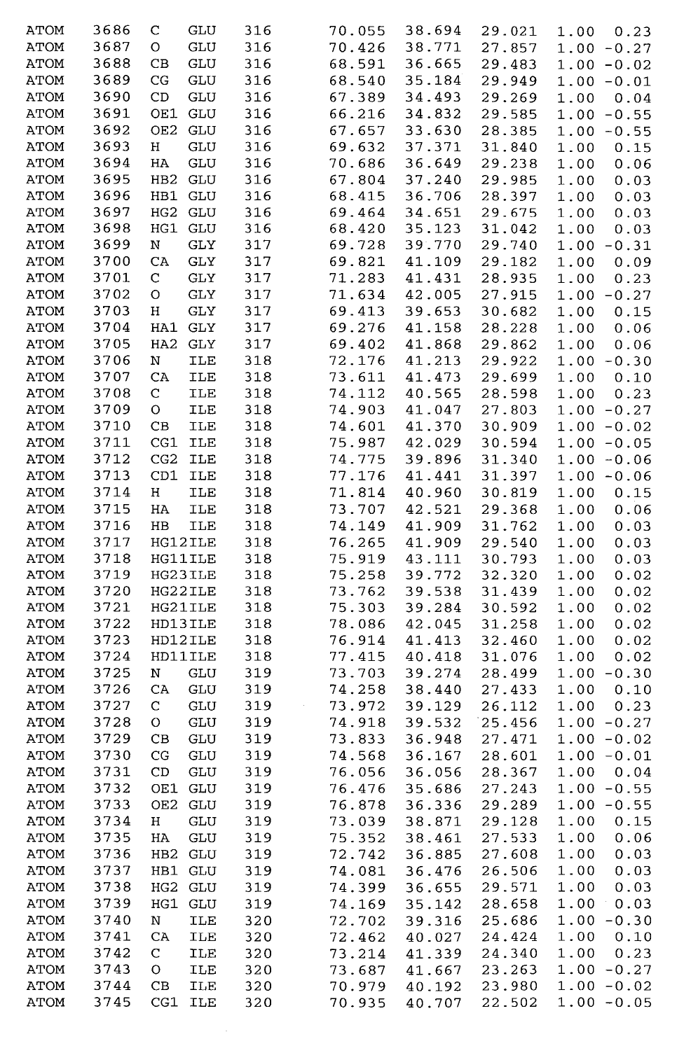

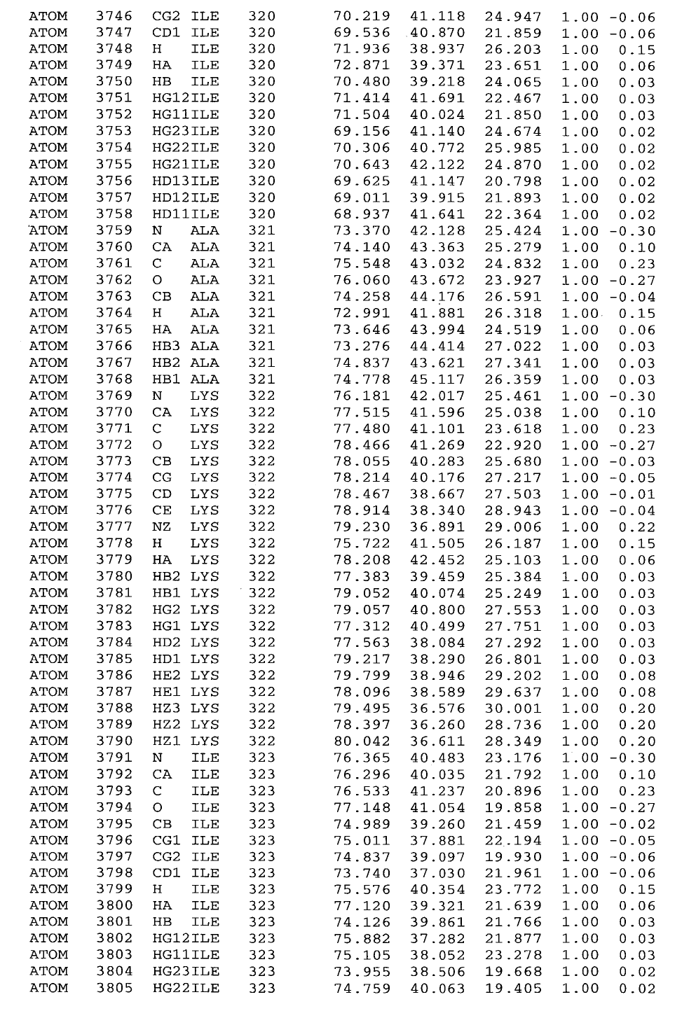

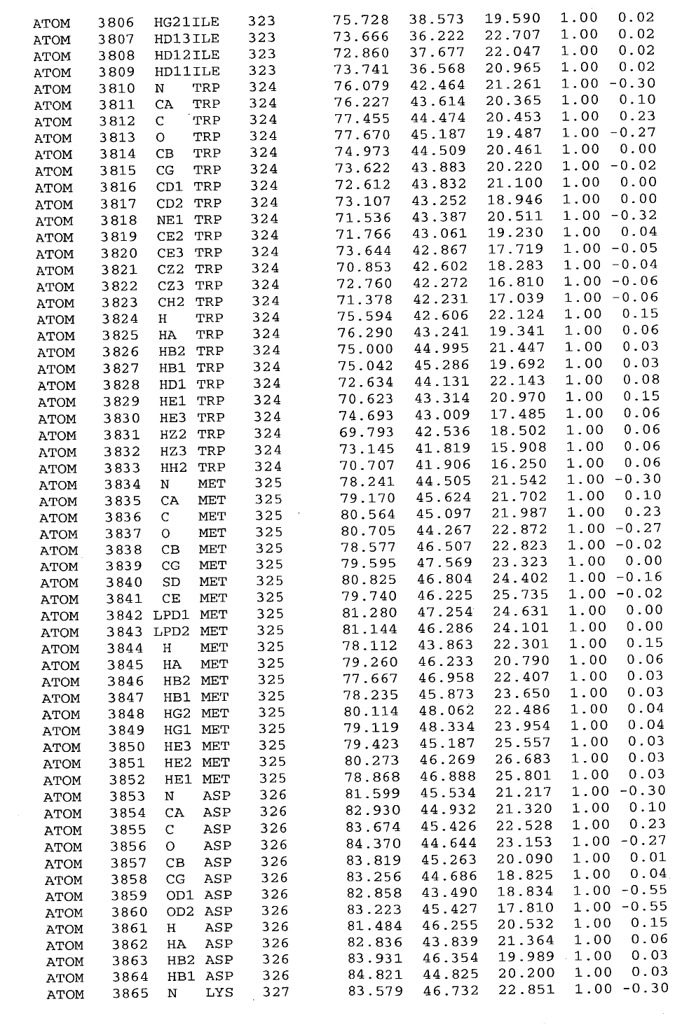

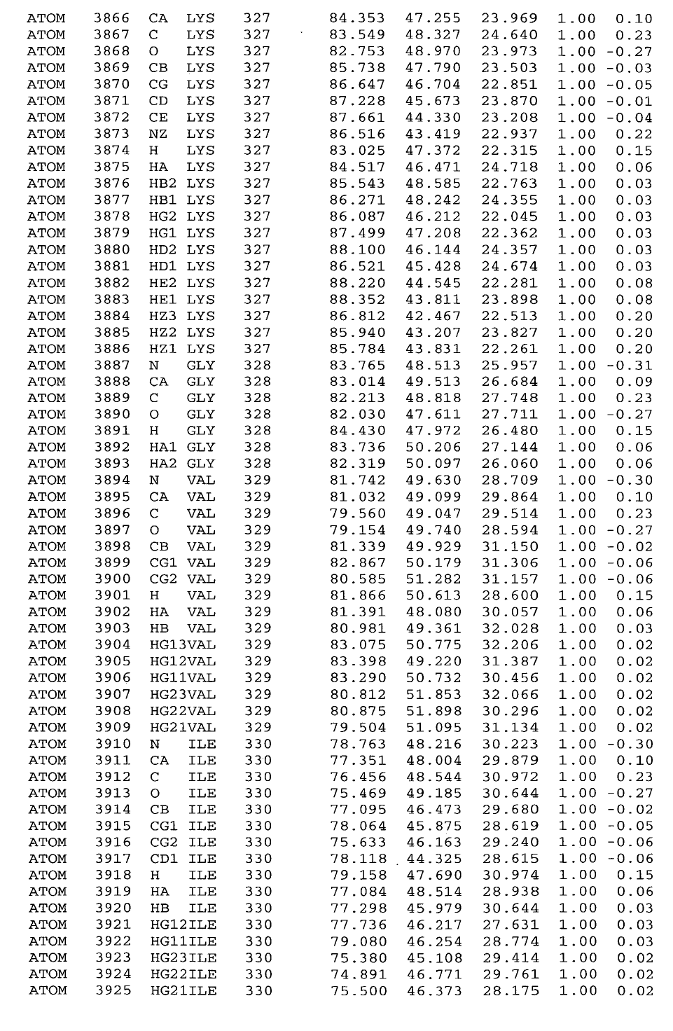

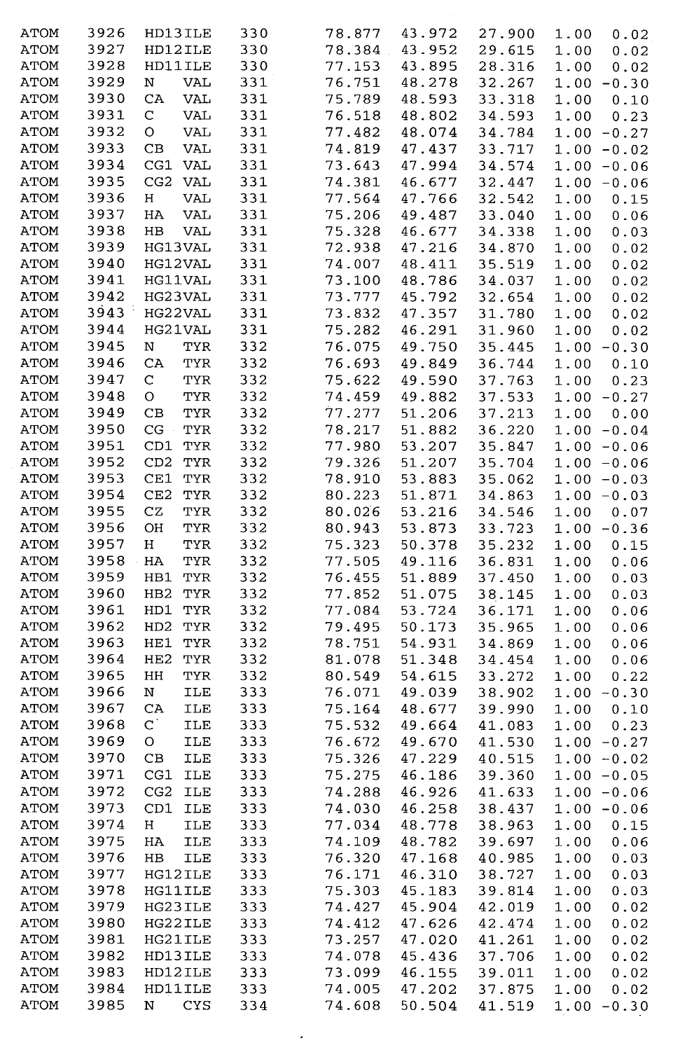

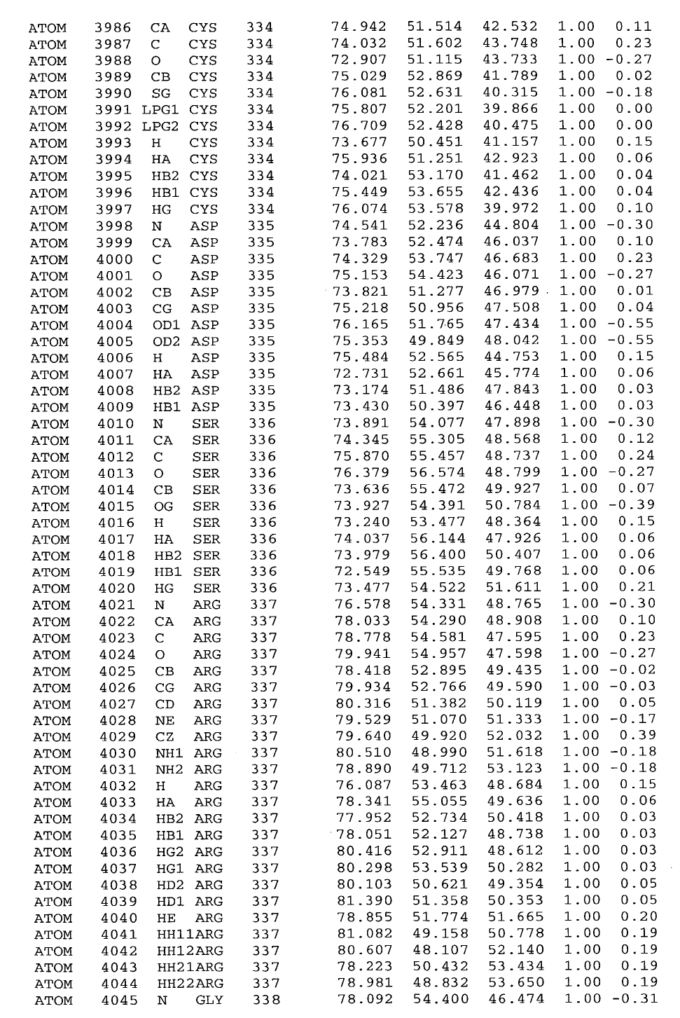

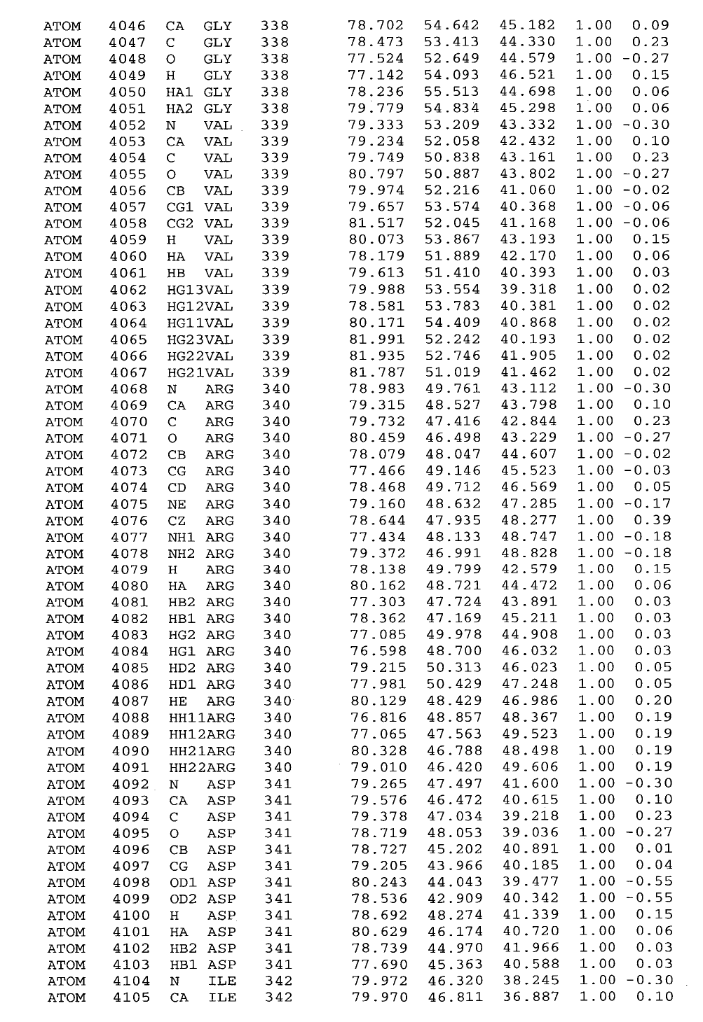

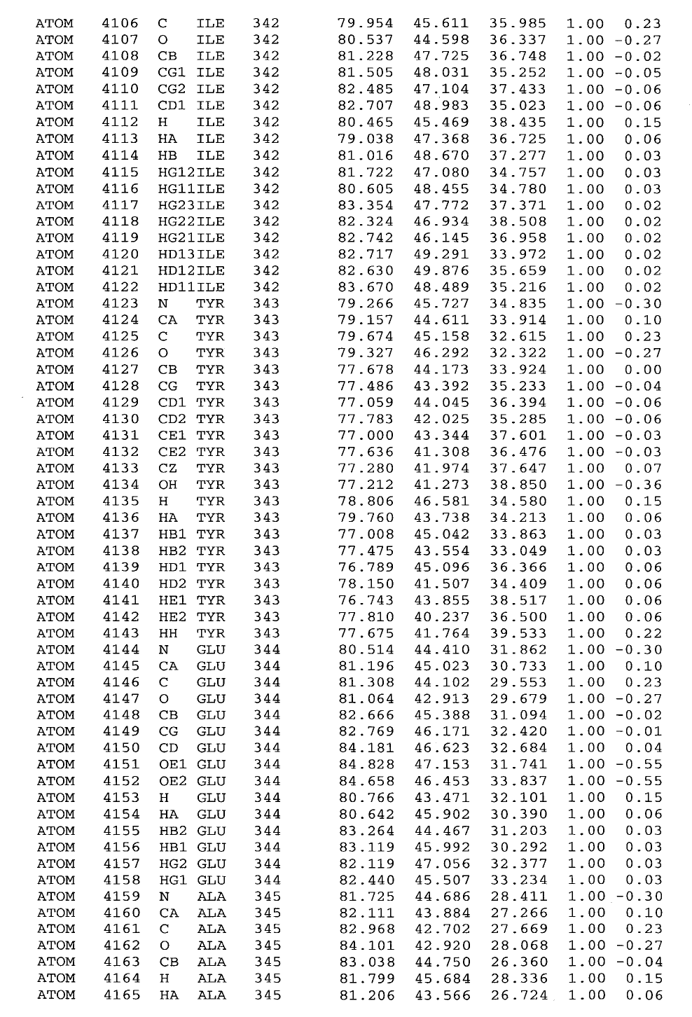

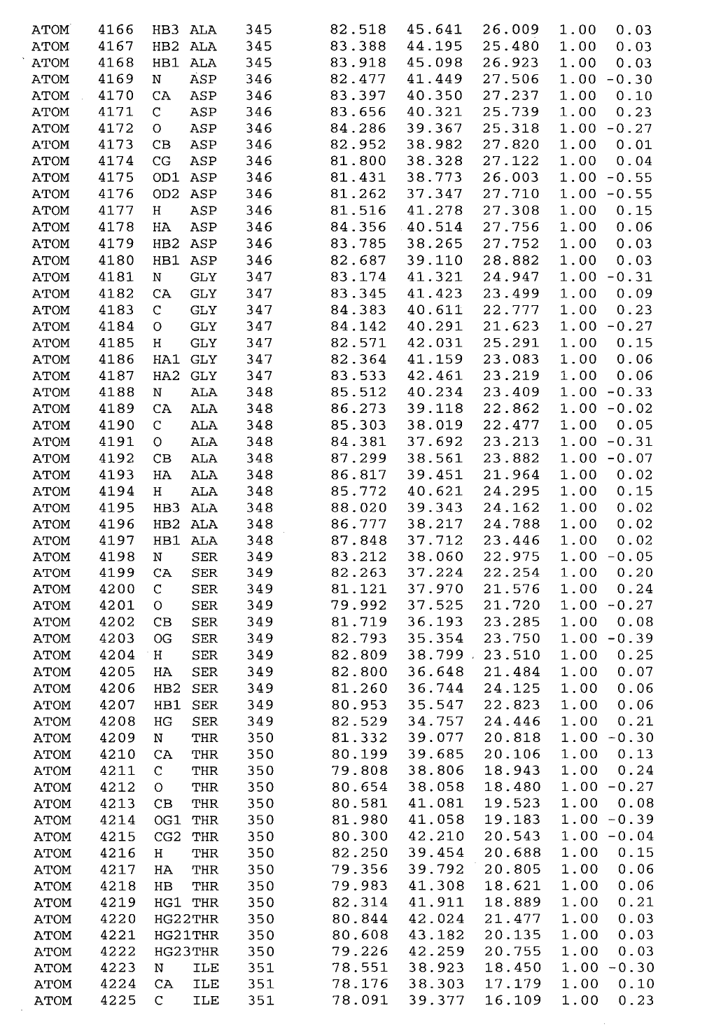

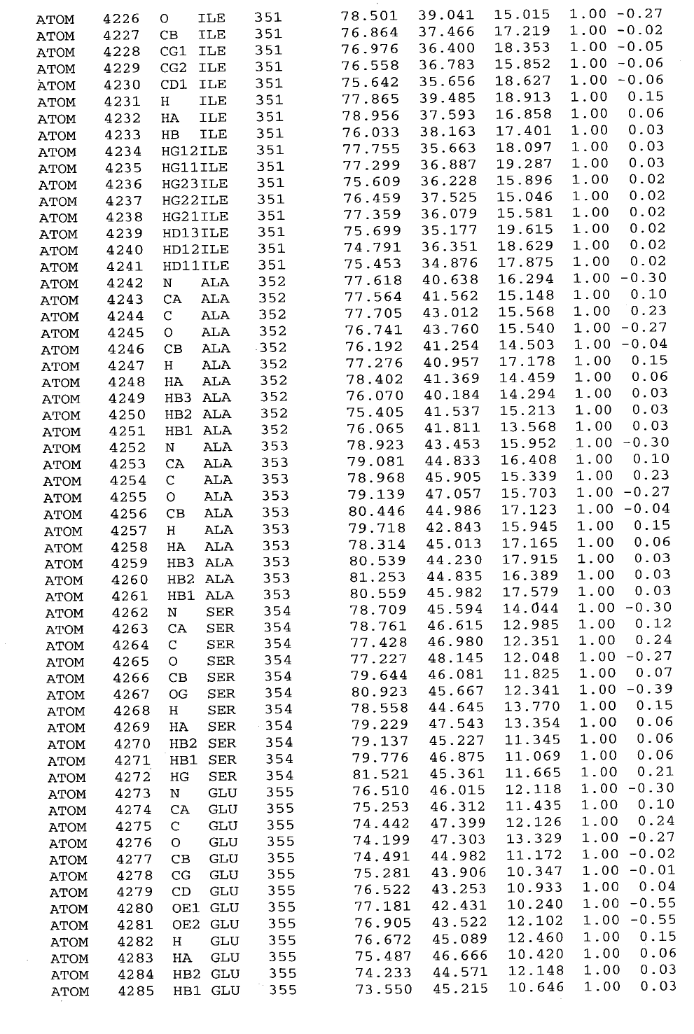

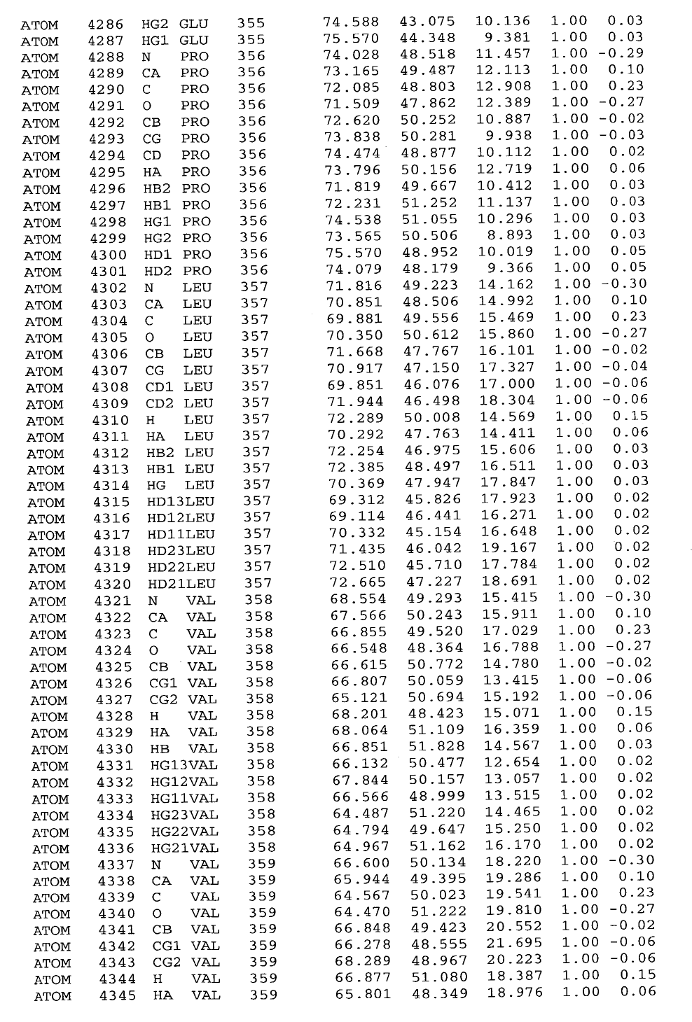

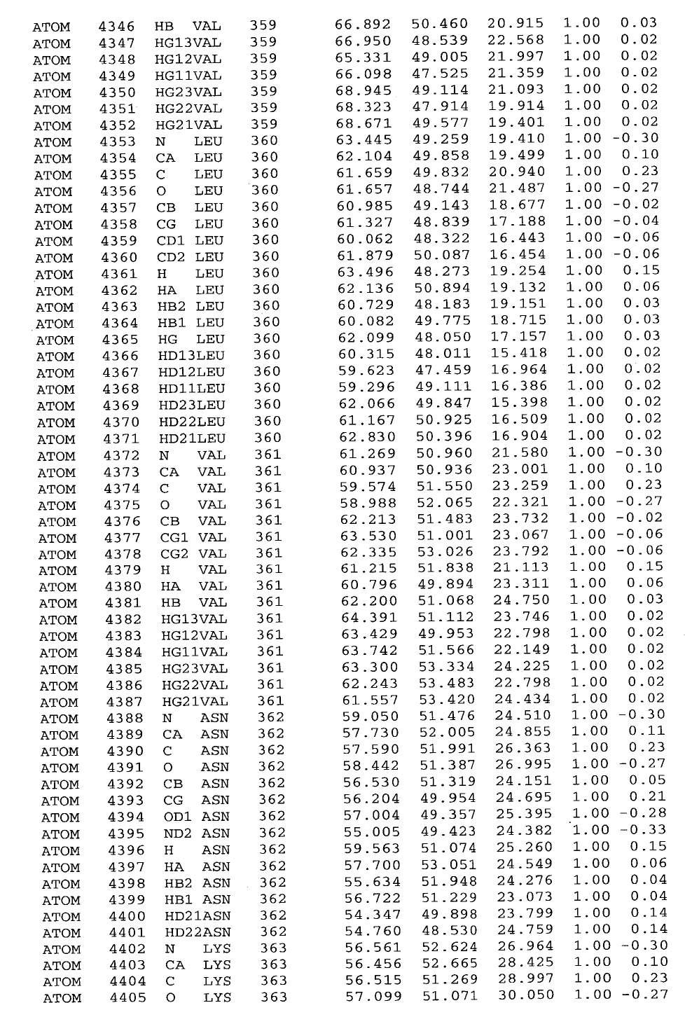

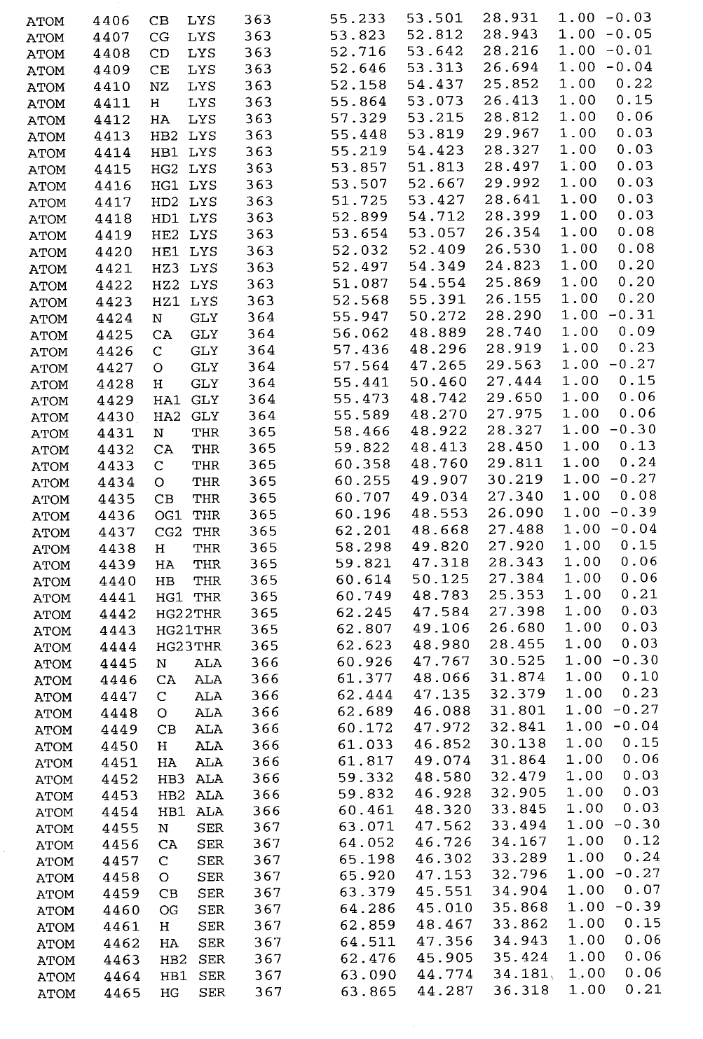

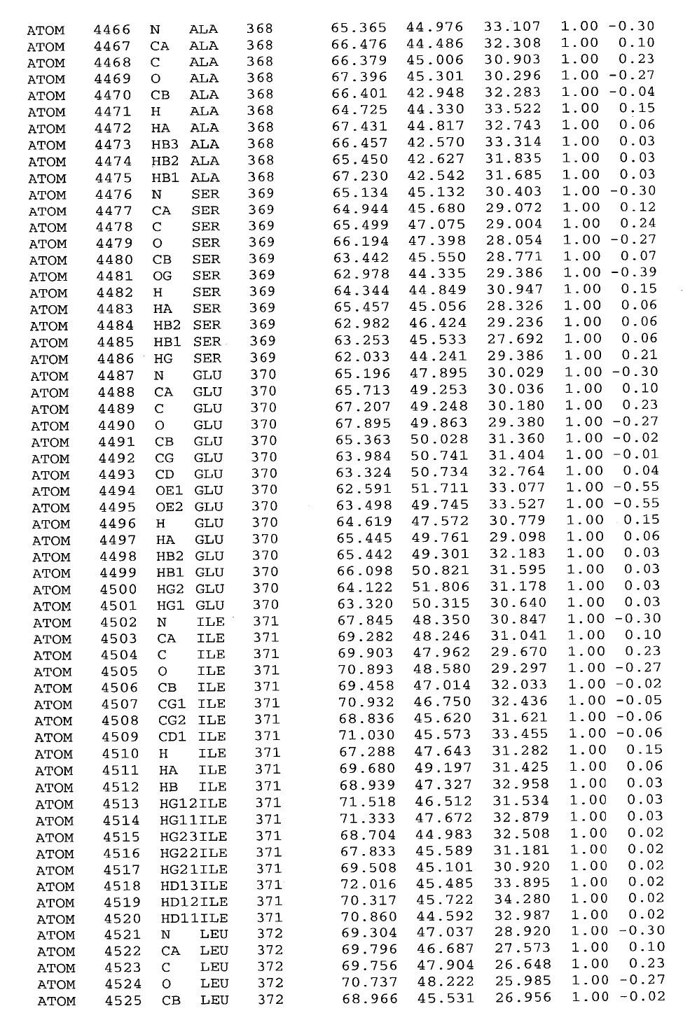

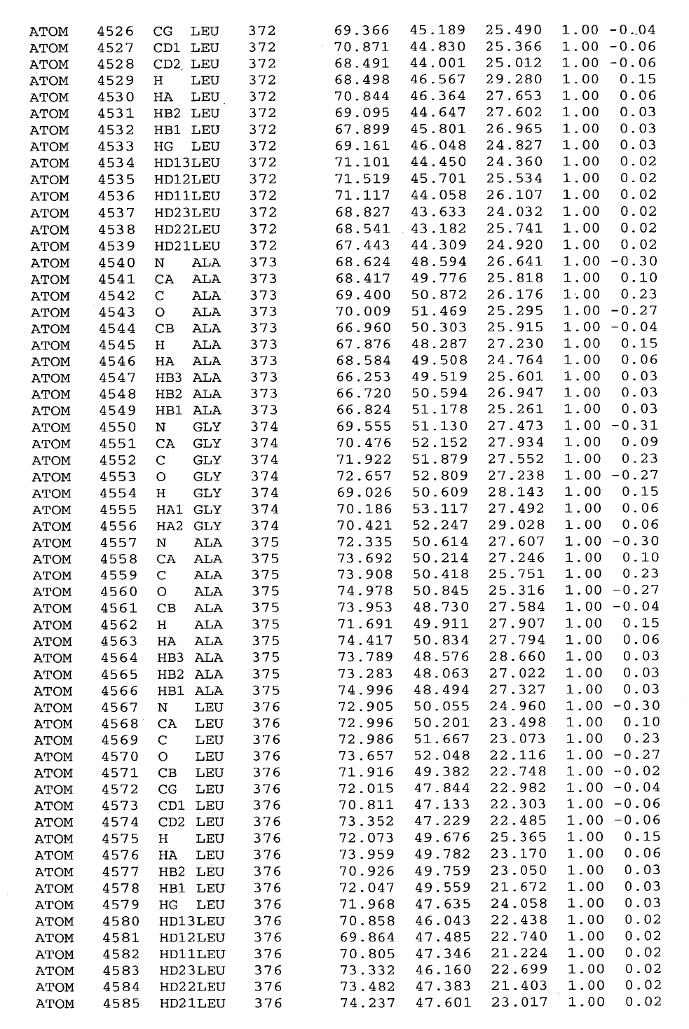

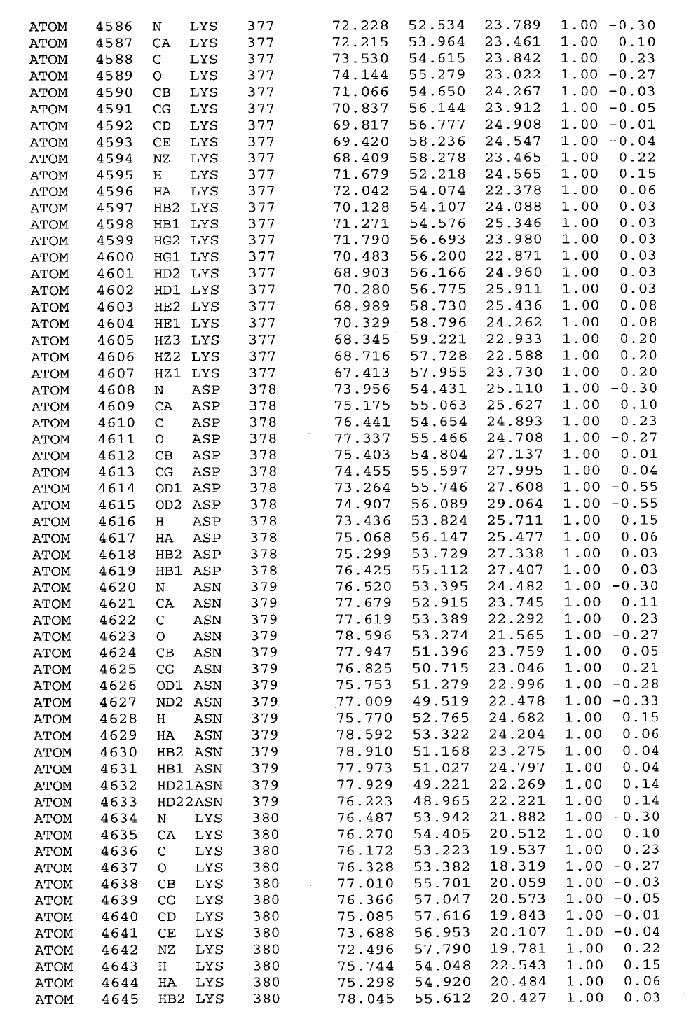

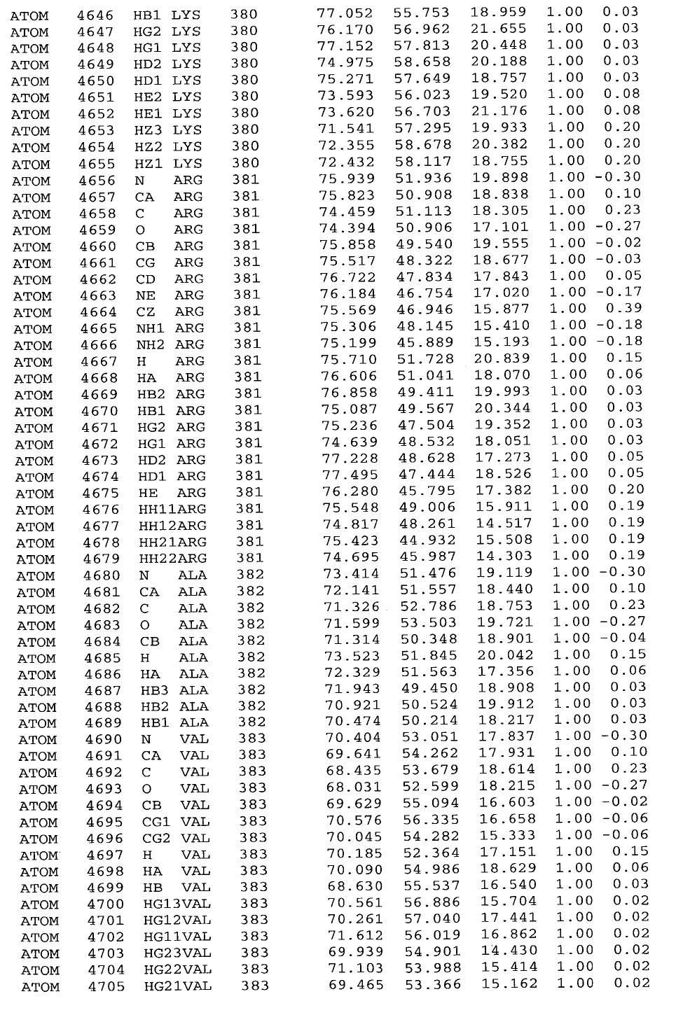

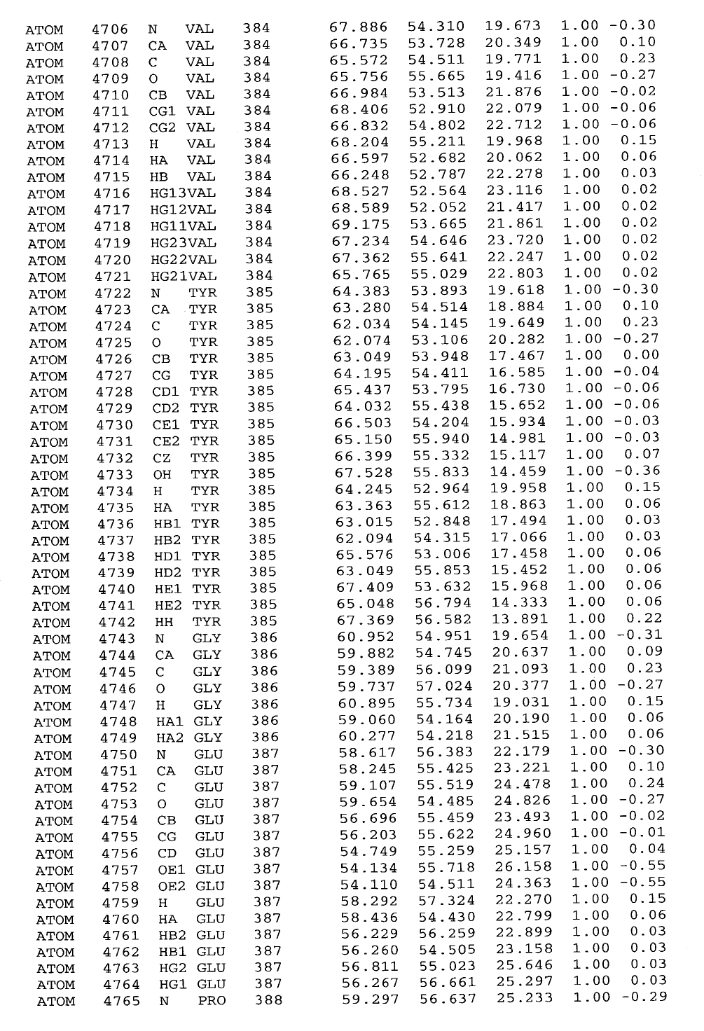

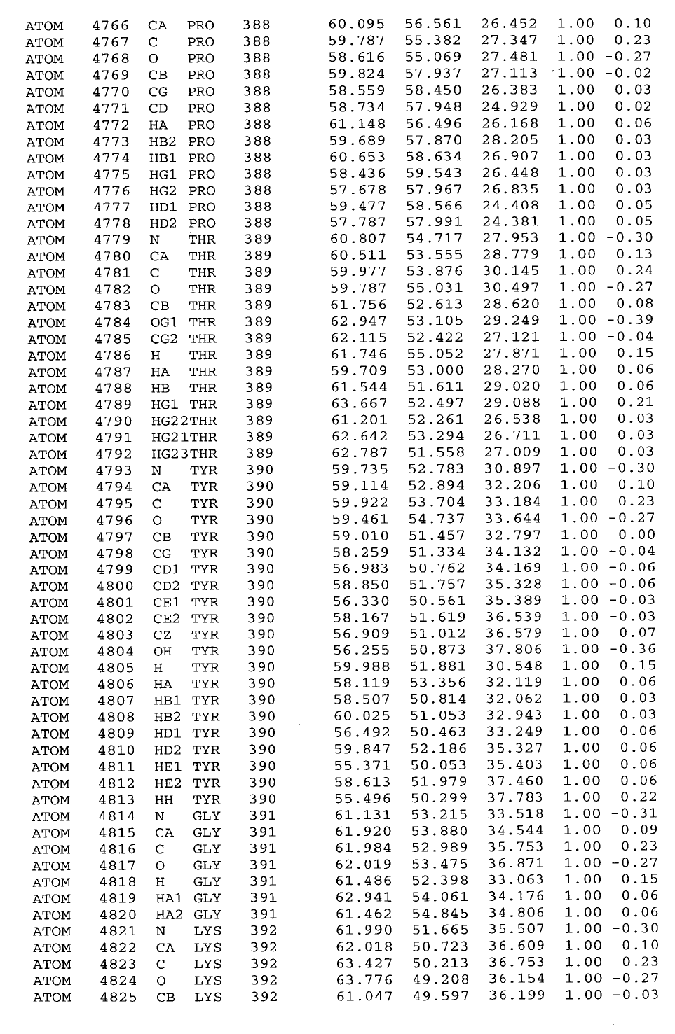

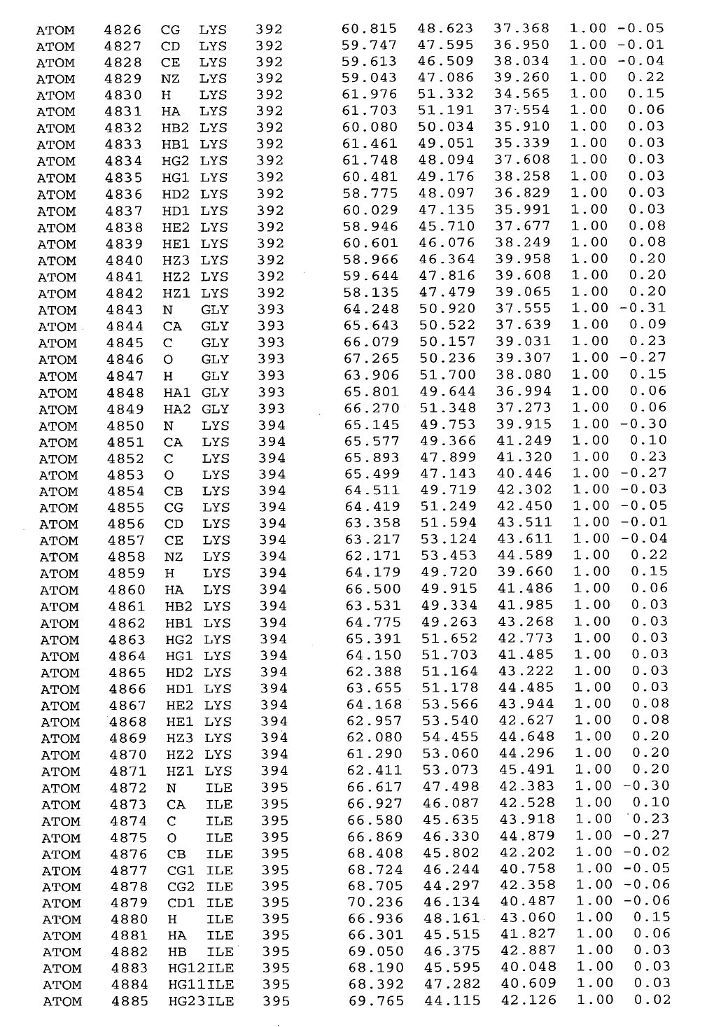

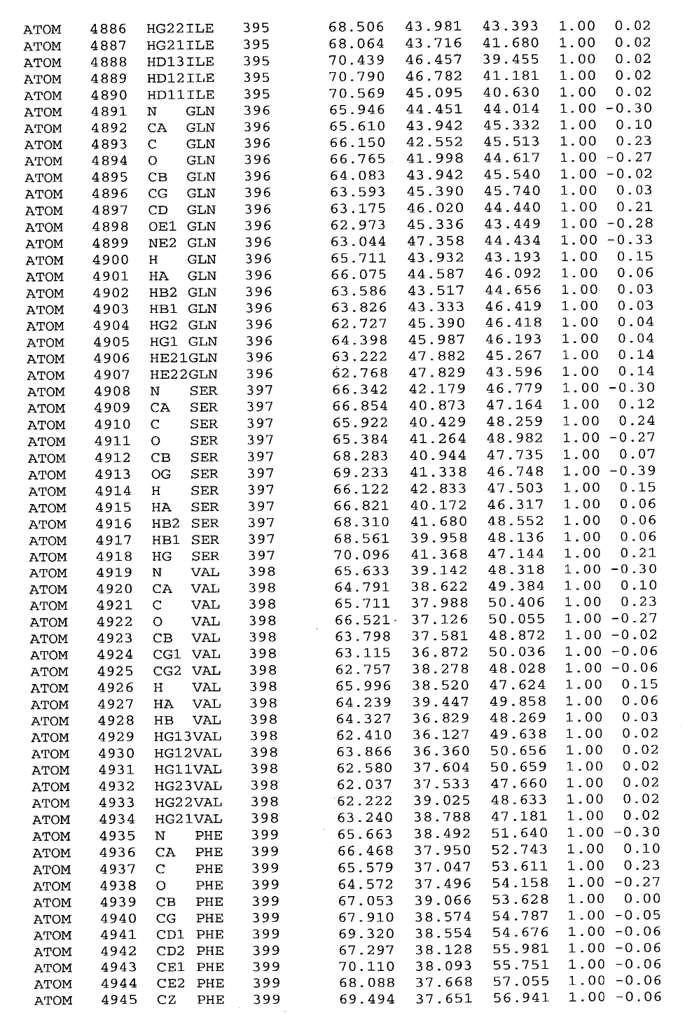

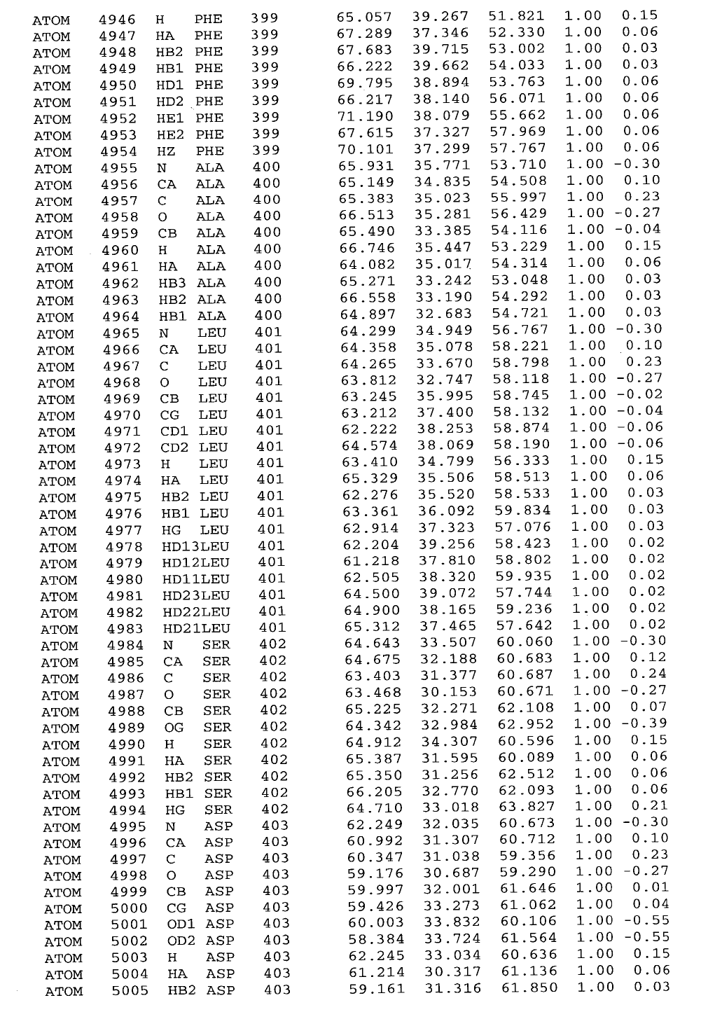

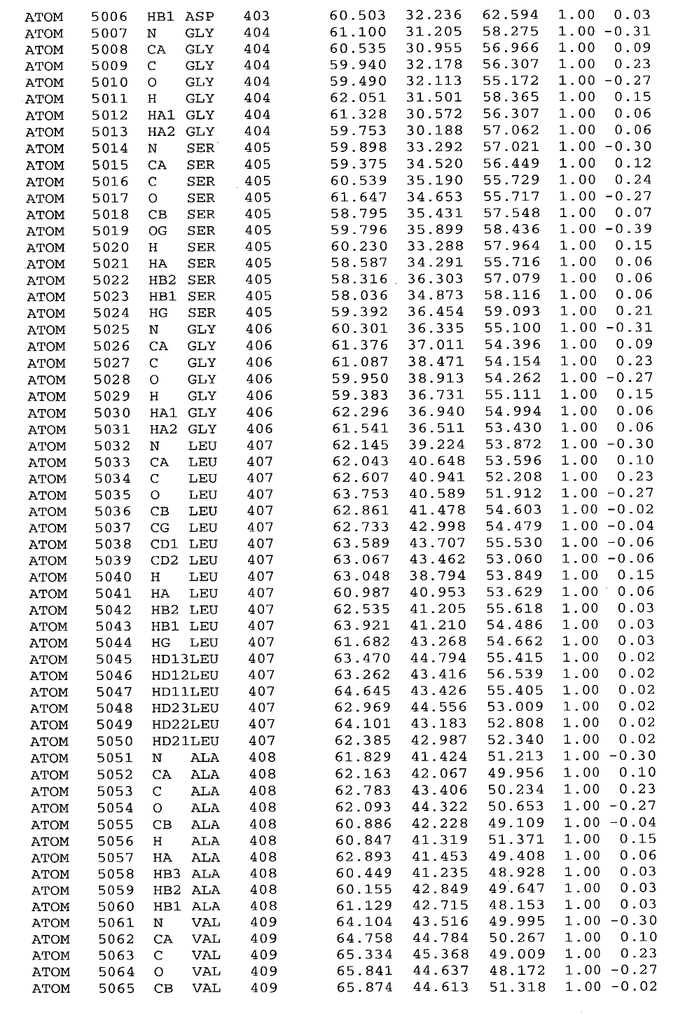

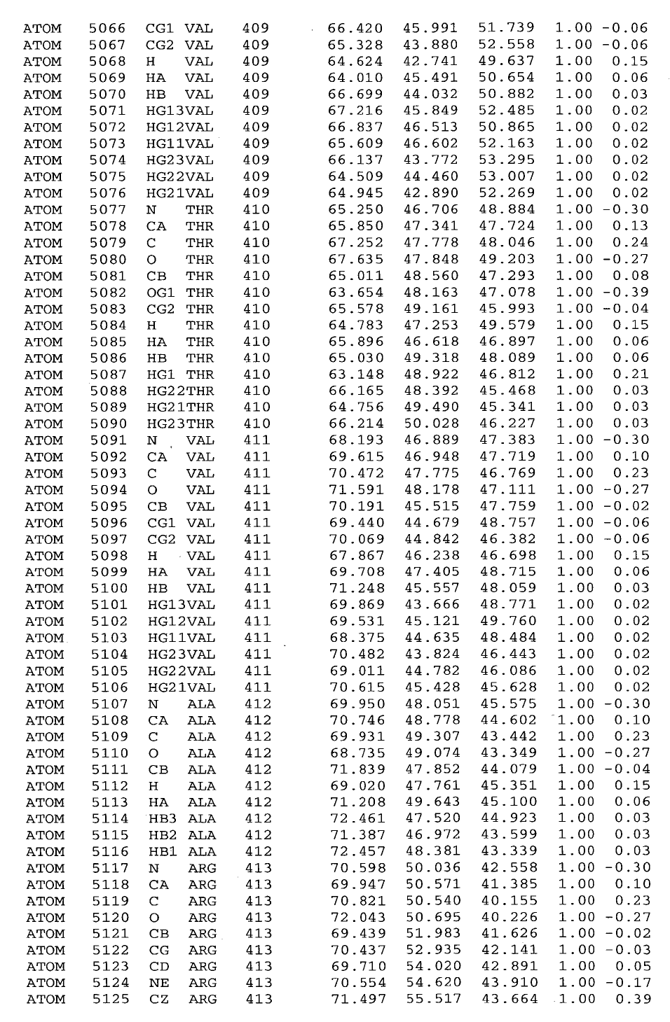

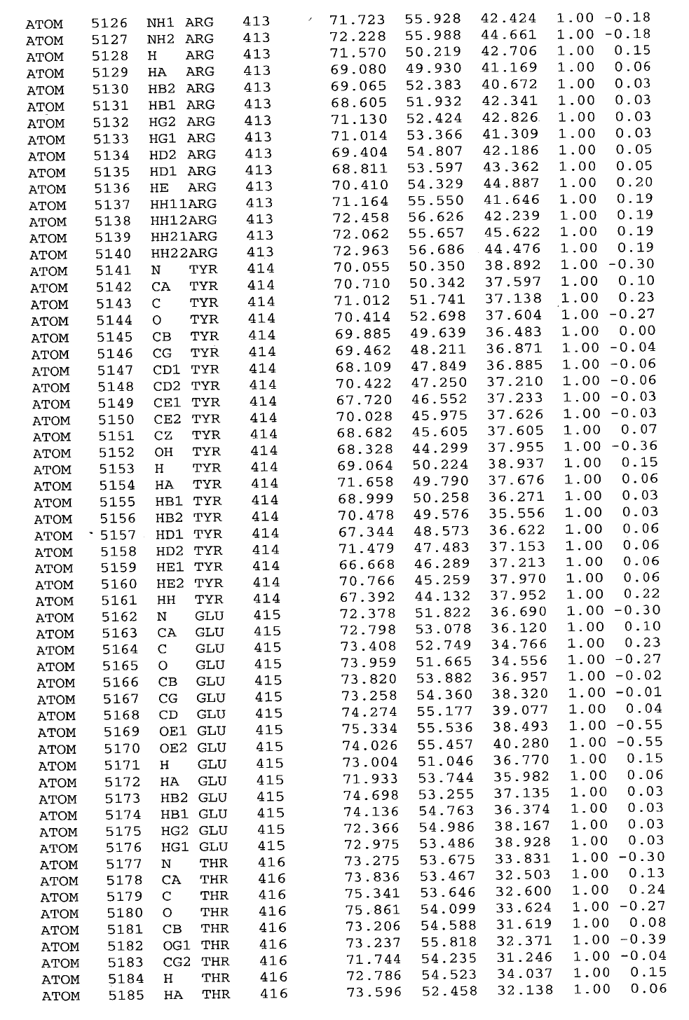

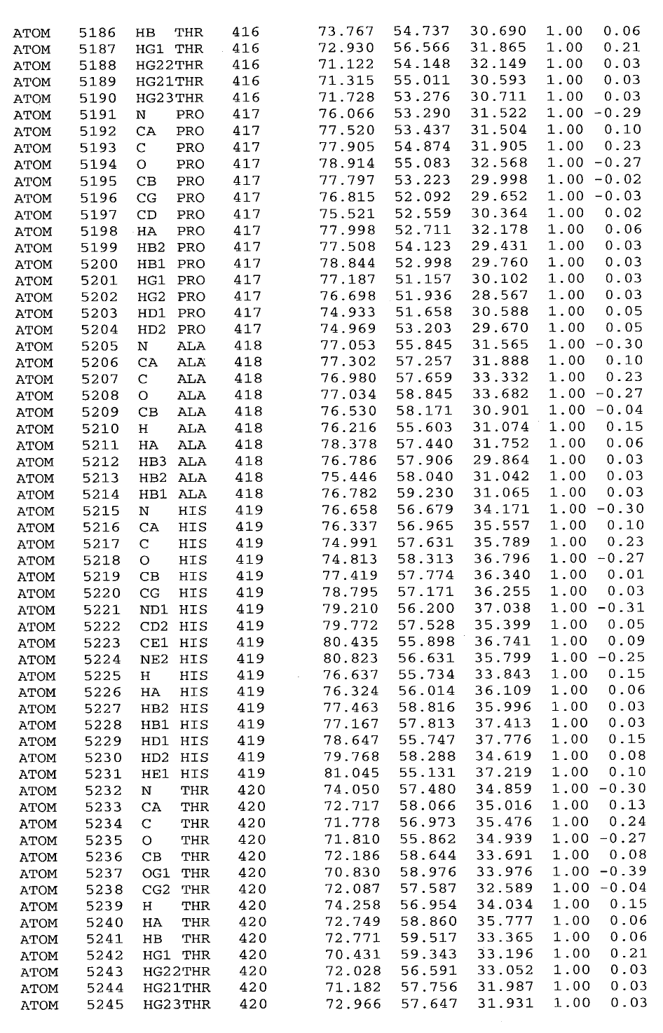

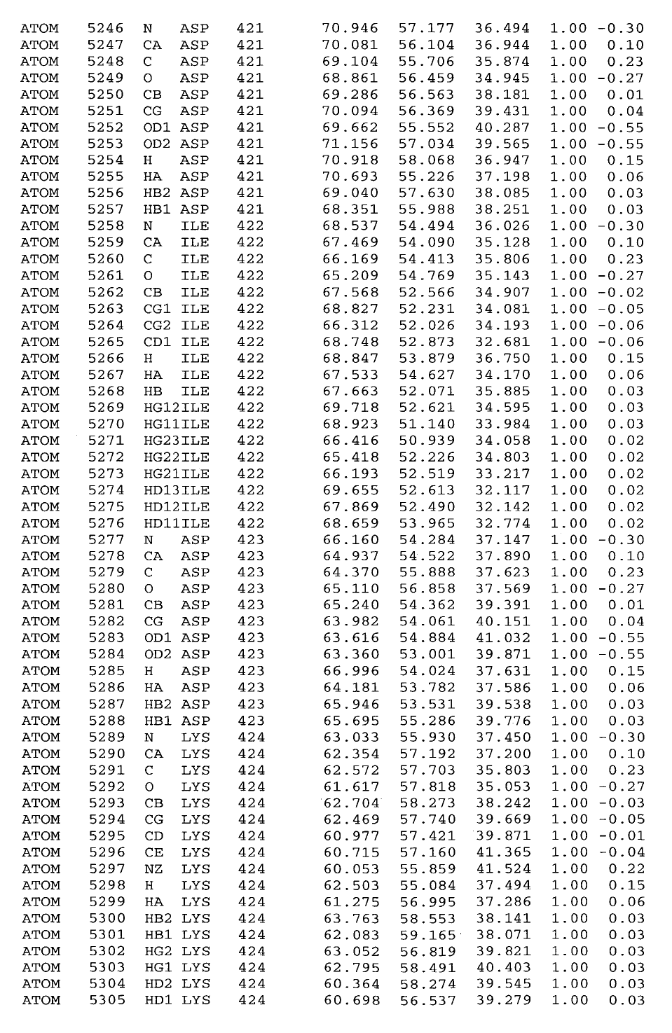

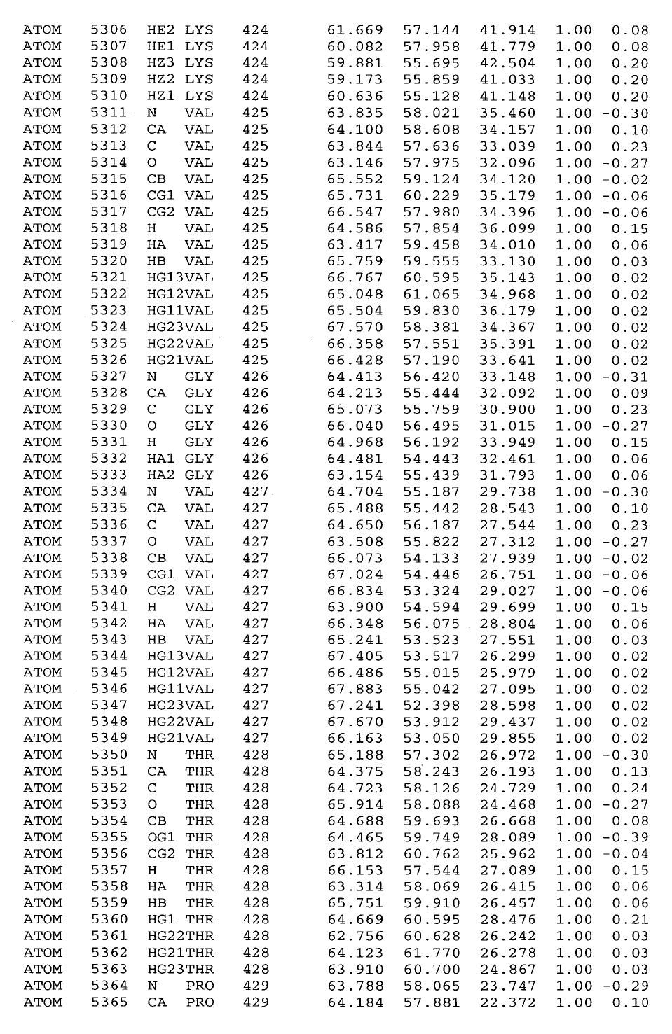

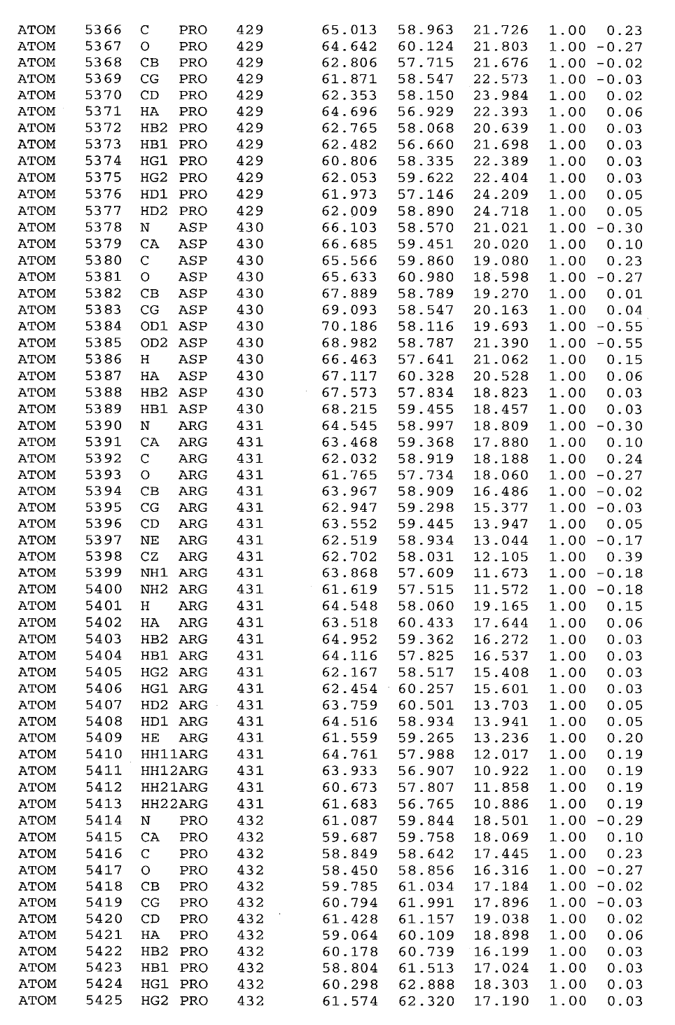

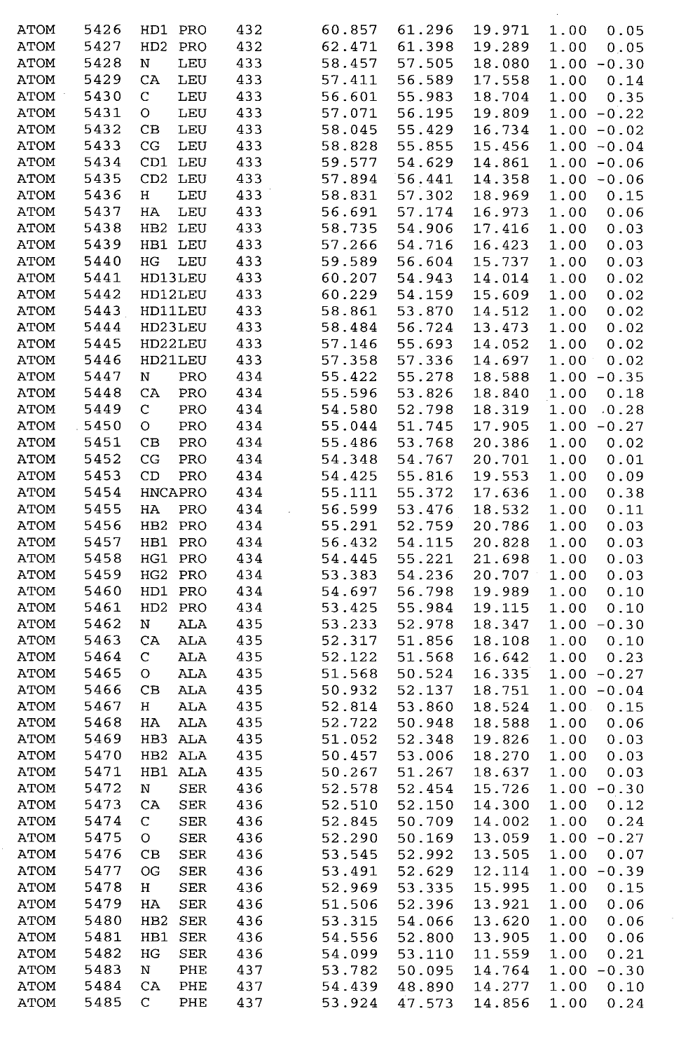

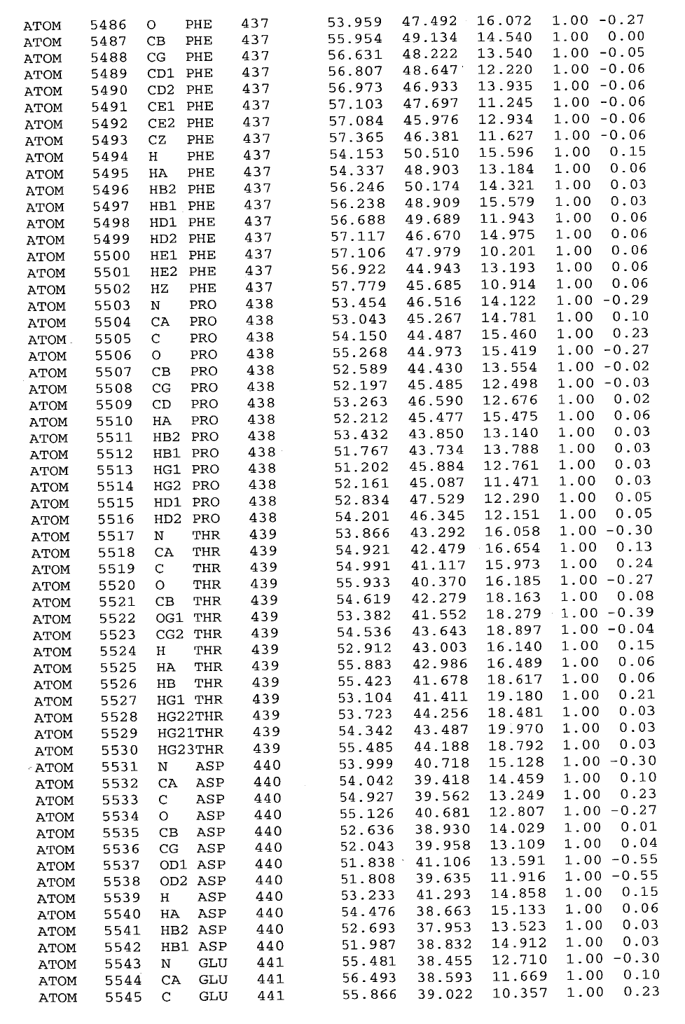

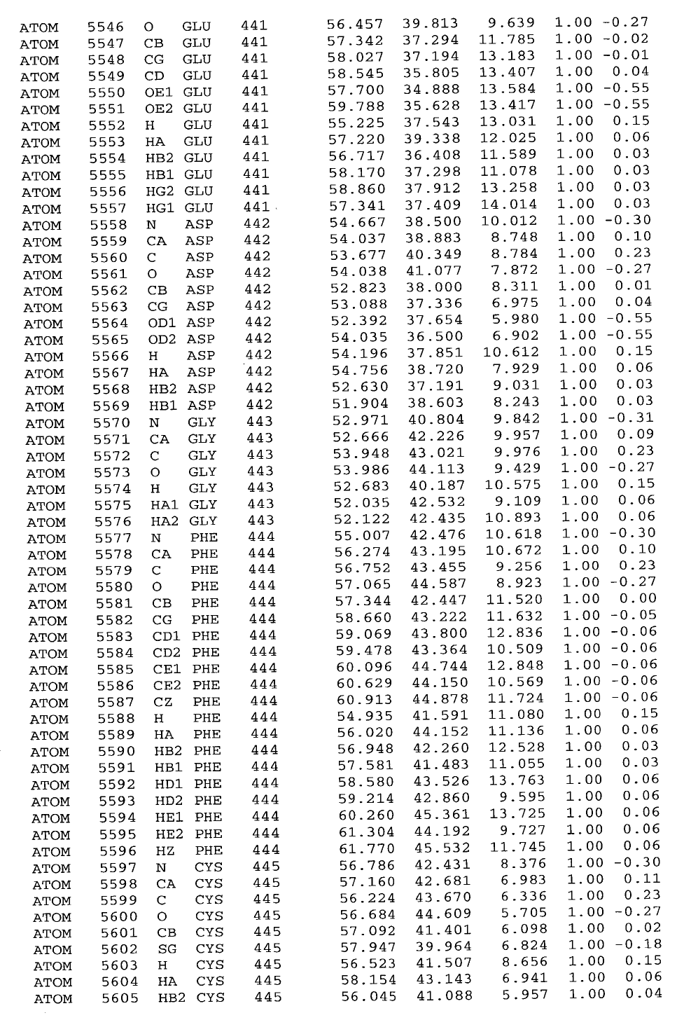

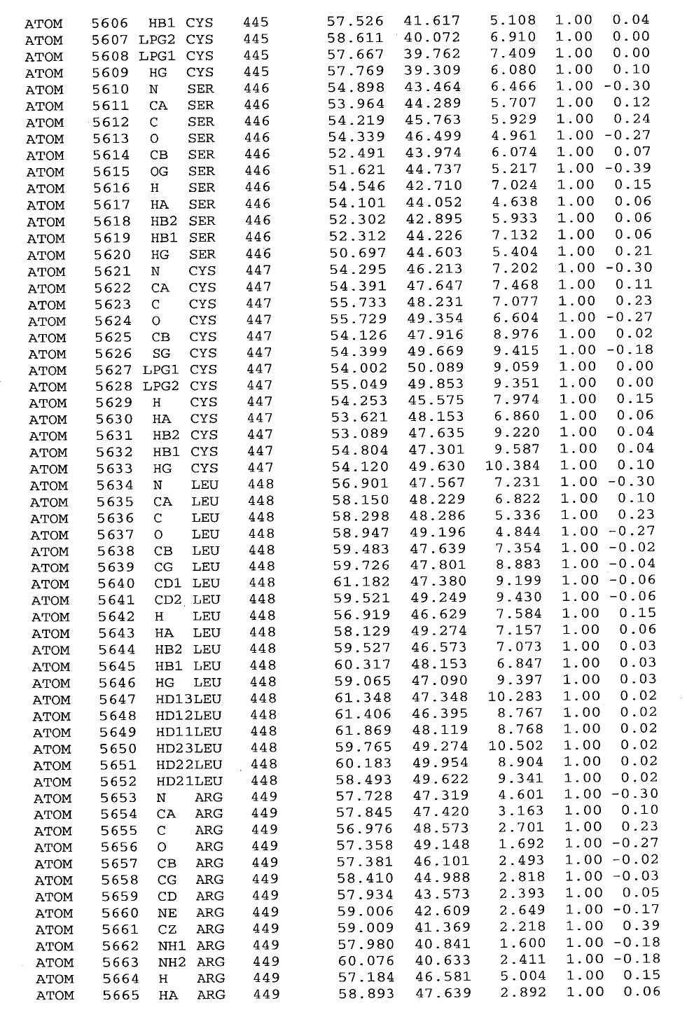

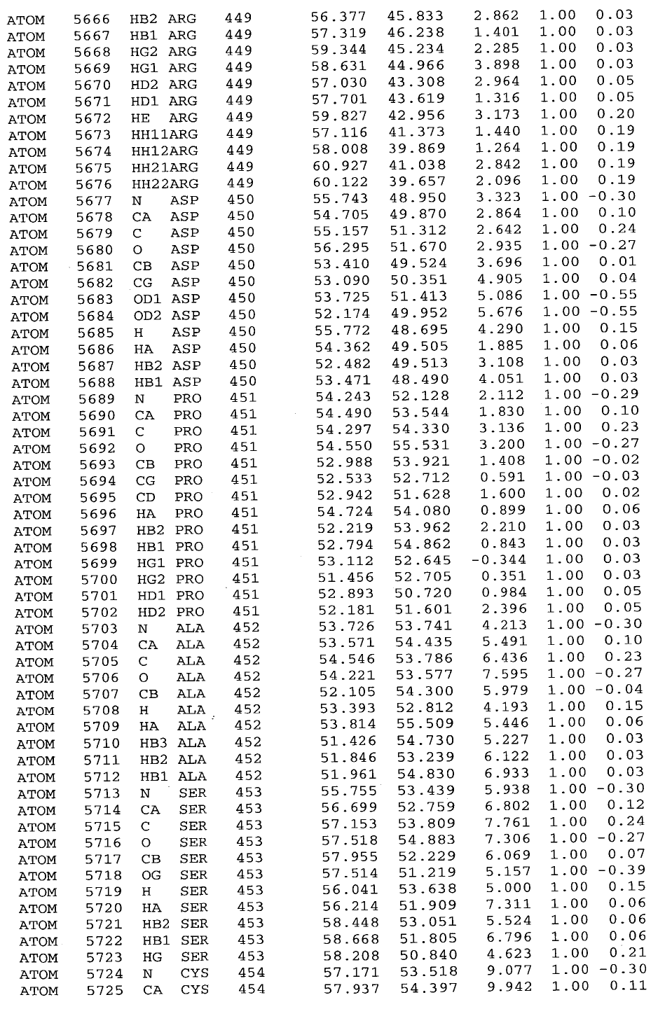

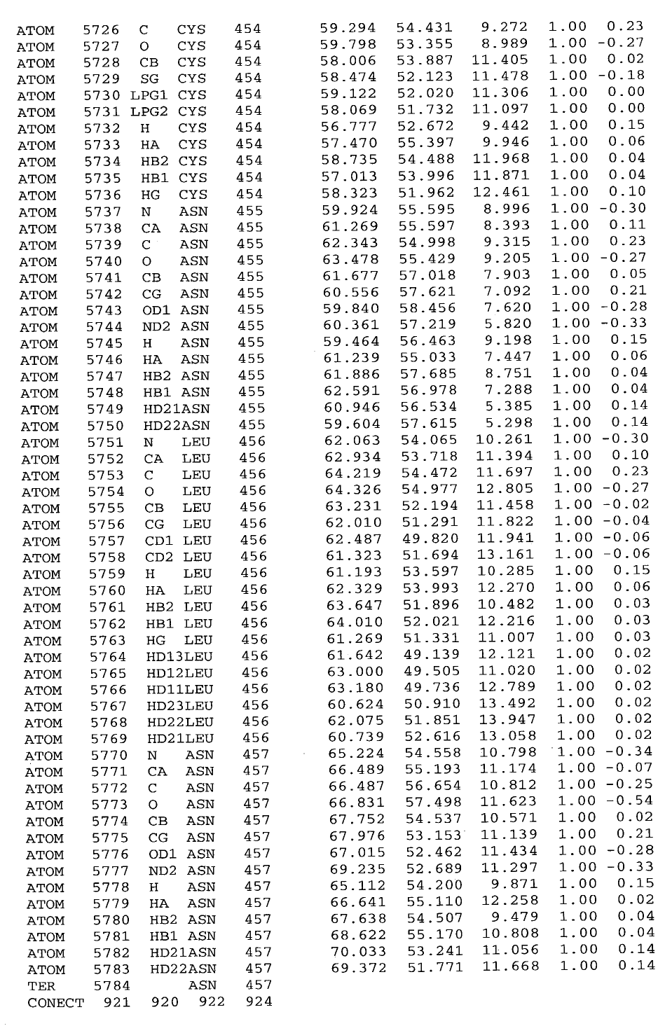

A three-dimensional model of wheat D1 protease was constructed based on the three-dimensional atomic coordinates of Scenedesmus obliquus D1 protease listed in FIG. 1. The amino acid sequence of D1 protease from wheat is presented in SEQ ID NO: 9. The amino acid sequence of this protein was found to be approximately 53% identical to that of the Scenedesmus obliquus D1 protease when compared with the GAP program (GCG), as shown in FIG. 3 using the default program values. Atomic coordinates of the Scenedesmus obliquus D1 protease were loaded into the molecular modeling package Sybyl®. By using the Biopolymer package within Sybyl®, amino acids of the Scenedesmus obliquus D1 protease were mutated to reflect the amino acid sequence of wheat D1 protease. Insertions and deletions were conducted using the annealing routine of Biopolymer. Finally, the model of wheat D1 protease was minimized by using the energy minimization routine of Sybyl® holding the protein backbone constant (in an aggregate), adding hydrogens fully to the structure, and adding charges. The predicted atomic coordinates of the resulting three-dimensional model are listed in FIG. 4. The model for wheat D1 protease may be used for inhibitor design by applying one of several methods for docking potential inhibitors within the constraints of the active site defined by the model.

### Example 14

Crystal Structure and Computer Modeling of D1P in Complex With A Irreversible Peptide Chloromethylketone Inhibitor

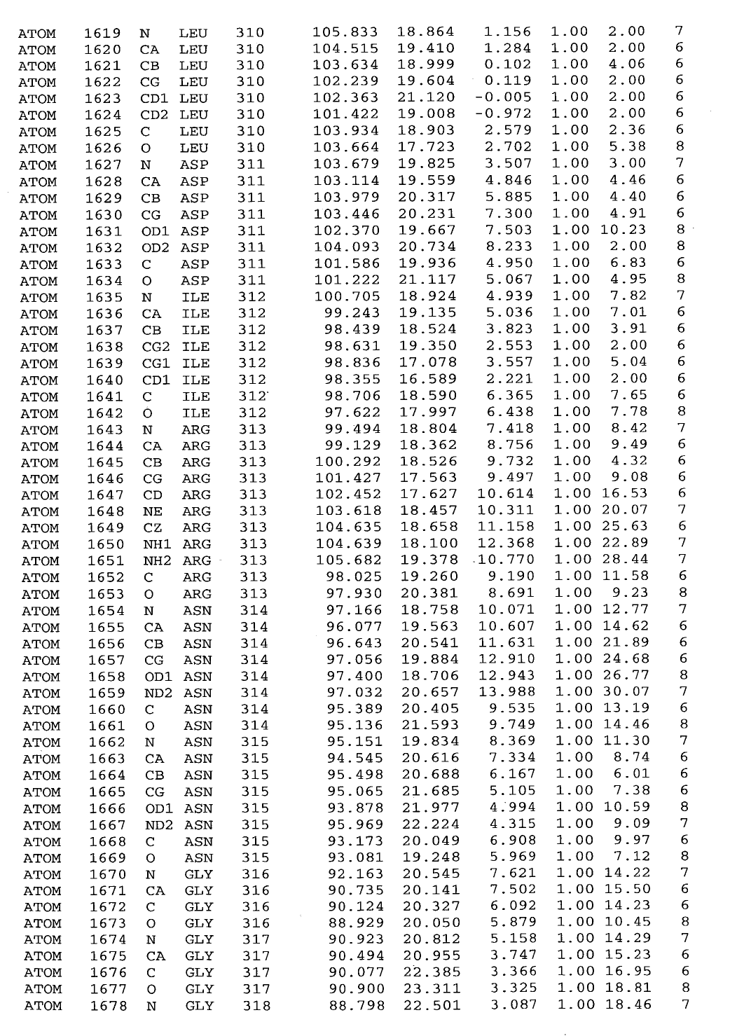

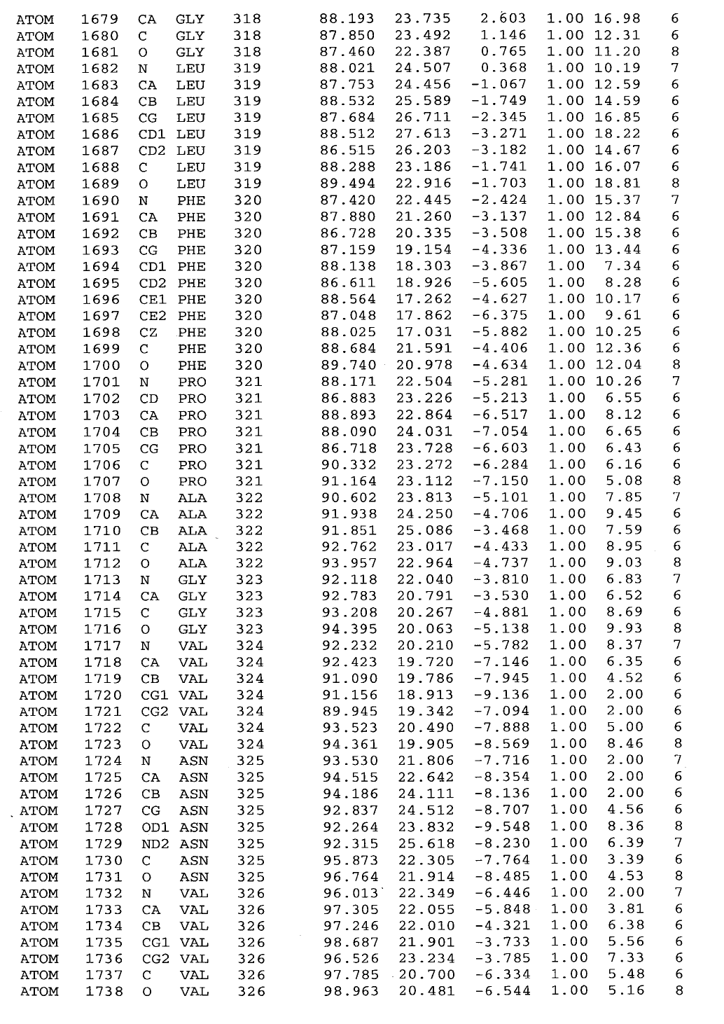

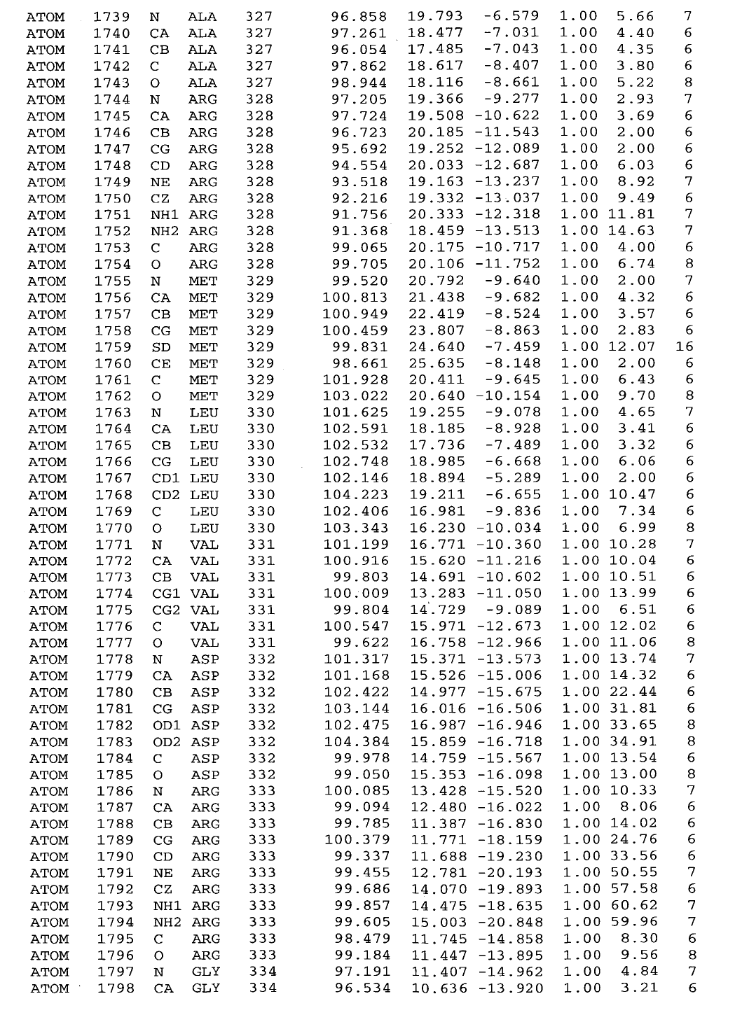

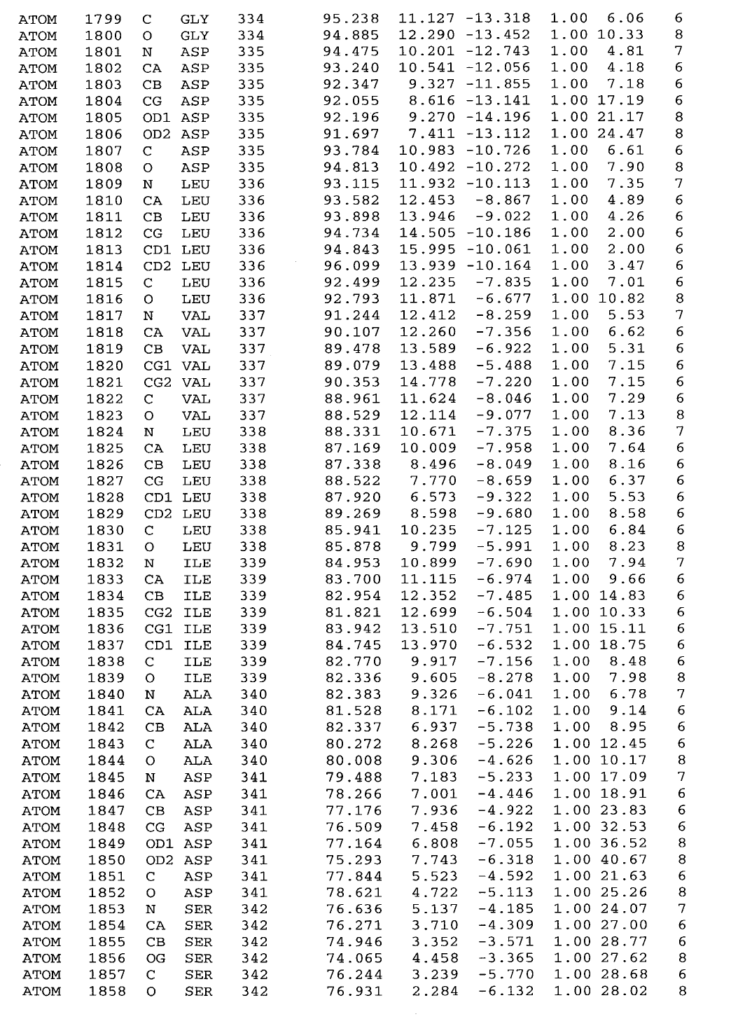

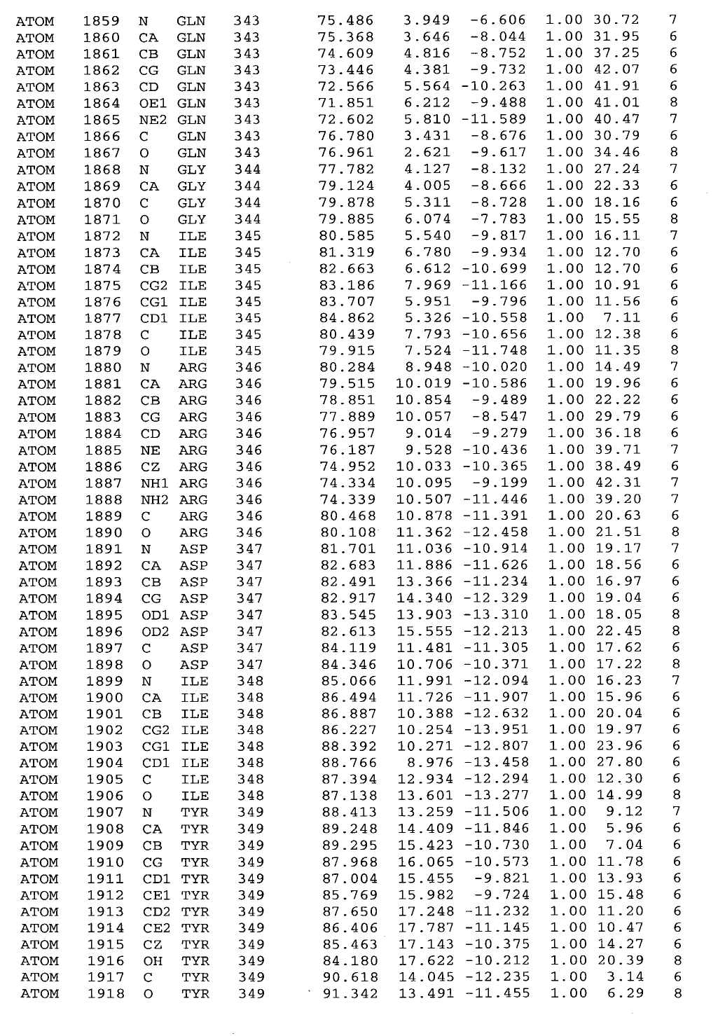

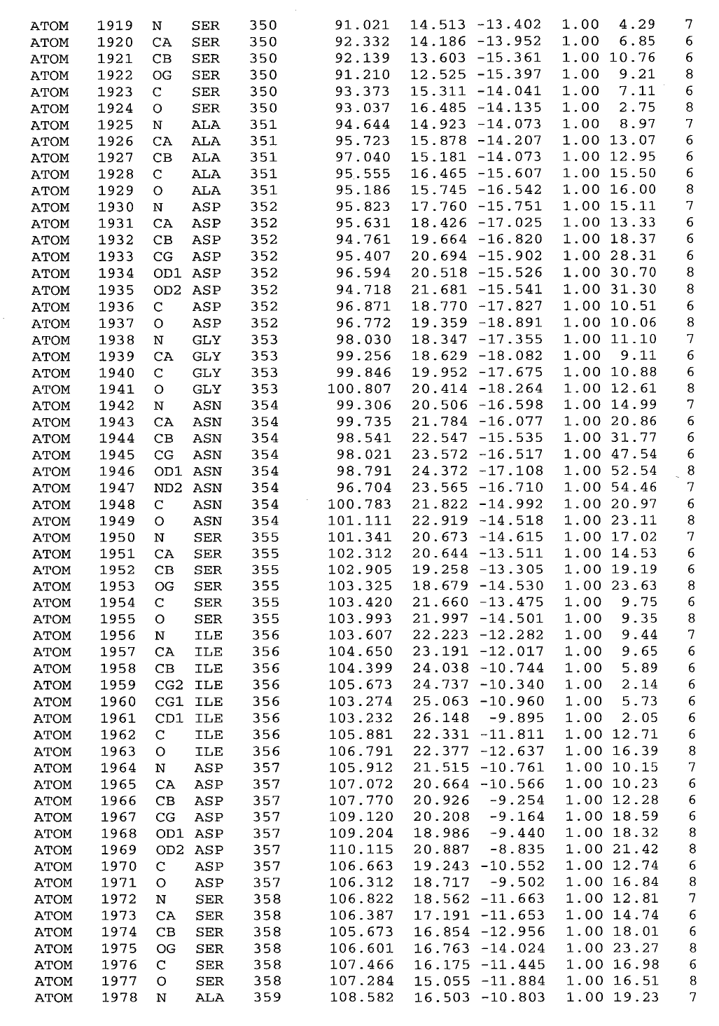

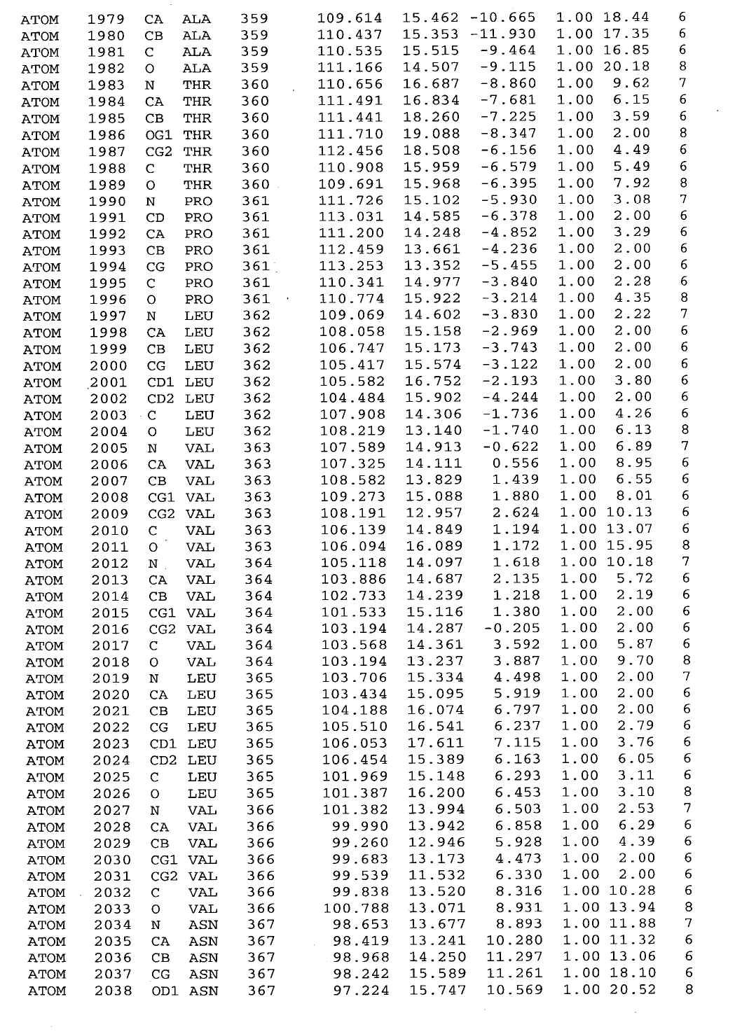

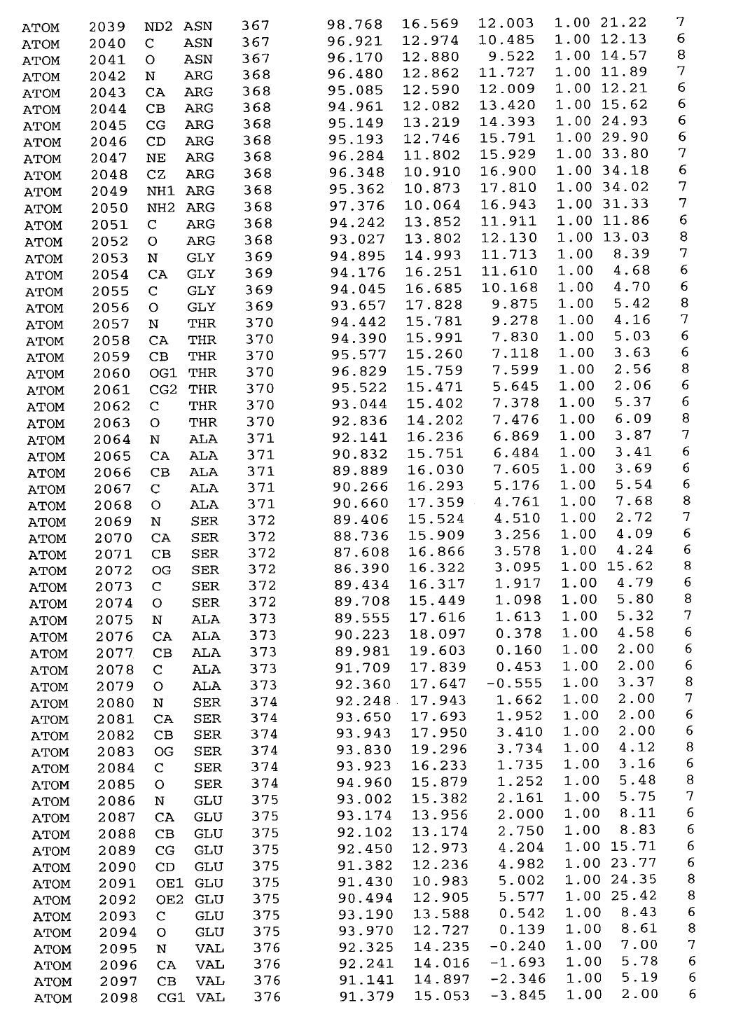

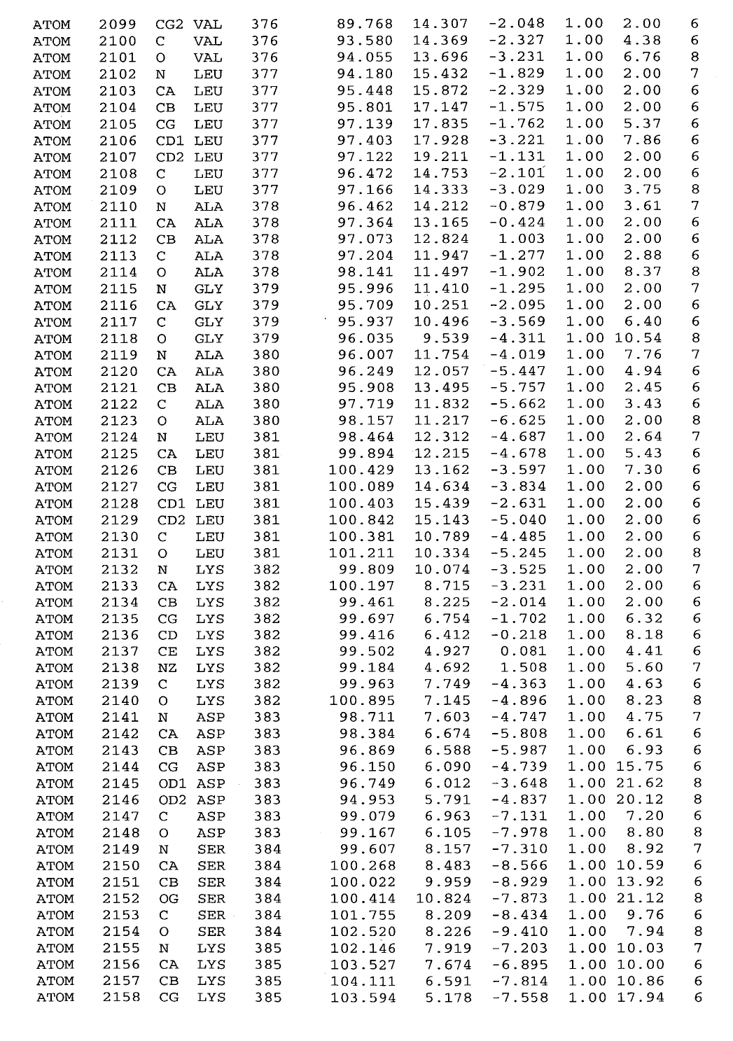

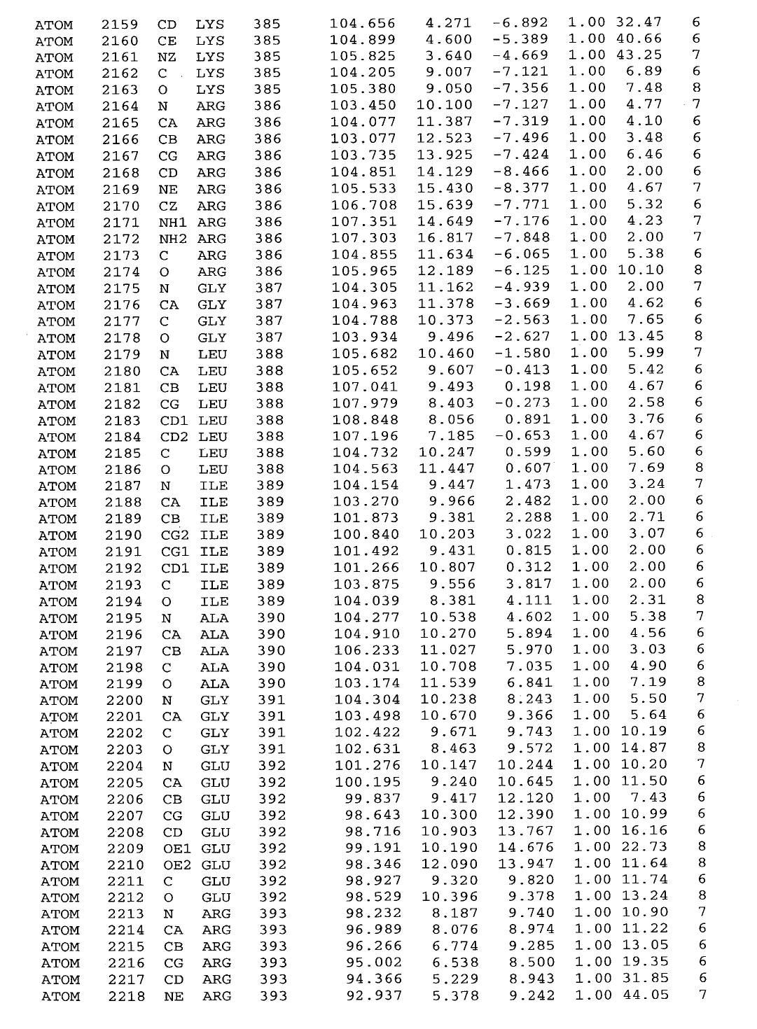

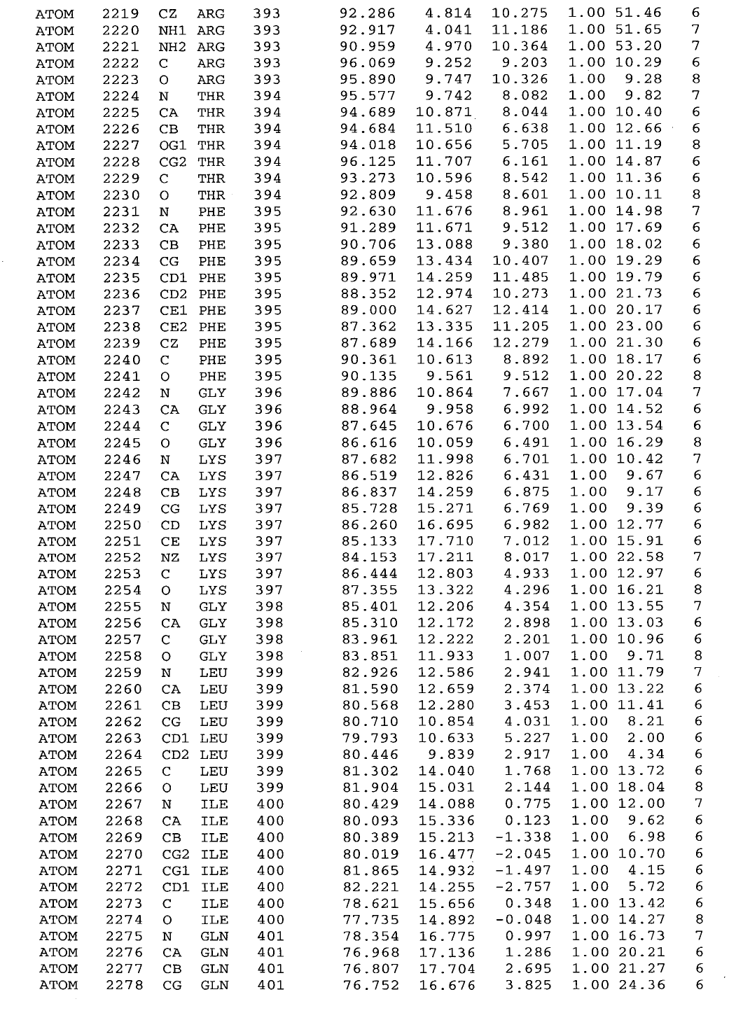

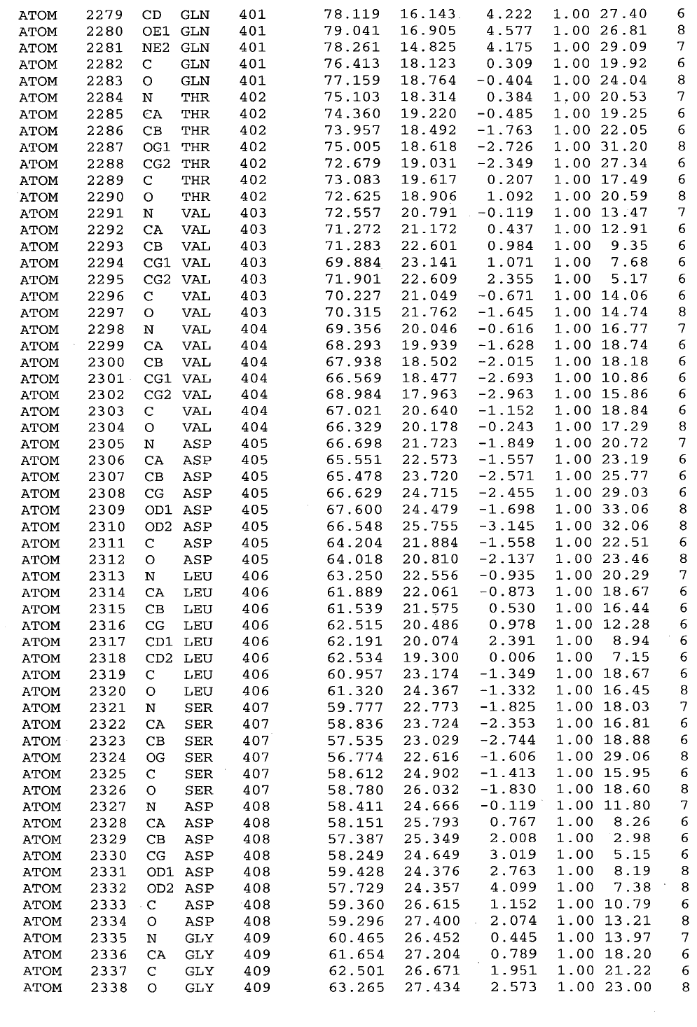

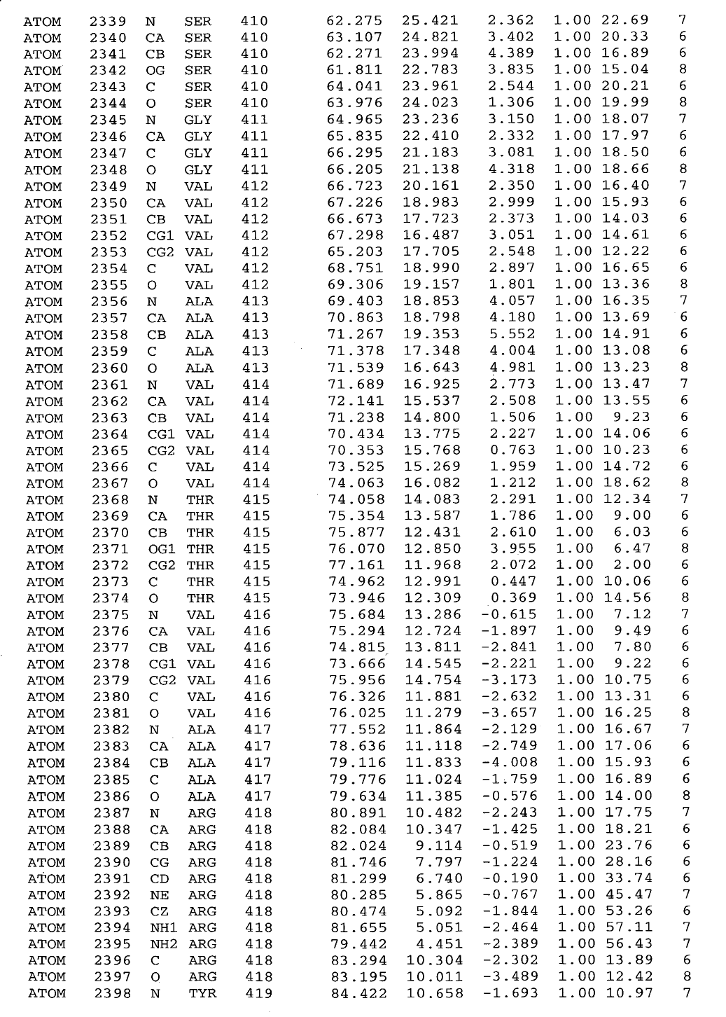

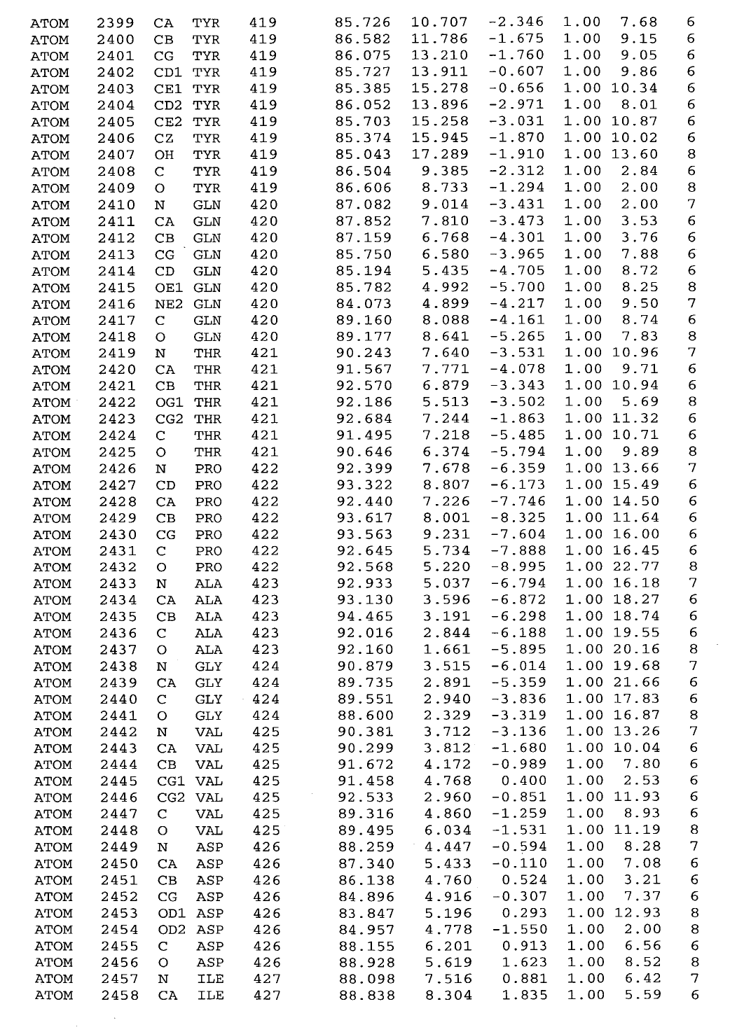

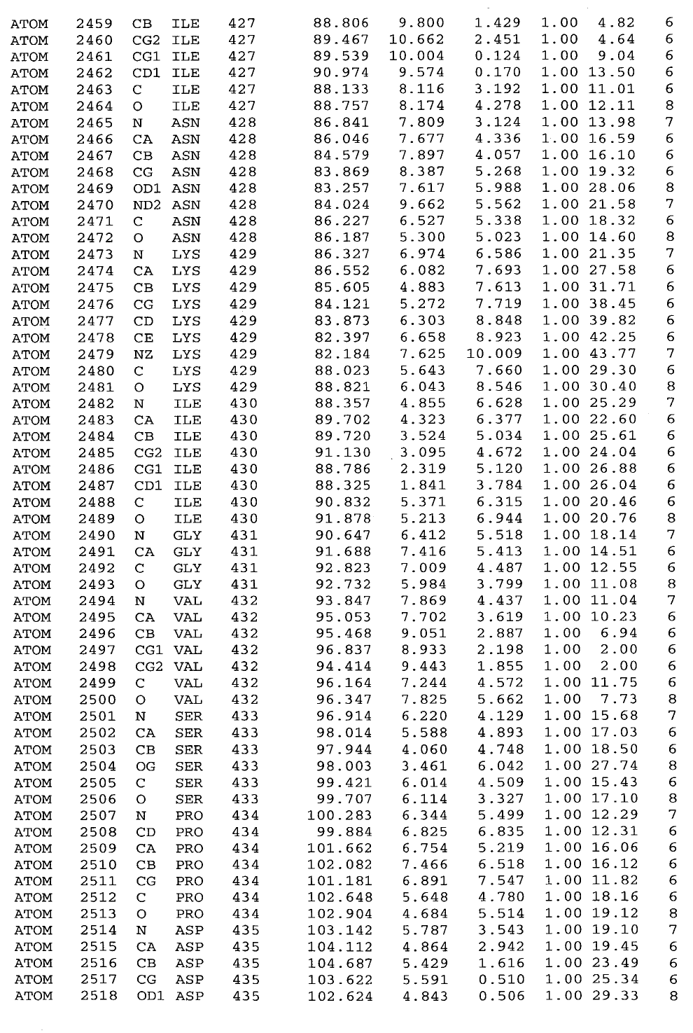

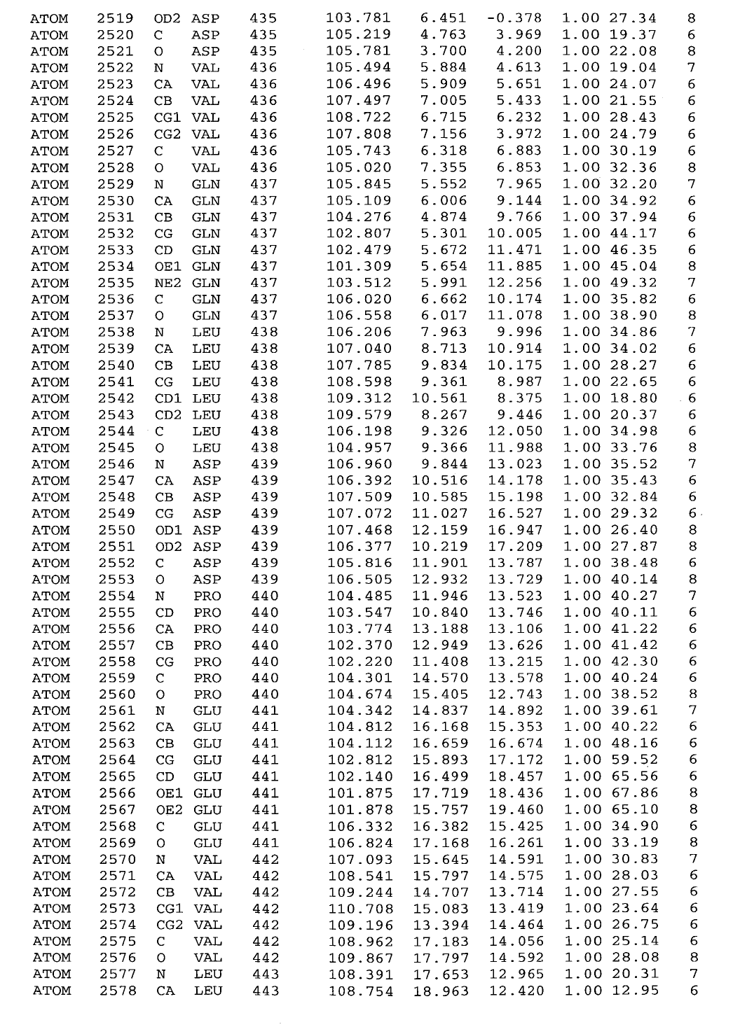

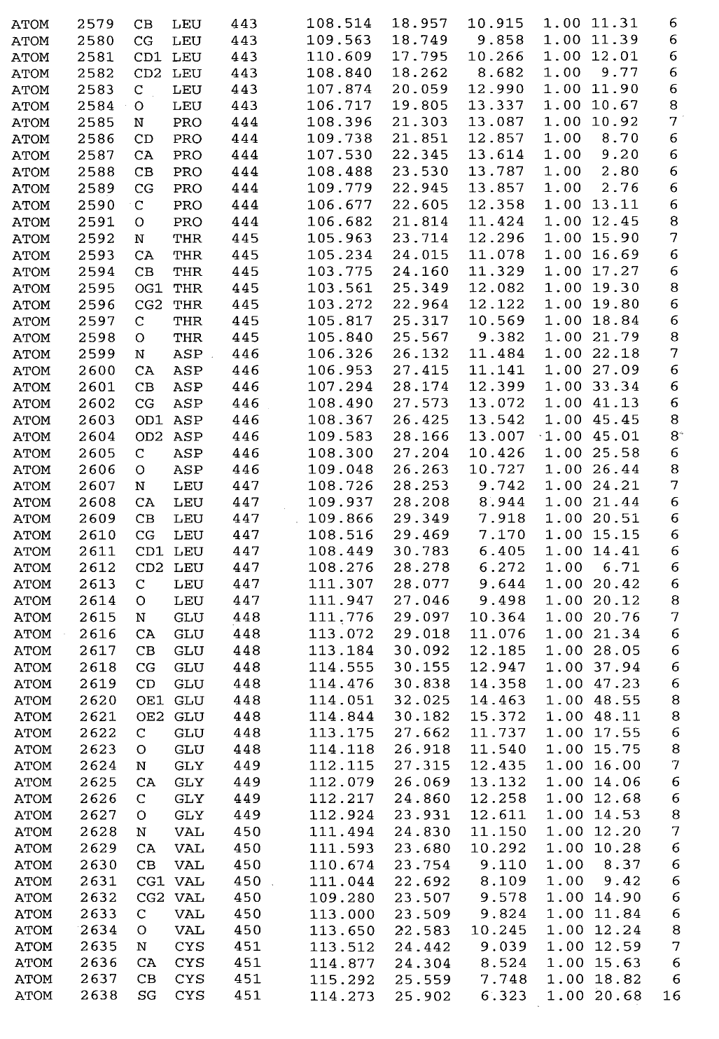

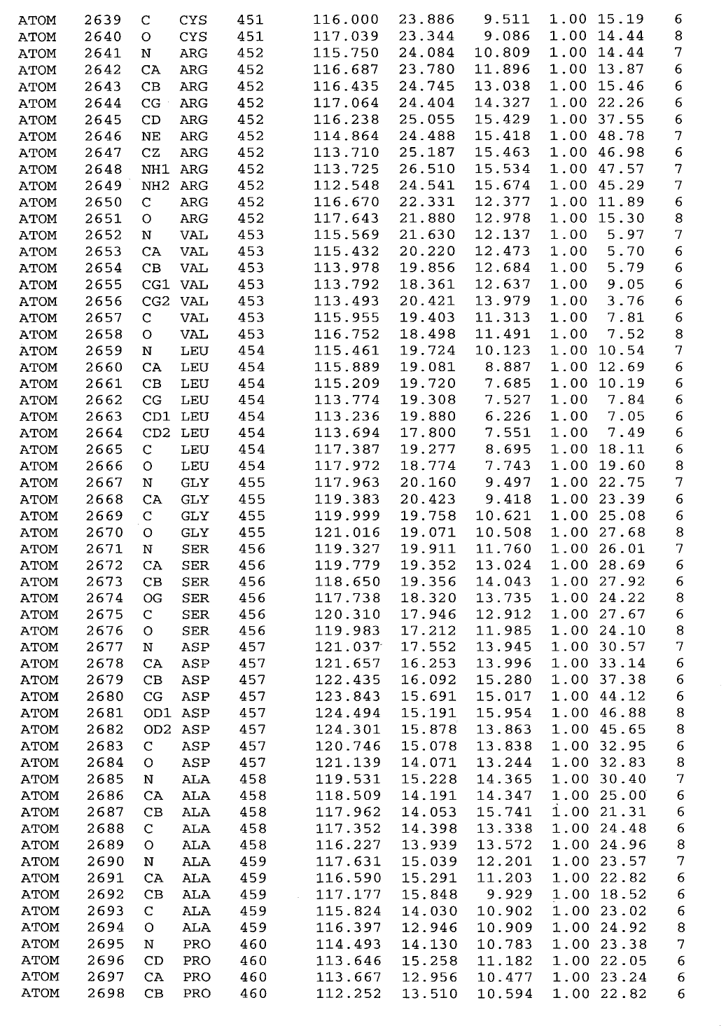

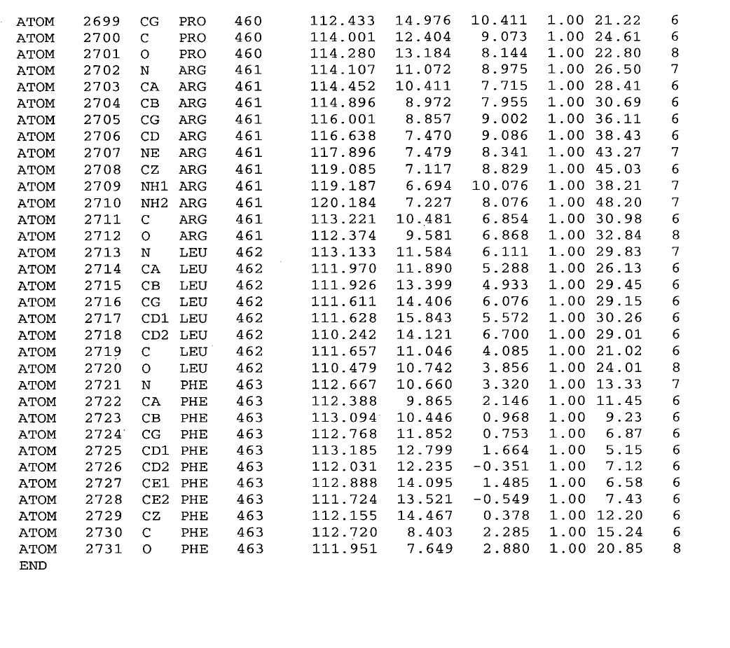

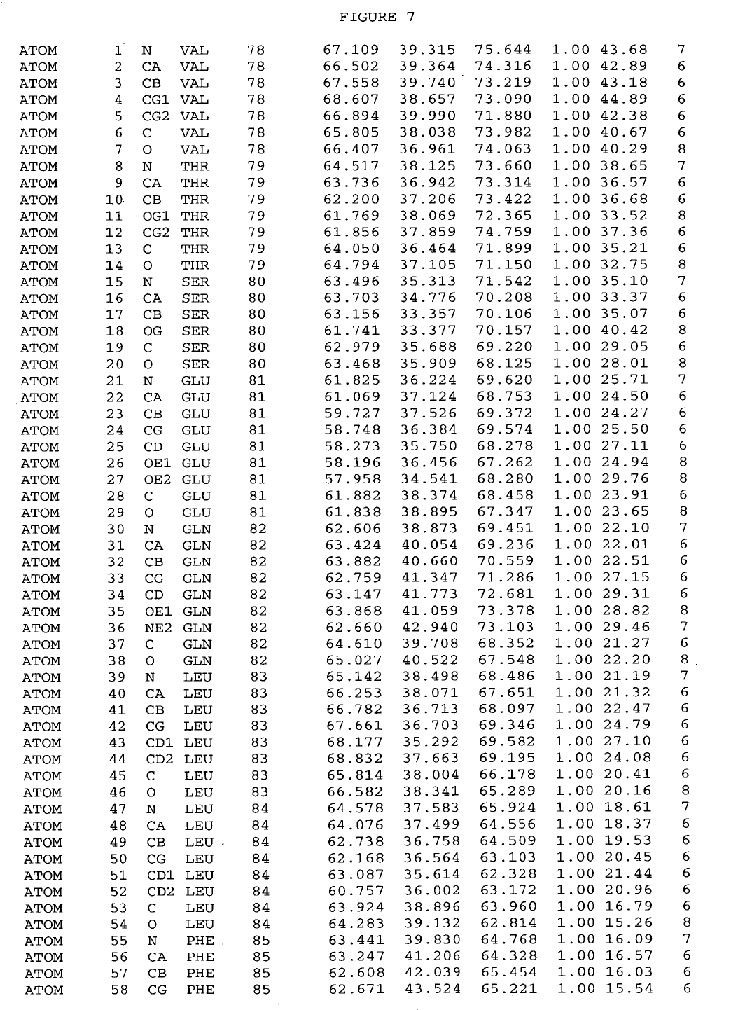

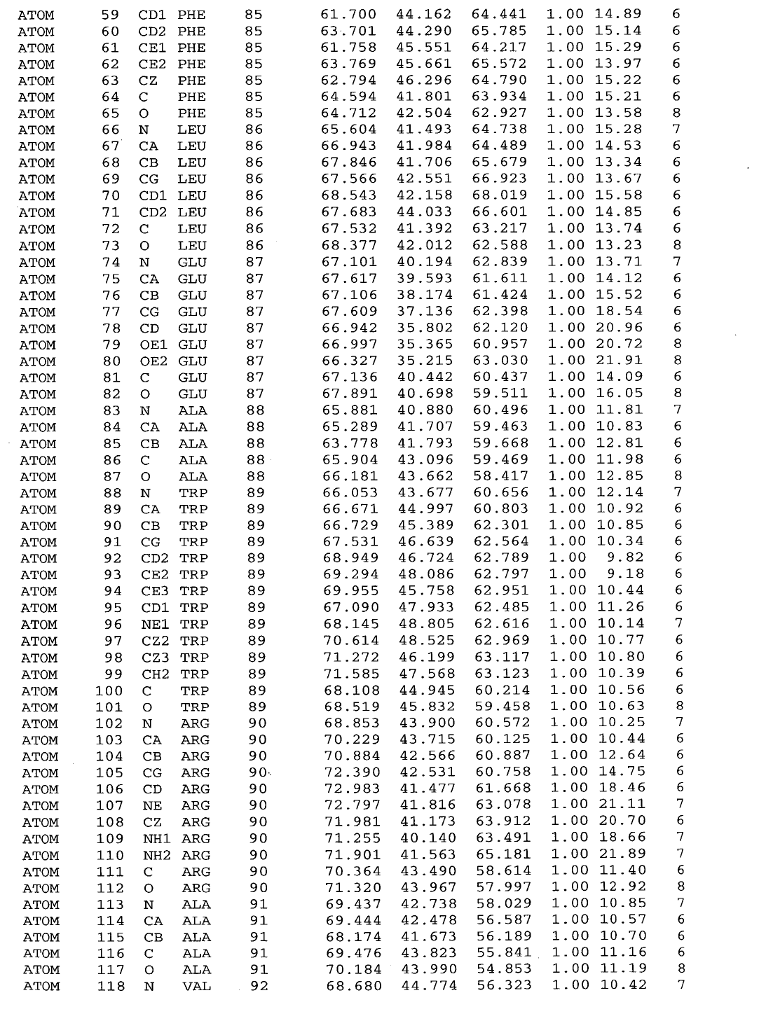

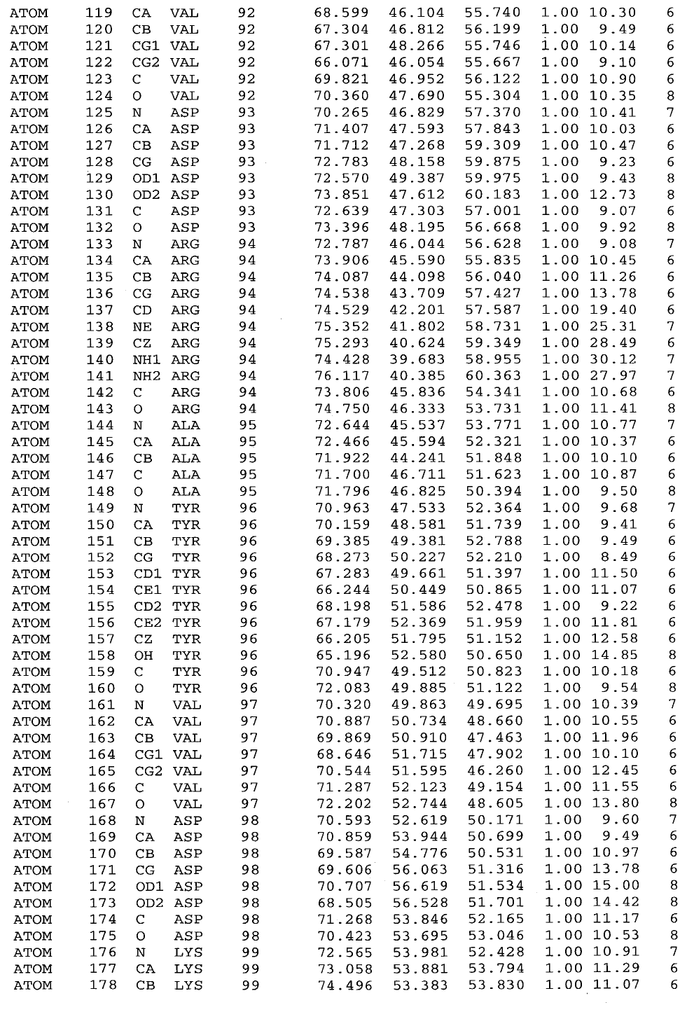

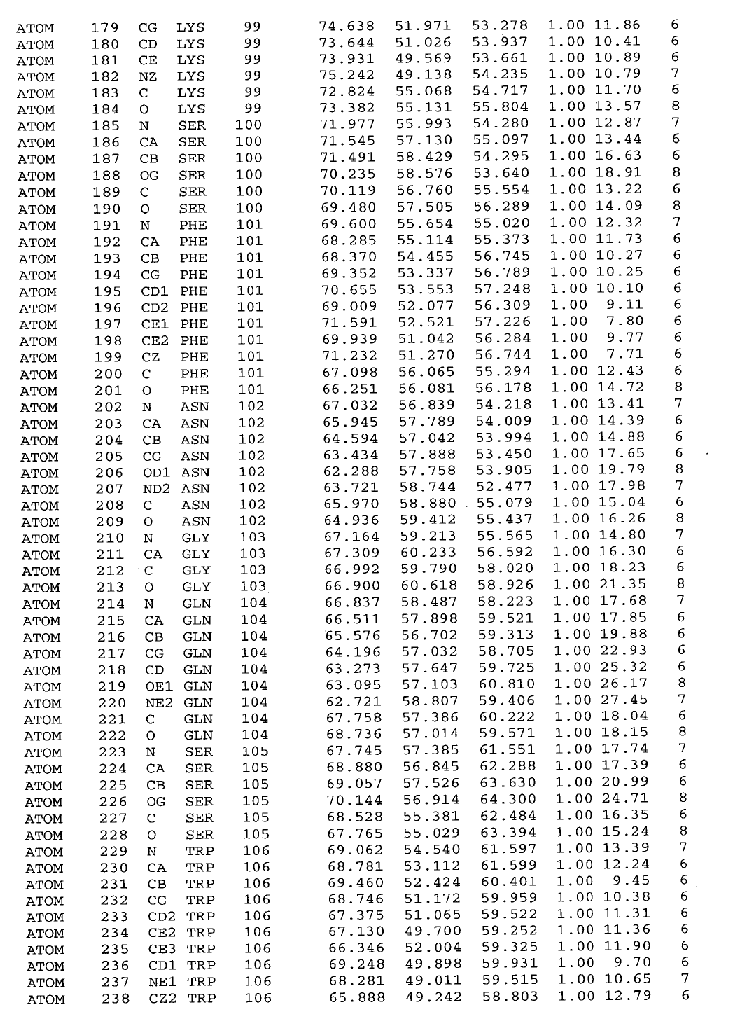

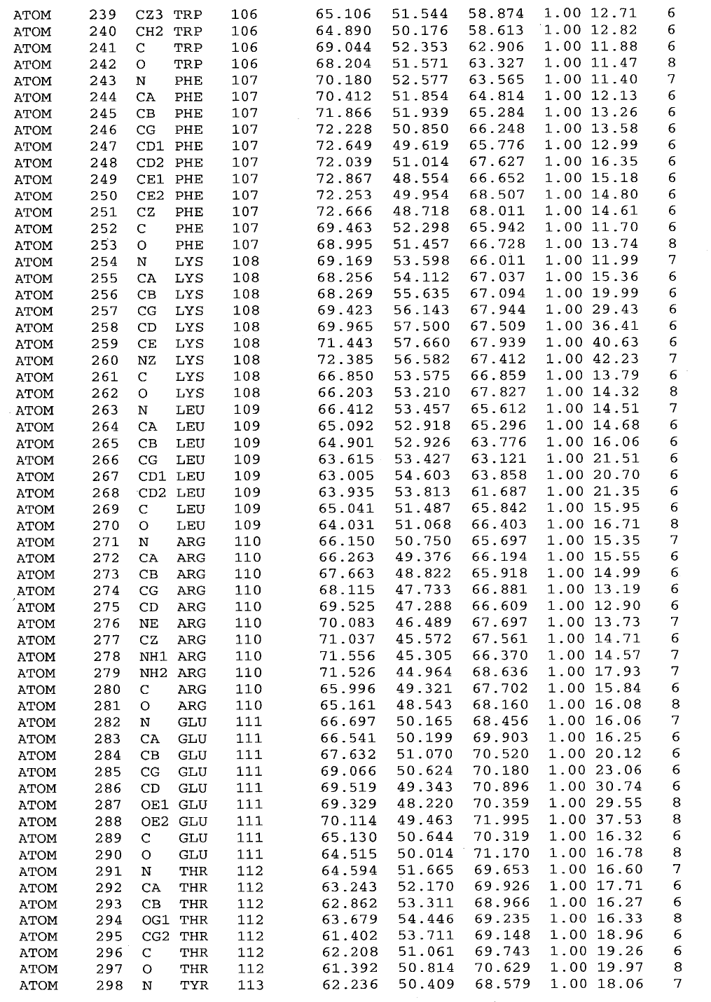

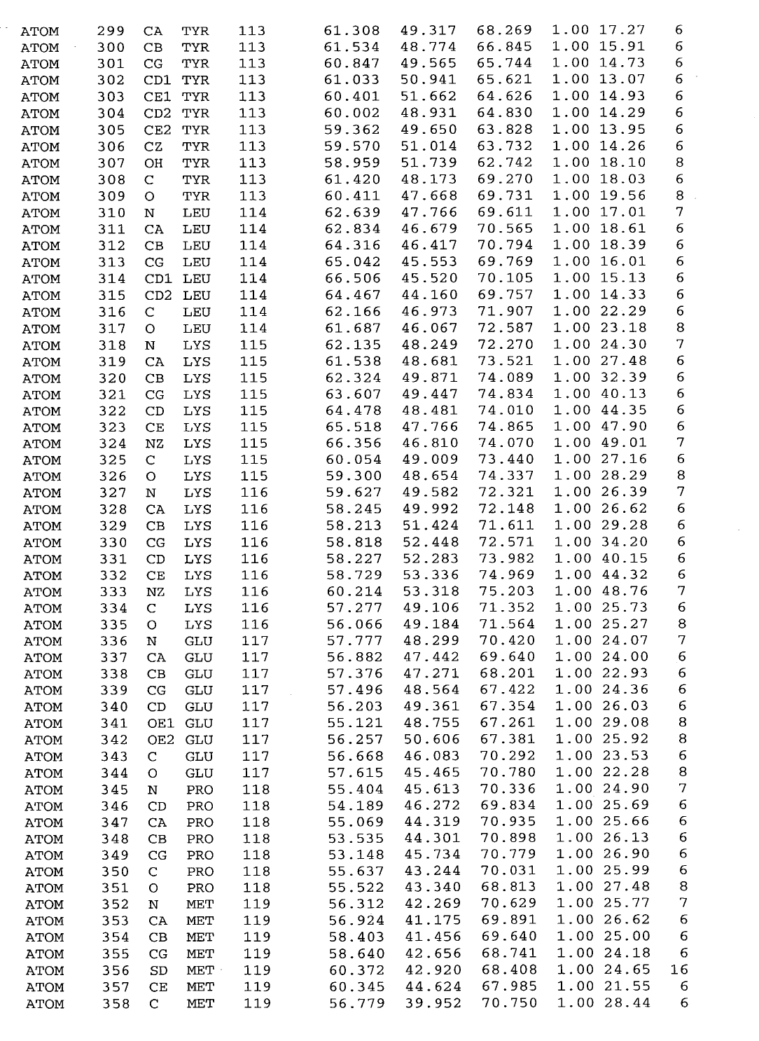

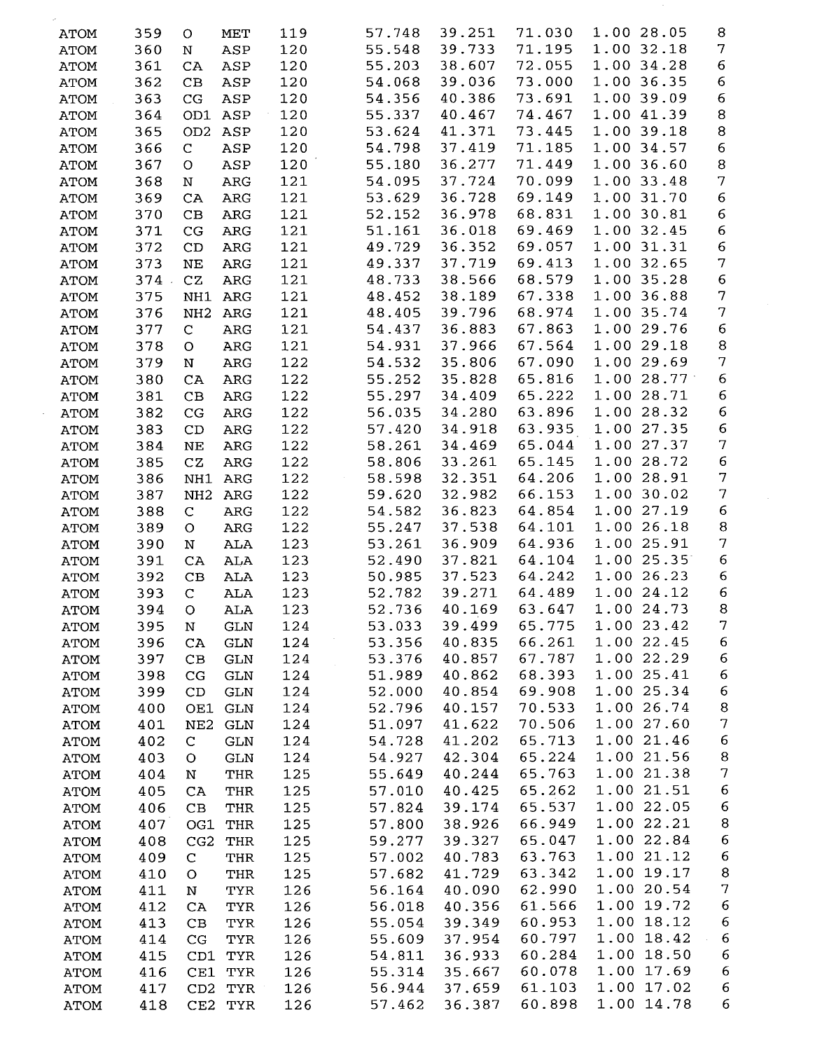

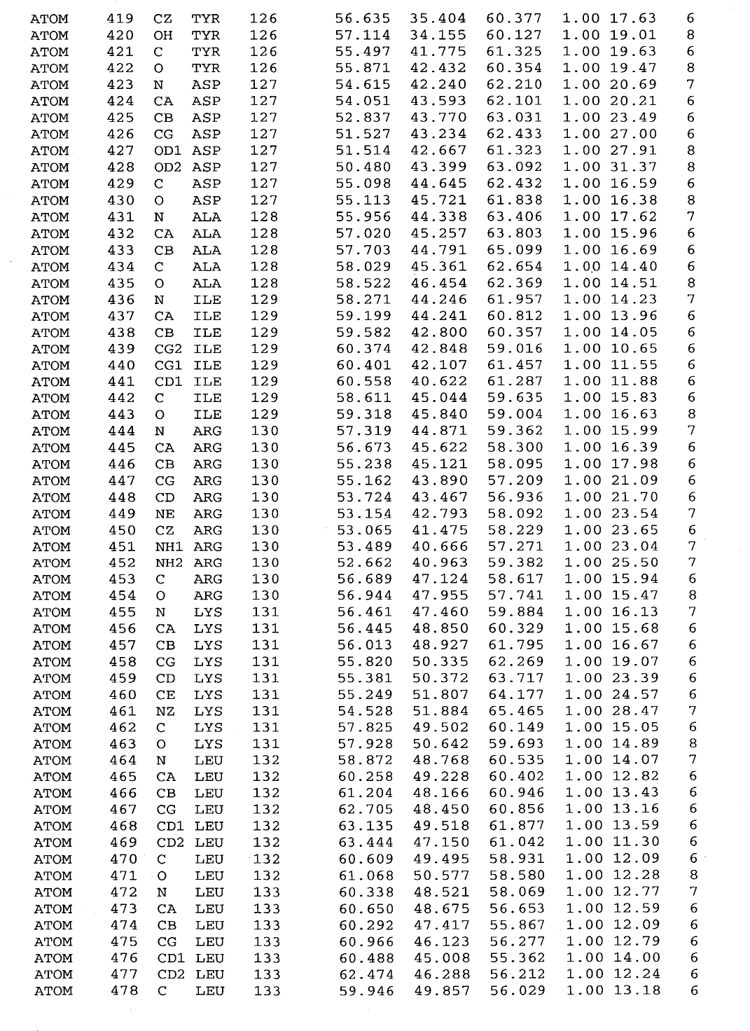

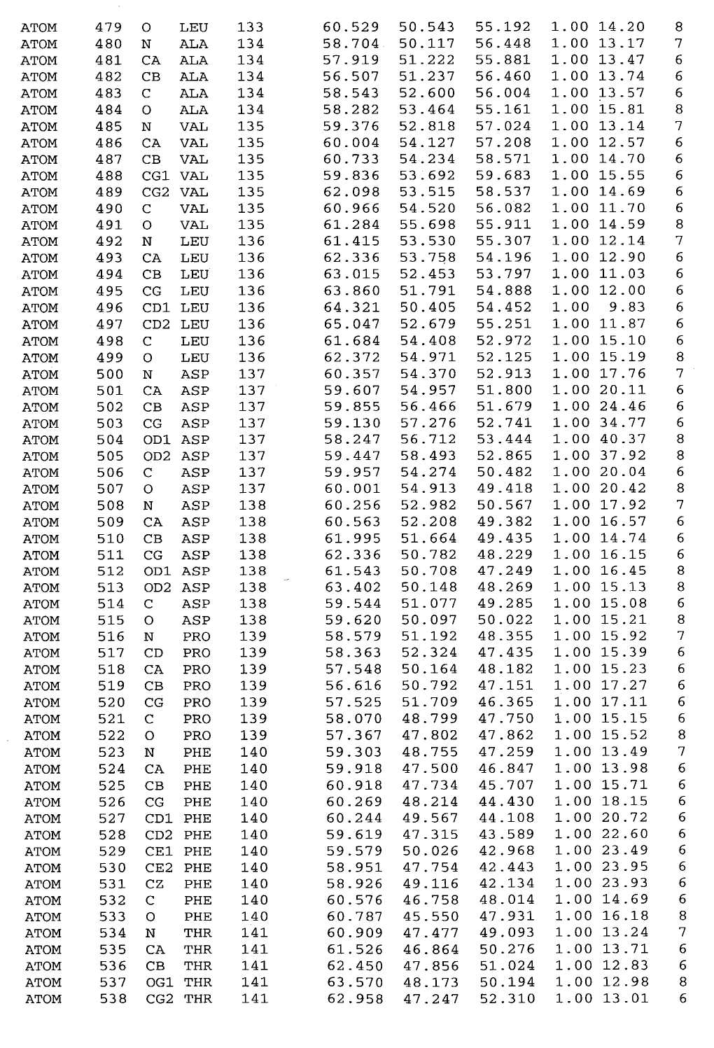

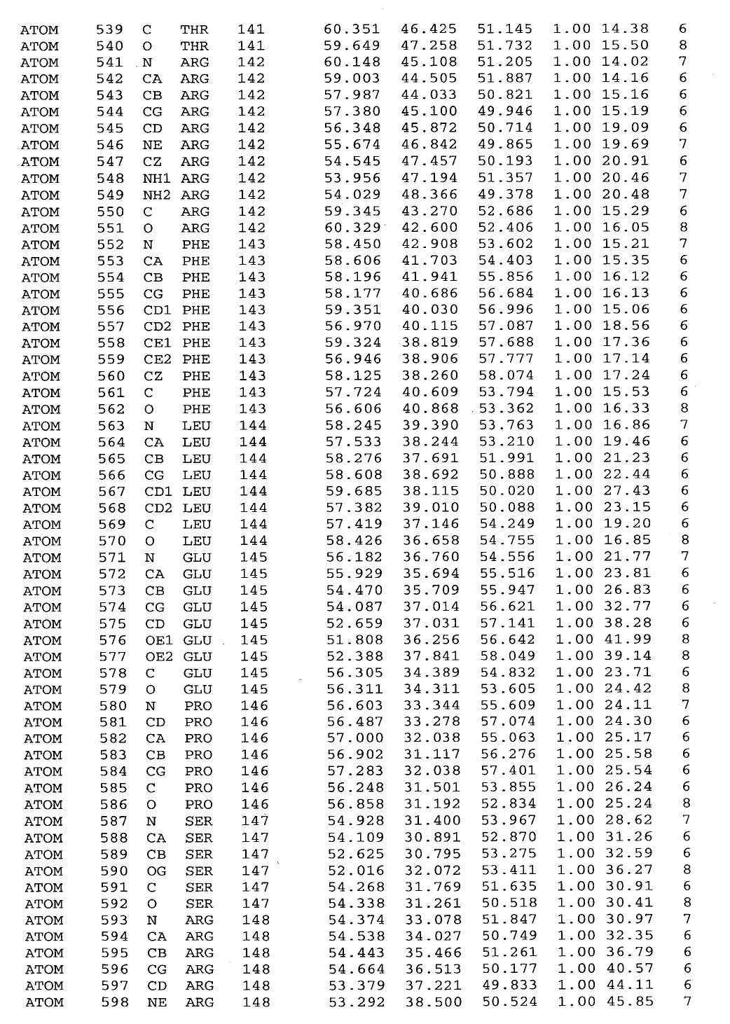

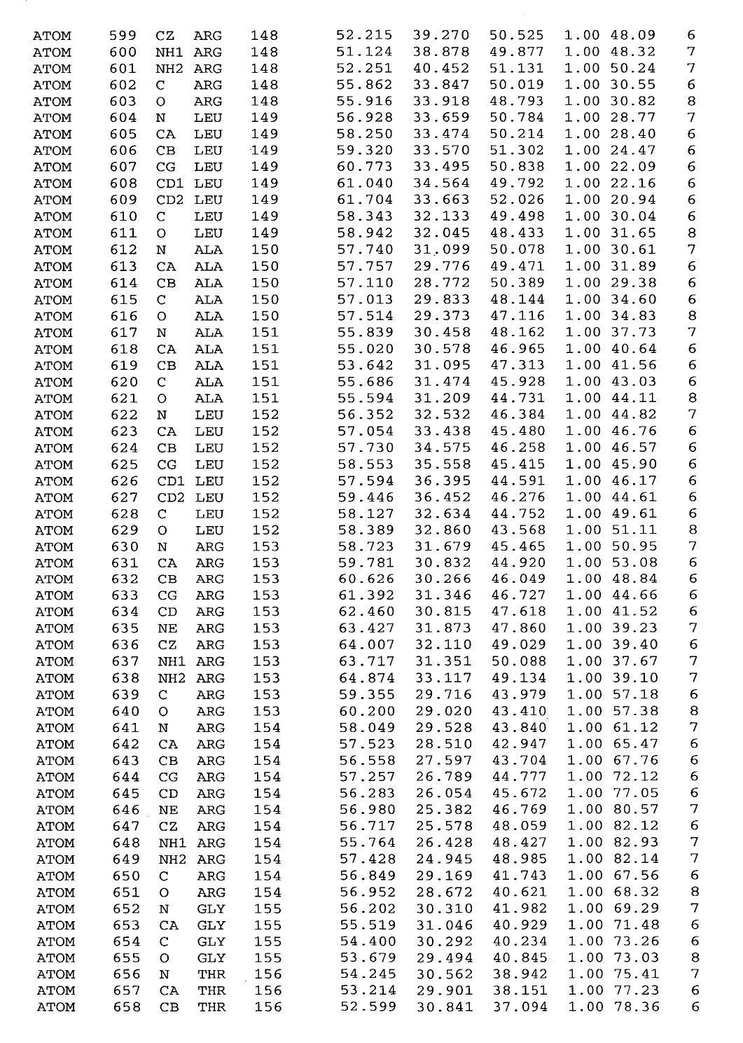

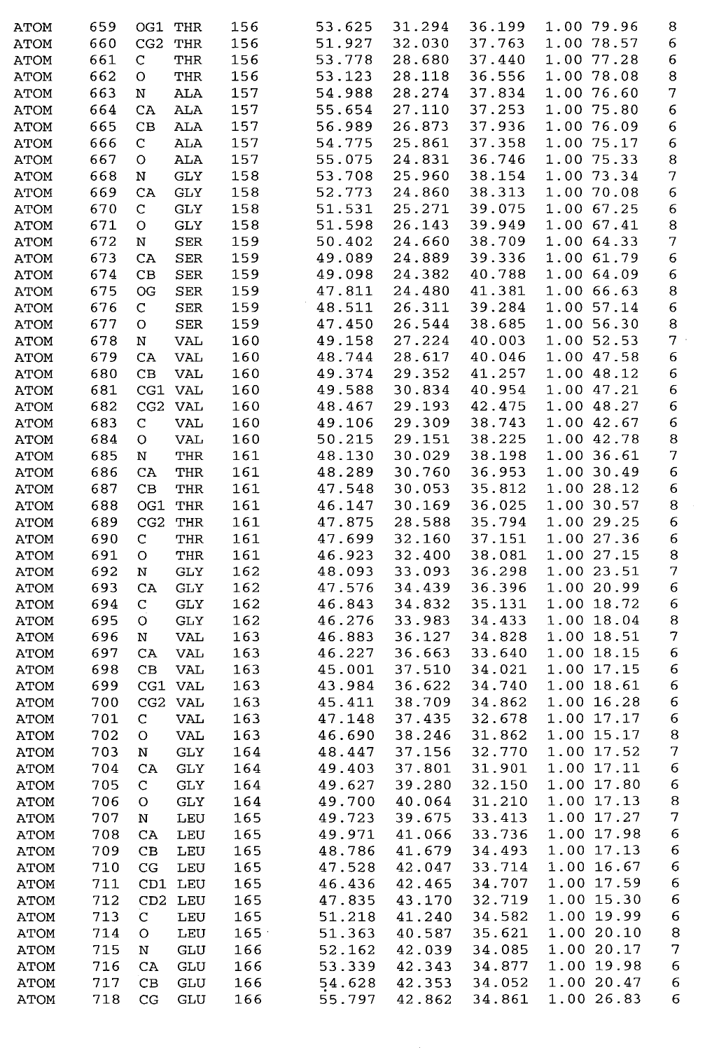

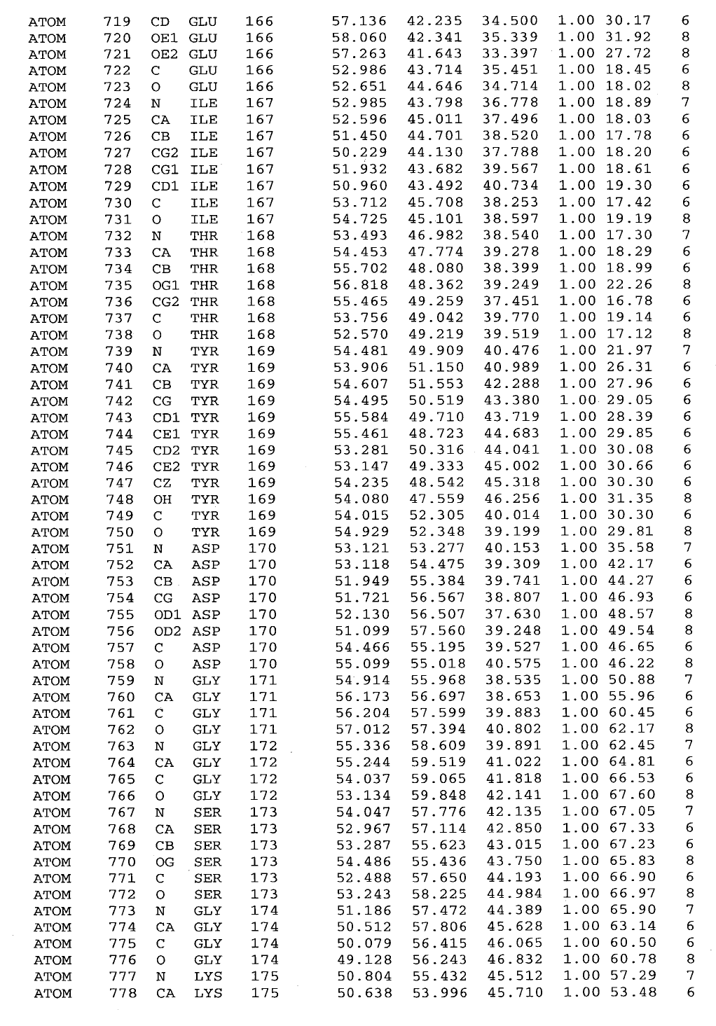

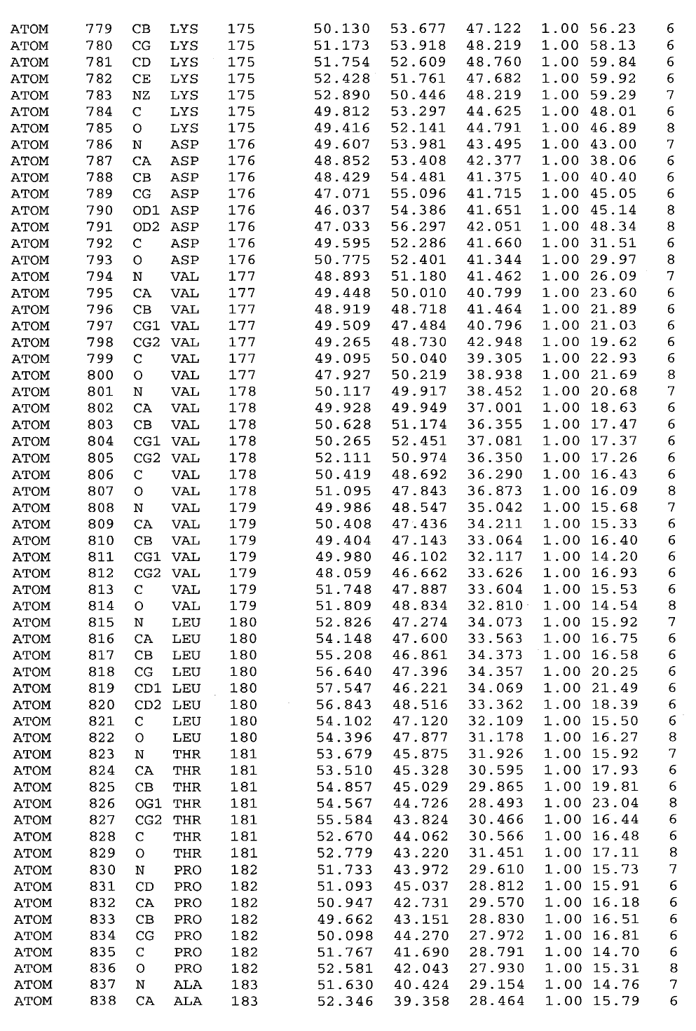

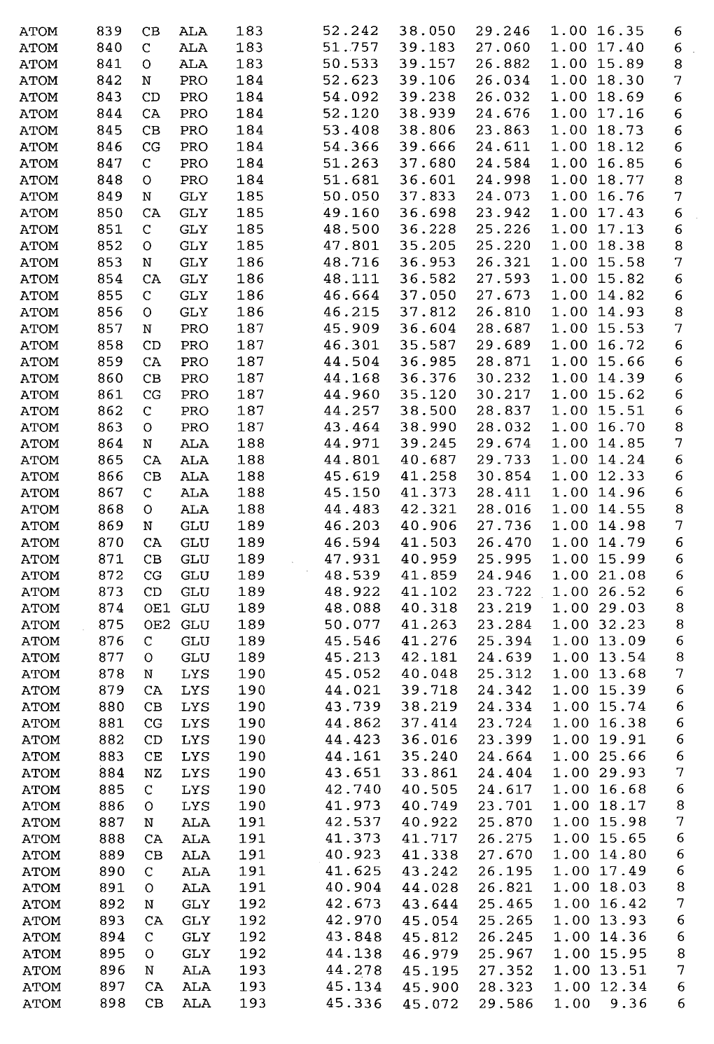

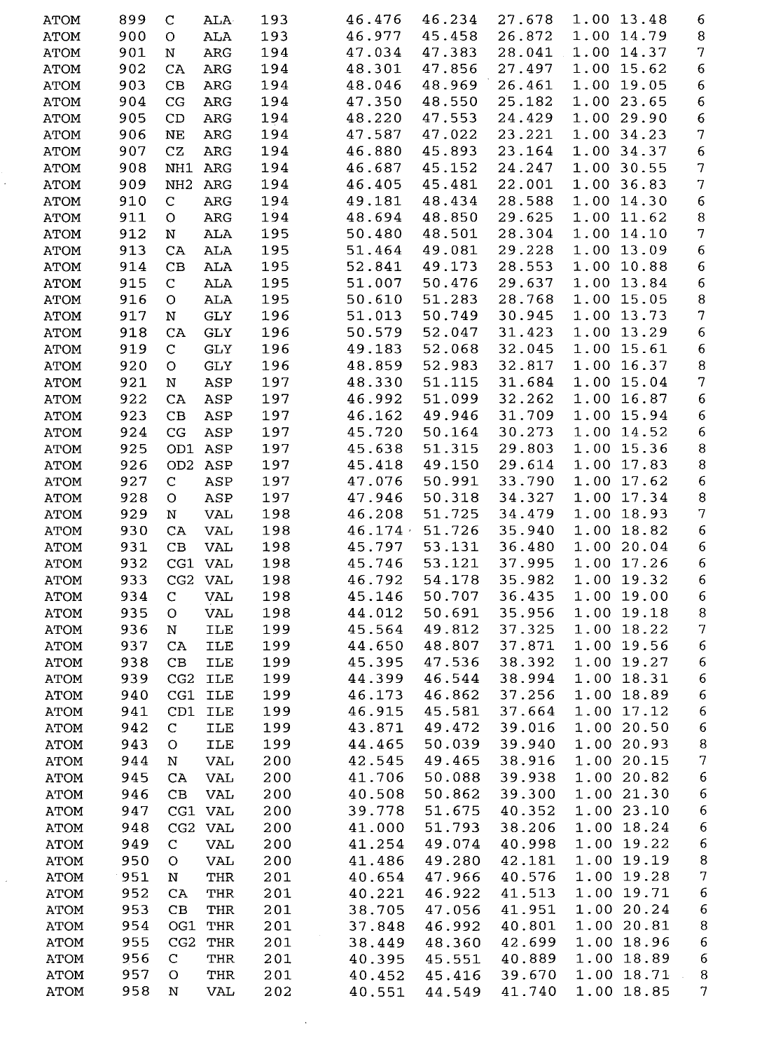

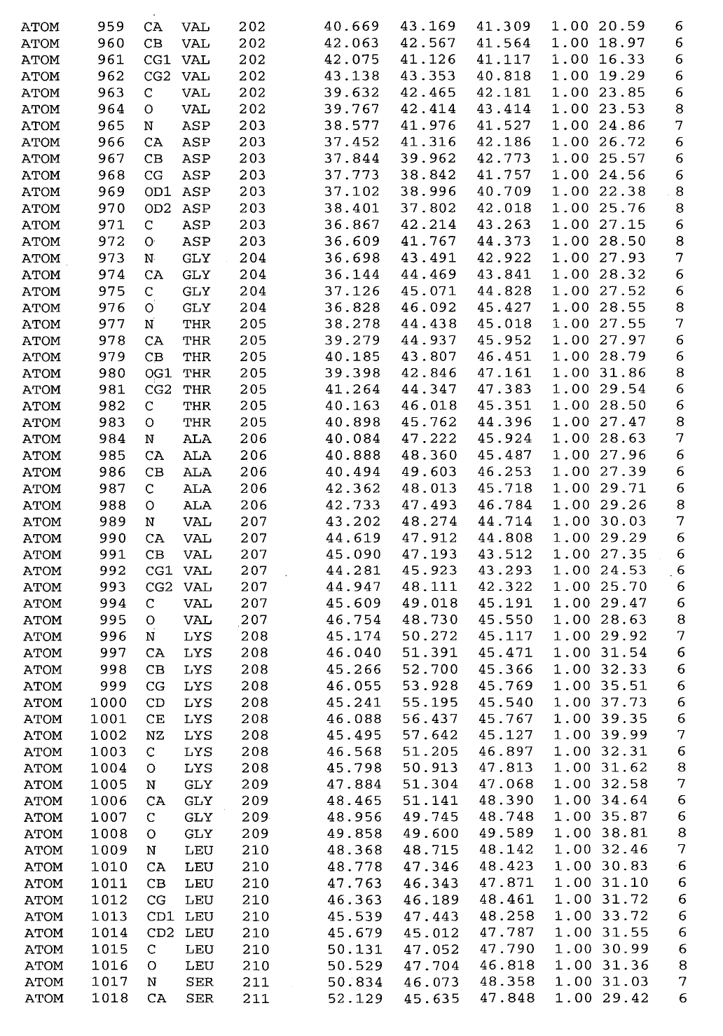

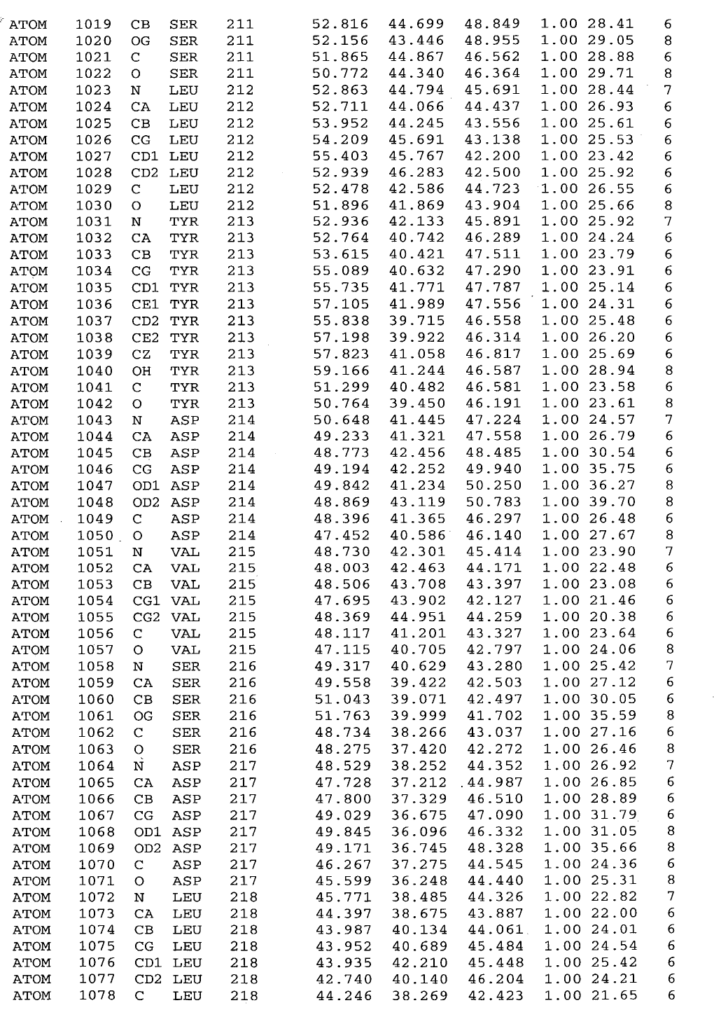

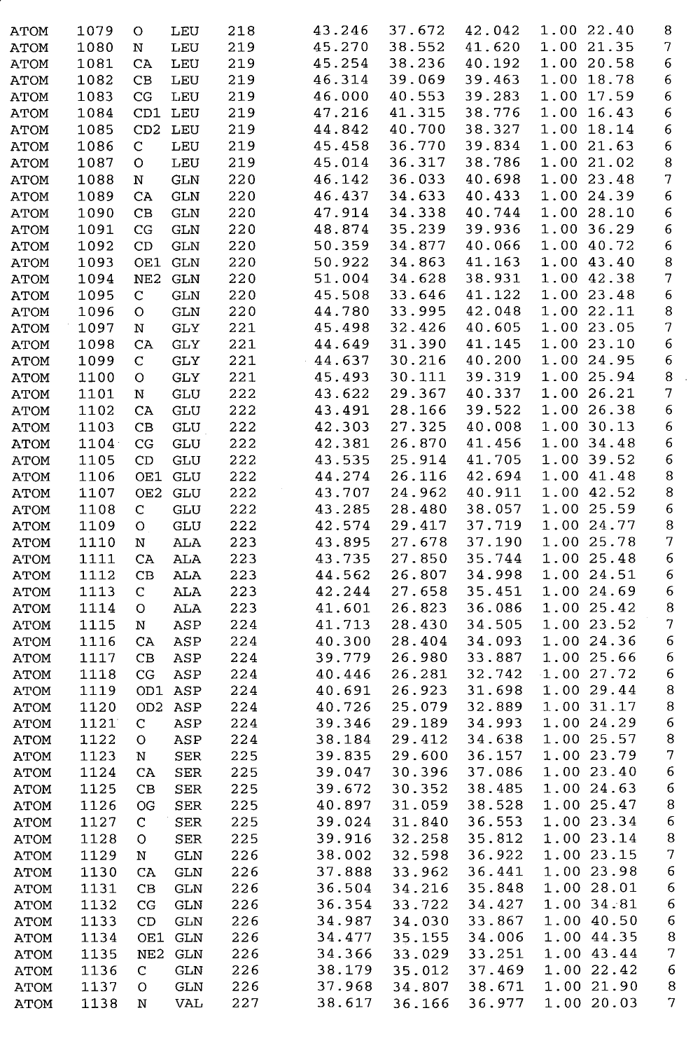

Crystals of D1 protease covalently modified by a peptide chloromethylketone with the sequence Leu-Asp-Leu-Ala, which mimics the P site of the substrate, have been obtained by hanging drop experiments as described in Example 8. In this case, the well solution consists of 20% (w/v) PEG 3000, 0.1 M Tris buffer at pH 7.0. The crystal form is similar to the C2I form with the cell dimension of a=111.8 Å, b=64.1 Å, c=63.2 Å and β=122.2°. The crystals diffract x-rays to 1.6 Å resolution. The structure was determined and refined by using the C2I form inhibitor-free structure as the starting model and using the same refinement protocol described in the Example 10. The working crystallographic R-value was 20.7% and the free R-value is 27.3% for data between 10.0 1.6 Å. The refined coordinates are presented in FIG. 7.

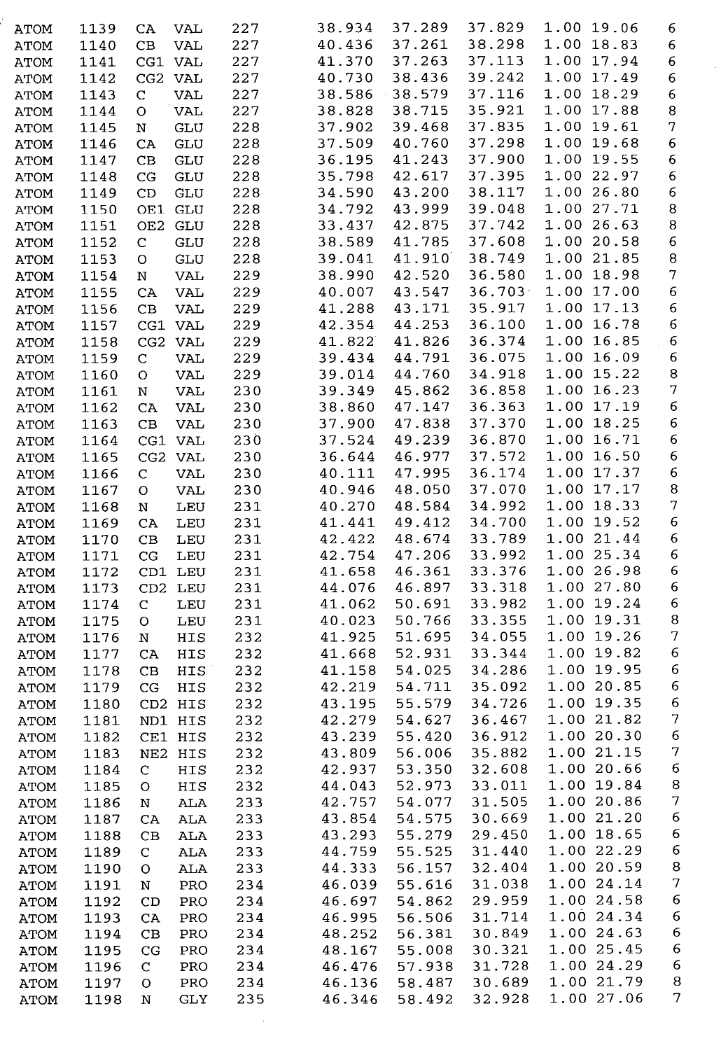

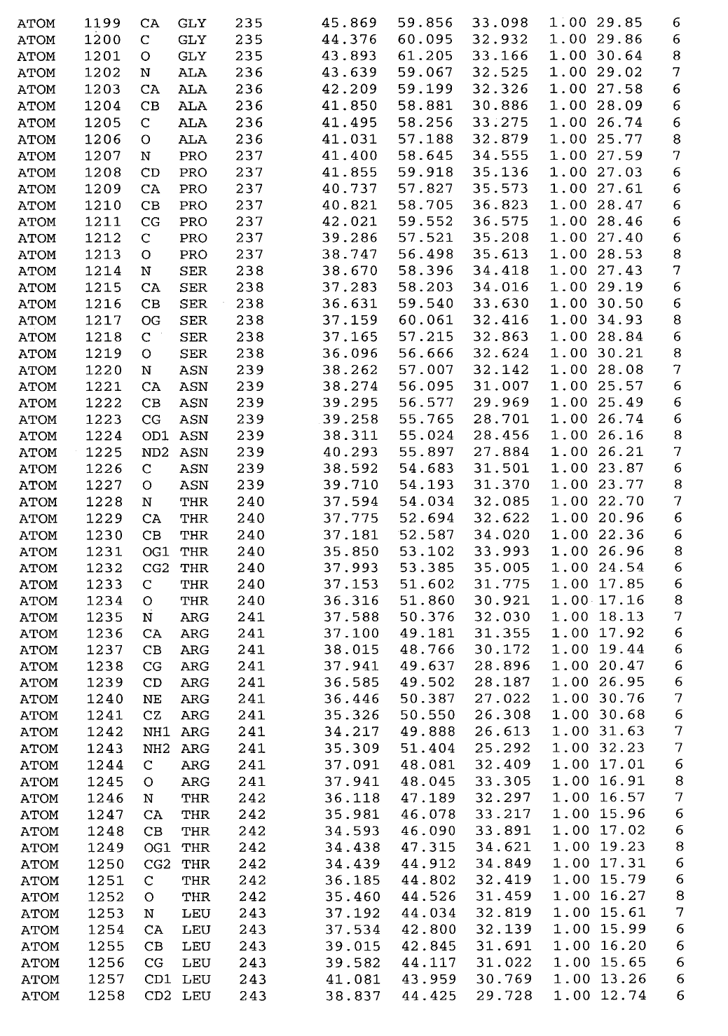

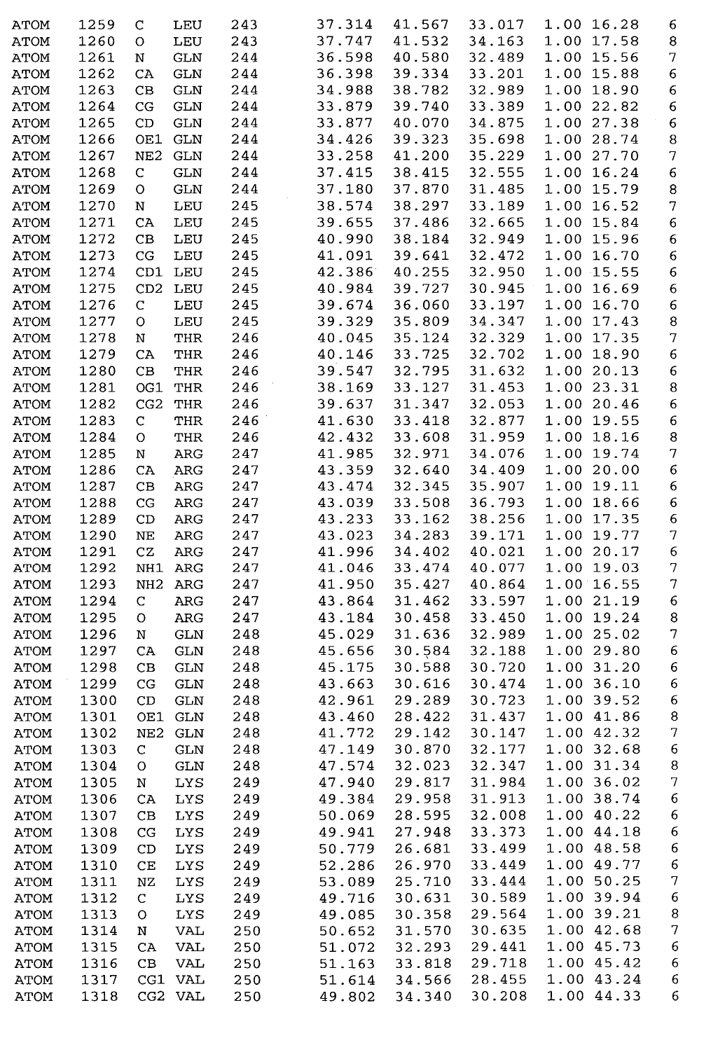

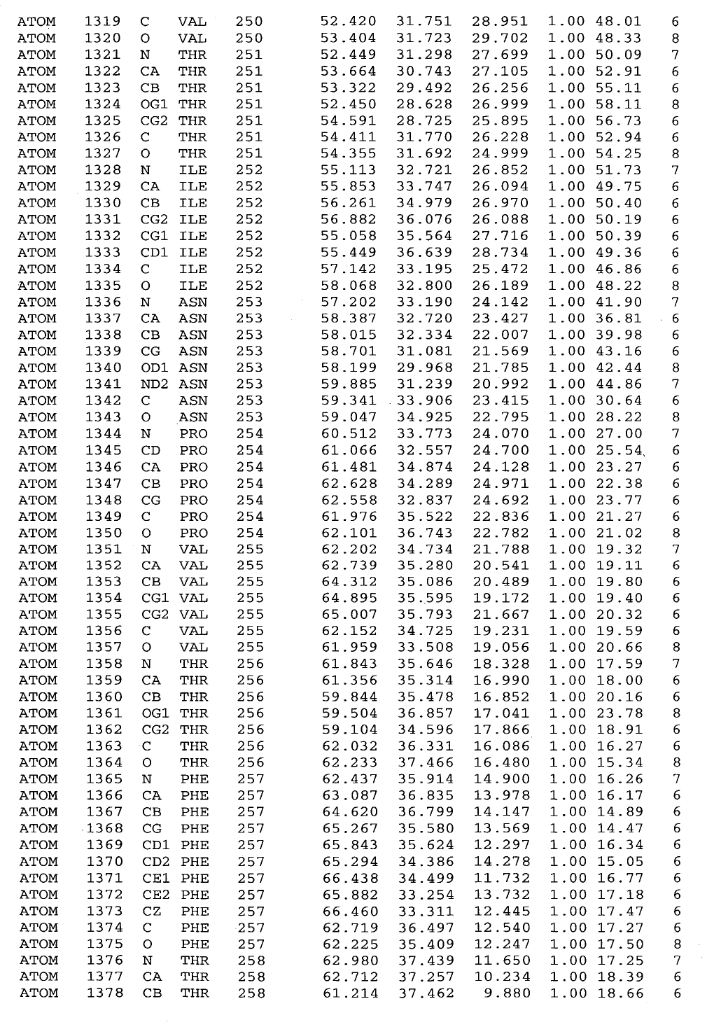

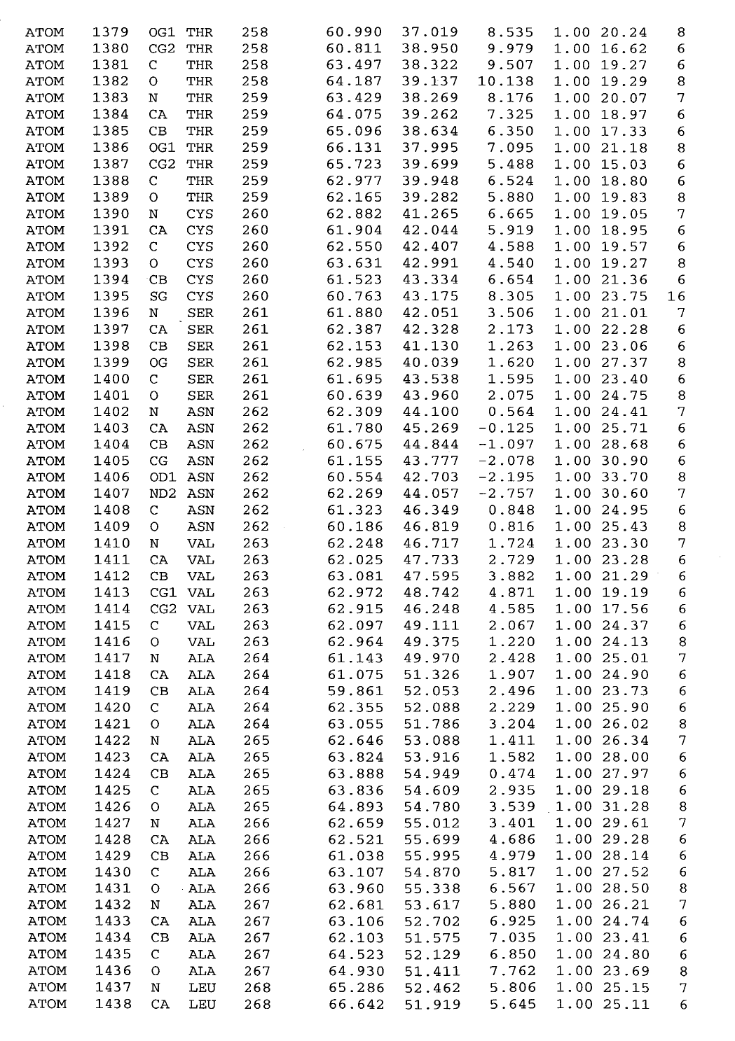

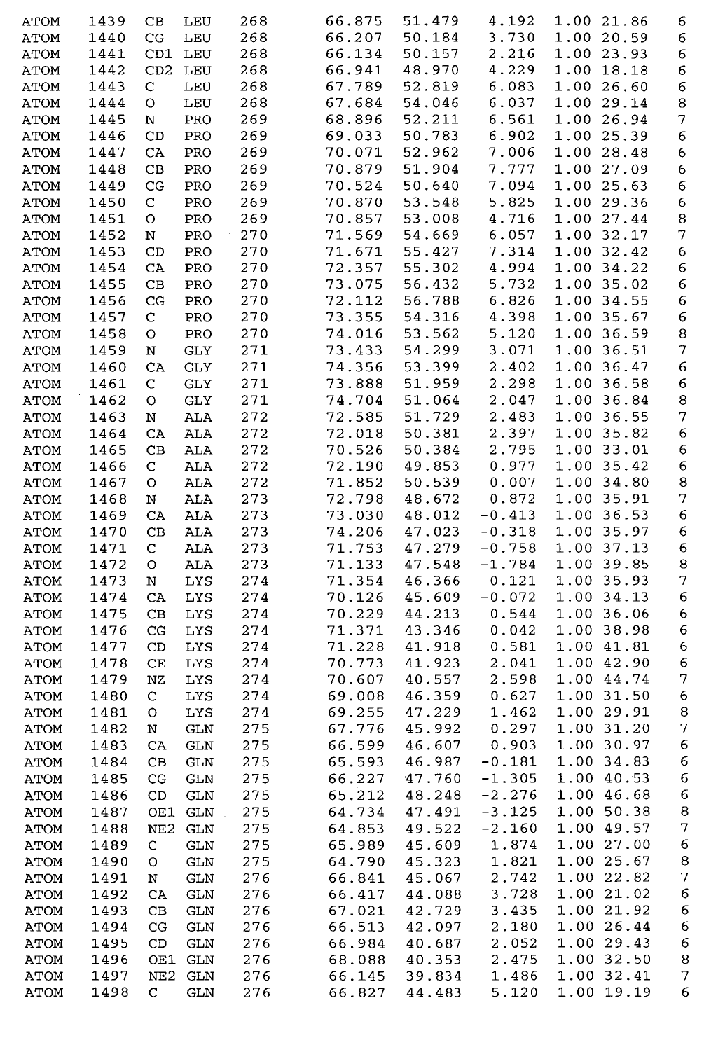

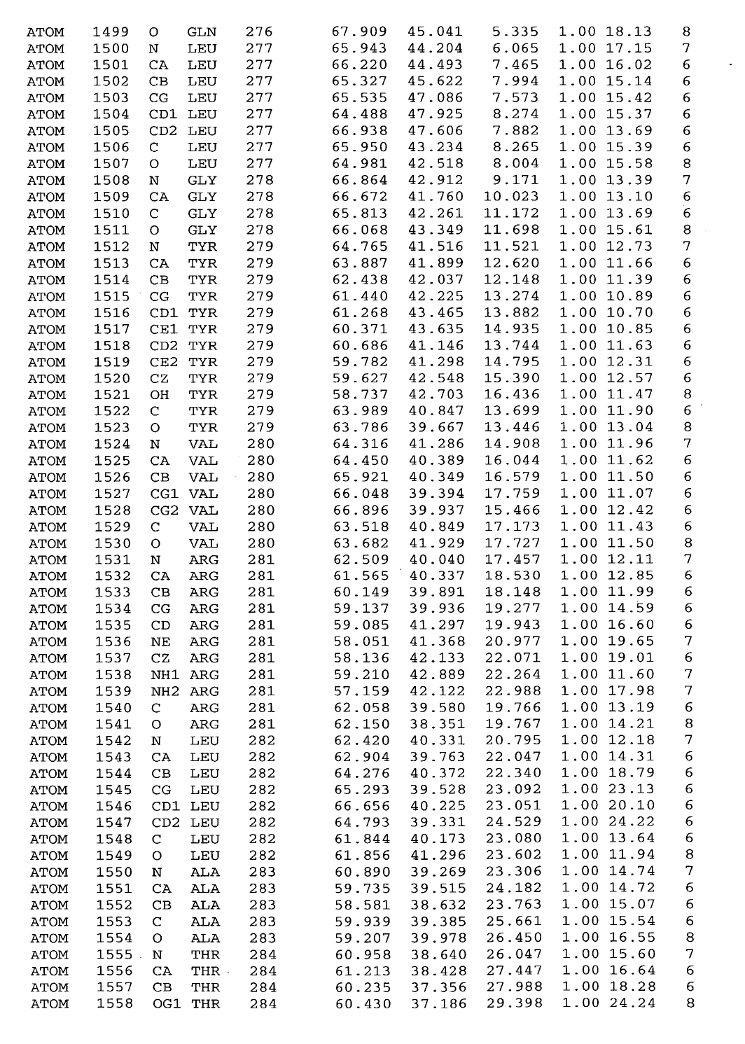

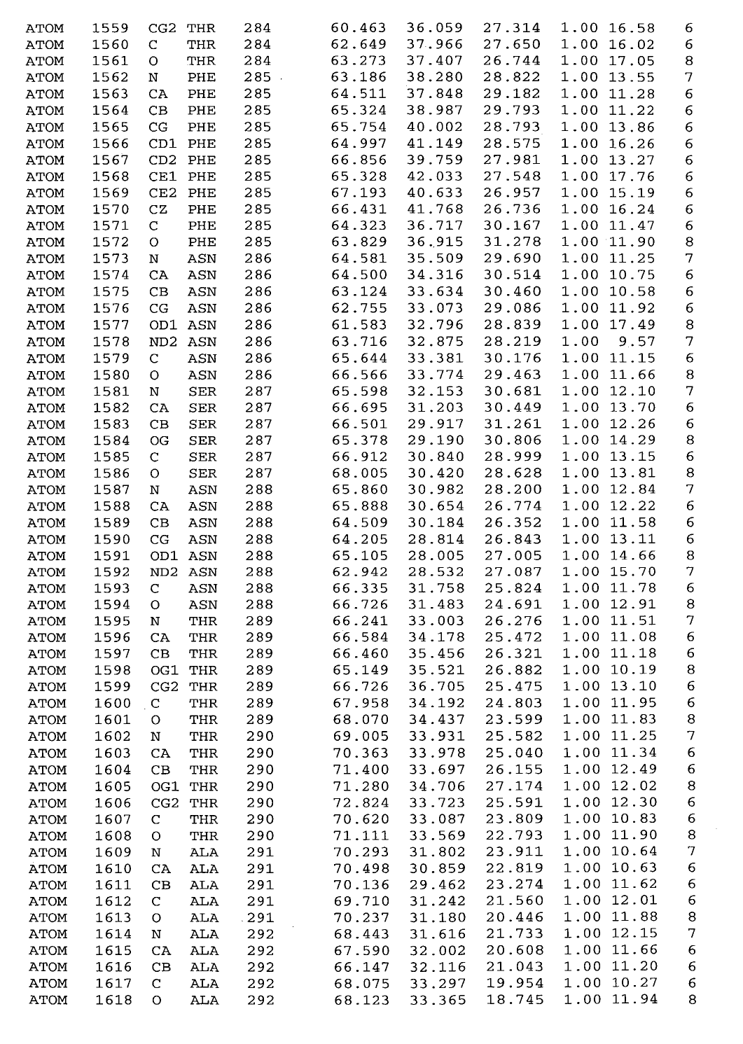

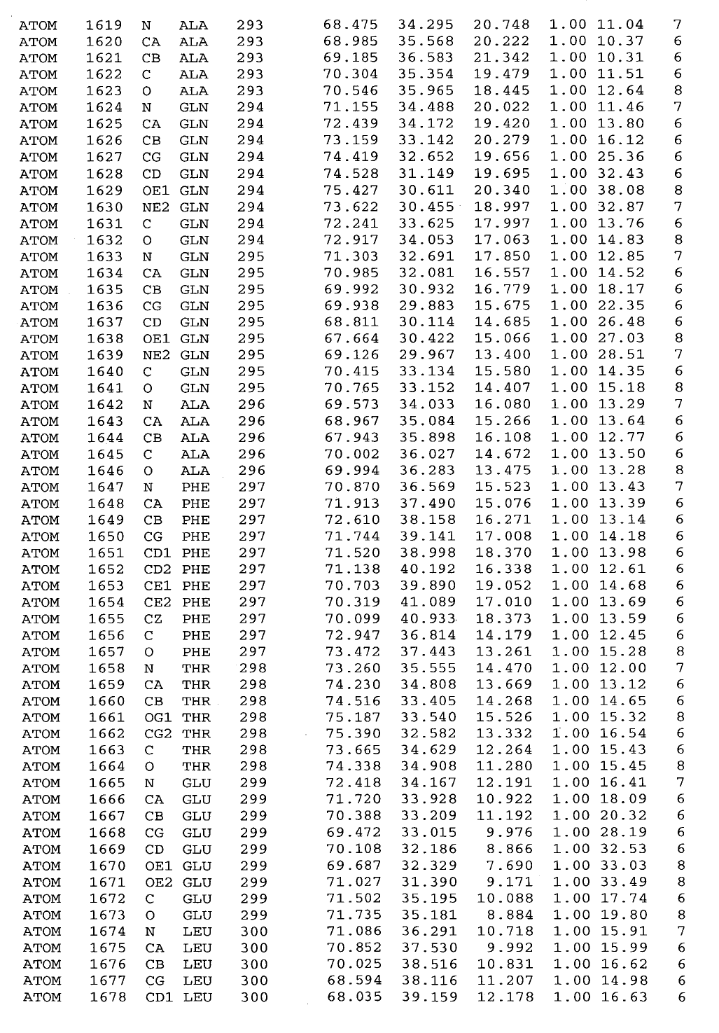

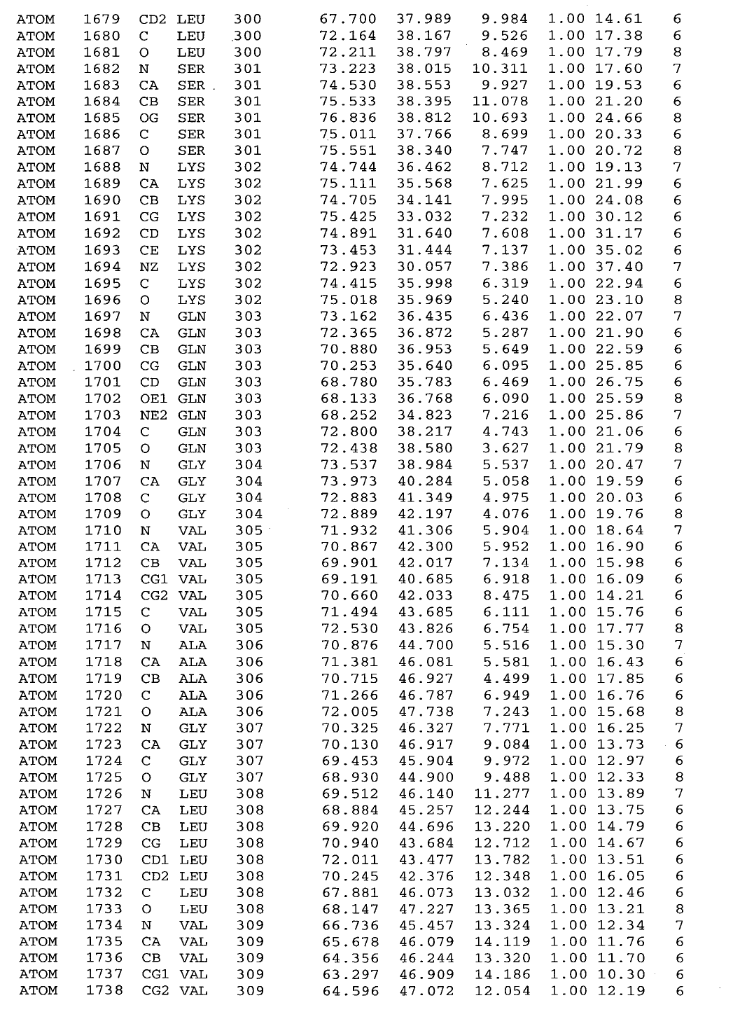

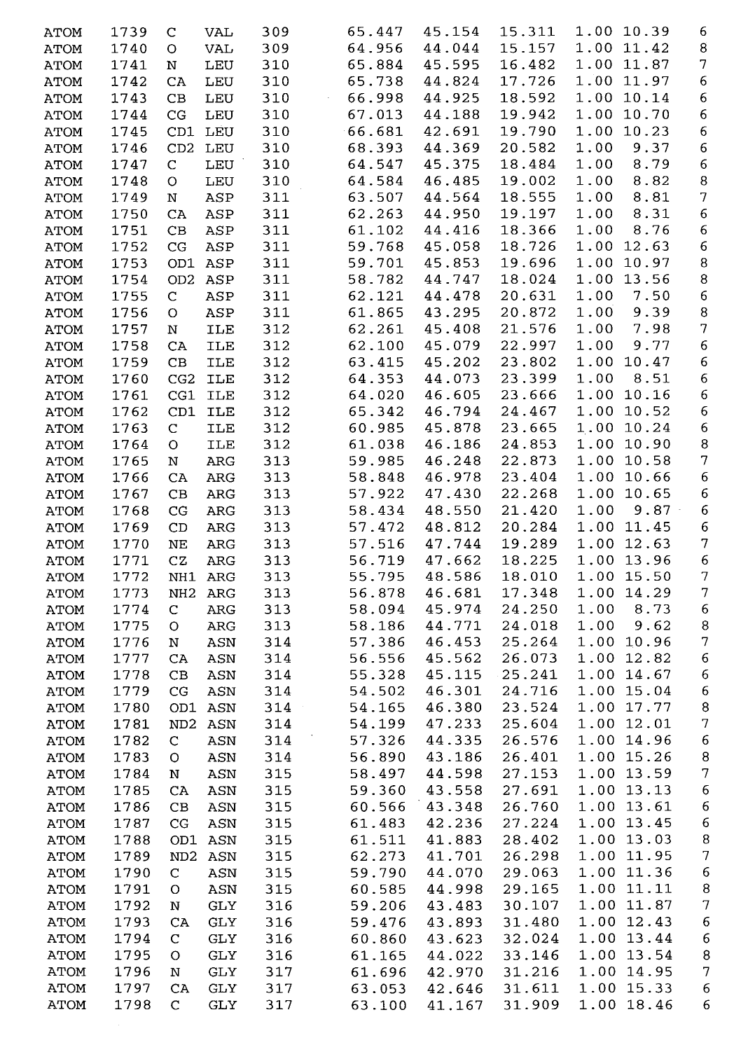

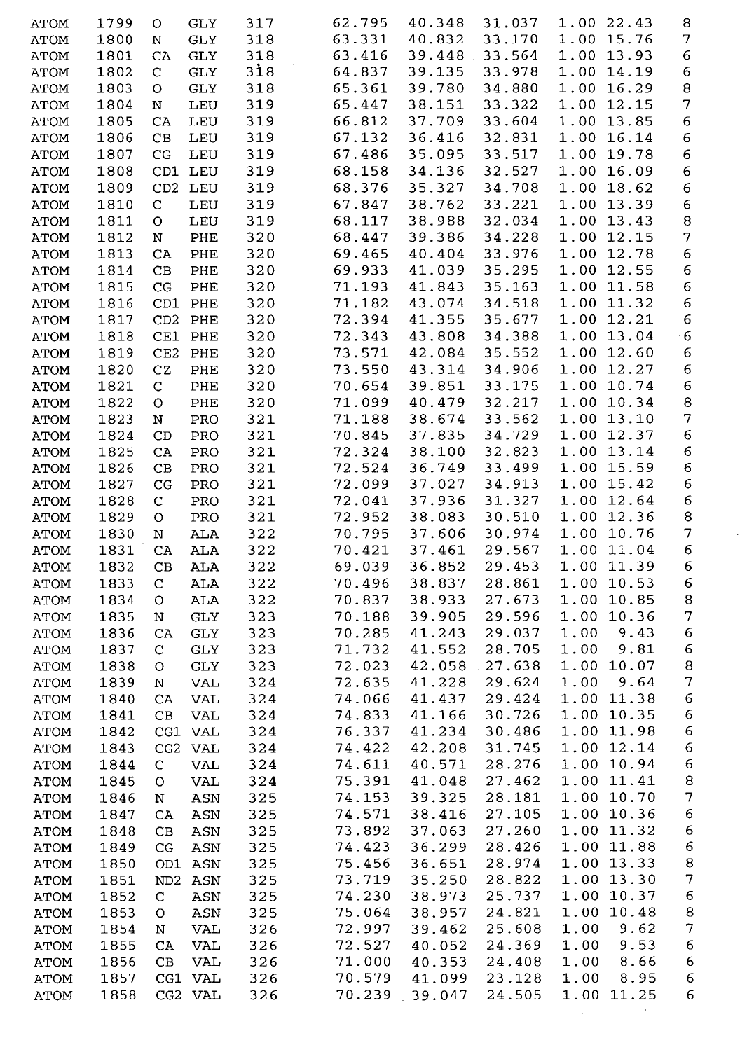

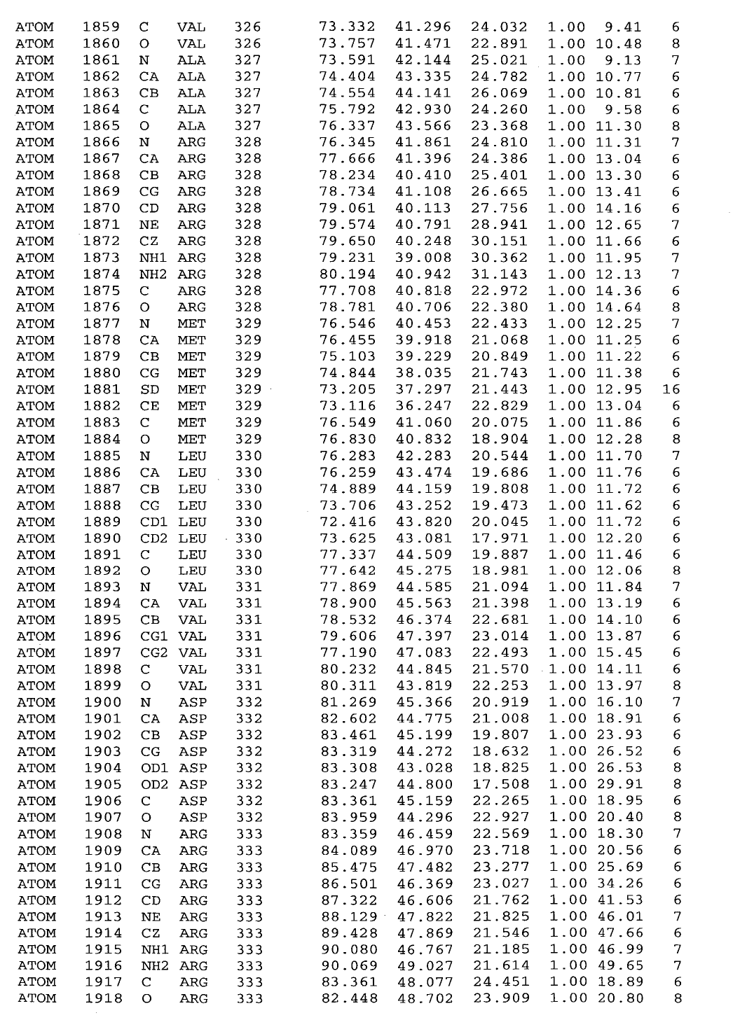

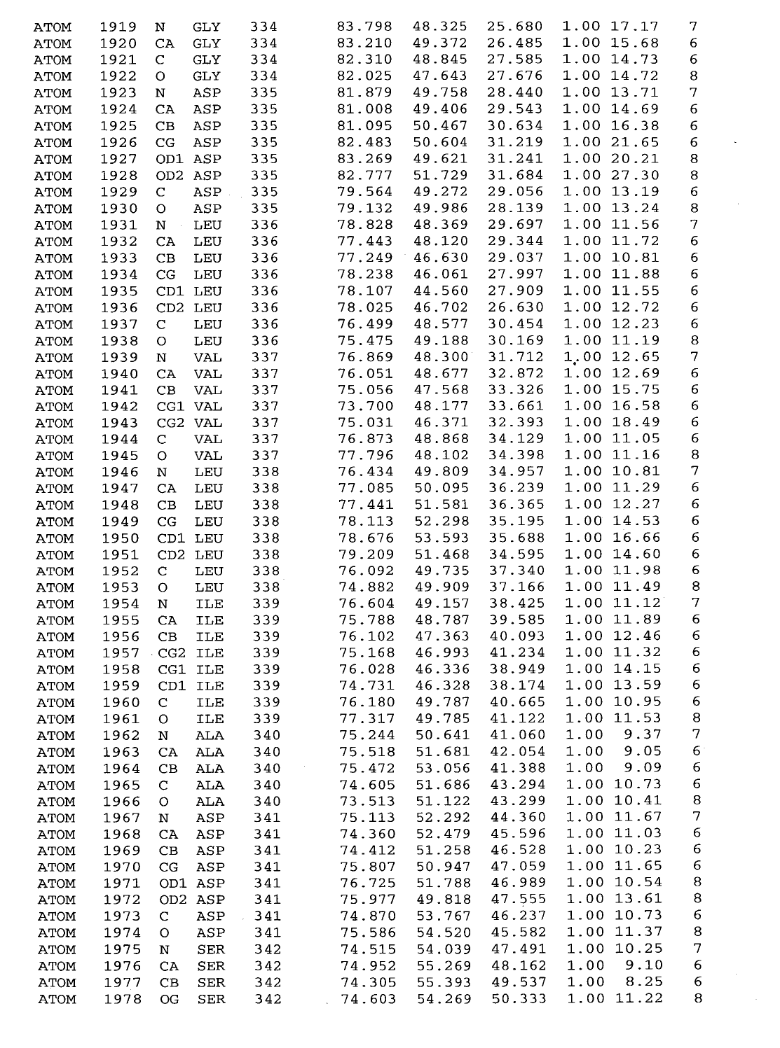

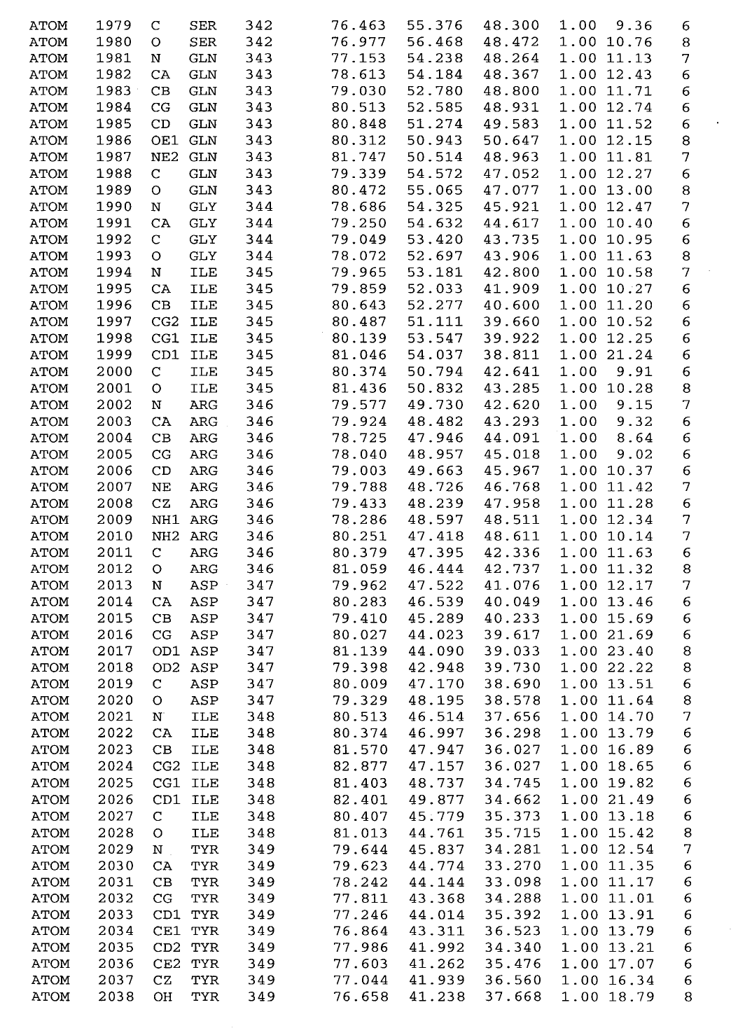

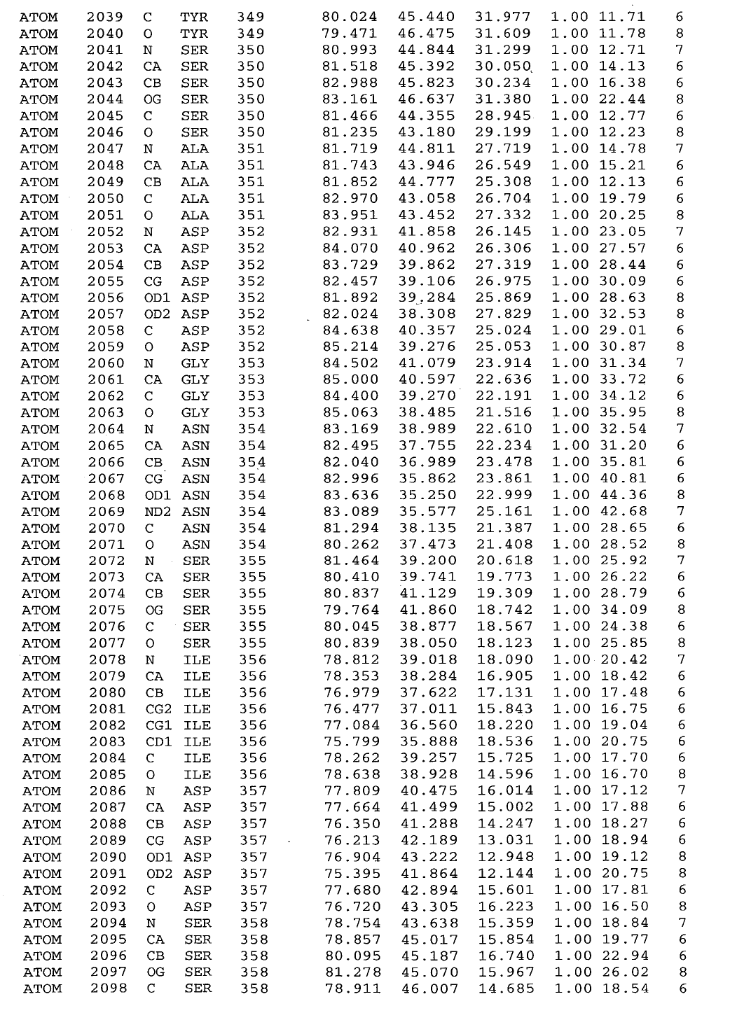

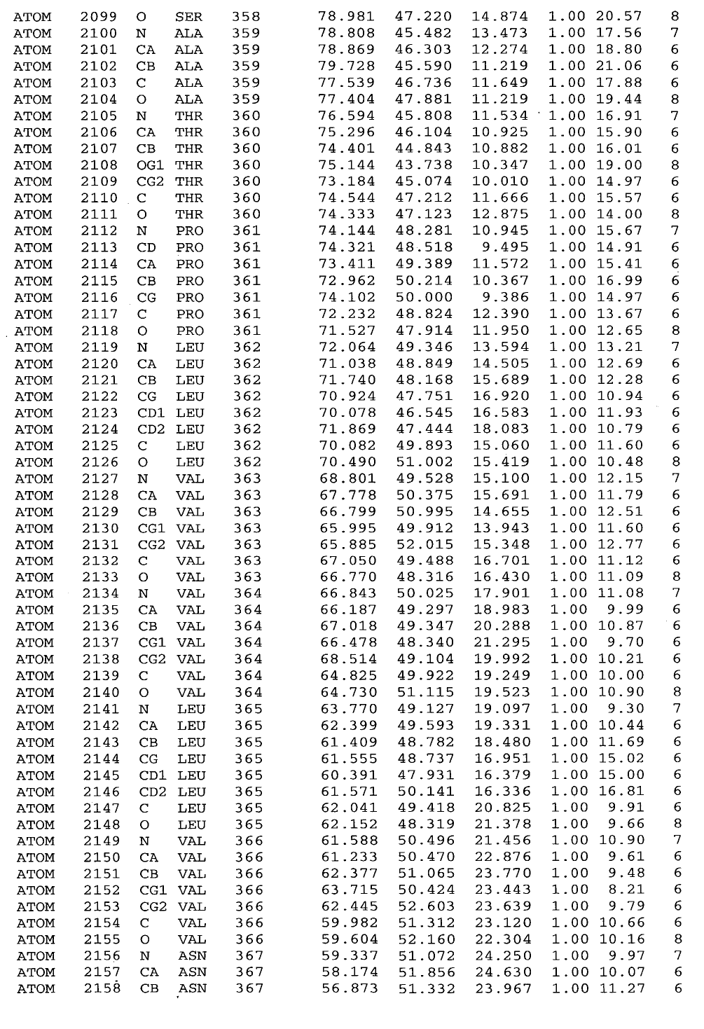

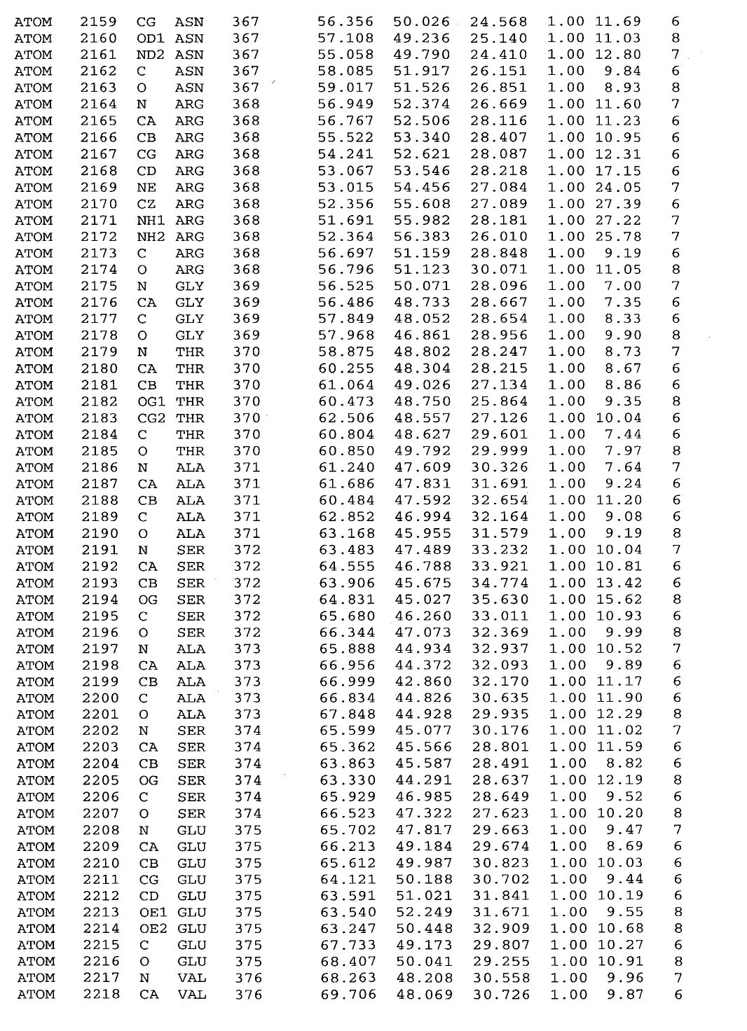

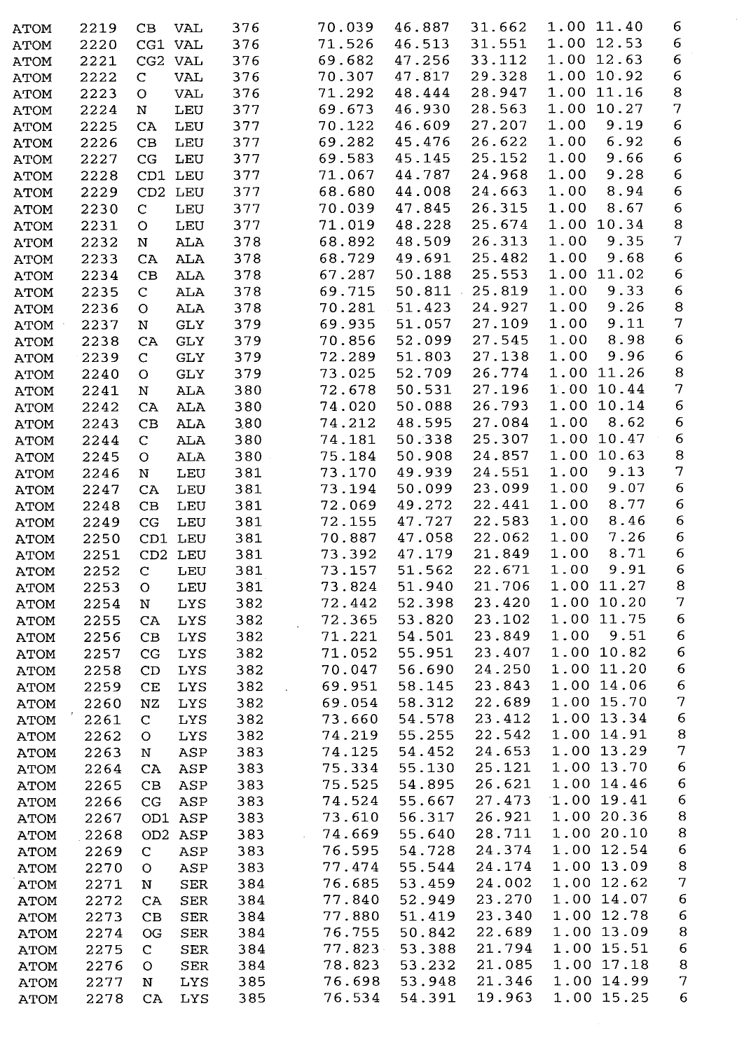

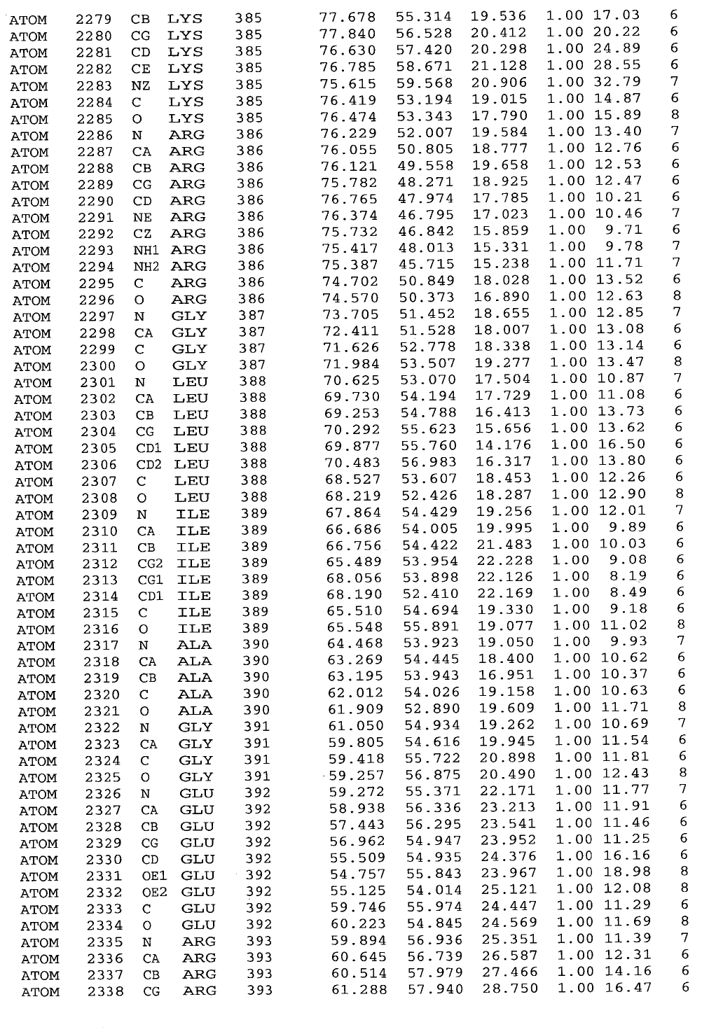

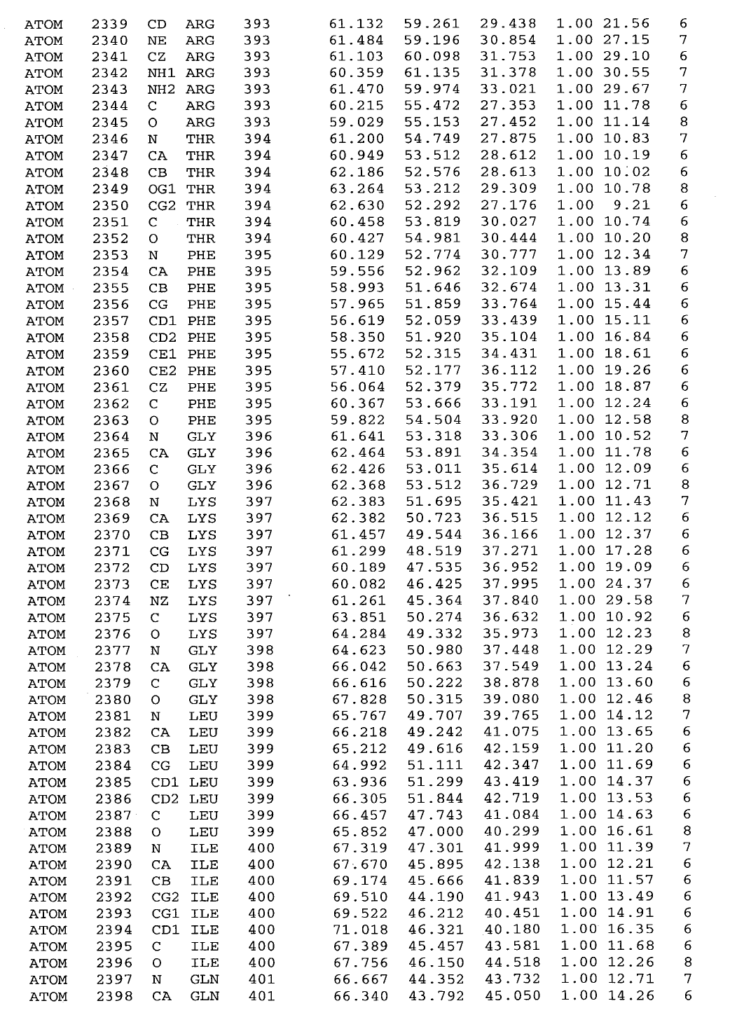

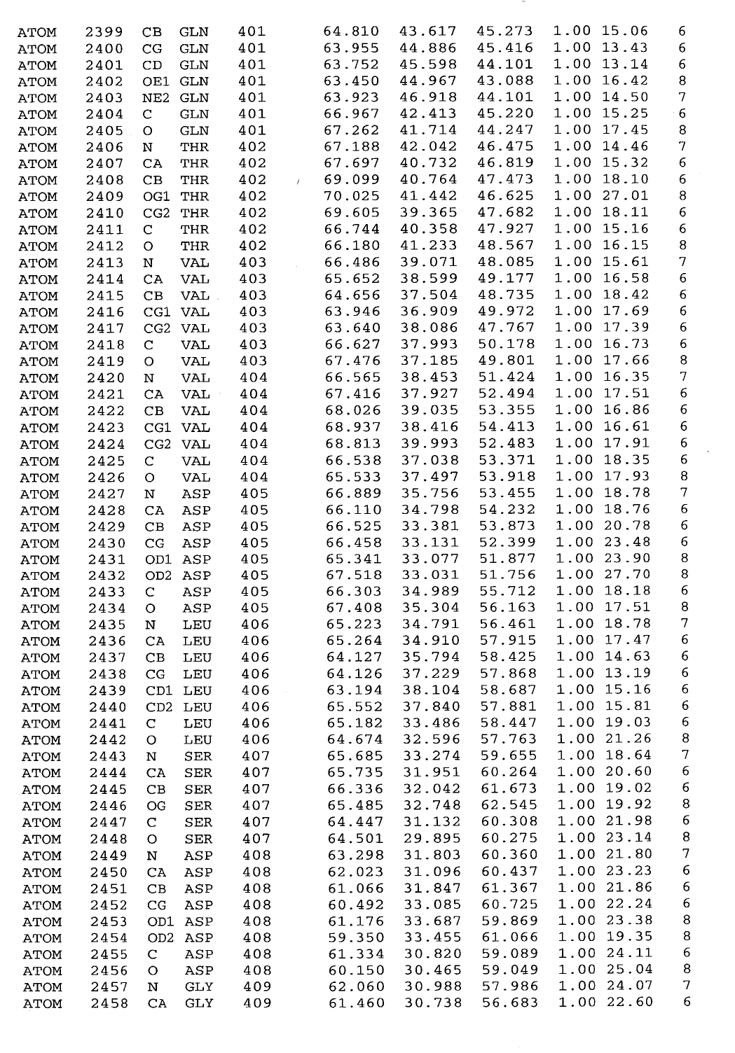

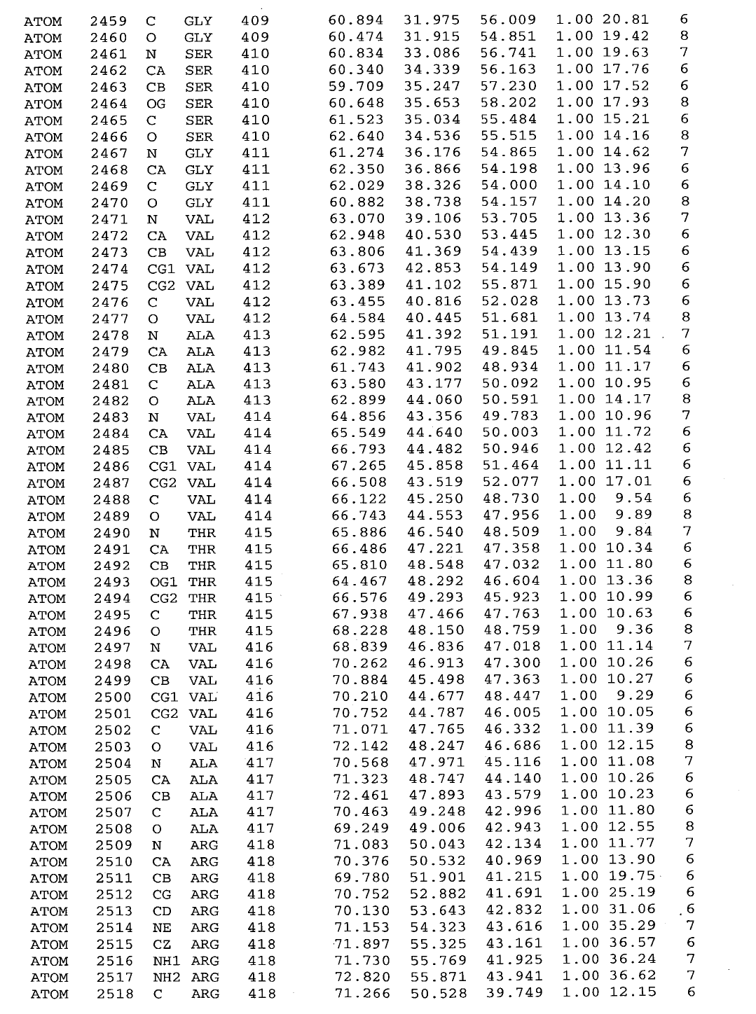

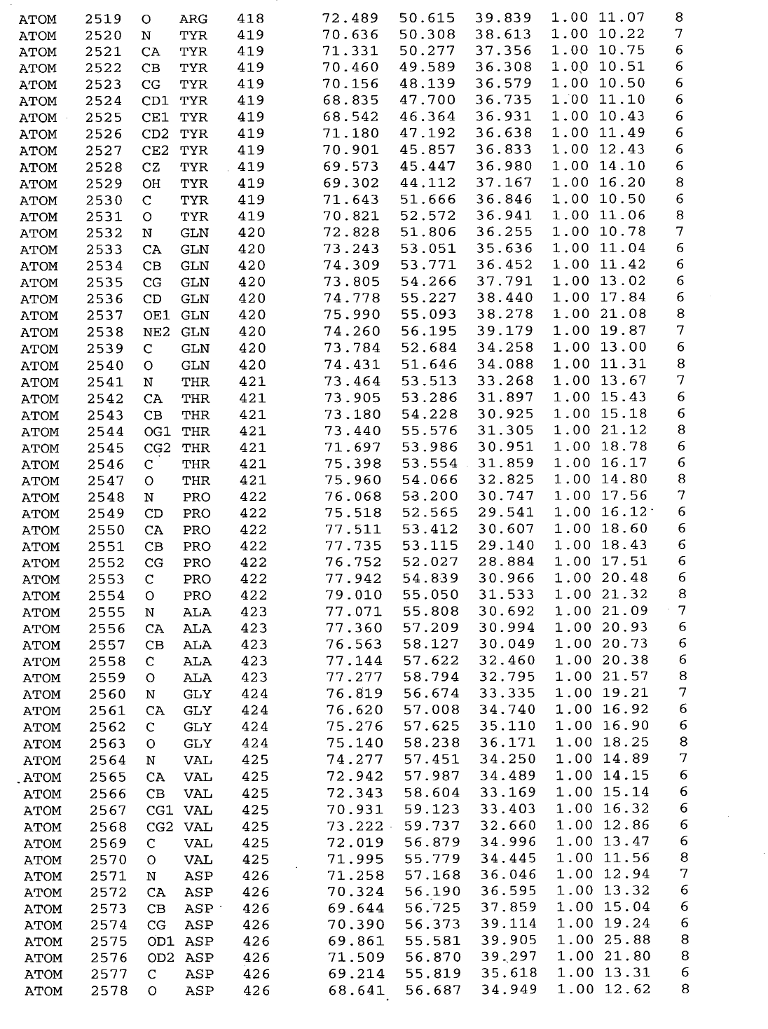

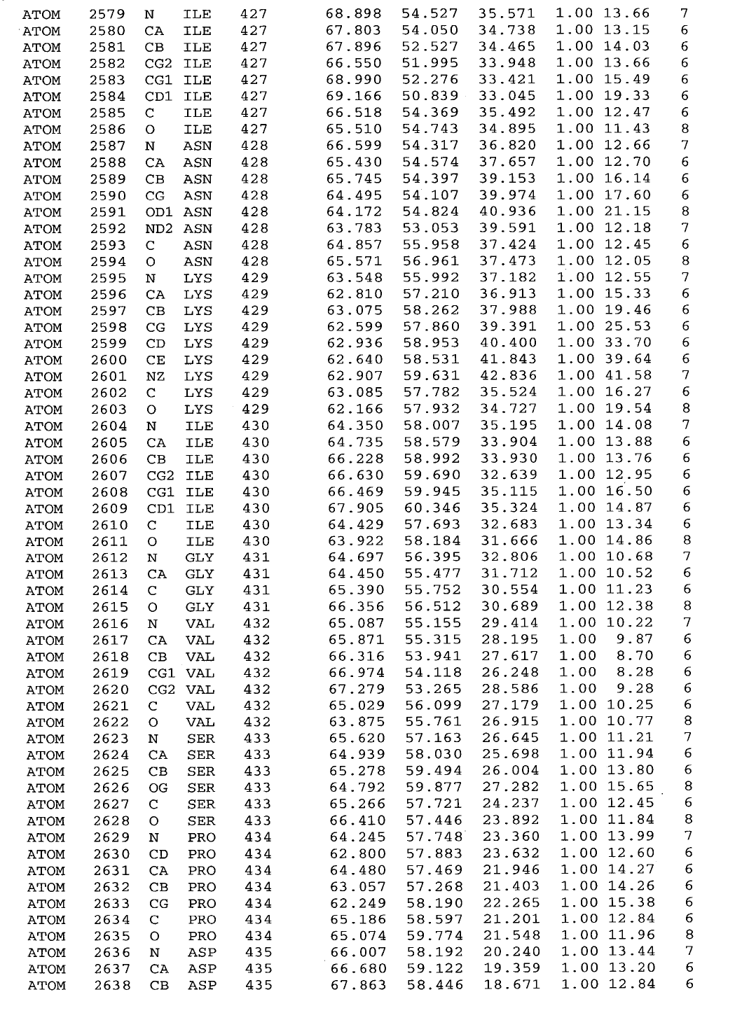

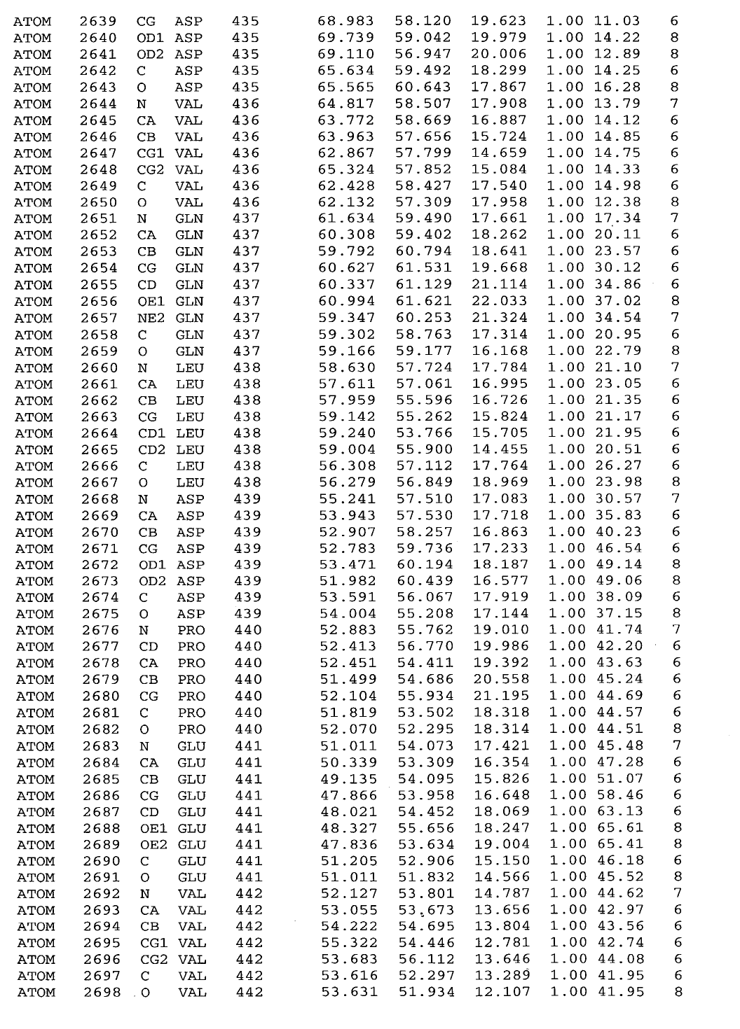

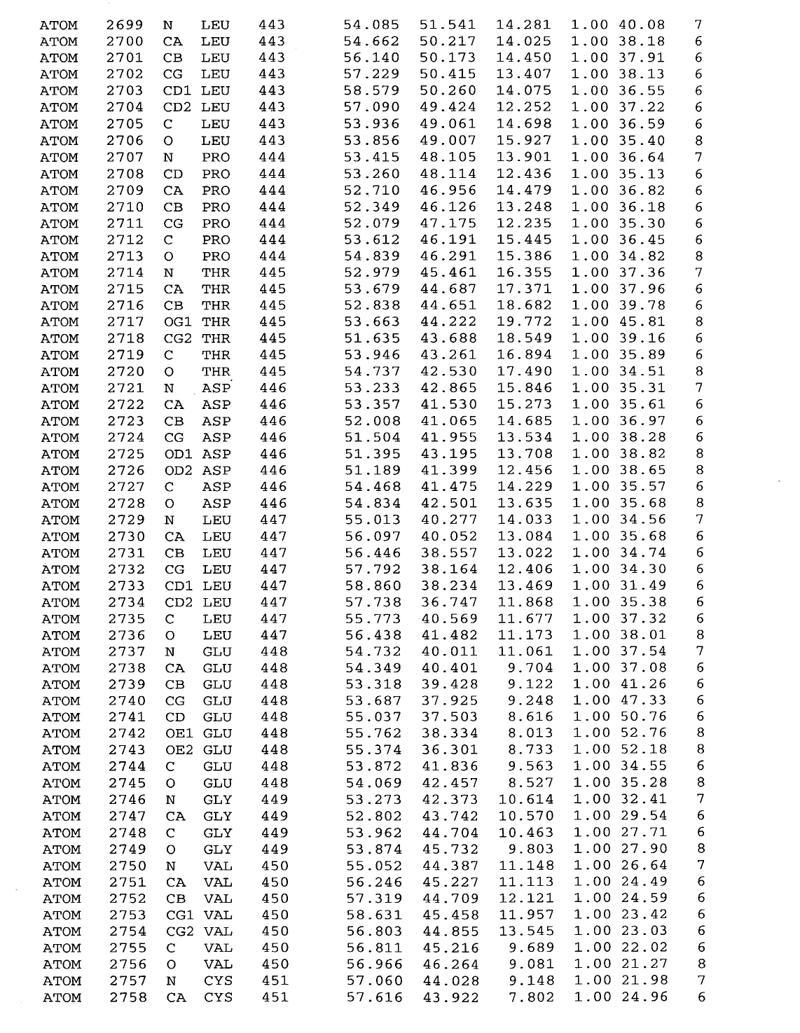

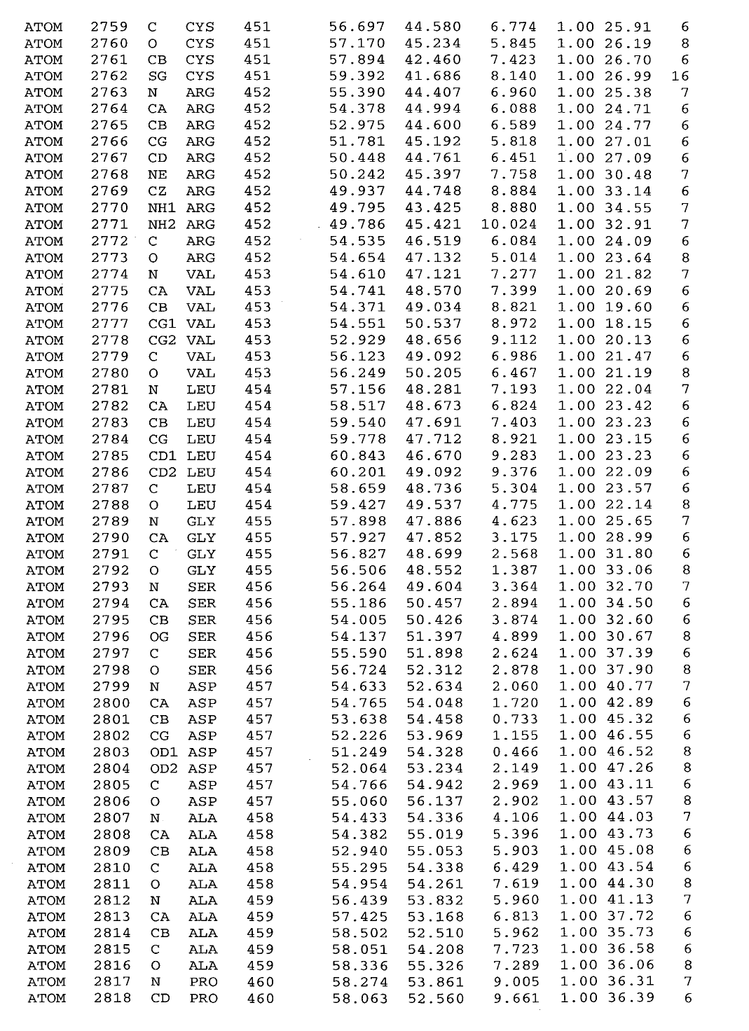

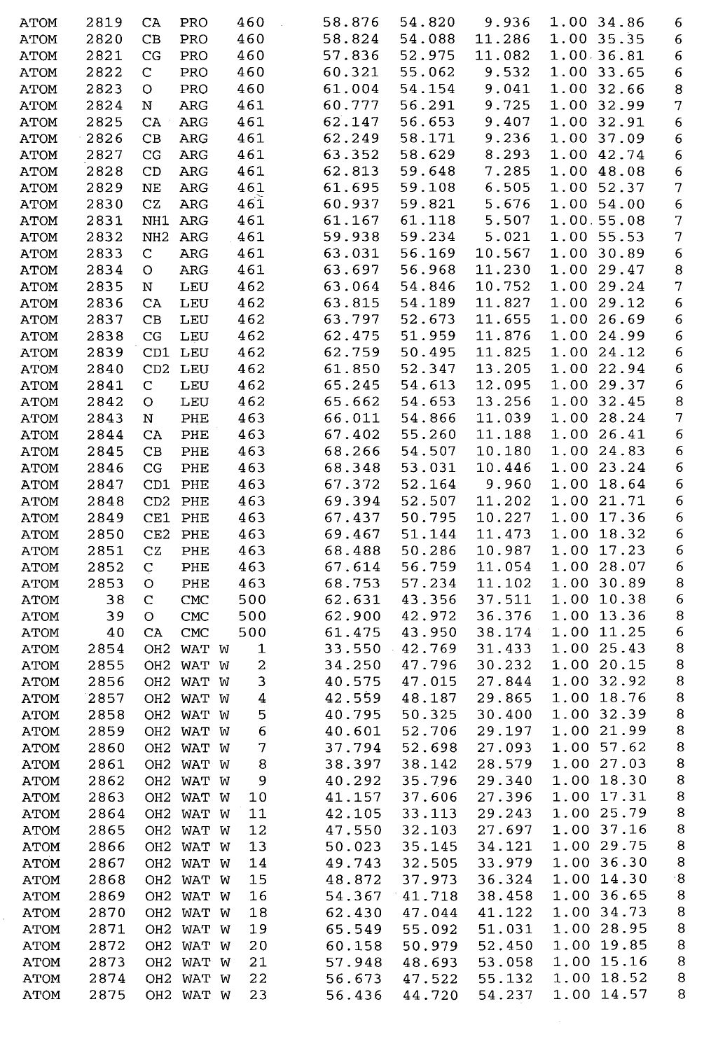

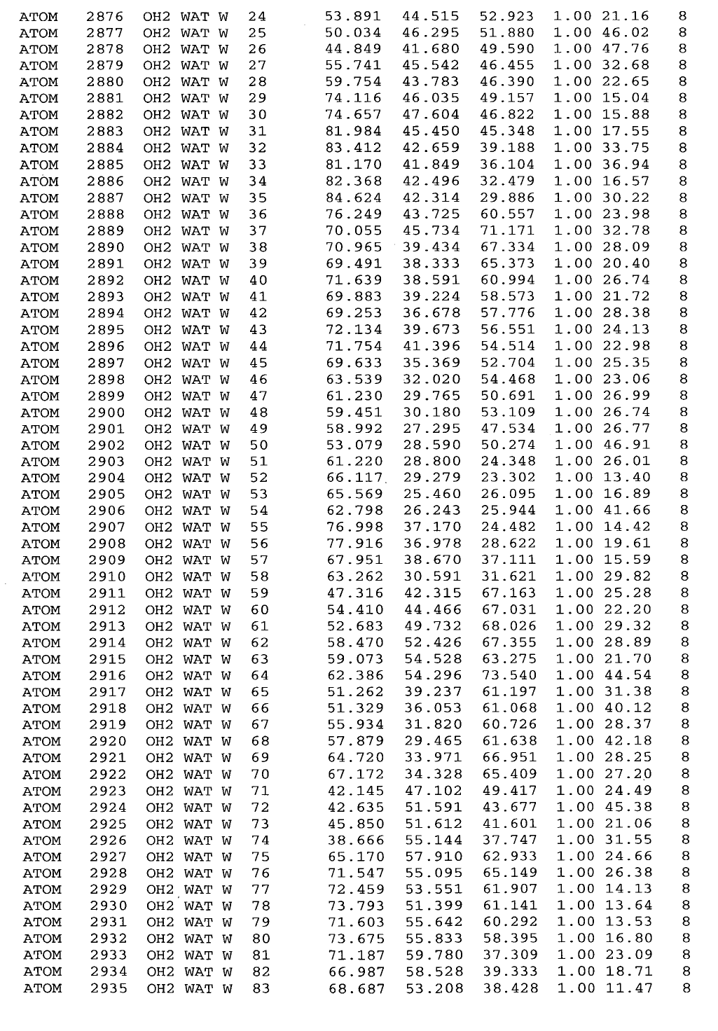

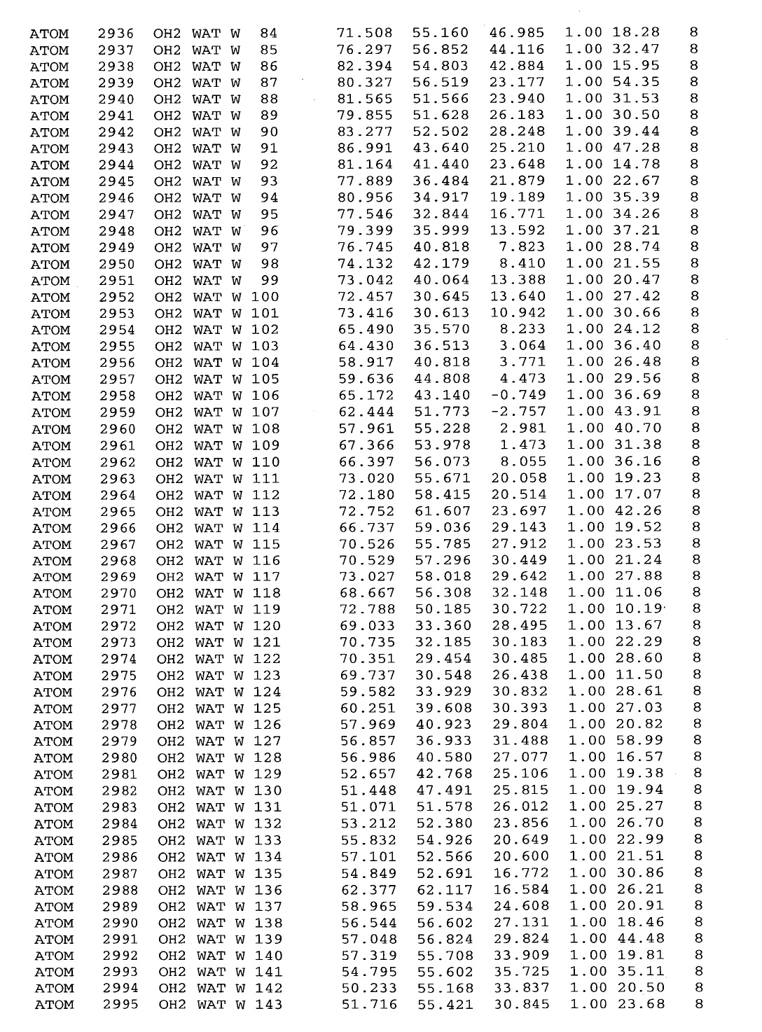

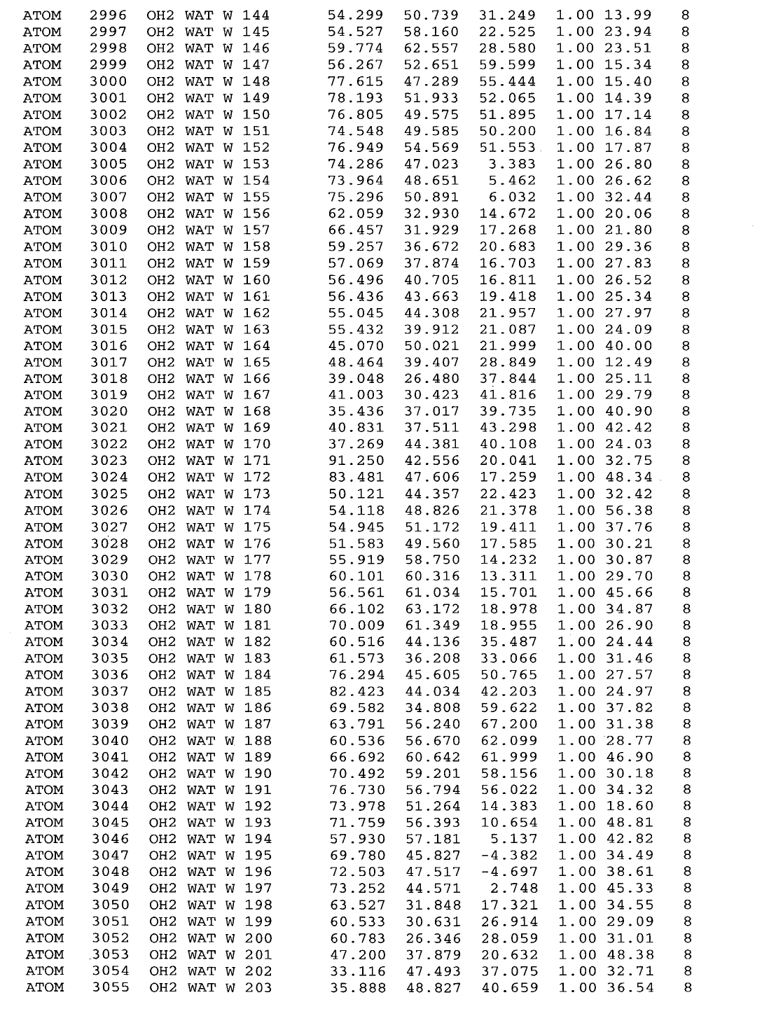

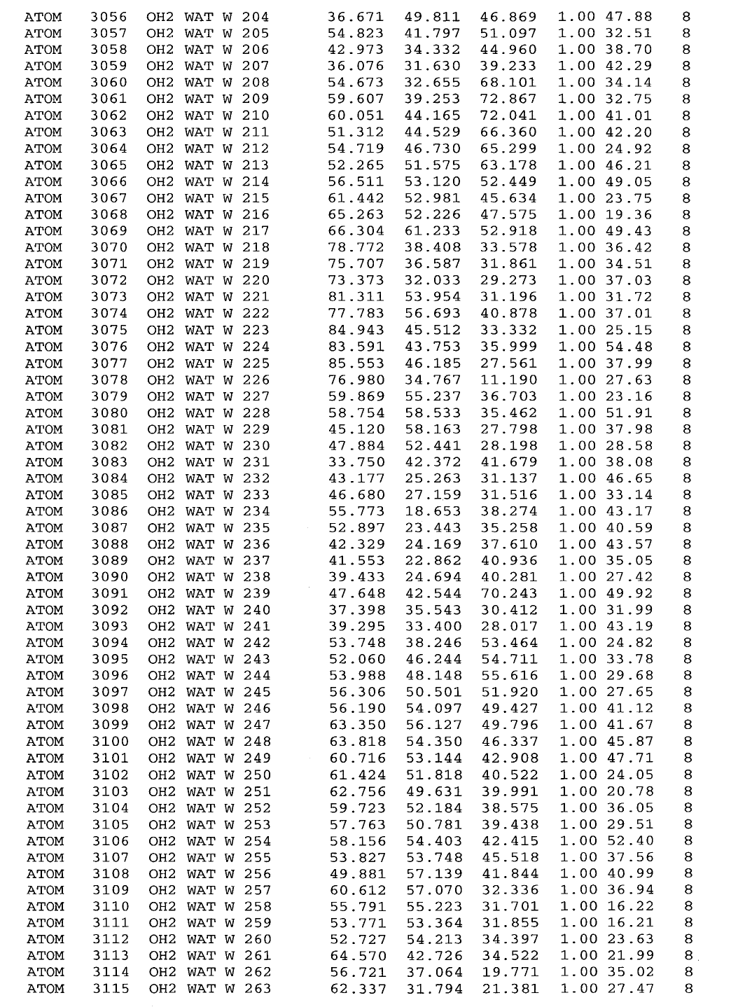

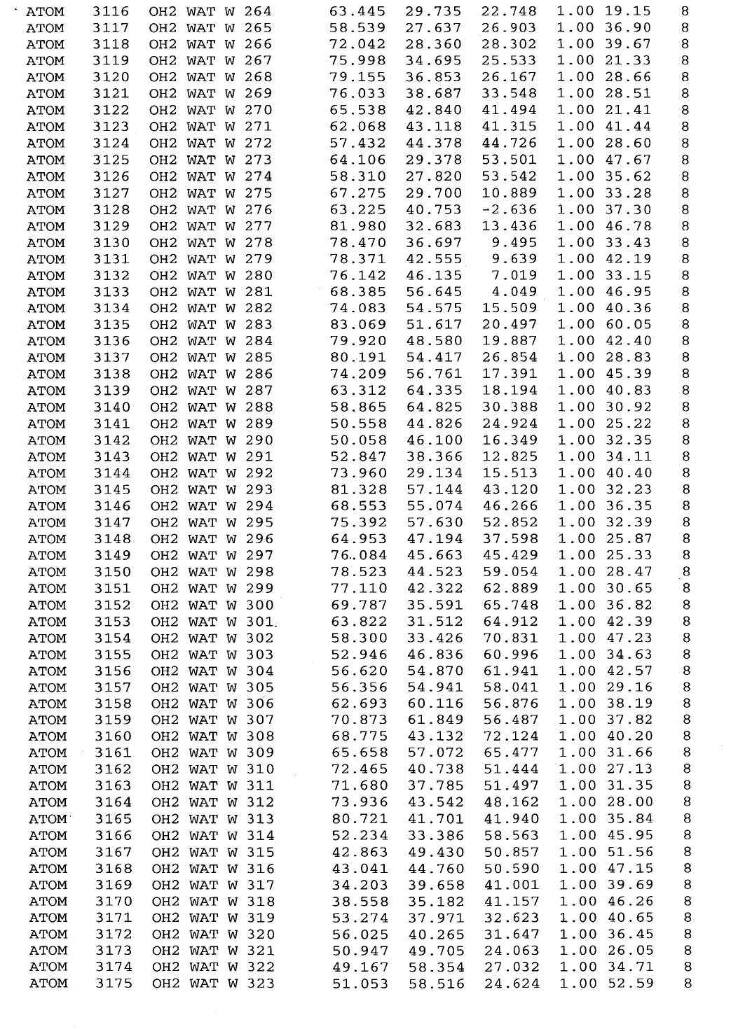

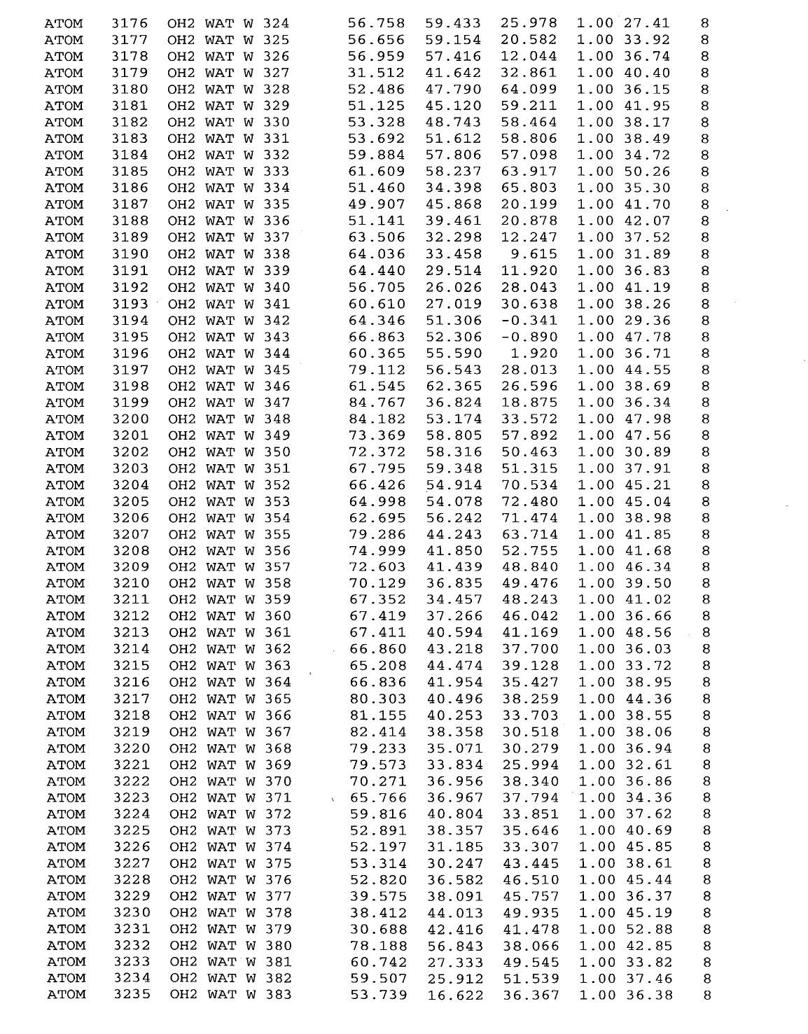

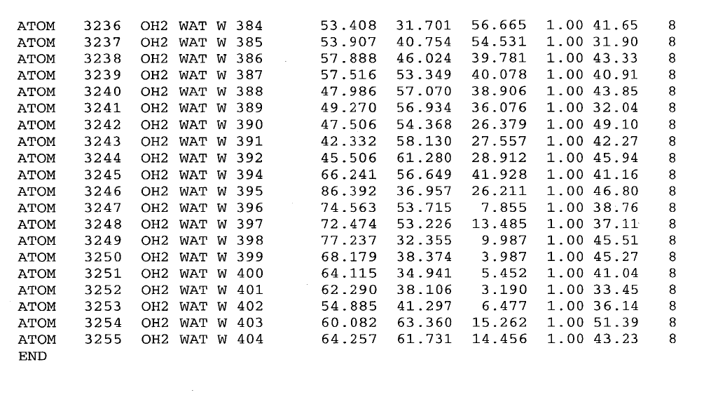

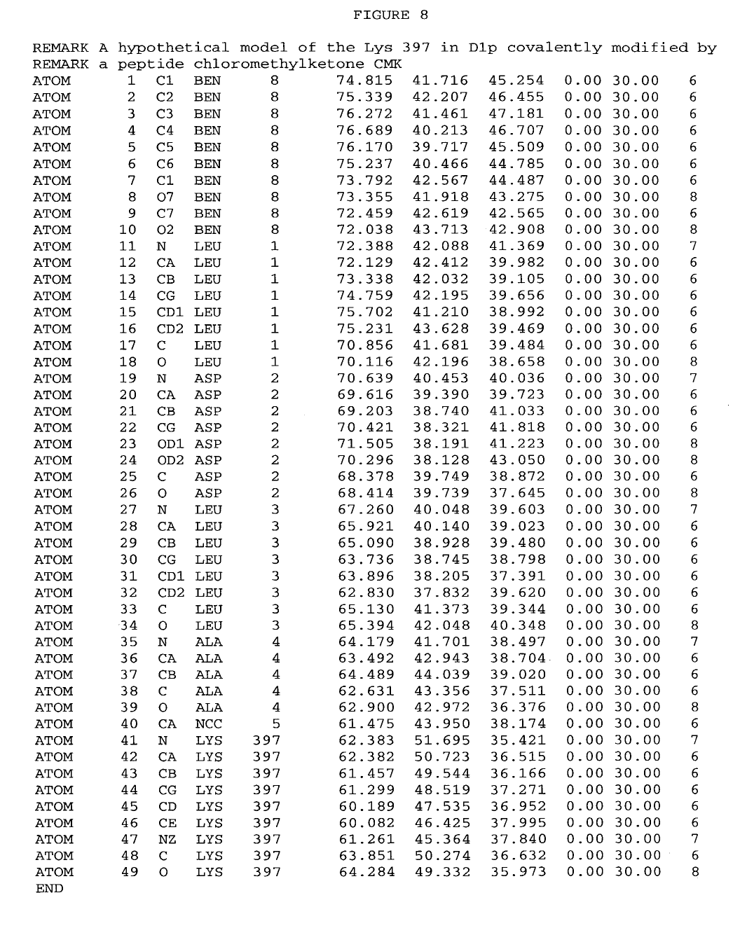

The electron density in the active site region of this structure indicates that the inhibitor is covalently bound to the Lys 397 residue. However, only three atoms closest to the NZ atom of the lysine side-chain can be seen in the electron density map. However, based on the conformation of the lysine side chain and the residual density produced by the disordered inhibitor, a hypothetical model of the chloromethylketone inhibitor has been built to identify the potential binding site of that part of the substrate mimicked by the inhibitor (FIG. 8). This model suggests that the P side of the substrate is bound to the large hydrophobic patch described earlier in the analysis of the active site section.

What is claimed is:

1. A computer readable medium having stored thereon atomic coordinate/x-ray diffraction data defining the three dimensional structure of Scenedesmus obliquus D1 protease or a fragment thereof.

2. A computer readable medium having stored thereon atomic coordinate data defining the three dimensional structure of wheat D1 protease or a fragment thereof.

3. The computer readable medium of claim 1 wherein the atomic coordinate/x-ray diffraction data are given in FIG. 1, FIG. 5 or FIG. 6.

4. The computer readable medium of claim 2 wherein the atomic coordinate data are given in FIG. 4.

5. A computer readable medium having stored thereon the computer model output data defining the three-dimensional structure of Scenedesmus obliquus D1 protease or a fragment thereof.

6. A computer readable medium having stored thereon the computer model output data defining the three-dimensional structure of a wheat D1 protease or a fragment thereof.

7. A computer readable medium having stored thereon atomic coordinate/x-ray diffraction data defining the three dimensional structure of a binary complex of D1 protease and a ligand that binds to D1 protease or a subunit thereof.

8. The computer readable medium of claim 7 wherein the ligand is an active site inhibitor of D1 protease.

9. The computer readable medium of claim 8 wherein the active site inhibitor is a tetrapeptide chloromethylketone.

10. The computer readable medium of claim 9 wherein the tetrapeptide chloromethylketone is Z-LDLA-CMK, wherein Z=carbobenzoxy, and CMK=chloromethylketone.

11. The computer readable medium of claim 10 wherein the atomic coordinate/x-ray diffraction data are given in FIGS. 7 or 8.

12. A computer readable medium having stored thereon the computer model output data defining the three dimensional structure of a ternary complex of D1 protease and a ligand that binds to D1 protease or a subunit thereof.

13. The computer readable medium of claim 12 wherein the ligand is an active site inhibitor of D1 protease.

14. The computer readable medium of claim 13 wherein the active site inhibitor is a tetrapeptide chloromethylketone.

15. A method for identifying a ligand of D1 protease or a fragment thereof the method comprising:
(a) providing a computer readable medium having stored thereon computer model output data defining the three dimensional structure of a of D1 protease;
(b) providing a computer readable medium having stored thereon computer model output data defining the three dimensional structure of a potential ligand that binds to D1 protease or a fragment thereof;
(c) providing a computer system comprising a computer and a computer algorithm, the computer system capable of processing the computer model output data of step (a) and step (b);
(d) processing the computer model output data of step (a) and step (b) using the computer system of step (c) wherein the processing calculates the ability of the potential ligand to bind to D1 protease or a fragment thereof; and
(e) identifying a potential ligand of D1 protease or a fragment thereof.

16. The method of claim 15 wherein the potential ligand of (b) is a tetrapeptide chloromethylketone.

17. The method of claim 16 wherein the tetrapeptide chloromethylketone is Z-LDLA-CMK, wherein Z=carbobenzoxy, and CMK=chloromethylketone.

18. A crystal of a D1 protease wherein the crystal effectively diffracts x-rays for the determination of the atomic coordinates of a D1 protease or a fragment thereof to a resolution equal to or better than 3.5 Angstroms and wherein the atomic coordinates of the crystal are given in FIG. 1, FIG. 4, FIG. 5, or FIG. 6.

19. The crystal of claim 18 wherein the crystal effectively diffracts x-rays for the determination of the atomic coordinates of the D1 protease to a resolution of about 1.8 Angstroms.

20. A method of identifying a D1 protease ligand comprising:
(a) selecting a potential ligand by performing rational compound design with the three-dimensional structure determined for the crystal of claim 19, wherein said selecting is performed in conjunction with computer modeling;
(b) contacting the potential ligand with the ligand binding domain of D1 protease; and
(c) detecting the binding of the potential ligand for the ligand binding domain;
wherein a potential ligand is selected on the basis of its having a greater affinity for the ligand binding domain of D1 protease than that of the natural substrate for the ligand binding domain of D1 protease.

21. A method of identifying a D1protease ligand comprising:
(a) performing molecular modeling using;
(i) the coordinate/x-ray diffraction data defining the three dimensional structure of Scenedesmus obliquus D1 protease or a fragment thereof; and
(ii) the amino acid sequence of a D1 protease enzyme;
wherein said modeling produces predicted coordinate data defining the three dimensional structure of the D1 protease enzyme;
(b) generating computer model output data from the predicted coordinate data defining the three dimensional structure of the D1 protease enzyme;
(c) providing a computer readable medium having stored thereon computer model output data of (b)
(d) providing a computer readable medium having stored thereon computer model output data defining the three dimensional structure of a potential ligand that binds to D1 protease or a fragment thereof;
(e) providing a computer system comprising a computer and a computer algorithm, the computer system capable of processing the computer model output data of step (c) and step (d);
(f) processing the computer model output data of step (c) and step (d) using the computer system of step (e) wherein the processing calculates the ability of the potential ligand to bind to D1 protease or a fragment thereof; and
(g) identifying a potential ligand of D1 protease or a fragment thereof.

22. The method of claim 21 wherein the molecular modeling is homology modeling.

23. The method of claim 21 wherein the molecular modeling is molecular replacement, and wherein at step (a) the molecular modeling further uses the x-ray diffraction data obtained from a crystal of said D1 protease enzyme.

24. The method of claim 21 wherein the potential ligand of (b) is a tetrapeptide chloromethylketone.

25. The method of claim 24 wherein the tetrapeptide chloromethylketone is Z-LDLA-CMK, wherein Z=carbobenzoxy, and CMK=chloromethylketone.

26. The method of claim 21 wherein the amino acid sequence of a D1 protease enzyme is isolated from organisms selected from the group consisting of higher plants, algae and cyanobacteria.

27. The method of claim 21 wherein the amino acid sequence of a D1 protease enzyme is isoalted from the group consisting of wheat, corn, soybean, barley, and rice.

28. A method of obtaining coordinate data defining the three dimensional structure of a D1 protease enzyme comprising performing homology modeling using;
(i) the coordinate/x-ray diffraction data defining the three dimensional structure of Scenedesmus obliquus D1 protease or a fragment thereof; and
(ii) the amino acid sequence of a D1 protease enzyme;
wherein said homology modeling produces predicted coordinate data defining the three dimensional structure of the D1 protease enzyme.

29. A method of obtaining coordinate data defining the three dimensional structure of a D1 protease enzyme comprising performing molecular replacement using;
(i) the coordinate/x-ray diffraction data defining the three dimensional structure of Scenedesmus D1 protease or a fragment thereof; and
(ii) the amino acid sequence of said D1 protease enzyme and
(iii) the x-ray diffraction data obtained from a crystal of said D1 protease enzyme;
wherein said molecular replacement produces the coordinate/x-ray diffraction data defining the three dimensional structure of the D1 protease enzyme.

30. The method of claims 28 or 29 wherein the amino acid sequence of a D1 protease enzyme is isolated from organisms selected from the group consisting of higher plants, algae and cyanobacteria.

31. The method of claim 30 wherein the amino acid sequence of a D1 protease enzyme is isolated from the group consisting of wheat, corn, soybean, barley, and rice.
